(19) United States
(12) Patent Application Publication
(10) Pub. No.: US 2011/0071101 A1
(43) Pub. Date: Mar. 24, 2011

(54) Nucleoside Phosphonate Analogs

(76) Inventors: Constantine G. Boojamra, San Francisco, CA (US); James M. Chen, San Ramon, CA (US); Xiaowu Chen, San Mateo, CA (US); Aesop Cho, Mountain View, CA (US); Lee S. Chong, Newark, CA (US); Maria Fardis, San Carlos, CA (US); Alan X. Huang, San Mateo, CA (US); Choung U. Kim, San Carlos, CA (US); Thorsten Kirschberg, Belmont, CA (US); Christopher P. Lee, San Francisco, CA (US); David A. Oare, Belmont, CA (US); Vidya K. Prasad, Burlingame, CA (US); Adrian S. Ray, San Mateo, CA (US); Sundaramoorthi Swaminathan, Burlingame, CA (US); William J. Watkins, Sunnyvale, CA (US)

(21) Appl. No.: 12/100,314

(22) Filed: Apr. 9, 2008

Related U.S. Application Data
(63) Continuation of application No. 10/832,810, filed on Apr. 26, 2004, now abandoned.
(60) Provisional application No. 60/465,400, filed on Apr. 25, 2003.
(60) Provisional application No. 60/465,587, filed on Apr. 25, 2003.
(60) Provisional application No. 60/465,463, filed on Apr. 25, 2003.
(60) Provisional application No. 60/465,602, filed on Apr. 25, 2003.
(60) Provisional application No. 60/465,633, filed on Apr. 25, 2003.
(60) Provisional application No. 60/465,550, filed on Apr. 25, 2003.
(60) Provisional application No. 60/465,610, filed on Apr. 25, 2003.
(60) Provisional application No. 60/465,720, filed on Apr. 25, 2003.
(60) Provisional application No. 60/465,634, filed on Apr. 25, 2003.
(60) Provisional application No. 60/465,537, filed on Apr. 25, 2003.
(60) Provisional application No. 60/465,698, filed on Apr. 25, 2003.
(60) Provisional application No. 60/465,667, filed on Apr. 25, 2003.
(60) Provisional application No. 60/465,554, filed on Apr. 25, 2003.
(60) Provisional application No. 60/465,553, filed on Apr. 25, 2003.
(60) Provisional application No. 60/465,561, filed on Apr. 25, 2003.
(60) Provisional application No. 60/465,548, filed on Apr. 25, 2003.
(60) Provisional application No. 60/465,696, filed on Apr. 25, 2003.
(60) Provisional application No. 60/465,347, filed on Apr. 25, 2003.
(60) Provisional application No. 60/465,600, filed on Apr. 25, 2003.
(60) Provisional application No. 60/465,591, filed on Apr. 25, 2003.
(60) Provisional application No. 60/465,684, filed on Apr. 25, 2003.
(60) Provisional application No. 60/465,821, filed on Apr. 25, 2003.
(60) Provisional application No. 60/465,608, filed on Apr. 25, 2003.
(60) Provisional application No. 60/465,584, filed on Apr. 25, 2003.
(60) Provisional application No. 60/465,759, filed on Apr. 25, 2003.
(60) Provisional application No. 60/465,467, filed on Apr. 25, 2003.
(60) Provisional application No. 60/465,559, filed on Apr. 25, 2003.
(60) Provisional application No. 60/465,544, filed on Apr. 25, 2003.
(60) Provisional application No. 60/465,574, filed on Apr. 25, 2003.
(60) Provisional application No. 60/495,490, filed on Aug. 15, 2003.
(60) Provisional application No. 60/495,805, filed on Aug. 15, 2003.
(60) Provisional application No. 60/495,684, filed on Aug. 15, 2003.
(60) Provisional application No. 60/495,600, filed on Aug. 15, 2003.
(60) Provisional application No. 60/495,564, filed on Aug. 15, 2003.
(60) Provisional application No. 60/495,772, filed on Aug. 15, 2003.
(60) Provisional application No. 60/495,592, filed on Aug. 15, 2003.
(60) Provisional application No. 60/495,453, filed on Aug. 15, 2003.
(60) Provisional application No. 60/495,491, filed on Aug. 15, 2003.
(60) Provisional application No. 60/495,964, filed on Aug. 15, 2003.
(60) Provisional application No. 60/495,317, filed on Aug. 15, 2003.
(60) Provisional application No. 60/495,696, filed on Aug. 15, 2003.
(60) Provisional application No. 60/495,760, filed on Aug. 15, 2003.
(60) Provisional application No. 60/495,334, filed on Aug. 15, 2003.
(60) Provisional application No. 60/495,671, filed on Aug. 15, 2003.
(60) Provisional application No. 60/495,349, filed on Aug. 15, 2003.
(60) Provisional application No. 60/495,273, filed on Aug. 15, 2003.
(60) Provisional application No. 60/495,763, filed on Aug. 15, 2003.
(60) Provisional application No. 60/495,343, filed on Aug. 15, 2003.
(60) Provisional application No. 60/495,344, filed on Aug. 15, 2003.
(60) Provisional application No. 60/495,278, filed on Aug. 15, 2003.
(60) Provisional application No. 60/495,277, filed on Aug. 15, 2003.
(60) Provisional application No. 60/495,631, filed on Aug. 15, 2003.
(60) Provisional application No. 60/495,633, filed on Aug. 15, 2003.
(60) Provisional application No. 60/495,539, filed on Aug. 15, 2003.
(60) Provisional application No. 60/495,525, filed on Aug. 15, 2003.
(60) Provisional application No. 60/495,387, filed on Aug. 15, 2003.
(60) Provisional application No. 60/495,417, filed on Aug. 15, 2003.
(60) Provisional application No. 60/510,245, filed on Oct. 10, 2003.
(60) Provisional application No. 60/513,932, filed on Oct. 24, 2003.
(60) Provisional application No. 60/513,926, filed on Oct. 24, 2003.
(60) Provisional application No. 60/514,159, filed on Oct. 24, 2003.
(60) Provisional application No. 60/514,083, filed on Oct. 24, 2003.
(60) Provisional application No. 60/513,949, filed on Oct. 24, 2003.
(60) Provisional application No. 60/514,144, filed on Oct. 24, 2003.
(60) Provisional application No. 60/531,940, filed on Dec. 22, 2003.

(51) Int. Cl. A61K 31/7068; C07H 19/06

(52) U.S. Cl. 514/52; 536/26.1

(57) ABSTRACT

The invention is related to phosphorus substituted nucleoside compounds and therapeutic methods that include the administration of such compounds, as well as to processes and intermediates useful for preparing such compounds.

### PRIORITY OF INVENTION

This application claims the benefit of priority under 35 U.S.C. §119(e) to U.S. Provisional Patent Application Ser. Nos. 60/465,400, 60/465,587, 60/465,463, 60/465,602, 60/465,633, 60/465,550, 60/465,610, 60/465,720, 60/465,634, 60/465,537, 60/465,698, 60/465,667, 60/465,554, 60/465,553, 60/465,561, 60/465,548, 60/465,696, 60/465,347, 60/465,600, 60/465,591, 60/465,684, 60/465,821, 60/465,608, 60/465,584, 60/465,759, 60/465,467, 60/465,559, 60/465,544, and 60/465,574, all filed Apr. 25, 2003; and to U.S. Provisional Patent Application Ser. Nos. 60/495,490, 60/495,805, 60/495,684, 60/495,600, 60/495,564, 60/495,772, 60/495,592, 60/495,453, 60/495,491, 60/495,964, 60/495,317, 60/495,696, 60/495,760, 60/495,334, 60/495,671, 60/495,349, 60/495,273, 60/495,763, 60/495,343, 60/495,344, 60/495,278, 60/495,277, 60/495,631, 60/495,633, 60/495,539, 60/495,525, 60/495,387, and 60/495,417, all filed Aug. 15, 2003; and to U.S. Provisional Patent Application Ser. No. 60/510,245, filed Oct. 10, 2003; and to U.S. Provisional Patent Application Ser. Nos. 60/513,932, 60/513,926, 60/514,159, 60/514,083, 60/513,949, and 60/514,144, all filed Oct. 24, 2003; and to U.S. Provisional Patent Application Ser. No. 60/531,940, filed Dec. 22, 2003. The entirety of all Provisional Applications listed above are incorporated herein by reference.

### FIELD OF THE INVENTION

The invention relates generally to nucleoside analog compounds with, e.g., nucleic acid synthesis inhibiting activity.

### BACKGROUND OF THE INVENTION

Improving the delivery of drugs and other agents to target cells and tissues has been the focus of considerable research for many years. Though many attempts have been made to develop effective methods for importing biologically active molecules into cells, both in vivo and in vitro, none has proved to be entirely satisfactory. Optimizing the association of the inhibitory drug with its intracellular target, while minimizing intercellular redistribution of the drug, e.g., to neighboring cells, is often difficult or inefficient.

Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., glucocorticoids and other anti-inflammatory drugs) that can be administered. By comparison, although oral administration of drugs is generally recognized as a convenient and economical method of administration, oral administration can result in either (a) uptake of the drug through the cellular and tissue barriers, e.g., blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment includes avoiding the general physiological effects of inappropriate delivery of such agents to other cells and tissues, such as uninfected cells.

Thus, there is a need for therapeutic agents having improved pharmacological properties and pharmacokinetic properties, including improved oral bioavailability, greater potency and extended effective half-life in vivo, e.g., drugs having improved activity for treating cancer and/or viral infections. New compounds should have fewer side effects, less complicated dosing schedules, and be orally active. In particular, there is a need for a less onerous dosage regimen, such as one pill, once per day.

Assay methods capable of determining the presence, absence or amounts of nucleoside analog activity, e.g., DNA and/or RNA synthesis inhibition, are of practical utility in the search for treatment as well as for diagnosing the presence of diseases such as cancer and viral infections.

### SUMMARY OF THE INVENTION

Intracellular targeting may be achieved by methods and compositions that allow accumulation or retention of biologically active agents inside cells. The present invention provides novel nucleoside analogs. Such novel nucleoside analogs possess all the utilities of the parent nucleoside analogs and optionally provide cellular accumulation as set forth below. In addition, the present invention provides compositions and methods for inhibiting DNA and/or RNA synthesis or therapeutic activity against conditions sensitive to such inhibition, e.g., cancer and/or viral infections.

The present invention relates generally to the accumulation or retention of therapeutic compounds inside cells. The invention is more particularly related to attaining high concentrations of phosphonate-containing molecules in target cells. Such effective targeting may be applicable to a variety of therapeutic formulations and procedures.

Compositions of the invention include nucleoside analog compounds having at least one phosphonate group. Accordingly, in one embodiment the invention provides a conjugate comprising a nucleoside linked to one or more phosphonate groups; or a pharmaceutically acceptable salt or solvate thereof. In one embodiment, the conjugate is isolated and purified.

In another embodiment, the invention provides a compound of any one of formulae 200-247:

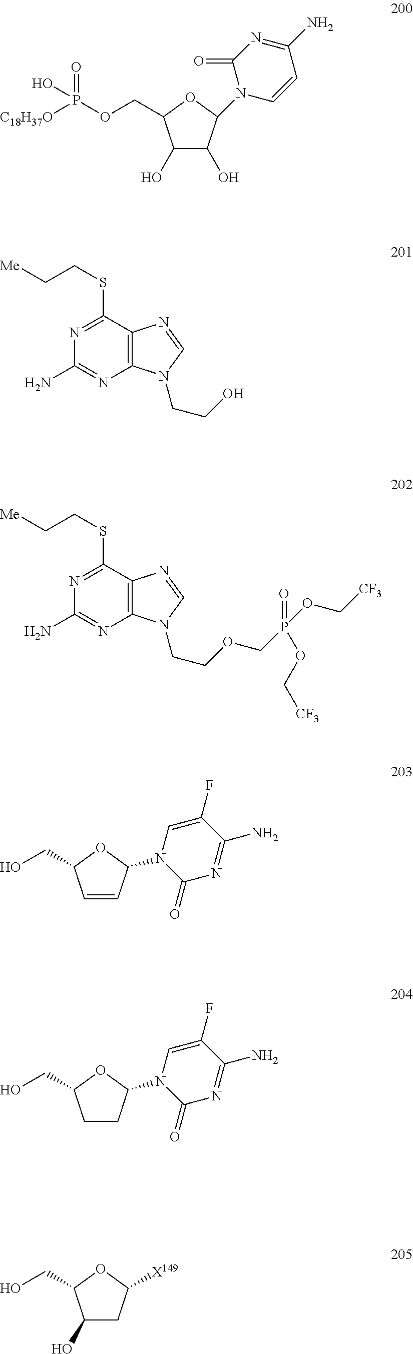

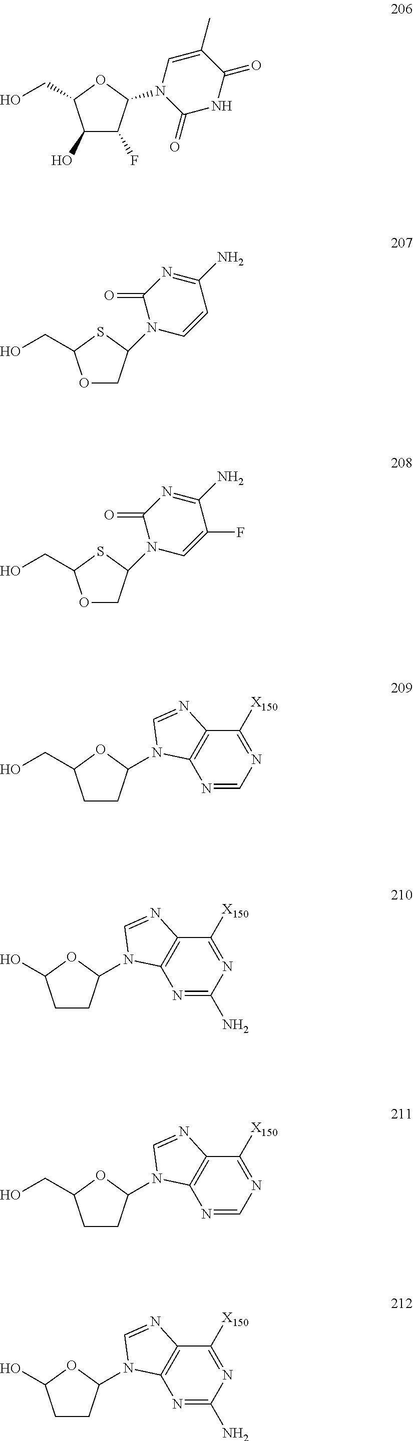

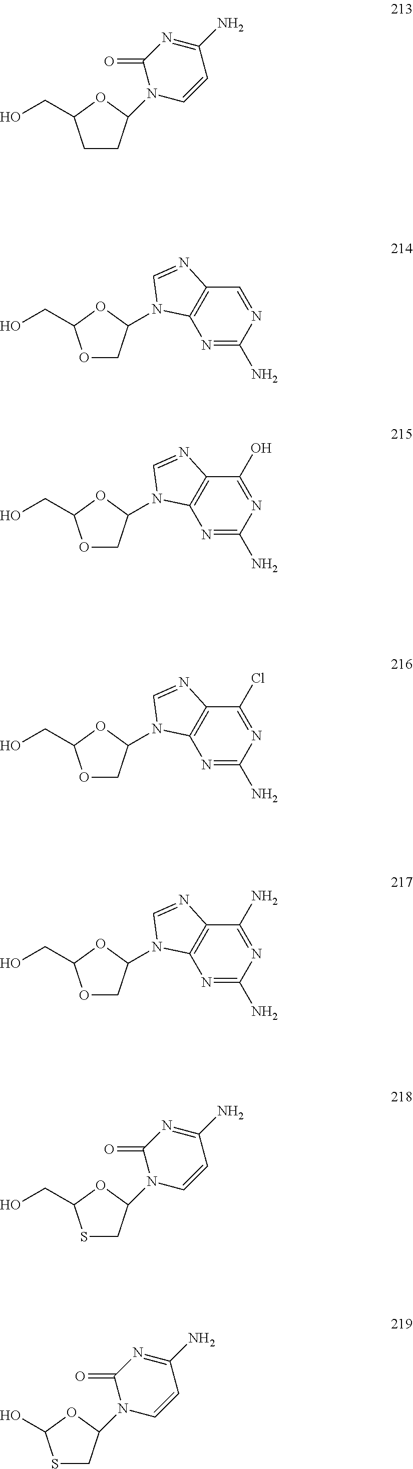

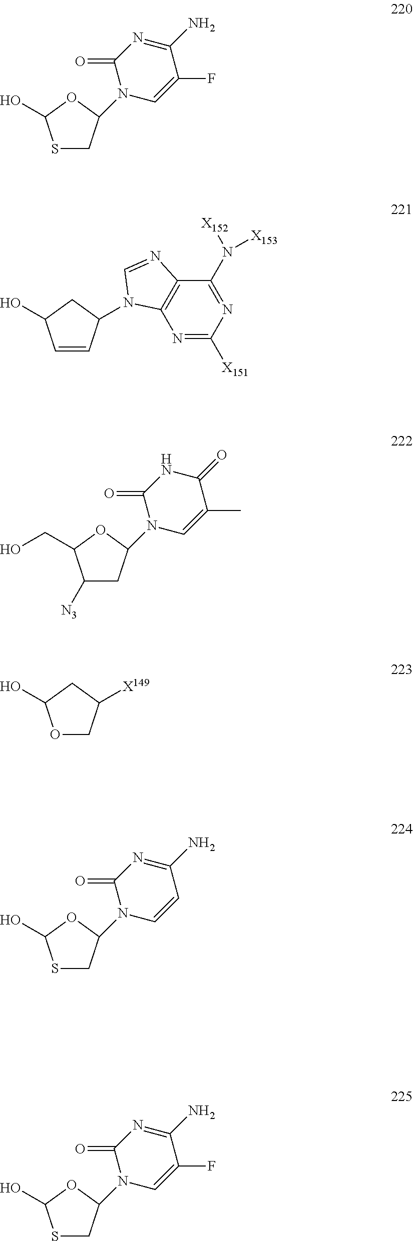

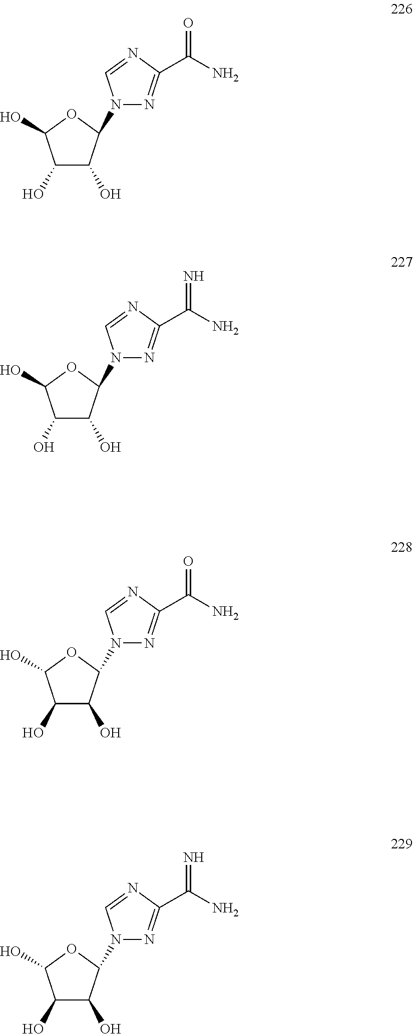

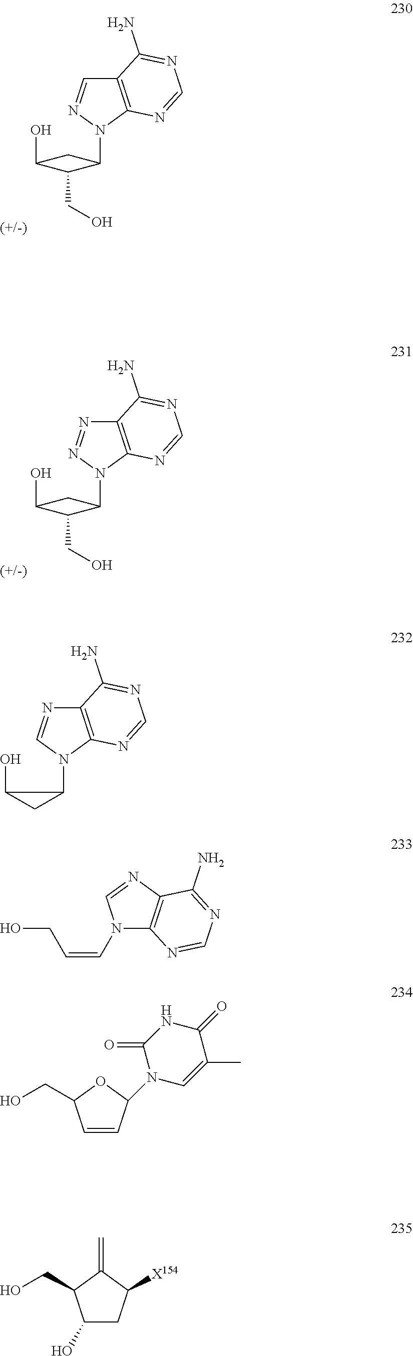

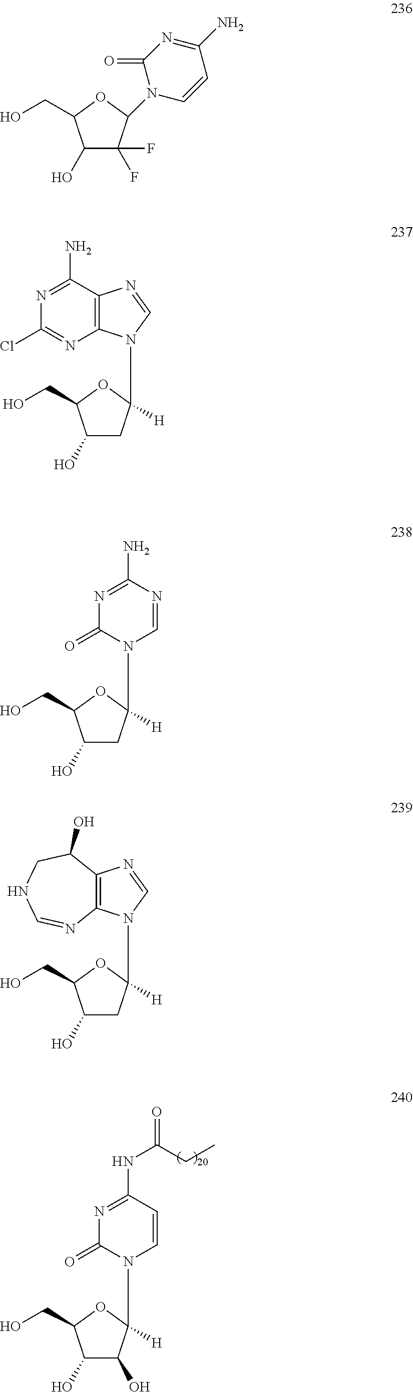

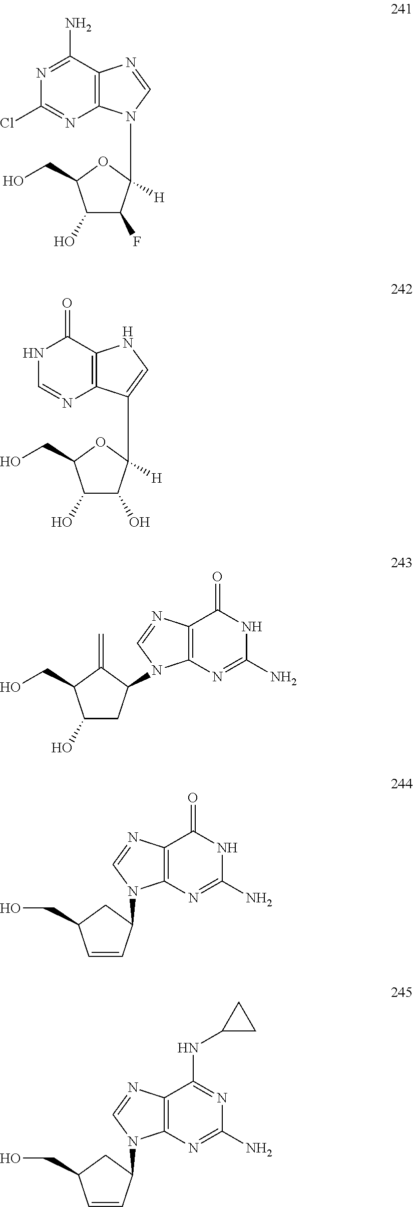

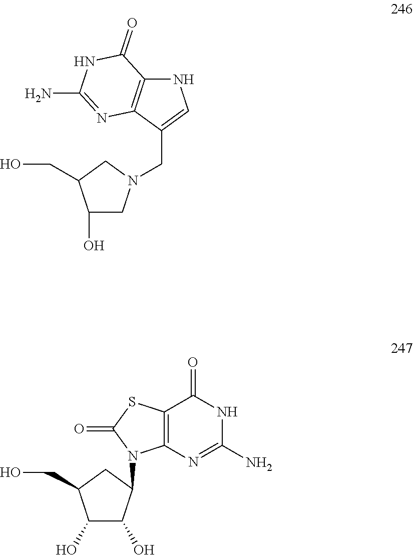

that is substituted with one or more groups A⁰,
wherein:

A⁰ is A¹, A² or W³ with the proviso that the conjugate includes at least one A¹;

A¹ is:

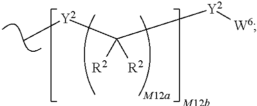

A² is:

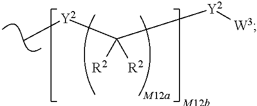

A³ is:

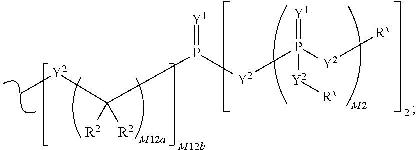

Y¹ is independently O, S, N(R^{x}), N(O)(R^{x}), N(OR^{x}), N(O)(OR^{x}), or N(N(R^{x})(R^{x});

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—; and when Y² joins two phosphorous atoms Y² can also be C(R²)(R²);

R^{x} is independently H, R¹, R², W³, a protecting group, or the formula:

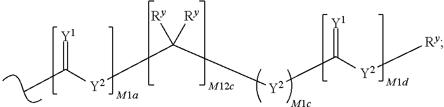

wherein:

R^{y} is independently H, W³, R² or a protecting group;

R¹ is independently H or alkyl of 1 to 18 carbon atoms;

R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

W³ is W⁴ or W⁵;

W⁴ is R⁵, —C(Y¹)R⁵, —C(Y¹)W⁵, —SO_{M2}R⁵, or —SO_{M2}W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1;

M12c is 0, 1, 2, 3, 4, 5, 6, 7, 8, 9, 10, 11 or 12;

X¹⁴⁹ is thymine, adenine, uracil, a 5-halouracil, a 5-alkyluracil, guanine, cytosine, a 5-halocytosine, 5-alkylcytosine, or 2,6-diaminopurine;

X¹⁵⁰ is OH, Cl, NH₂, H, Me, or MeO;

X¹⁵¹ is H, NH₂, or NH-alkyl;

X¹⁵² and X¹⁵³ are independently H, alkyl, or cyclopropyl; and

X¹⁵⁴ is thymine, adenine, guanine, cytosine, uracil, inosine, or diaminopurine.

In another embodiment the invention provides a conjugate which has the formula:

[DRUG]-(A⁰)ₙₙ

wherein:

DRUG is a compound of any one of formulae 200-247;

nn is 1, 2, or 3

A⁰ is A¹, A² or W³ with the proviso that the conjugate includes at least one A¹;

A¹ is:

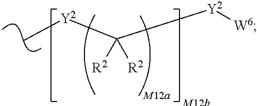

A² is:

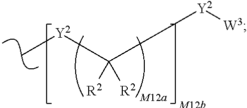

A³ is:

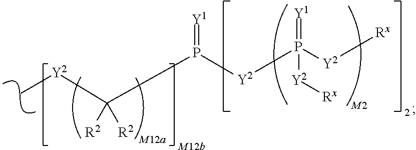

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(O)(OR^{x}), N(N(R^{x})(R^{x})), —S(O)_{M2}—, or —S(O)_{M2}—S(O)_{M2}—; and when Y² joins two phosphorous atoms Y² can also be C(R²)(R²);

R^{x} is independently H, R¹, R², W³, a protecting group, or the formula:

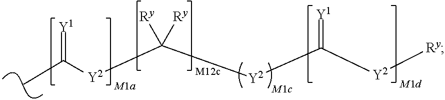

wherein:

R^{y} is independently H, W³, R² or a protecting group;

R¹ is independently H or alkyl of 1 to 18 carbon atoms;

R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

W³ is W⁴ or W⁵;

W⁴ is R⁵, —C(Y¹)R⁵, —C(Y¹)W⁵, —SO_{M2}R⁵, or —SO_{M2}W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1;

M12c is 0, 1, 2, 3, 4, 5, 6, 7, 8, 9, 10, 11 or 12.

X¹⁴⁹ is thymine, adenine, uracil, a 5-halouracil, a 5-alkyluracil, guanine, cytosine, a 5-halocytosine, 5-alkylcytosine, or 2,6-diaminopurine;

X¹⁵⁹ is OH, Cl, NH₂, H, Me, or MeO;

X¹⁵¹ is H, NH₂, or NH-alkyl;

X¹⁵² and X¹⁵³ are independently H, alkyl, or cyclopropyl; and

X¹⁵⁴ is thymine, adenine, guanine, cytosine, uracil, inosine, or diaminopurine.

In another embodiment, the invention provides a conjugate of any one of formulae 1-71:

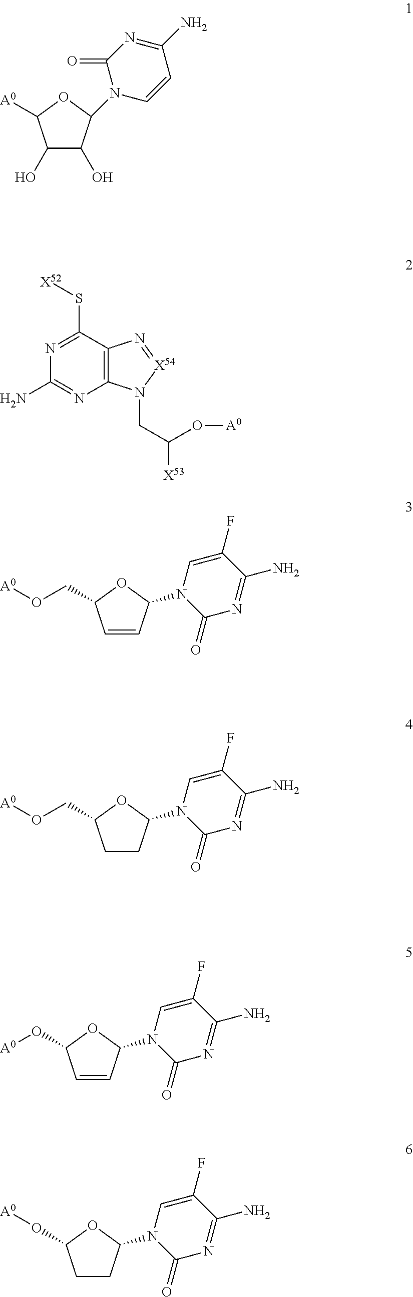

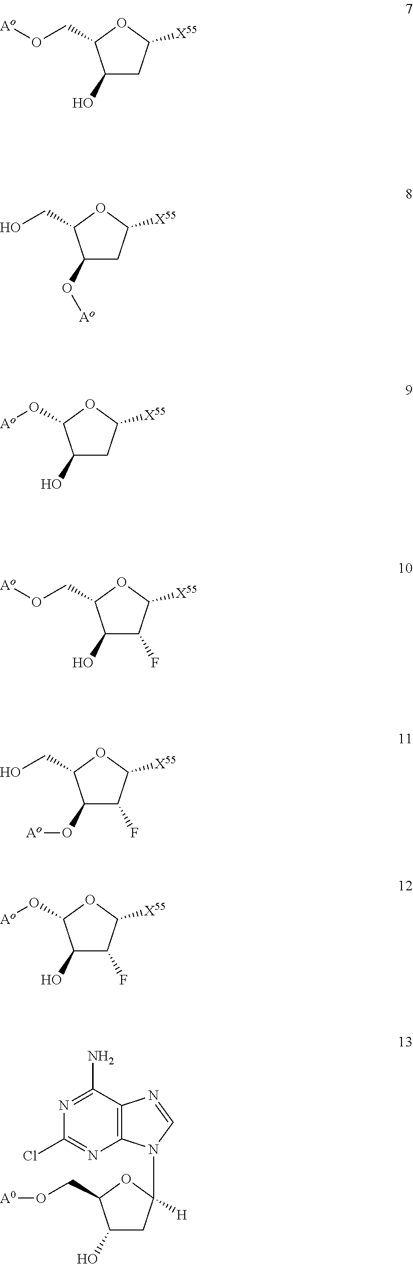

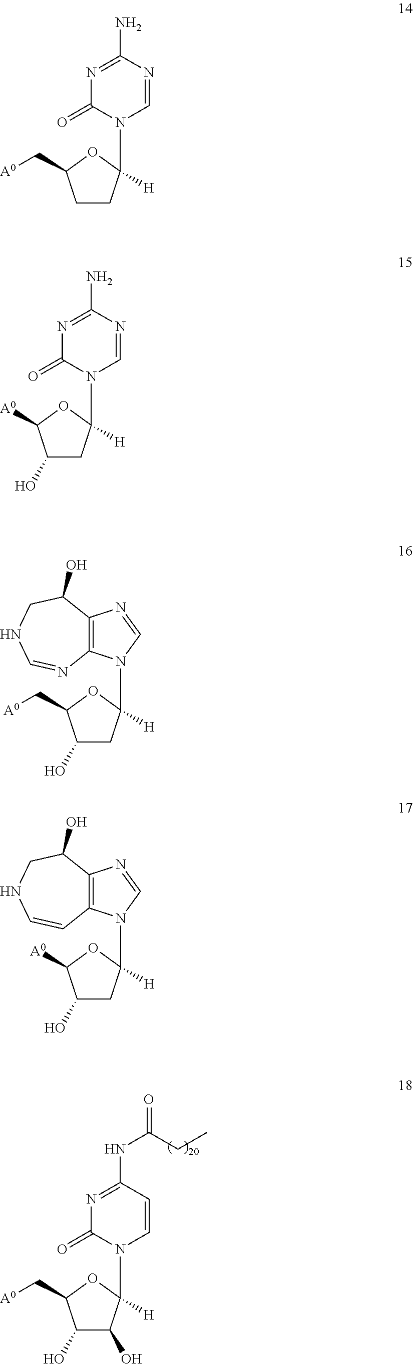

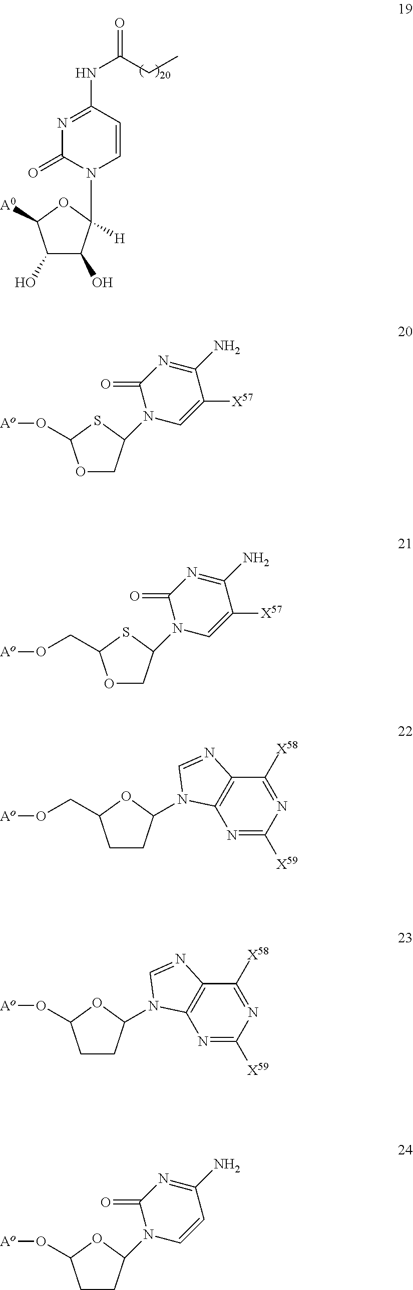

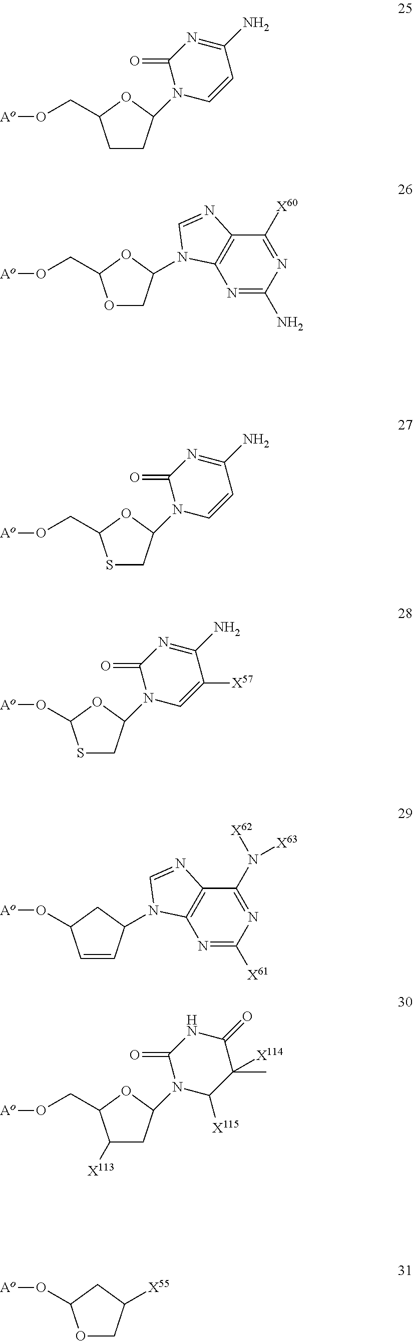

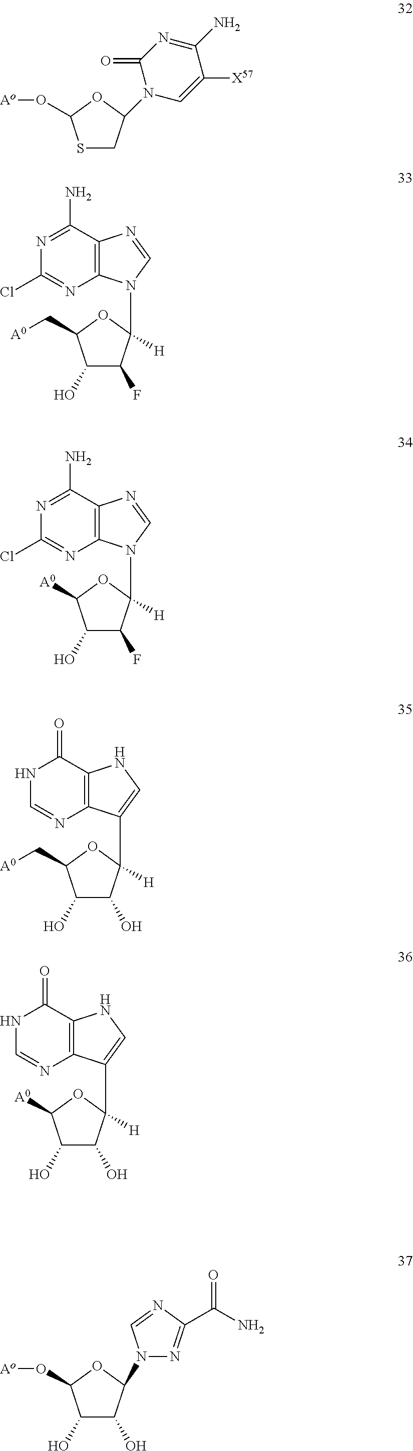

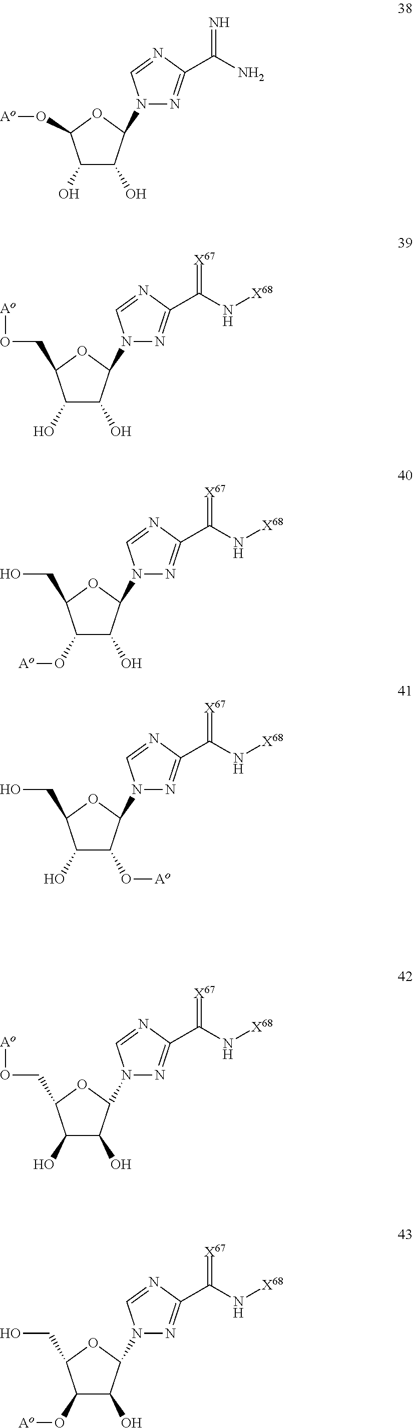

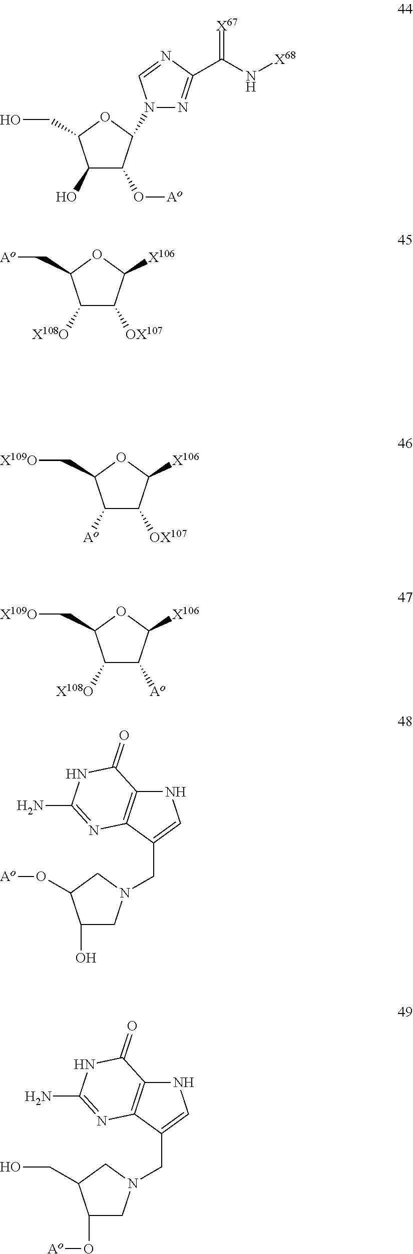

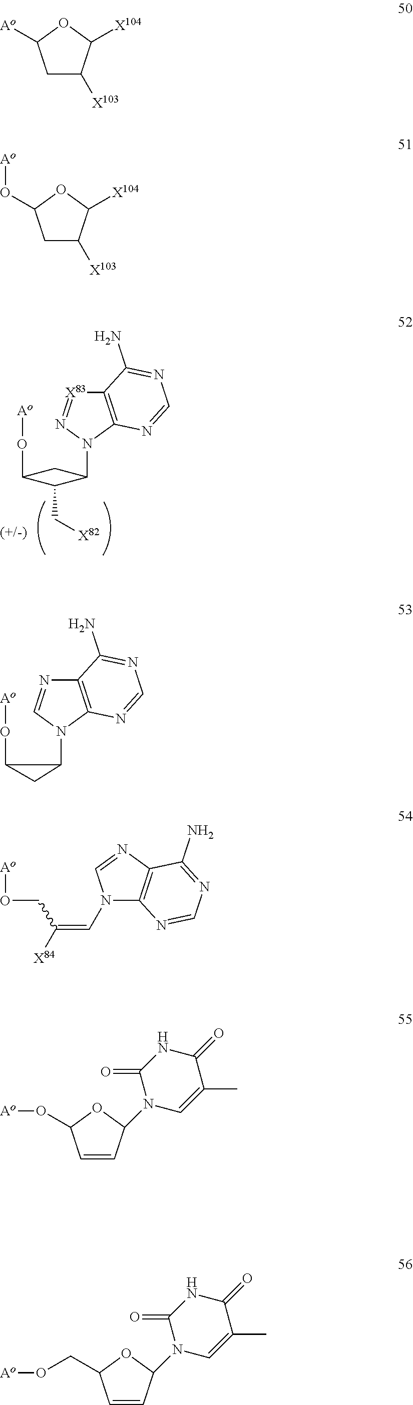

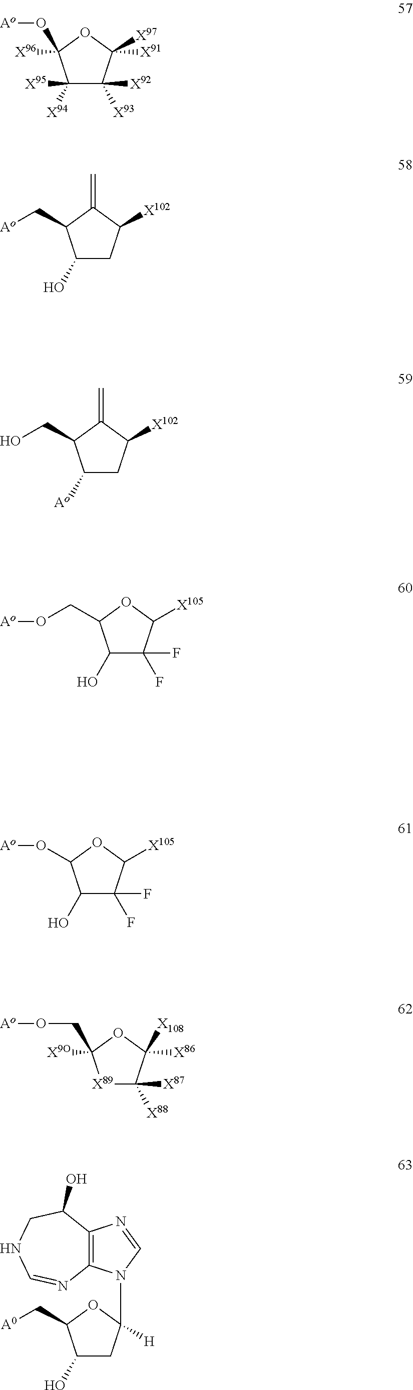

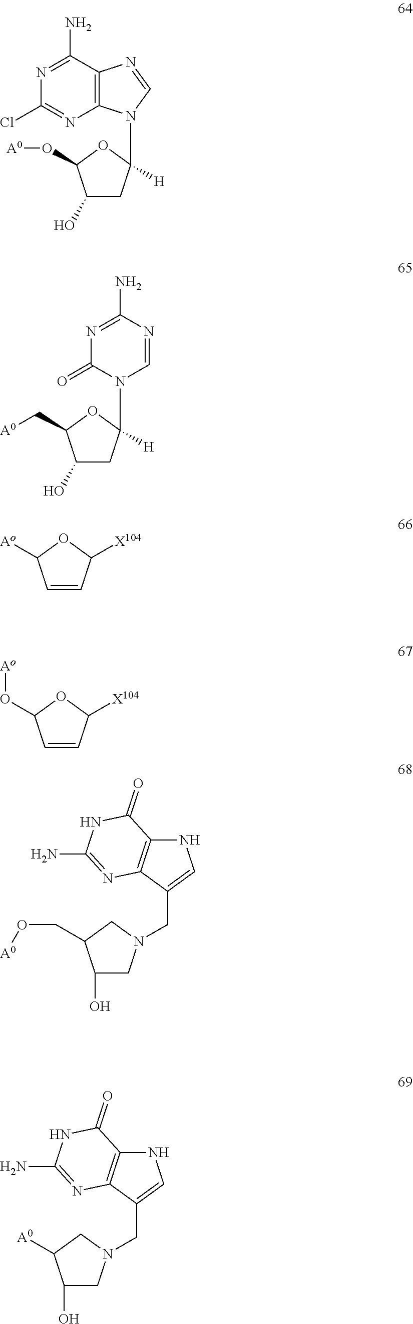

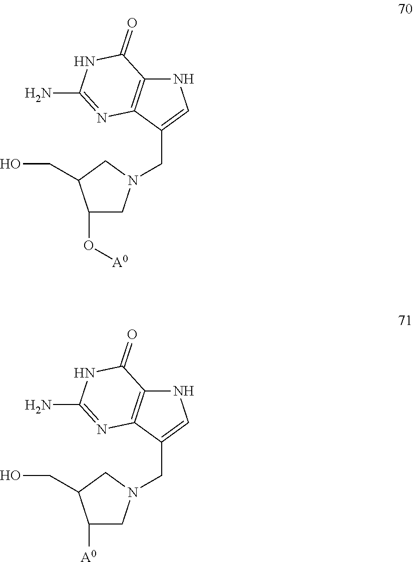

wherein:

A⁰ is A¹;

A¹ is:

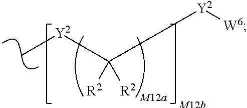

A³ is:

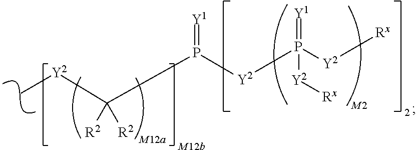

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—; and when Y² joins two phosphorous atoms Y² can also be C(R²)(R²);

R^{x} is independently H, R², W³, a protecting group, or the formula:

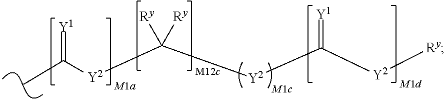

R^{y} is independently H, W³, R² or a protecting group;

R¹ is independently H or alkyl of 1 to 18 carbon atoms;

R² is independently H, R³ or R⁴ wherein each R⁴ is independently substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1;

M12c is 0, 1, 2, 3, 4, 5, 6, 7, 8, 9, 10, 11 or 12;

X⁵² is C₁-C₆ alkyl or C₇-C₁₀ arylalkyl group;

X⁵³ is H, alkyl or substituted alkyl;

X⁵⁴ is CH or N;

X⁵⁵ is thymine, adenine, uracil, a 5-halouracil, a 5-alkyluracil, guanine, cytosine, a 5-halo cytosine, a 5-alkyl cytosine, or 2,6-diaminopurine;

X⁵⁷ is H or F;

X⁵⁸ is OH, Cl, NH₂, H, Me, or MeO;

X⁵⁹ is H or NH₂;

X⁶⁰ is OH, Cl, NH₂, or H;

X⁶¹ is H, NH₂, or NH-alkyl;

X⁶² and X⁶³ are independently H, alkyl, or cyclopropyl;

X⁶⁷ is O or NH;

X⁶⁸ is H, acetate, benzyl, benzyloxycarbonyl, or an amino protecting group;

X⁸² is OH, F, or cyano;

X⁸³ is N or CH;

X⁸⁴ is a cis-hydrogen or a trans-hydrogen;

X⁸⁶ is H, methyl, hydroxymethyl, or fluoromethyl;

X⁸⁷ and X⁸⁸ are each independently H or C₁₋₄ alkyl, which alkyl is optionally substituted with OH, amino, C₁₋₄alkoxy, C₁₋₄ alkylthio, or one to three halogen atoms;

X⁸⁹ is —O— or —S(O)n-, where n is 0, 1, or 2;

X⁹⁰ is H, methyl, hydroxymethyl, or fluoromethyl;

X⁹¹ is H hydroxy, alkyl, azido, cyano, alkenyl, alkynyl, bromovinyl, —C(O)O(alkyl), —O(acyl), alkoxy, alkenyloxy, chloro, bromo, fluoro, iodo, NO₂, NH₂, —NH(lower alkyl), —NH(acyl), —N(lower alkyl)₂, —N(acyl)₂;

X⁹² is H, C₂₋₄alkenyl, C₂₋₄alkynyl, or C₁₋₄ alkyl optionally substituted with amino, hydroxy, or 1 to 3 fluorine atoms;

one of X⁹³ and X⁹⁴ is hydroxy or C₁₋₄ alkoxy and the other of X⁹³ and X⁹⁴ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

X⁹³ is H, C₂₋₄ alkenyl, C₂₋₄ alkynyl, or C₁₋₄ alkyl optionally substituted with amino, hydroxy, or 1 to 3 fluorine atoms, and one of X⁹² and X⁹⁴ is hydroxy or C₁₋₄alkoxy and the other of X⁹² and X⁹⁴ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

X⁹² and X⁹³ together with the carbon atom to which they are attached form a 3- to 6 membered saturated monocyclic ring system optionally containing a heteroatom selected from O, S, and NC₀₋₄ alkyl;

X⁹⁵ is H, OH, SH, NH₂, C₁₋₄ alkylamino, di(C₁₋₄alkyl)amino, C₃₋₆cycloalkylamino, halo, C₁₋₄alkyl, C₁₋₄ alkoxy, or CF₃; or X⁹² and X⁹⁵ can optionally together be a bond linking the two carbons to which they are attached;

X⁹⁶ is H, methyl, hydroxymethyl, or fluoromethyl;

X⁹⁷ is selected from the group consisting of

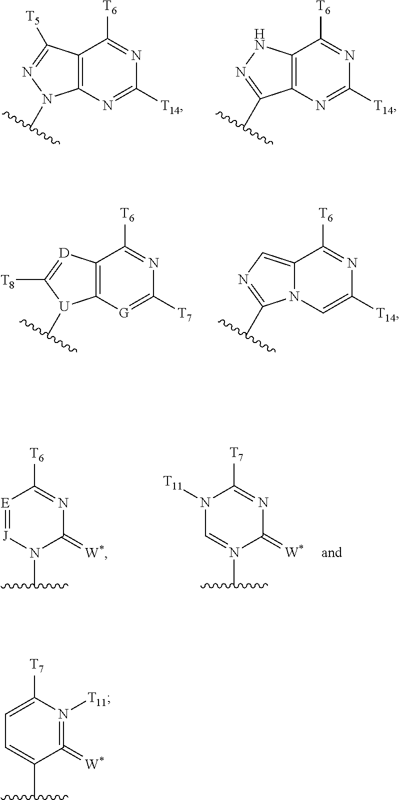

U, G, and J are each independently CH or N;

D is N, CH, C—CN, C—NO₂, C—C₁₋₃ alkyl, C—NHCONH₂, C—CONT₁₁T₁₁, C—CSNT₁₁T₁₁, C—COOT₁₁, C—C(═NH)NH₂, C-hydroxy, C—C₁₋₃ alkoxy, C-amino, C—C₁₋₄ alkylamino, C-di(C₁₋₄alkyl)amino, C-halogen, C-(1,3-oxazol-2-yl), C-(1,3 thiazol-2-yl), or C-(imidazol-2-yl); wherein alkyl is unsubstituted or substituted with one to three groups independently selected from halogen, amino, hydroxy, carboxy, and C₁₋₃ alkoxy;

E is N or CT₅;

W³ is O or S;

T₁ is H, C₂₋₄alkenyl, C₂₋₄alkynyl, or C₁₋₄alkyl optionally substituted with amino, hydroxy, or 1 to 3 fluorine atoms and one of T₂ and T₃ is hydroxy or C₁₋₄ alkoxy and the other of T₂ and T₃ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

T₂ is H, C₂₋₄alkenyl, C₂₋₄alkynyl, or C₁₋₄alkyl optionally substituted with amino, hydroxy, or 1 to 3 fluorine atoms and one of T₁ and T₃ is hydroxy or C₁₋₄alkoxy and the other of T₁ and T₃ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

T₁ and T₂ together with the carbon atom to which they are attached form a 3- to 6 membered saturated monocyclic ring system optionally containing a heteroatom selected from O, S, and NC₀₋₄ alkyl;

T₄ and T₆ are each independently H, OH, SH, NH₂, C₁₋₄ alkylamino, alkyl)amino, C₃₋₆ cycloalkylamino, halo, C₁₋₄ alkyl, C₁₋₄ alkoxy, or CF₃;

T₅ is H, C₁₋₆alkenyl, C₂₋₆alkynyl, C₁₋₄alkylamino, CF₃, or halogen; T₁₄ is H, CF₃, C₁₋₄ alkyl, amino, C₁₋₄alkylamino, C₃₋₆cycloalkylamino, or di(C₁₋₄alkyl)amino;

T₇ is H, amino, C₁₋₄alkylamino, C₃₋₆ cycloalkylamino, or di(C₁₋₄alkyl)amino;

each T₁₁ is independently H or C₁₋₆ alkyl;

T₈ is H, halo, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

X¹⁰² is thymine, adenine, guanine, cytosine, uracil, inosine, or diaminopurine;

X¹⁰³ is OH, OR, NR₂, CN, NO₂, F, Cl, Br, or I;

X¹⁰⁴ is adenine, guanine, cytosine, uracil, thymine, 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, or pyrazolo[3,4-d]pyrimidine;

X¹⁰⁵ is guanine, cytosine, uracil, thymine;

X¹⁰⁶ is

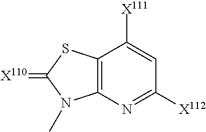

wherein X¹¹⁰ and X¹¹¹ are independently O or S and X¹¹² is H, amino, hydroxy, or a halogen selected from Cl and Br;

X¹⁰⁷ and X¹⁰⁸ are independently selected from H or a C₁-C₁₈ acyl; and X¹⁰⁹ is H, a C₁-C₁₈ acyl, or

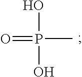

or X¹⁰⁷ is H and together X¹⁰⁸ and X¹⁰⁹ are

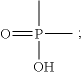

X¹¹³ is R³;

X¹¹⁴ is R⁴; and

X¹¹⁵ is R⁵.

The invention provides a pharmaceutical composition comprising an effective amount of a compound of the invention, or a pharmaceutically acceptable salt thereof, in combination with a pharmaceutically acceptable diluent or carrier.

This invention pertains to a method of increasing cellular accumulation and retention of drug compounds, thus improving their therapeutic and diagnostic value, comprising linking the compound to one or more phosphonate groups.

The invention also provides a method of inhibiting DNA and/or RNA synthesis, comprising administering to a mammal afflicted with a condition amenable to treatment via DNA and/or RNA synthesis, e.g., cancer and viral infection, an amount of a compound of the invention, effective to inhibit inhibit DNA and/or RNA synthesis.

The invention also provides a compound of the invention for use in medical therapy (preferably for use in treating cancer or viral infection), as well as the use of a compound of the invention for the manufacture of a medicament useful for the treatment of cancer or viral infection.

The invention also provides processes and novel intermediates disclosed herein which are useful for preparing compounds of the invention. Some of the compounds of the invention are useful to prepare other compounds of the invention.

In another aspect of the invention, the DNA and/or RNA synthesis is inhibited by a method comprising the step of treating a sample with a compound or composition of the invention.

### DETAILED DESCRIPTION OF THE INVENTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

### Definitions

Unless stated otherwise, the following terms and phrases as used herein are intended to have the following meanings:

When tradenames are used herein, applicants intend to independently include the tradename product and the active pharmaceutical ingredient(s) of the tradename product.

“Bioavailability” is the degree to which the pharmaceutically active agent becomes available to the target tissue after the agent's introduction into the body. Enhancement of the bioavailability of a pharmaceutically active agent can provide a more efficient and effective treatment for patients because, for a given dose, more of the pharmaceutically active agent will be available at the targeted tissue sites.

The terms “phosphonate” and “phosphonate group” include functional groups or moieties within a molecule that comprises a phosphorous that is 1) single-bonded to a carbon, 2) double-bonded to a heteroatom, 3) single-bonded to a heteroatom, and 4) single-bonded to another heteroatom, wherein each heteroatom can be the same or different. The terms “phosphonate” and “phosphonate group” also include functional groups or moieties that comprise a phosphorous in the same oxidation state as the phosphorous described above, as well as functional groups or moieties that comprise a prodrug moiety that can separate from a compound so that the compound retains a phosphorous having the characteriatics described above. For example, the terms “phosphonate” and “phosphonate group” include phosphonic acid, phosphonic monoester, phosphonic diester, phosphonamidate, and phosphonthioate functional groups. In one specific embodiment of the invention, the terms “phosphonate” and “phosphonate group” include functional groups or moieties within a molecule that comprises a phosphorous that is 1) single-bonded to a carbon, 2) double-bonded to an oxygen, 3) single-bonded to an oxygen, and 4) single-bonded to another oxygen, as well as functional groups or moieties that comprise a prodrug moiety that can separate from a compound so that the compound retains a phosphorous having such characteriatics. In another specific embodiment of the invention, the terms “phosphonate” and “phosphonate group” include functional groups or moieties within a molecule that comprises a phosphorous that is 1) single-bonded to a carbon, 2) double-bonded to an oxygen, 3) single-bonded to an oxygen or nitrogen, and 4) single-bonded to another oxygen or nitrogen, as well as functional groups or moieties that comprise a prodrug moiety that can separate from a compound so that the compound retains a phosphorous having such characteriatics.

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Prodrug moiety” refers to a labile functional group which separates from the active inhibitory compound during metabolism, systemically, inside a cell, by hydrolysis, enzymatic cleavage, or by some other process (Bundgaard, Hans, “Design and Application of Prodrugs” in A Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy. A prodrug moiety may include an active metabolite or drug itself.

Exemplary prodrug moieties include the hydrolytically sensitive or labile acyloxymethyl esters —CH₂OC(═O)R⁹ and acyloxymethyl carbonates —CH₂OC(═O)OR⁹ where R⁹ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al. (1983) J. Pharm. Sci. 72: 324; also U.S. Pat. Nos. 4,816,570, 4,968,788, 5,663,159 and 5,792,756. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the combinations of the invention. An exemplary acyloxymethyl ester is pivaloyloxymethoxy, (POM) —CH₂OC(═O)C(CH₃)₃. An exemplary acyloxymethyl carbonate prodrug moiety is pivaloyloxymethylcarbonate (POC) —CH₂OC(═O)OC(CH₃)₃.

The phosphonate group may be a phosphonate prodrug moiety. The prodrug moiety may be sensitive to hydrolysis, such as, but not limited to a pivaloyloxymethyl carbonate (POC) or POM group. Alternatively, the prodrug moiety may be sensitive to enzymatic potentiated cleavage, such as a lactate ester or a phosphonamidate-ester group.

Aryl esters of phosphorus groups, especially phenyl esters, are reported to enhance oral bioavailability (De Lombaert et al. (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphonic acid. In some cases, substituents at the ortho-orpara-position may accelerate the hydrolysis. Benzyl analogs with an acylated phenol or an alkylated phenol may generate the phenolic compound through the action of enzymes, e.g., esterases, oxidases, etc., which in turn undergoes cleavage at the benzylic C—O bond to generate the phosphoric acid and the quinone methide intermediate. Examples of this class of prodrugs are described by Mitchell et al. (1992) J. Chem. Soc. Perkin Trans. II 2345; Glazier WO 91/19721. Still other benzylic prodrugs have been described containing a carboxylic ester-containing group attached to the benzylic methylene (Glazier WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al. (1993) Antiviral Res., 22: 155-174; Benzaria et al. (1996) J. Med. Chem. 39: 4958). Cyclic phosphonate esters have also been described as prodrugs of phosphorus-containing compounds (Erion et al., U.S. Pat. No. 6,312,662).

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Any reference to any of the compounds of the invention also includes a reference to a physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to, acetylenic (—C≡CH) and propargyl (—CH₂C≡CH),

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to, methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to, 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to, acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Substituted alkyl”, “substituted aryl”, and “substituted arylalkyl” mean alkyl, aryl, and arylalkyl respectively, in which one or more hydrogen atoms are each independently replaced with a non-hydrogen substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O^{—})₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently —H, alkyl, aryl, heterocycle, protecting group or prodrug moiety. Alkylene, alkenylene, and alkynylene groups may also be similarly substituted.

“Heterocycle” as used herein includes by way of example and not limitation these heterocycles described in Paquette, Leo A.; Principles of Modem Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S).

Examples of heterocycles include by way of example and not limitation pyridyl, dihydroypyridyl, tetrahydropyridyl(piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

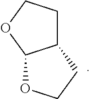

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” refers to a saturated, unsaturated or aromatic ring having 3 to 7 carbon atoms as a monocycle, 7 to 12 carbon atoms as a bicycle, and up to about 20 carbon atoms as a polycycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

“Linker” or “link” refers to a chemical moiety comprising a covalent bond or a chain or group of atoms that covalently attaches a phosphonate group to a drug. Linkers include portions of substituents A¹ and A³, which include moieties such as: repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes preventing the disease or condition from occurring, inhibiting the disease or condition, eliminating the disease or condition, and/or relieving one or more symptoms of the disease or condition.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

### Protecting Groups

In the context of the present invention, protecting groups include prodrug moieties and chemical protecting groups.

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phospbonic acid, phosphonate, sulfonic acid and other protecting groups for acids see Greene as set forth below. Such groups include by way of example and not limitation, esters, amides, hydrazides, and the like.

### Ether- and Ester-Forming Protecting Groups

Ester-forming groups include: (1) phosphonate ester-forming groups, such as phosphonamidate esters, phosphorothioate esters, phosphonate esters, and phosphon-bis-amidates; (2) carboxyl ester-forming groups, and (3) sulphur ester-forming groups, such as sulphonate, sulfate, and sulfinate.

The phosphonate moieties of the compounds of the invention may or may not be prodrug moieties, i.e. they may or may be susceptible to hydrolytic or enzymatic cleavage or modification. Certain phosphonate moieties are stable under most or nearly all metabolic conditions. For example, a dialkylphosphonate, where the alkyl groups are two or more carbons, may have appreciable stability in vivo due to a slow rate of hydrolysis.

Within the context of phosphonate prodrug moieties, a large number of structurally-diverse prodrugs have been described for phosphonic acids (Freeman and Ross in Progress in Medicinal Chemistry 34: 112-147 (1997) and are included within the scope of the present invention. An exemplary phosphonate ester-forming group is the phenyl carbocycle in substructure A₃ having the formula:

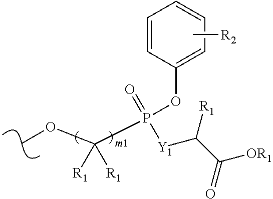

wherein R₁ may be H or C₁-C₁₂ alkyl; m1 is 1, 2, 3, 4, 5, 6, 7 or 8, and the phenyl carbocycle is substituted with 0 to 3 R₂ groups. Where Y₁ is O, a lactate ester is formed, and where Y₁ is N(R₂), N(OR₂) or N(N(R₂)₂, a phosphonamidate ester results.

In its ester-forming role, a protecting group typically is bound to any acidic group such as, by way of example and not limitation, a —CO₂H or —C(S)OH group, thereby resulting in —CO₂R^{x} where R^{x} is defined herein. Also, R^{x} for example includes the enumerated ester groups of WO 95/07920.

Examples of protecting groups include:

C₃-C₁₂ heterocycle (described above) or aryl. These aromatic groups optionally are polycyclic or monocyclic. Examples include phenyl, spiryl, 2- and 3-pyrrolyl, 2- and 3-thienyl, 2- and 4-imidazolyl, 2-, 4- and 5-oxazolyl, 3- and 4-isoxazolyl, 2-, 4- and 5-thiazolyl, 3-, 4- and 5-isothiazolyl, 3- and 4-pyrazolyl, 1-, 2-, 3- and 4-pyridinyl, and 1-, 2-, 4- and 5-pyrimidinyl,

C₃-C₁₂ heterocycle or aryl substituted with halo, R¹, R¹—O—C₁-C₁₂ alkylene, C₁-C₁₂ alkoxy, CN, NO₂, OH, carboxy, carboxyester, thiol, thioester, C₁-C₁₂ haloalkyl (1-6 halogen atoms), C₂-C₁₂ alkenyl or C₂-C₁₂ alkynyl. Such groups include 2-, 3- and 4-alkoxyphenyl (C₁-C₁₂ alkyl), 2-, 3- and 4-methoxyphenyl, 2-, 3- and 4-ethoxyphenyl, 2,3-, 2,4-, 2,5-, 2,6-, 3,4- and 3,5-diethoxyphenyl, 2- and 3-carboethoxy-4-hydroxyphenyl, 2- and 3-ethoxy-4-hydroxyphenyl, 2- and 3-ethoxy-5-hydroxyphenyl, 2- and 3-ethoxy-6-hydroxyphenyl, 2-, 3- and 4-O-acetylphenyl, 2-, 3- and 4-dimethylaminophenyl, 2-, 3- and 4-methylmercaptophenyl, 2-, 3- and 4-halophenyl (including 2-, 3- and 4-fluorophenyl and 2-, 3- and 4-chlorophenyl), 2,3-, 2,4-, 2,5-, 2,6-, 3,4- and 3,5-dimethylphenyl, 2,3-, 2,4-, 2,5-, 2,6-, 3,4- and 3,5-biscarboxyethylphenyl, 2,3-, 2,4-, 2,5-, 2,6-, 3,4- and 3,5-dimethoxyphenyl, 2,3-, 2,4-, 2,5-, 2,6-, 3,4- and 3,5-dihalophenyl (including 2,4-difluorophenyl and 3,5-difluorophenyl), 2-, 3- and 4-haloalkylphenyl (1 to 5 halogen atoms, C₁-C₁₂ alkyl including 4-trifluoromethylphenyl), 2-, 3- and 4-cyanophenyl, 2-, 3- and 4-nitrophenyl, 2-, 3- and 4-haloalkylbenzyl (1 to 5 halogen atoms, C₁-C₁₂ alkyl including 4-trifluoromethylbenzyl and 2-, 3- and 4-trichloromethylphenyl and 2-, 3- and 4-trichloromethylphenyl), 4-N-methylpiperidinyl, 3-N-methylpiperidinyl, 1-ethylpiperazinyl, benzyl, alkylsalicylphenyl (C₁-C₄ alkyl, including 2-, 3- and 4-ethylsalicylphenyl), 2-,3- and 4-acetylphenyl, 1,8-dihydroxynaphthyl (—C₁₀H₆—OH) and aryloxy ethyl [C₆-C₉ aryl (including phenoxy ethyl)], 2,2′-dihydroxybiphenyl, 2-, 3- and 4-N,N-dialkylaminophenol, —C₆H₄CH₂—N(CH₃)₂, trimethoxybenzyl, triethoxybenzyl, 2-alkyl pyridinyl (C₁₋₄ alkyl);

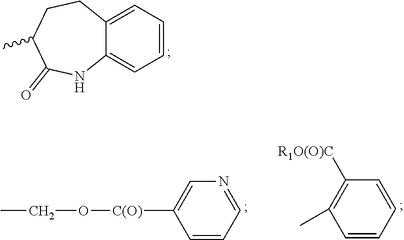

C₄-C₈ esters of 2-carboxyphenyl; and C₁-C₄ alkylene-C₃-C₆ aryl (including benzyl, —CH₂-pyrrolyl, —CH₂-thienyl, —CH₂-imidazolyl, —CH₂-oxazolyl, —CH₂-isoxazolyl, —CH₂-thiazolyl, —CH₂-isothiazolyl, —CH₂-pyrazolyl, —CH₂-pyridinyl and —CH₂-pyrimidinyl) substituted in the aryl moiety by 3 to 5 halogen atoms or 1 to 2 atoms or groups selected from halogen, C₁-C₁₂ alkoxy (including methoxy and ethoxy), cyano, nitro, OH, C₁-C₁₂ haloalkyl (1 to 6 halogen atoms; including —CH₂CCl₃), C₁-C₁₂ alkyl (including methyl and ethyl), C₂-C₁₂ alkenyl or C₂-C₁₂ alkynyl; alkoxy ethyl [C₁-C₆ alkyl including —CH₂—CH₂—O—CH₃ (methoxy ethyl)]; alkyl substituted by any of the groups set forth above for aryl, in particular OH or by 1 to 3 halo atoms (including —CH₃, —CH(CH₃)₂, —C(CH₃)₃, —CH₂CH₃, —(CH₂)₂CH₃, —(CH₂)₃CH₃, —(CH₂)₄CH₃, —(CH₂)₅CH₃, —CH₂CH₂F, —CH₂CH₂Cl, —CH₂CF₃, and —CH₂CCl₃);

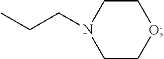

—N-2-propylmorpholino, 2,3-dihydro-6-hydroxyindene, sesamol, catechol monoester, —CH₂—C(O)—N(R¹)₂, —CH₂—S(O)(R¹), —CH₂—S(O)₂(R¹), —CH₂—CH(OC(O)CH₂R¹)—CH₂(OC(O)CH₂R¹), cholesteryl, enolpyruvate (HOOC—C(═CH₂)—), glycerol;

a 5 or 6 carbon monosaccharide, disaccharide or oligosaccharide (3 to 9 monosaccharide residues);

triglycerides such as α-D-β-diglycerides (wherein the fatty acids composing glyceride lipids generally are naturally occurring saturated or unsaturated C₆₋₂₆, C₆₋₁₈ or C₆₋₁₀ fatty acids such as linoleic, lauric, myristic, palmitic, stearic, oleic, palmitoleic, linolenic and the like fatty acids) linked to acyl of the parental compounds herein through a glyceryl oxygen of the triglyceride;

phospholipids linked to the carboxyl group through the phosphate of the phospholipid;

phthalidyl (shown in FIG. 1 of Clayton et al., Antimicrob. Agents Chemo. (1974) 5(6):670-671;

cyclic carbonates such as (5-R_{d}-2-oxo-1,3-dioxolen-4-yl) methyl esters (Sakamoto et al., Chem. Pharm. Bull. (1984) 32(6)2241-2248) where R_{d} is R₁, R₄ or aryl; and

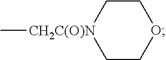

The hydroxyl groups of the compounds of this invention optionally are substituted with one of groups III, IV or V disclosed in WO 94/21604, or with isopropyl.

Table A lists examples of protecting group ester moieties that for example can be bonded via oxygen to —C(O)O— and —P(O)(O—)₂ groups. Several amidates also are shown, which are bound directly to —C(O)— or —P(O)₂. Esters of structures 1-5, 8-10 and 16, 17, 19-22 are synthesized by reacting the compound herein having a free hydroxyl with the corresponding halide (chloride or acyl chloride and the like) and N,N-dicyclohexyl-N-morpholine carboxamidine (or another base such as DBU, triethylamine, CsCO₃, N,N-dimethylaniline and the like) in DMF (or other solvent such as acetonitrile or N-methylpyrrolidone). When the compound to be protected is a phosphonate, the esters of structures 5-7, 11, 12, 21, and 23-26 are synthesized by reaction of the alcohol or alkoxide salt (or the corresponding amines in the case of compounds such as 13, 14 and 15) with the monochlorophosphonate or dichlorophosphonate (or another activated phosphonate).

TABLE A

| | |
|---|---|
| 1. | —CH₂—C(O)—N(R₁)₂ * |
| 2. | —CH₂—S(O)(R₁) |
| 3. | —CH₂—S(O)₂(R₁) |
| 4. | —CH₂—O—C(O)—CH₂—C₆H₅ |
| 5. | 3-cholesteryl |
| 6. | 3-pyridyl |
| 7. | N-ethylmorpholino |
| 8. | —CH₂—O—C(O)—C₆H₅ |
| 9. | —CH₂—O—C(O)—CH₂CH₃ |
| 10. | —CH₂—O—C(O)—C(CH₃)₃ |
| 11. | —CH₂—CCl₃ |
| 12. | —C₆H₅ |
| 13. | —NH—CH₂—C(O)O—CH₂CH₃ |
| 14. | —N(CH₃)—CH₂—C(O)O—CH₂CH₃ |
| 15. | —NHR₁ |
| 16. | —CH₂—O—C(O)—C₁₀H₁₅ |
| 17. | —CH₂—O—C(O)—CH(CH₃)₂ |
| 18. | —CH₂—C#H(OC(O)CH₂R₁)—CH₂—(OC(O)CH₂R₁)* |
| 19. | 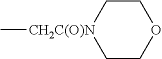 |
| 20. | 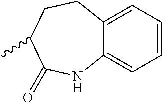 |
| 21. | 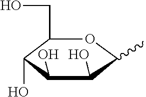 |
| 22. | 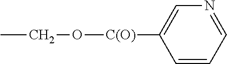 |
| 23. | 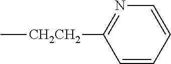 |
| 24. | 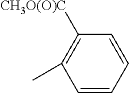 |
| 25. | 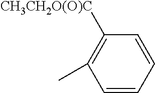 |
| 26. | 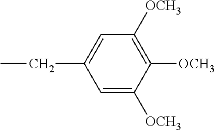 |

#chiral center is (R), (S) or racemate.

Other esters that are suitable for use herein are described in EP 632048.

Protecting groups also includes “double ester” forming profunctionalities such as —CH₂OC(O)OCH₃,

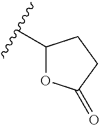

—CH₂SCOCH₃, —CH₂OCON(CH₃)₂, or alkyl- or aryl-acyloxyalkyl groups of the structure —CH(R¹ or W⁵)O((CO)R³⁷) or —CH(R¹ or W⁵)((CO)OR³⁸) (linked to oxygen of the acidic group) wherein R³⁷ and R³⁸ are alkyl, aryl, or alkylaryl groups (see U.S. Pat. No. 4,968,788). Frequently R³⁷ and R³⁸ are bulky groups such as branched alkyl, ortho-substituted aryl, meta-substituted aryl, or combinations thereof, including normal, secondary, iso- and tertiary alkyls of 1-6 carbon atoms. An example is the pivaloyloxymethyl group. These are of particular use with prodrugs for oral administration. Examples of such useful protecting groups are alkylacyloxymethyl esters and their derivatives, including —CH(CH₂CH₂OCH₃)OC(O)C(CH₃)₃,

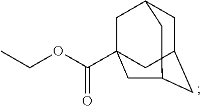

—CH₂OC(O)C₁₀H₁₅, —CH₂OC(O)C(CH₃)₃, —CH(CH₂OCH₃)OC(O)C(CH₃)₃, —CH(CH(CH₃)₂)OC(O)C(CH₃)₃, —CH₂OC(O)CH₂CH(CH₃)₂, —CH₂OC(O)C₆H₁₁, —CH₂OC(O)C₆H₅, —CH₂OC(O)C₁₀H₁₅, —CH₂OC(O)CH₂CH₃, —CH₂OC(O)CH(CH₃)₂, —CH₂OC(O)C(CH₃)₃ and —CH₂OC(O)CH₂C₆H₅.

In some claims the protected acidic group is an ester of the acidic group and is the residue of a hydroxyl-containing functionality. In other claims, an amino compound is used to protect the acid functionality. The residues of suitable hydroxyl or amino-containing functionalities are set forth above or are found in WO 95/07920. Of particular interest are the residues of amino acids, amino acid esters, polypeptides, or aryl alcohols. Typical amino acid, polypeptide and carboxyl-esterified amino acid residues are described on pages 11-18 and related text of WO 95/07920 as groups L1 or L2. WO 95/07920 expressly teaches the amidates of phosphonic acids, but it will be understood that such amidates are formed with any of the acid groups set forth herein and the amino acid residues set forth in WO 95/07920.

Typical esters for protecting acidic functionalities are also described in WO 95/07920, again understanding that the same esters can be formed with the acidic groups herein as with the phosphonate of the '920 publication. Typical ester groups are defined at least on WO 95/07920 pages 89-93 (under R³¹ or R³⁵), the table on page 105, and pages 21-23 (as R). Of particular interest are esters of unsubstituted aryl such as phenyl or arylalkyl such benzyl, or hydroxy-, halo-, alkoxy-, carboxy- and/or alkylestercarboxy-substituted aryl or alkylaryl, especially phenyl, ortho-ethoxyphenyl, or C₁-C₄ alkylestercarboxyphenyl (salicylate C₁-C₁₂ alkylesters).

The protected acidic groups, particularly when using the esters or amides of WO 95/07920, are useful as prodrugs for oral administration. However, it is not essential that the acidic group be protected in order for the compounds of this invention to be effectively administered by the oral route. When the compounds of the invention having protected groups, in particular amino acid amidates or substituted and unsubstituted aryl esters are administered systemically or orally they are capable of hydrolytic cleavage in vivo to yield the free acid.

One or more of the acidic hydroxyls are protected. If more than one acidic hydroxyl is protected then the same or a different protecting group is employed, e.g., the esters may be different or the same, or a mixed amidate and ester may be used.

Typical hydroxy protecting groups described in Greene (pages 14-118) include substituted methyl and alkyl ethers, substituted benzyl ethers, silyl ethers, esters including sulfonic acid esters, and carbonates. For example:
Ethers (methyl, t-butyl, allyl);
Substituted Methyl Ethers (Methoxymethyl, Methylthiomethyl, t-Butylthiomethyl, (Phenyldimethylsilyl)methoxymethyl, Benzyloxymethyl, p-Methoxybenzyloxymethyl, (4-Methoxyphenoxy)methyl, Guaiacolmethyl, t-Butoxymethyl, 4-Pentenyloxymethyl, Siloxymethyl, 2-Methoxyethoxymethyl, 2,2,2-Trichloroethoxymethyl, Bis(2-chloroethoxy)methyl, 2-(Trimethylsilyl)ethoxymethyl, Tetrahydropyranyl, 3-Bromotetrahydropyranyl, Tetrahydropthiopyranyl, 1-Methoxycyclohexyl, 4-Methoxytetrahydropyranyl, 4-Methoxytetrahydrothiopyranyl, 4-Methoxytetrahydropthiopyranyl S,S-Dioxido, 1-[(2-Chloro-4-methyl)phenyl]-4-methoxypiperidin-4-yl, 1,4-Dioxan-2-yl, Tetrahydrofuranyl, Tetrahydrothiofuranyl, 2,3,3a,4,5,6,7,7a-Octahydro-7,8,8-trimethyl-4,7-methanobenzofuran-2-yl));
Substituted Ethyl Ethers (1-Ethoxyethyl, 1-(2-Chloroethoxy)ethyl, 1-Methyl-1-methoxyethyl, 1-Methyl-1-benzyloxyethyl, 1-Methyl-1-benzyloxy-2-fluoroethyl, 2,2,2-Trichloroethyl, 2-Trimethylsilylethyl, 2-(Phenylselenyl)ethyl,
p-Chlorophenyl, p-Methoxyphenyl, 2,4-Dinitrophenyl, Benzyl);
Substituted Benzyl Ethers (p-Methoxybenzyl, 3,4-Dimethoxybenzyl, o-Nitrobenzyl, p-Nitrobenzyl, p-Halobenzyl, 2,6-Dichlorobenzyl, p-Cyanobenzyl, p-Phenylbenzyl, 2- and 4-Picolyl, 3-Methyl-2-picolyl N-Oxido, Diphenylmethyl, p,p′-Dinitrobenzhydryl, 5-Dibenzosuberyl, Triphenylmethyl, α-Naphthyldiphenylmethyl, p-methoxyphenyldiphenylmethyl, Di(p-methoxyphenyl)phenylmethyl, Tri(p-methoxyphenyl)methyl, 4-(4′-Bromophenacyloxy)phenyldiphenylmethyl, 4,4′,4″-Tris(4,5-dichlorophthalimidophenyl)methyl, 4,4′,4″-Tris(levulinoyloxyphenyl)methyl, 4,4′,4″-Tris(benzoyloxyphenyl)methyl, 3-(Imidazol-1-ylmethyl)bis(4′,4″-dimethoxyphenyl)methyl, 1,1-Bis(4-methoxyphenyl)-1′-pyrenylmethyl, 9-Anthryl, 9-(9-Phenyl)xanthenyl, 9-(9-Phenyl-10-oxo)anthryl, 1,3-Benzodithiolan-2-yl, Benzisothiazolyl S,S-Dioxido);
Silyl Ethers (Trimethylsilyl, Triethylsilyl, Triisopropylsilyl, Dimethylisopropylsilyl, Diethylisopropylsilyl, Dimethylthexylsilyl, t-Butyldimethylsilyl, t-Butyldiphenylsilyl, Tribenzylsilyl, Tri-p-xylylsilyl, Triphenylsilyl, Diphenylmethylsilyl, t-Butylmethoxyphenylsilyl);
Esters (Formate, Benzoylformate, Acetate, Choroacetate, Dichloroacetate, Trichloroacetate, Trifluoroacetate, Methoxyacetate, Triphenylmethoxyacetate, Phenoxyacetate, p-Chlorophenoxyacetate, p-poly-Phenylacetate, 3-Phenylpropionate, 4-Oxopentanoate(Levulinate), 4,4-(Ethylenedithio)pentanoate, Pivaloate, Adamantoate, Crotonate, 4-Methoxycrotonate, Benzoate, p-Phenylbenzoate, 2,4,6-Trimethylbenzoate(Mesitoate));
Carbonates (Methyl, 9-Fluorenylmethyl, Ethyl, 2,2,2-Trichloroethyl, 2-(Trimethylsilyl)ethyl, 2-(Phenylsulfonyl)ethyl, 2-(Triphenylphosphonio)ethyl, Isobutyl, Vinyl, Allyl, p-Nitrophenyl, Benzyl, p-Methoxybenzyl, 3,4-Dimethoxybenzyl, o-Nitrobenzyl, p-Nitrobenzyl, S-Benzyl Thiocarbonate, 4-Ethoxy-1-naphthyl, Methyl Dithiocarbonate);
Groups With Assisted Cleavage (2-Iodobenzoate, 4-Azidobutyrate, 4-Nitro-4-methylpentanoate, o-(Dibromomethyl)benzoate, 2-Formylbenzenesulfonate, 2-(Methylthiomethoxy)ethyl Carbonate, 4-(Methylthiomethoxy)butyrate, 2-(Methylthiomethoxymethyl)benzoate); Miscellaneous Esters(2,6-Dichloro-4-methylphenoxyacetate, 2,6-Dichloro-4-(1,1,3,3 tetramethylbutyl)phenoxyacetate, 2,4-Bis(1,1-dimethylpropyl)phenoxyacetate, Chlorodiphenylacetate, Isobutyrate, Monosuccinate, (E)-2-Methyl-2-butenoate(Tigloate), o-(Methoxycarbonyl)benzoate, p-poly-Benzoate, α-Naphthoate, Nitrate, Alkyl N,N,N′,N′-Tetramethylphosphorodiamidate, N-Phenylcarbamate, Borate, Dimethylphosphinothioyl, 2,4-Dinitrophenylsulfenate); and
Sulfonates (Sulfate, Methanesulfonate (Mesylate), Benzylsulfonate, Tosylate).

Typical 1,2-diol protecting groups (thus, generally where two OH groups are taken together with the protecting functionality) are described in Greene at pages 118-142 and include Cyclic Acetals and Ketals (Methylene, Ethylidene, 1-t-Butylethylidene, 1-Phenylethylidene, (4-Methoxyphenyl)ethylidene, 2,2,2-Trichloroethylidene, Acetonide(Isopropylidene), Cyclopentylidene, Cyclohexylidene, Cycloheptylidene, Benzylidene, p-Methoxybenzylidene, 2,4-Dimethoxybenzylidene, 3,4-Dimethoxybenzylidene, 2-Nitrobenzylidene); Cyclic Ortho Esters (Methoxymethylene, Ethoxymethylene, Dimethoxymethylene, 1-Methoxyethylidene, 1-Ethoxyethylidine, 1,2-Dimethoxyethylidene, α-Methoxybenzylidene, 1-(N,N-Dimethylamino)ethylidene Derivative, α-(N,N-Dimethylamino)benzylidene Derivative, 2-Oxacyclopentylidene); Silyl Derivatives (Di-t-butylsilylene Group, 1,3-(1,1,3,3-Tetraisopropyldisiloxanylidene), and Tetra-t-butoxydisiloxane-1,3-diylidene), Cyclic Carbonates, Cyclic Boronates, Ethyl Boronate and Phenyl Boronate.

More typically, 1,2-diol protecting groups include those shown in Table B, still more typically, epoxides, acetonides, cyclic ketals and aryl acetals.

TABLE B

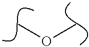

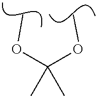

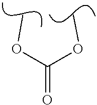

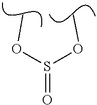

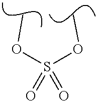

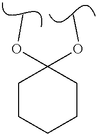

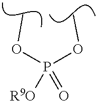

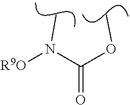

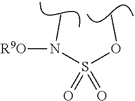

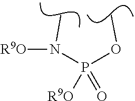

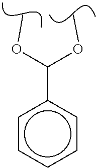

wherein R⁹ is C₁-C₆ alkyl.

### Amino Protecting Groups

Another set of protecting groups include any of the typical amino protecting groups described by Greene at pages 315-385. They include:
Carbamates: (methyl and ethyl, 9-fluorenylmethyl, 9(2-sulfo)fluorenylmethyl, 9-(2,7-dibromo)fluorenylmethyl, 2,7-di-t-butyl-[9-(10,10-dioxo-10,10,10,10-tetrahydrothioxanthyl)]methyl, 4-methoxyphenacyl);
Substituted Ethyl: (2,2,2-trichoroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridyl)ethyl, 2-(N,N-dicyclohexylcarboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxybenzyl, p-nitrobenzyl, p-bromobenzyl, p-chlorobenzyl, 2,4-dichlorobenzyl, 4-methylsulfinylbenzyl, 9-anthrylmethyl, diphenylmethyl);
Groups With Assisted Cleavage: (2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-choro-p-acyloxybenzyl, p-(dihydroxyboryl)benzyl, 5-benzisoxazolylmethyl, 2-(trifluoromethyl)-6-chromonylmethyl);
Groups Capable of Photolytic Cleavage: (m-nitrophenyl, 3,5-dimethoxybenzyl, o-nitrobenzyl, 3,4-dimethoxy-6-nitrobenzyl, phenyl(o-nitrophenyl)methyl); Urea-Type Derivatives (phenothiazinyl-(10)-carbonyl, N′-p-toluenesulfonylaminocarbonyl, N′-phenylaminothiocarbonyl);
Miscellaneous Carbamates: (t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethylcarboxamido)benzyl, 1,1-dimethyl-3-(N,N-dimethylcarboxamido)propyl, 1,1-dimethylpropynyl, di(2-pyridyl)methyl, 2-furanylmethyl, 2-Iodoethyl, Isobornyl, Isobutyl, Isonicotinyl, p-(p′-Methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, 2,4,6-trimethylbenzyl);
Amides: (N-formyl, N-acetyl, N-choroacetyl, N-trichoroacetyl, N-trifluoroacetyl, N-phenylacetyl, N-3-phenylpropionyl, N-picolinoyl, N-3-pyridylcarboxamide, N-benzoylphenylalanyl, N-benzoyl, N-p-phenylbenzoyl);
Amides With Assisted Cleavage: (N-o-nitrophenylacetyl, N-o-nitrophenoxyacetyl, N-acetoacetyl, (N′-dithiobenzyloxycarbonylamino)acetyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine, N-o-nitrobenzoyl, N-o-(benzoyloxymethyl)benzoyl, 4,5-diphenyl-3-oxazolin-2-one);
Cyclic Imide Derivatives: (N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1,3-dimethyl-1,3,5-triazacyclohexan-2-one, 5-substituted 1,3-dibenzyl-1,3-5-triazacyclohexan-2-one, 1-substituted 3,5-dinitro-4-pyridonyl);
N-Alkyl and N-Aryl Amines: (N-methyl, N-allyl, N-[2-(trimethylsilyl)ethoxy]methyl, N-3-acetoxypropyl, N-(1-isopropyl-4-nitro-2-oxo-3-pyrrolin-3-yl), Quaternary Ammonium Salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, N-2-picolylamine N-oxide);
Imine Derivatives: (N-1,1-dimethylthiomethylene, N-benzylidene, N-p-methoxybenylidene, N-diphenylmethylene, N-[(2-pyridyl)mesityl]methylene, N,(N′,N′-dimethylaminomethylene, N,N′-isopropylidene, N-p-nitrobenzylidene, N-salicylidene N-5-chlorosalicylidene, N-(5-chloro-2-hydroxyphenyl)phenylmethylene, N-cyclohexylidene);
Enamine Derivatives: (N-(5,5-dimethyl-3-oxo-1-cyclohexenyl));
N-Metal Derivatives (N-borane derivatives, N-diphenylborinic acid derivatives, N-[phenyl(pentacarbonylchromium- or -tungsten)]carbenyl, N-copper or N-zinc chelate);
N—N Derivatives: (N-nitro, N-nitroso, N-oxide);
N—P Derivatives: (N-diphenylphosphinyl, N-dimethylthiophosphinyl, N-diphenylthiophosphinyl, N-dialkyl phosphoryl, N-dibenzyl phosphoryl, N-diphenyl phosphoryl);
N—Si Derivatives, N—S Derivatives, and N-Sulfenyl Derivatives: (N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-3-nitropyridinesulfenyl); and N-sulfonyl Derivatives (N-p-toluenesulfonyl, N-benzenesulfonyl, N-2,3,6-trimethyl-4-methoxybenzenesulfonyl, N-2,4,6-trimethoxybenzenesulfonyl, N-2,6-dimethyl-4-methoxybenzenesulfonyl, N-pentamethylbenzenesulfonyl, N-2,3,5,6,-tetramethyl-4-methoxybenzenesulfonyl, N-4-methoxybenzenesulfonyl, N-2,4,6-trimethylbenzenesulfonyl, N-2,6-dimethoxy-4-methylbenzenesulfonyl, N-2,2,5,7,8-pentamethylchroman-6-sulfonyl, N-methanesulfonyl, N—O-trimethylsilyethanesulfonyl, N-9-anthracenesulfonyl, N-4-(4′,8′-dimethoxynaphthylmethyl)benzenesulfonyl, N-benzylsulfonyl, N-trifluoromethylsulfonyl, N-phenacylsulfonyl).

More typically, protected amino groups include carbamates and amides, still more typically, —NHC(O)R¹ or —N═CR¹N(R¹)₂. Another protecting group, also useful as a prodrug for amino or —NH(R⁵), is:

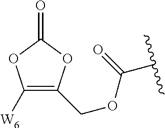

See for example Alexander, J. et al. (1996) J. Med. Chem. 39:480-486.

### Amino Acid and Polypeptide Protecting Group and Conjugates

An amino acid or polypeptide protecting group of a compound of the invention has the structure R¹⁵NHCH(R¹⁶)C(O)—, where R¹⁵ is H, an amino acid or polypeptide residue, or R⁵, and R¹⁶ is defined below.

R¹⁶ is lower alkyl or lower alkyl (C₁-C₆) substituted with amino, carboxyl, amide, carboxyl ester, hydroxyl, C₆-C₇ aryl, guanidinyl, imidazolyl, indolyl, sulfhydryl, sulfoxide, and/or alkylphosphate. R¹⁰ also is taken together with the amino acid a N to form a proline residue (R¹⁰═—CH₂)₃—). However, R¹⁰ is generally the side group of a naturally-occurring amino acid such as H, —CH₃, —CH(CH₃)₂, —CH₂—CH(CH₃)₂, —CHCH₃—CH₂—CH₃, —CH₂—C₆H₅, —CH₂CH₂—S—CH₃, —CH₂OH, —CH(OH)—CH₃, —CH₂—SH, —CH₂—C₆H₄OH, —CH₂—CO—NH₂, —CH₂—CH₂—CO—NH₂, —CH₂—COOH, —CH₂—CH₂—COOH, —(CH₂)₄—NH₂ and —(CH₂)₃—NH—C(NH₂)—NH₂. R₁₀ also includes 1-guanidinoprop-3-yl, benzyl, 4-hydroxybenzyl, imidazol-4-yl, indol-3-yl, methoxyphenyl and ethoxyphenyl.

Another set of protecting groups include the residue of an amino-containing compound, in particular an amino acid, a polypeptide, a protecting group, —NHSO₂R, NHC(O)R, —N(R)₂, NH₂ or —NH(R)(H), whereby for example a carboxylic acid is reacted, i.e. coupled, with the amine to form an amide, as in C(O)NR₂. A phosphonic acid may be reacted with the amine to form a phosphonamidate, as in —P(O)(OR)(NR₂).

In general, amino acids have the structure R¹⁷C(O)CH(R¹⁶)NH—, where R¹⁷ is —OH, —OR, an amino acid or a polypeptide residue. Amino acids are low molecular weight compounds, on the order of less than about 1000 MW and which contain at least one amino or imino group and at least one carboxyl group. Generally the amino acids will be found in nature, i.e., can be detected in biological material such as bacteria or other microbes, plants, animals or man. Suitable amino acids typically are alpha amino acids, i.e. compounds characterized by one amino or imino nitrogen atom separated from the carbon atom of one carboxyl group by a single substituted or unsubstituted alpha carbon atom. Of particular interest are hydrophobic residues such as mono-or di-alkyl or aryl amino acids, cycloalkylamino acids and the like. These residues contribute to cell permeability by increasing the partition coefficient of the parental drug. Typically, the residue does not contain a sulfhydryl or guanidino substituent.

Naturally-occurring amino acid residues are those residues found naturally in plants, animals or microbes, especially proteins thereof. Polypeptides most typically will be substantially composed of such naturally-occurring amino acid residues. These amino acids are glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine and hydroxyproline. Additionally, unnatural amino acids, for example, valanine, phenylglycine and homoarginine are also included. Commonly encountered amino acids that are not gene-encoded may also be used in the present invention. All of the amino acids used in the present invention may be either the D- or L-optical isomer. In addition, other peptidomimetics are also useful in the present invention. For a general review, see Spatola, A. F., in Chemistry and Biochemistry of Amino Acids, Peptides and Proteins, B. Weinstein, eds., Marcel Dekker, New York, p. 267 (1983).

When protecting groups are single amino acid residues or polypeptides they optionally are substituted at R³ of substituents A¹, A² or A³ in a compound of the invention. These conjugates are produced by forming an amide bond between a carboxyl group of the amino acid (or C-terminal amino acid of a polypeptide for example). Similarly, conjugates are formed between R³ and an amino group of an amino acid or polypeptide. Generally, only one of any site in the parental molecule is amidated with an amino acid as described herein, although it is within the scope of this invention to introduce amino acids at more than one permitted site. Usually, a carboxyl group of R³ is amidated with an amino acid. In general, the α-amino or α-carboxyl group of the amino acid or the terminal amino or carboxyl group of a polypeptide are bonded to the parental functionalities, i.e., carboxyl or amino groups in the amino acid side chains generally are not used to form the amide bonds with the parental compound (although these groups may need to be protected during synthesis of the conjugates as described further below).

With respect to the carboxyl-containing side chains of amino acids or polypeptides it will be understood that the carboxyl group optionally will be blocked, e.g., by R¹, esterified with R⁵ or amidated. Similarly, the amino side chains R¹⁶ optionally will be blocked with R¹ or substituted with R⁵.

Such ester or amide bonds with side chain amino or carboxyl groups, like the esters or amides with the parental molecule, optionally are hydrolyzable in vivo or in vitro under acidic (pH <3) or basic (pH >10) conditions.

Alternatively, they are substantially stable in the gastrointestinal tract of humans but are hydrolyzed enzymatically in blood or in intracellular environments. The esters or amino acid or polypeptide amidates also are useful as intermediates for the preparation of the parental molecule containing free amino or carboxyl groups. The free acid or base of the parental compound, for example, is readily formed from the esters or amino acid or polypeptide conjugates of this invention by conventional hydrolysis procedures.

When an amino acid residue contains one or more chiral centers, any of the D, L, meso, threo or erythro (as appropriate) racemates, scalemates or mixtures thereof may be used. In general, if the intermediates are to be hydrolyzed non-enzymatically (as would be the case where the amides are used as chemical intermediates for the free acids or free amines), D isomers are useful. On the other hand, L isomers are more versatile since they can be susceptible to both non-enzymatic and enzymatic hydrolysis, and are more efficiently transported by amino acid or dipeptidyl transport systems in the gastrointestinal tract.

Examples of suitable amino acids whose residues are represented by R^{x} or R^{y} include the following:

Glycine;

Aminopolycarboxylic acids, e.g., aspartic acid, β-hydroxyaspartic acid, glutamic acid, β-hydroxyglutamic acid, β-methylaspartic acid, β-methylglutamic acid, β,β-dimethylaspartic acid, γ-hydroxyglutamic acid, β,γ-dihydroxyglutamic acid, β-phenylglutamic acid, β-methyleneglutamic acid, 3-aminoadipic acid, 2-aminopimelic acid, 2-aminosuberic acid and 2-aminosebacic acid;

Amino acid amides such as glutamine and asparagine;

Polyamino- or polybasic-monocarboxylic acids such as arginine, lysine, β-aminoalanine, γ-aminobutyrine, ornithine, citruline, homoarginine, homocitrulline, hydroxylysine, allohydroxylsine and diaminobutyric acid;

Other basic amino acid residues such as histidine;

Diaminodicarboxylic acids such as α,α′-diaminosuccinic acid, α, α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid;

Imino acids such as proline, hydroxyproline, allohydroxyproline, γ-methylproline, pipecolic acid, 5-hydroxypipecolic acid, and azetidine-2-carboxylic acid;

A mono- or di-alkyl (typically C₁-C₈ branched or normal) amino acid such as alanine, valine, leucine, allylglycine, butyrine, norvaline, norleucine, heptyline, α-methylserine, α-amino-α-methyl-γ-hydroxyvaleric acid, α-amino-α-methyl-δ-hydroxyvaleric acid, α-amino-α-methyl-ε-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, α-aminodiisoamyacetic acid, α-methylaspartic acid, α-methylglutamic acid, 1-aminocyclopropane-1-carboxylic acid, isoleucine, alloisoleucine, tert-leucine, β-methyltryptophan and α-amino-β-ethyl-β-phenylpropionic acid;

β-phenylserinyl;

Aliphatic α-amino-β-hydroxy acids such as serine, β-hydroxyleucine, β-hydroxynorleucine, β-hydroxynorvaline, and α-amino-β-hydroxystearic acid;

α-Amino, α-, γ-, δ- or ε-hydroxy acids such as homoserine, δ-hydroxynorvaline, γ-hydroxynorvaline and ε-hydroxynorleucine residues; canavine and canaline; γ-hydroxyornithine;

2-hexosaminic acids such as D-glucosaminic acid or D-galactosaminic acid;

α-Amino-β-thiols such as penicillamine, β-thiolnorvaline or β-thiolbutyrine;

Other sulfur containing amino acid residues including cysteine; homocystine, β-phenylmethionine, methionine, S-allyl-L-cysteine sulfoxide, 2-thiolhistidine, cystathionine, and thiol ethers of cysteine or homocysteine;

Phenylalanine, tryptophan and ring-substituted α-amino acids such as the phenyl- or cyclohexylamino acids α-aminophenylacetic acid, α-aminocyclohexylacetic acid and α-amino-β-cyclohexylpropionic acid; phenylalanine analogues and derivatives comprising aryl, lower alkyl, hydroxy, guanidino, oxyalkylether, nitro, sulfur or halo-substituted phenyl (e.g., tyrosine, methyltyrosine and o-chloro-, p-chloro-, 3,4-dichloro, o-, in- or p-methyl-, 2,4,6-trimethyl-, 2-ethoxy-5-nitro-, 2-hydroxy-5-nitro- and p-nitro-phenylalanine); furyl-, thienyl-, pyridyl-, pyrimidinyl-, purinyl- or naphthyl-alanines; and tryptophan analogues and derivatives including kynurenine, 3-hydroxykynurenine, 2-hydroxytryptophan and 4-carboxytryptophan;

α-Amino substituted amino acids including sarcosine (N-methylglycine), N-benzylglycine, N-methylalanine, N-benzylalanine, N-methylphenylalanine, N-benzylphenylalanine, N-methylvaline and N-benzylvaline; and

α-Hydroxy and substituted α-hydroxy amino acids including serine, threonine, allothreonine, phosphoserine and phosphothreonine.

Polypeptides are polymers of amino acids in which a carboxyl group of one amino acid monomer is bonded to an amino or imino group of the next amino acid monomer by an amide bond. Polypeptides include dipeptides, low molecular weight polypeptides (about 1500-5000 MW) and proteins. Proteins optionally contain 3, 5, 10, 50, 75, 100 or more residues, and suitably are substantially sequence-homologous with human, animal, plant or microbial proteins. They include enzymes (e.g., hydrogen peroxidase) as well as immunogens such as KLH, or antibodies or proteins of any type against which one wishes to raise an immune response. The nature and identity of the polypeptide may vary widely.

The polypeptide amidates are useful as immunogens in raising antibodies against either the polypeptide (if it is not immunogenic in the animal to which it is administered) or against the epitopes on the remainder of the compound of this invention.

Antibodies capable of binding to the parental non-peptidyl compound are used to separate the parental compound from mixtures, for example in diagnosis or manufacturing of the parental compound. The conjugates of parental compound and polypeptide generally are more immunogenic than the polypeptides in closely homologous animals, and therefore make the polypeptide more immunogenic for facilitating raising antibodies against it. Accordingly, the polypeptide or protein may not need to be immunogenic in an animal typically used to raise antibodies, e.g., rabbit, mouse, horse, or rat, but the final product conjugate should be immunogenic in at least one of such animals. The polypeptide optionally contains a peptidolytic enzyme cleavage site at the peptide bond between the first and second residues adjacent to the acidic heteroatom. Such cleavage sites are flanked by enzymatic recognition structures, e.g., a particular sequence of residues recognized by a peptidolytic enzyme.

Peptidolytic enzymes for cleaving the polypeptide conjugates of this invention are well known, and in particular include carboxypeptidases. Carboxypeptidases digest polypeptides by removing C-terminal residues, and are specific in many instances for particular C-terminal sequences. Such enzymes and their substrate requirements in general are well known. For example, a dipeptide (having a given pair of residues and a free carboxyl terminus) is covalently bonded through its α-amino group to the phosphorus or carbon atoms of the compounds herein. In claims where W₁ is phosphonate it is expected that this peptide will be cleaved by the appropriate peptidolytic enzyme, leaving the carboxyl of the proximal amino acid residue to autocatalytically cleave the phosphonoamidate bond.

Suitable dipeptidyl groups (designated by their single letter code) are AA, AR, AN, AD, AC, AE, AQ, AG, AH, AI, AL, AK, AM, AF, AP, AS, AT, AW, AY, AV, RA, RR, RN, RD, RC, RE, RQ, RG, RH, RI, RL, RK, RM, RF, RP, RS, RT, RW, RY, RV, NA, NR, NN, ND, NC, NE, NQ, NG, NH, NI, NL, NK, NM, NF, NP, NS, NT, NW, NY, NV, DA, DR, DN, DD, DC, DE, DQ, DG, DH, DI, DL, DK, DM, DF, DP, DS, DT, DW, DY, DV, CA, CR, CN, CD, CC, CE, CQ, CG, CH, CI, CL, CK, CM, CF, CP, CS, CT, CW, CY, CV, EA, ER, EN, ED, EC, EE, EQ, EG, EH, EI, EL, EK, EM, EF, EP, ES, ET, EW, EY, EV, QA, QR, QN, QD, QC, QE, QQ, QG, QH, QI, QL, QK, QM, QF, QP, QS, QT, QW, QY, QV, GA, GR, GN, GD, GC, GE, GQ, GG, GH, GI, GL, GK, GM, GF, GP, GS, GT, GW, GY, GV, HA, HR, HN, HD, HC, HE, HQ, HG, HH, HI, HL, HK, HM, HF, HP, HS, HT, HW, HY, HV, IA, IR, IN, ID, IC, IE, IQ, IG, IH, II, IL, IK, IM, IF, IP, IS, IT, IW, IY, IV, LA, LR, LN, LD, LC, LE, LQ, LG, LH, LI, LL, LK, LM, LF, LP, LS, LT, LW, LY, LV, KA, KR, KN, KD, KC, KE, KQ, KG, KH, KI, KL, KK, KM, KF, KP, KS, KT, KW, KY, KV, MA, MR, MN, MD, MC, ME, MQ, MG, MH, MI, ML, MK, MM, MF, MP, MS, MT, MW, MY, MV, FA, FR, FN, FD, FC, FE, FQ, FG, FH, FI, FL, FK, FM, FF, FP, FS, FT, FW, FY, FV, PA, PR, PN, PD, PC, PE, PQ, PG, PH, PI, PL, PK, PM, PF, PP, PS, PT, PW, PY, PV, SA, SR, SN, SD, SC, SE, SQ, SG, SH, SI, SL, SK, SM, SF, SP, SS, ST, SW, SY, SV, TA, TR, TN, TD, TC, TE, TQ, TG, TH, TI, TL, TK, TM, TF, TP, TS, TT, TW, TY, TV, WA, WR, WN, WD, WC, WE, WQ, WG, WH, WI, WL, WK, WM, WF, WP, WS, WT, WW, WY, WV, YA, YR, YN, YD, YC, YE, YQ, YG, YH, YI, YL, YK, YM, YF, YP, YS, YT, YW, YY, YV, VA, VR, VN, VD, VC, VE, VQ, VG, VH, VI, VL, VK, VM, VF, VP, VS, VT, VW, VY and VV.

Tripeptide residues are also useful as protecting groups. When a phosphonate is to be protected, the sequence —X⁴-pro-X⁵— (where X⁴ is any amino acid residue and X⁵ is an amino acid residue, a carboxyl ester of proline, or hydrogen) will be cleaved by luminal carboxypeptidase to yield X⁴ with a free carboxyl, which in turn is expected to autocatalytically cleave the phosphonoamidate bond. The carboxy group of X⁵ optionally is esterified with benzyl.

Dipeptide or tripeptide species can be selected on the basis of known transport properties and/or susceptibility to peptidases that can affect transport to intestinal mucosal or other cell types. Dipeptides and tripeptides lacking an α-amino group are transport substrates for the peptide transporter found in brush border membrane of intestinal mucosal cells (Bai, J. P. F., (1992) Pharm Res. 9:969-978). Transport competent peptides can thus be used to enhance bioavailability of the amidate compounds. Di- or tripeptides having one or more amino acids in the D configuration are also compatible with peptide transport and can be utilized in the amidate compounds of this invention. Amino acids in the D configuration can be used to reduce the susceptibility of a di- or tripeptide to hydrolysis by proteases common to the brush border such as aminopeptidase N. In addition, di- or tripeptides alternatively are selected on the basis of their relative resistance to hydrolysis by proteases found in the lumen of the intestine. For example, tripeptides or polypeptides lacking asp and/or glu are poor substrates for aminopeptidase A, di- or tripeptides lacking amino acid residues on the N-terminal side of hydrophobic amino acids (leu, tyr, phe, val, trp) are poor substrates for endopeptidase, and peptides lacking a pro residue at the penultimate position at a free carboxyl terminus are poor substrates for carboxypeptidase P. Similar considerations can also be applied to the selection of peptides that are either relatively resistant or relatively susceptible to hydrolysis by cytosolic, renal, hepatic, serum or other peptidases. Such poorly cleaved polypeptide amidates are immunogens or are useful for bonding to proteins in order to prepare immunogens.

### Specific Embodiments of the Invention

Specific values described for radicals, substituents, and ranges, as well as specific embodiments of the invention described herein, are for illustration only; they do not exclude other defined values or other values within defined ranges.

In one specific embodiment of the invention, the conjugate is a compound that is substituted with one or more phosphonate groups either directly or indirectly through a linker; and that is optionally substituted with one or more groups A⁰; or a pharmaceutically acceptable salt thereof, wherein:

A⁰ is A¹, A² or W³;

A¹ is:

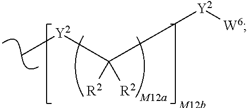

A² is:

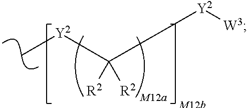

A³ is:

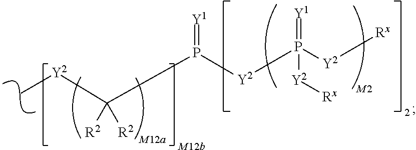

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—;

R^{x} is independently H, R¹, W³, a protecting group, or the formula:

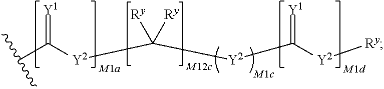

wherein:

R^{y} is independently H, W³, R² or a protecting group;

R¹ is independently H or alkyl of 1 to 18 carbon atoms;

R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1; and

M12c is 0, 1, 2, 3, 4, 5, 6, 7, 8, 9, 10, 11 or 12.

In another specific embodiment of the invention A¹ is of the formula:

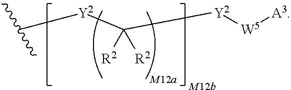

In another specific embodiment of the invention A¹ is of the formula:

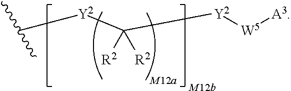

In another specific embodiment of the invention A¹ is of the formula:

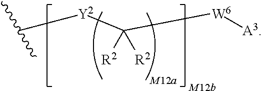

In another specific embodiment of the invention A¹ is of the formula:

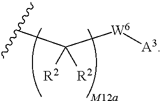

In another specific embodiment of the invention A¹ is of the formula:

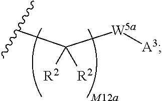

and W^{5a} is a carbocycle or a heterocycle where W^{5a} is independently substituted with 0 or 1 R² groups. A specific value for M12a is 1.

In another specific embodiment of the invention A¹ is of the formula:

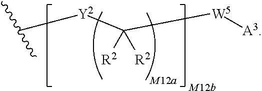

In another specific embodiment of the invention A¹ is of the formula:

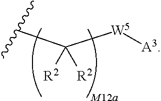

In another specific embodiment of the invention A¹ is of the formula:

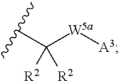

wherein W^{5a} is a carbocycle independently substituted with 0 or 1 R² groups.

In another specific embodiment of the invention A¹ is of the formula:

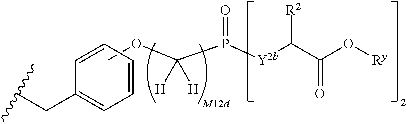

wherein Y^{2b} is O or N(R²); and M12d is 1, 2, 3, 4, 5, 6, 7 or 8.

In another specific embodiment of the invention A¹ is of the formula:

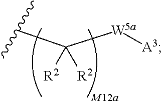

wherein W^{5a} is a carbocycle independently substituted with 0 or 1 R² groups;

In another specific embodiment of the invention A¹ is of the formula:

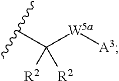

wherein W^{5a} is a carbocycle or heterocycle where W^{5a} is independently substituted with 0 or 1 R² groups.

In another specific embodiment of the invention A¹ is of the formula:

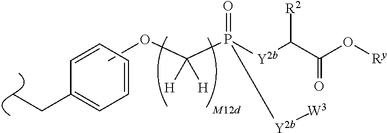

wherein Y^{2b} is O or N(R²); and M12d is 1, 2, 3, 4, 5, 6, 7 or 8.

In a specific embodiment of the invention A² is of the formula:

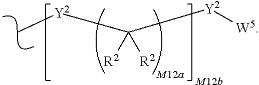

In another specific embodiment of the invention A² is of the formula:

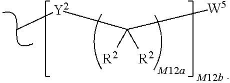

In another specific embodiment of the invention M12b is 1.

In another specific embodiment of the invention e M12b is 0, Y² is a bond and W⁵ is a carbocycle or heterocycle where W⁵ is optionally and independently substituted with 1, 2, or 3 R² groups.

In another specific embodiment of the invention A² is of the formula:

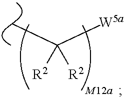

wherein W^{5a} is a carbocycle or heterocycle where W^{5a} is optionally and independently substituted with 1, 2, or 3 R² groups.

In another specific embodiment of the invention M12a is 1.

In another specific embodiment of the invention A² is selected from phenyl, substituted phenyl, benzyl, substituted benzyl, pyridyl and substituted pyridyl.

In another specific embodiment of the invention A² is of the formula:

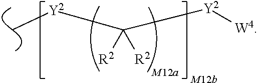

In another specific embodiment of the invention A² is of the formula:

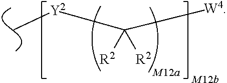

In another specific embodiment of the invention M12b is 1.

In a specific embodiment of the invention A³ is of the formula:

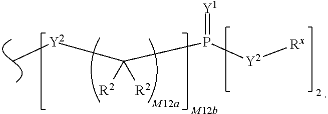

In another specific embodiment of the invention A³ is of the formula:

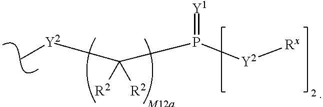

In another specific embodiment of the invention A³ is of the formula:

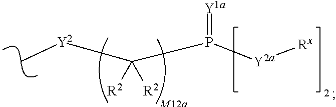

wherein Y^{1a} is O or S; and Y^{2a} is O, N(R^{x}) or S.

In another specific embodiment of the invention A³ is of the formula:

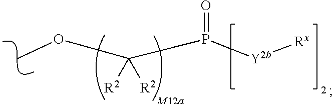

wherein Y^{2b} is O or N(R^{x}).

In another specific embodiment of the invention A³ is of the formula:

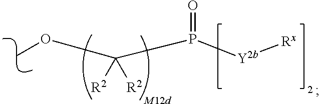

wherein Y^{2b} is O or N(R^{x}); and M12d is 1, 2, 3, 4, 5, 6, 7 or 8.

In another specific embodiment of the invention A³ is of the formula:

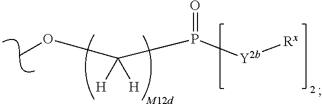

wherein Y^{2b} is O or N(R^{x}); and M12d is 1, 2, 3, 4, 5, 6, 7 or 8.

In another specific embodiment of the invention M12d is 1.

In another specific embodiment of the invention A³ is of the formula:

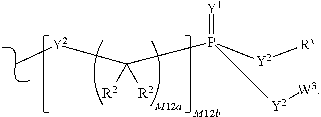

In another specific embodiment of the invention A³ is of the formula:

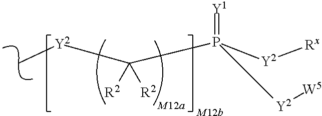

In another specific embodiment of the invention W⁵ is a carbocycle.

In another specific embodiment of the invention A³ is of the formula:

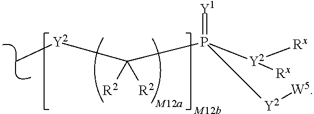

In another specific embodiment of the invention W⁵ is phenyl.

In another specific embodiment of the invention A³ is of the formula:

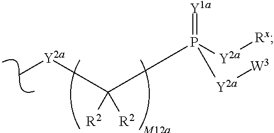

wherein Y^{1a} is O or S; and Y^{2a} is O, N(R^{x}) or S.

In another specific embodiment of the invention A³ is of the formula:

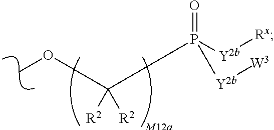

wherein Y^{2b} is O or N(R^{x}).

In another specific embodiment of the invention A³ is of the formula:

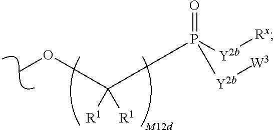

wherein Y^{2b} is O or N(R^{x}); and M12d is 1, 2, 3, 4, 5, 6, 7 or 8.

In another specific embodiment of the invention R¹ is H.

In another specific embodiment of the invention A³ is of the formula:

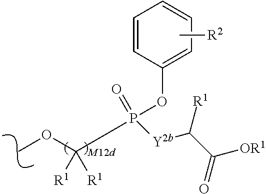

wherein the phenyl carbocycle is substituted with 0, 1, 2, or 3 R² groups.

In another specific embodiment of the invention A³ is of the formula:

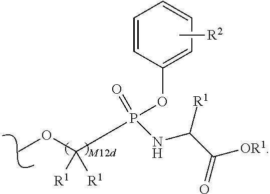

In another specific embodiment of the invention A³ is of the formula:

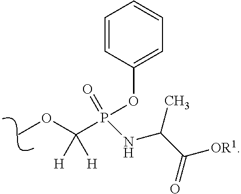

In another specific embodiment of the invention A³ is of the formula:

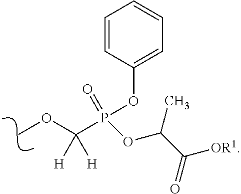

In another specific embodiment of the invention A³ is of the formula:

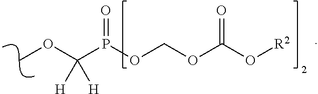

In another specific embodiment of the invention A³ is of the formula:

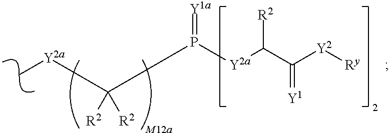

wherein Y^{1a} is O or S; and Y^{2a} is O, N(R²) or S.

In another specific embodiment of the invention A³ is of the formula:

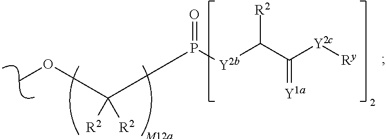

wherein Y^{1a} is O or S; Y^{2b} is O or N(R²); and Y^{2c} is O, N(R^{y}) or S.

In another specific embodiment of the invention A³ is of the formula:

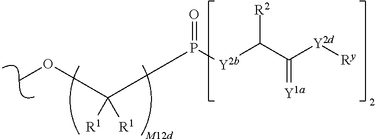

wherein Y^{1a} is O or S; Y^{2b} is O or N(R²); Y^{2d} is O or N(R^{y}); and M12d is 1, 2, 3, 4, 5, 6, 7 or 8.

In another specific embodiment of the invention A³ is of the formula:

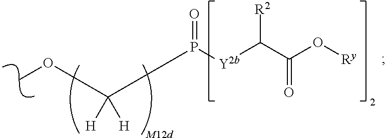

wherein Y^{2b} is O or N(R²); and M12d is 1, 2, 3, 4, 5, 6, 7 or 8.

In another specific embodiment of the invention A³ is of the formula:

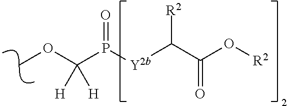

wherein Y^{2b} is O or N(R²).

In another specific embodiment of the invention A³ is of the formula:

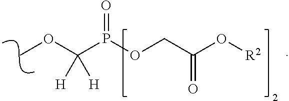

In another specific embodiment of the invention A³ is of the formula:

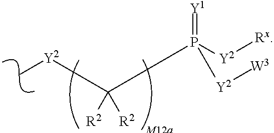

In another specific embodiment of the invention A³ is of the formula:

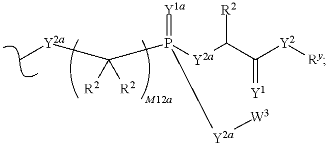

wherein Y^{1a} is O or S; and Y^{2a} is O, N(R²) or S.

In another specific embodiment of the invention A³ is of the formula:

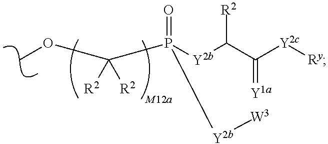

wherein Y^{1a} is O or S; Y^{2b} is O or N(R²); and Y^{2c} is O, N(R^{y}) or S.

In another specific embodiment of the invention A³ is of the formula:

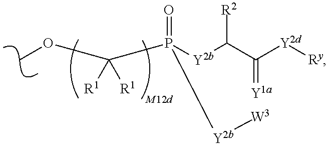

wherein Y^{1a} is O or S; Y^{2b} is O or N(R²); Y^{2d} is O or N(R^{y}); and M12d is 1, 2, 3, 4, 5, 6, 7 or 8.

In another specific embodiment of the invention A³ is of the formula:

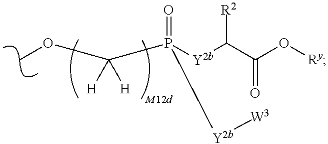

wherein Y^{2b} is O or N(R²); and M12d is 1, 2, 3, 4, 5, 6, 7 or 8.

In another specific embodiment of the invention A³ is of the formula:

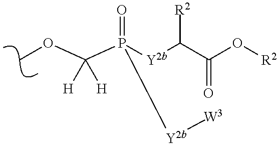

wherein Y^{2b} is O or N(R²).

In another specific embodiment of the invention A³ is of the formula:

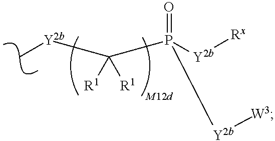

wherein: Y^{2b} is O or N(R^{x}); and M12d is 1, 2, 3, 4, 5, 6, 7 or 8.

In another specific embodiment of the invention A³ is of the formula:

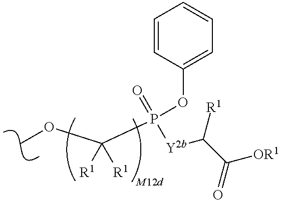

wherein the phenyl carbocycle is substituted with 0, 1, 2, or 3 R² groups.

In another specific embodiment of the invention A³ is of the formula:

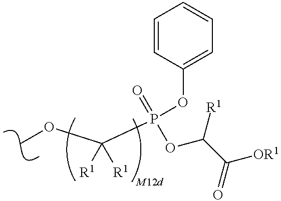

wherein the phenyl carbocycle is substituted with 0, 1, 2, or 3 R² groups.

In another specific embodiment of the invention A³ is of the formula:

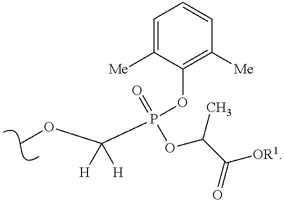

In a specific embodiment of the invention A⁰ is of the formula:

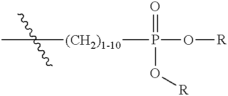

wherein each R is independently (C₁-C₆)alkyl.

In a specific embodiment of the invention R^{x} is independently H, R¹, W³, a protecting group, or the formula:

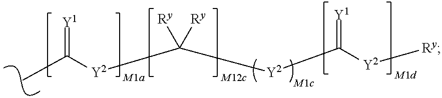

wherein:

R^{y} is independently H, W³, R² or a protecting group;

R¹ is independently H or alkyl of 1 to 18 carbon atoms;

R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups.

In a specific embodiment of the invention R^{x} is of the formula:

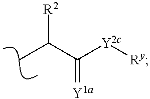

wherein Y^{1a} is O or S; and Y^{2c} is O, N(R^{y}) or S.

In a specific embodiment of the invention R^{x} is of the formula:

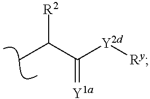

wherein Y^{1a} is O or S; and Y^{2d} is O or N(R^{y}).

In a specific embodiment of the invention R^{x} is of the formula:

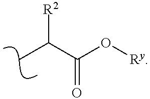

In a specific embodiment of the invention R^{y} is hydrogen or alkyl of 1 to 10 carbons.

In a specific embodiment of the invention R^{x} is of the formula:

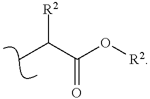

In a specific embodiment of the invention R^{x} is of the formula:

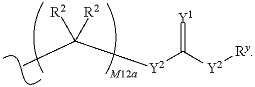

In a specific embodiment of the invention R^{x} is of the formula:

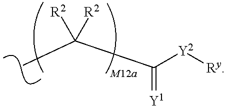

In a specific embodiment of the invention Y¹ is O or S.

In a specific embodiment of the invention Y² is O, N(R^{y}) or S.

In one specific embodiment of the invention R^{x} is a group of the formula:

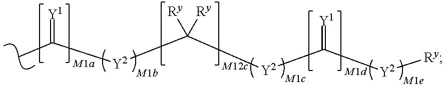

wherein:

m1a, m1b, m1c, m1d and m1e are independently 0 or 1;

m12c is 0, 1, 2, 3, 4, 5, 6, 7, 8, 9, 10, 11 or 12;

R^{y} is H, W³, R² or a protecting group;

provided that:

if m1a, m12c, and m1d are 0, then m1b, m1c and m1e are 0;

if m1a and m12c are 0 and m1d is not 0, then m1b and m1c are 0;

if m1a and m1d are 0 and m12c is not 0, then m1b and at least one of m1c and m1e are 0;

if m1a is 0 and m12c and m1d are not 0, then m1b is 0;

if m12c and m1d are 0 and m1a is not 0, then at least two of m1b, m1c and m1e are 0;

if m12c is 0 and m1a and m1d are not 0, then at least one of m1b and m1c are 0; and

if m1d is 0 and m1a and m12c are not 0, then at least one of m1c and m1e are 0.

In another specific embodiment, the invention provides a compound of the formula:

[DRUG]-(A⁰ )ₙₙ

or a pharmaceutically acceptable salt thereof wherein,

DRUG is a compound of any one of formulae 200-247

nn is 1, 2, or 3;

A⁰ is A¹, A² or W³ with the proviso that the compound includes at least one A¹;

A¹ is:

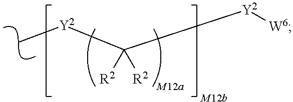

A² is:

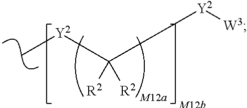

A³ is:

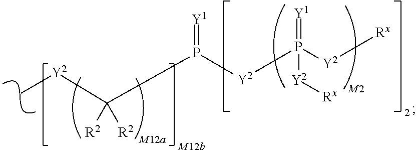

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—;

R^{x} is independently H, R¹, W³, a protecting group, or the formula:

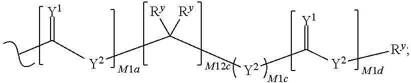

wherein:

R^{y} is independently H, W³, R² or a protecting group;

R¹ is independently H or alkyl of 1 to 18 carbon atoms;

R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1;

M12c is 0, 1, 2, 3, 4, 5, 6, 7, 8, 9, 10, 11 or 12;

X¹⁴⁹ is thymine, adenine, uracil, a 5-halouracil, a 5-alkyluracil guanine, cytosine, a 5-halocytosine, 5-alkylcytosine, or 2,6-diaminopurine;

X¹⁵⁰ is OH, Cl, NH₂, H, Me, or MeO;

X¹⁵¹ is H, NH₂, or NH-alkyl;

X¹⁵² and X¹⁵³ are independently H, alkyl, or cyclopropyl; and

X¹⁵⁴ is thymine, adenine, guanine, cytosine, uracil, inosine, or diaminopurine.

The present invention also provides a compound of any one of formulae 1-71 wherein:

A⁰ is A¹;

A¹ is:

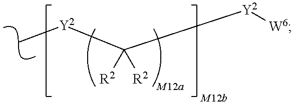

A³ is:

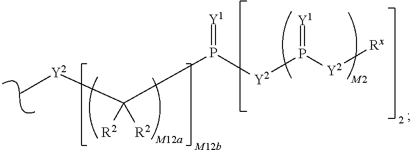

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—;

R^{x} is independently H, W³, a protecting group, or the formula:

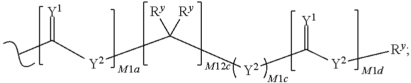

R^{y} is independently H, W³, R² or a protecting group;

R¹ is independently H or alkyl of 1 to 18 carbon atoms;

R² is independently H, R³ or R⁴ wherein each R⁴ is independently substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1; and

M12c is 0, 1, 2, 3, 4, 5, 6, 7, 8, 9, 10, 11 or 12.

X⁵² is C₁-C₆ alkyl or C₇-C₁₀ arylalkyl group;

X⁵³ is H, alkyl or substituted alkyl;

X⁵⁴ is CH or N;

X⁵⁵ is thymine, adenine, uracil, a 5-halouracil, a 5-alkyluracil, guanine, cytosine, a 5-halo cytosine, a 5-alkyl cytosine, or 2,6-diaminopurine;

X⁵⁷ is H or F;

X⁵⁸ is OH, Cl, NH₂, H, Me, or MeO;

X⁵⁹ is H or NH₂;

X⁶⁰ is OH, Cl, NH₂, or H;

X⁶¹ is H, NH₂, or NH-alkyl;

X⁶² and X⁶³ are independently H, alkyl, or cyclopropyl;

X⁶⁷ is O or NH;

X⁶⁸ is H, acetate, benzyl, benzyloxycarbonyl, or an amino protecting group;

X⁸² is OH, F, or cyano;

X⁸³ is N or CH;

X⁸⁴ is a cis-hydrogen or a trans-hydrogen;

X⁸⁶ is H, methyl, hydroxymethyl, or fluoromethyl;

X⁸⁷ and X⁸⁸ are each independently H or C₁₋₄ alkyl, which alkyl is optionally substituted with OH, amino, C₁₋₄ alkoxy, C₁₋₄ alkylthio, or one to three halogen atoms;

X⁸⁹ is —O— or —S(O)n-, where n is 0, 1, or 2;

X⁹⁰ is H, methyl, hydroxymethyl, or fluoromethyl;

X⁹¹ is H hydroxy, alkyl, azido, cyano, alkenyl, alkynyl, bromovinyl, —C(O)O(alkyl), —O(acyl), alkoxy, alkenyloxy, chloro, bromo, fluoro, iodo, NO₂, NH₂, —NH(lower alkyl), —NH(acyl), —N(lower alkyl)₂, —N(acyl)₂;

X⁹² is H, C₂₋₄alkenyl, C₂₋₄alkynyl, or C₁₋₄ alkyl optionally substituted with amino, hydroxy, or 1 to 3 fluorine atoms;

one of X⁹³ and X⁹⁴ is hydroxy or C₁₋₄ alkoxy and the other of X⁹³ and X⁹⁴ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

X⁹³ is H, C₂₋₄ alkenyl, C₂₋₄ alkynyl, or C₁₋₄ alkyl optionally substituted with amino, hydroxy, or 1 to 3 fluorine atoms, and one of X⁹² and X⁹⁴ is hydroxy or C₁₋₄alkoxy and the other of X⁹² and X⁹⁴ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

X⁹² and X⁹³ together with the carbon atom to which they are attached form a 3- to 6 membered saturated monocyclic ring system optionally containing a heteroatom selected from O, S, and NC₀₋₄ alkyl;

X⁹⁵ is H, OH, SH, NH₂, C₁₋₄ alkylamino, di(C₁₋₄alkyl)amino, C₃₋₆cycloalkylamino, halo, C₁₋₄alkyl, C₁₋₄ alkoxy, or CF₃; or X⁹² and X⁹⁵ can optionally together be a bond linking the two carbons to which they are attached;

X⁹⁶ is H, methyl, hydroxymethyl, or fluoromethyl;

X⁹⁷ is selected from the group consisting of

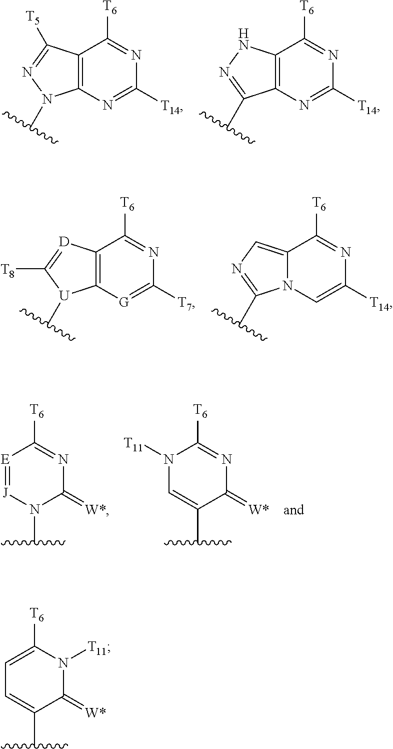

U, G, and J are each independently CH or N;

D is N, CH, C—CN, C—NO₂, C—C₁₋₃ alkyl, C—NHCONH₂, C—CONT₁₁T₁₁, C—CSNT₁₁T₁₁, C—COOT₁₁, C—C(═NH)NH₂, C-hydroxy, C—C₁₋₃alkoxy, C-amino, C—C₁₋₄ alkylamino, C-di(C₁₋₄alkyl)amino, C-halogen, C-(1,3-oxazol-2-yl), C-(1,3 thiazol-2-yl), or C-(imidazol-2-yl); wherein alkyl is unsubstituted or substituted with one to three groups independently selected from halogen, amino, hydroxy, carboxy, and C₁₋₃ alkoxy;

E is N or CT₅;

W^{a} is O or S;

T₁ is H, C₂₋₄alkenyl, C₂₋₄alkynyl, or C₁₋₄alkyl optionally substituted with amino, hydroxy, or 1 to 3 fluorine atoms and one of T₂ and T₃ is hydroxy or C₁₋₄ alkoxy and the other of T₂ and T₃ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

T₂ is H, C₂₋₄alkenyl, C₂₋₄alkynyl, or C₁₋₄alkyl optionally substituted with amino, hydroxy, or 1 to 3 fluorine atoms and one of T₁ and T₃ is hydroxy or C₁₋₄alkoxy and the other of T₁ and T₃ is selected from the group consisting of H; hydroxy; halo; C₁₋₄ alkyl optionally substituted with 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄alkylthio; C₁₋₈ alkylcarbonyloxy; aryloxycarbonyl; azido; amino; C₁₋₄ alkylamino; and di(C₁₋₄ alkyl)amino; or

T₁ and T₂ together with the carbon atom to which they are attached form a 3- to 6 membered saturated monocyclic ring system optionally containing a heteroatom selected from O, S, and NC₀₋₄ alkyl;

T₄ and T₆ are each independently H, OH, SH, NH₂, C₁₋₄ alkylamino, di(C₁₋₄ alkyl)amino, C₃₋₆ cycloalkylamino, halo, C₁₋₄ alkyl, C₁₋₄ alkoxy, or CF₃;

T₅ is H, C₁₋₆alkyl, C₂₋₆alkenyl, C₂₋₆alkynyl, C₁₋₄alkylamino, CF₃, or halogen; T₁₄ is H, CF₃, C₁₋₄ alkyl, amino, C₁₋₄alkylamino, C₃₋₆cycloalkylamino, or di(C₁₋₄alkyl)amino;

T₇ is H, amino, C₁₋₄alkylamino, C₃₋₆ cycloalkylamino, or di(C₁₋₄alkyl)amino;

each T₁₁ is independently H or C₁₋₆ alkyl;

T₈ is H, halo, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

X¹⁰² is thymine, adenine, guanine, cytosine, uracil, inosine, or diaminopurine;

X¹⁰³ is OH, OR, NR₂, CN, NO₂, F, Cl, Br, or I;

X¹⁰⁴ is adenine, guanine, cytosine, uracil, thymine, 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, or pyrazolo[3,4-d]pyrimidine;

X¹⁰⁵ is guanine, cytosine, uracil, thymine;

X¹⁰⁶ is

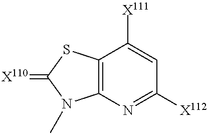

wherein X¹¹⁰ and X¹¹¹ are independently O or S and X¹¹² is H, amino, hydroxy,
or a halogen selected from Cl and Br;

X¹⁰⁷ and X¹⁰⁸ are independently selected from H or a C₁-C₁₈ acyl; and X¹⁰⁹ is H, a C₁-C₁₈ acyl, or

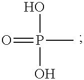

or X¹⁰⁷ is H and together X¹⁰⁸ and X¹⁰⁹ are

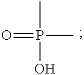

X¹¹³ is R³;

X¹¹⁴ is R⁴; and

X¹¹⁵ is R⁵.

In another specific embodiment, the invention provides a compound of the formula:

### [DRUG][L-P(═Y¹)—Y²—R^{x}]ₙₙ

or a pharmaceutically acceptable salt thereof wherein,

DRUG is a compound of any one of 200-247;

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—;

R^{x} is independently H. W³, a protecting group, or the formula:

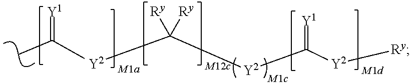

R^{y} is independently H, W³, R² or a protecting group;

R² is independently H, R³ or R⁴ wherein each R⁴ is independently substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

M2 is 1, 2, or 3;

M1a, M1c, and M1d are independently 0 or 1;

M12c is 0, 1, 2, 3, 4, 5, 6, 7, 8, 9, 10, 11 or 12;

nn is 1, 2, or 3;

L is a linking group;

X¹⁴⁹ is thymine, adenine, uracil, a 5-halouracil, a 5-alkyluracil, guanine, cytosine, a 5-halocytosine, 5-alkylcytosine, or 2,6-diaminopurine;

X¹⁵⁰ is OH, Cl, NH₂, H, Me, or MeO;

X¹⁵¹ is H, NH₂, or NH-alkyl;

X¹⁵² and X¹⁵³ are independently H, alkyl, or cyclopropyl; and

X¹⁵⁴ is thymine, adenine, guanine, cytosine, uracil, inosine, or diaminopurine.

In another specific embodiment, the invention provides a compound of which is a compound of the formula:)

[DRUG]-(A⁰)ₙₙ

or a pharmaceutically acceptable salt thereof wherein,

DRUG is a compound of any one of formulae 200-247;

nn is 1, 2, or 3;

A⁰ is A¹, A², or W³ with the proviso that the compound includes at least one A¹;

A¹ is:

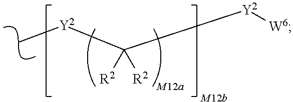

A² is:

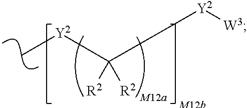

A³ is:

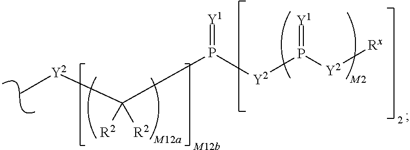

Y¹ is independently O, S, N(R^{x}), N(O)(R^{x}), N(OR^{x}), N(O)(OR^{x}), or N(N(R^{x})(R^{x}));

Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—;

R^{x} is independently H, W³, a protecting group, or the formula:

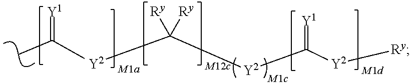

R^{y} is independently H, W³, R² or a protecting group;

R² is independently H, R³ or R⁴ wherein each R⁴ is independently substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y¹;

R^{3c} is —R^{x}, —N(R^{x})(R^{x}), —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x})(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));

R^{3d} is —C(Y¹)R^{x}, —C(Y¹)OR^{x} or —C(Y¹)(N(R^{x})(R^{x}));

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W⁶ is W³ independently substituted with 1, 2, or 3 A³ groups;

M2 is 0, 1 or 2;

M12a is 1, 2, 3, 4, 5, 6, 7, 8, 9, 10, 11 or 12;

M12b is 0, 1, 2, 3, 4, 5, 6, 7, 8, 9, 10, 11 or 12;

M1a, M1c, and M1d are independently 0 or 1;

M12c is 0, 1, 2, 3, 4, 5, 6, 7, 8, 9, 10, 11 or 12;

X¹⁴⁹ is thymine, adenine, uracil, a 5-halouracil, a 5-alkyluracil, guanine, cytosine, a 5-halocytosine, 5-alkylcytosine, or 2,6-diaminopurine;

X¹⁵⁰ is OH, Cl, NH₂, H, Me, or MeO;

X¹⁵¹ is H, NH₂, or NH-alkyl;

X¹⁵² and X¹⁵³ are independently H, alkyl, or cyclopropyl; and

X¹⁵⁴ is thymine, adenine, guanine, cytosine, uracil, inosine, or diaminopurine.

In compounds of the invention W⁵ carbocycles and W⁵ heterocycles may be independently substituted with 0 to 3 R² groups. W⁵ may be a saturated, unsaturated or aromatic ring comprising a mono- or bicyclic carbocycle or heterocycle. W⁵ may have 3 to 10 ring atoms, e.g., 3 to 7 ring atoms. The W⁵ rings are saturated when containing 3 ring atoms, saturated or mono-unsaturated when containing 4 ring atoms, saturated, or mono- or di-unsaturated when containing 5 ring atoms, and saturated, mono- or di-unsaturated, or aromatic when containing 6 ring atoms.

A W⁵ heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). W⁵ heterocyclic monocycles may have 3 to 6 ring atoms (2 to 5 carbon atoms and 1 to 2 heteroatoms selected from N, O, and S); or 5 or 6 ring atoms (3 to 5 carbon atoms and 1 to 2 heteroatoms selected from N and S). W⁵ heterocyclic bicycles have 7 to 10 ring atoms (6 to 9 carbon atoms and 1 to 2 heteroatoms selected from N, O, and S) arranged as a bicyclo[4,5], [5,5], [5,6], or [6,6] system; or 9 to 10 ring atoms (8 to 9 carbon atoms and 1 to 2 hetero atoms selected from N and S) arranged as a bicyclo[5,6] or [6,6] system. The W⁵ heterocycle may be bonded to Y² through a carbon, nitrogen, sulfur or other atom by a stable covalent bond.

W⁵ heterocycles include for example, pyridyl, dihydropyridyl isomers, piperidine, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl. W⁵ also includes, but is not limited to, examples such as:

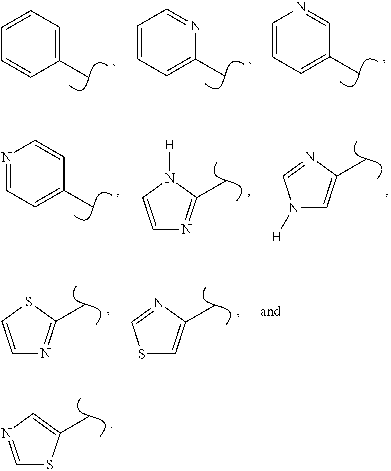

W⁵ carbocycles and heterocycles may be independently substituted with 0 to 3 R² groups, as defined above. For example, substituted W⁵ carbocycles include:

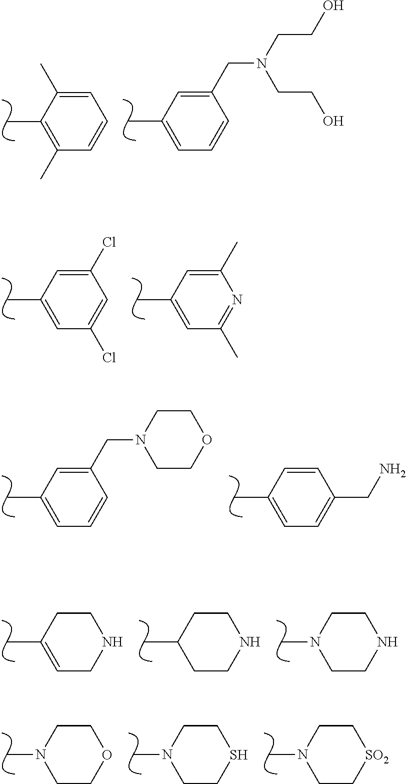

Examples of substituted phenyl carbocycles include:

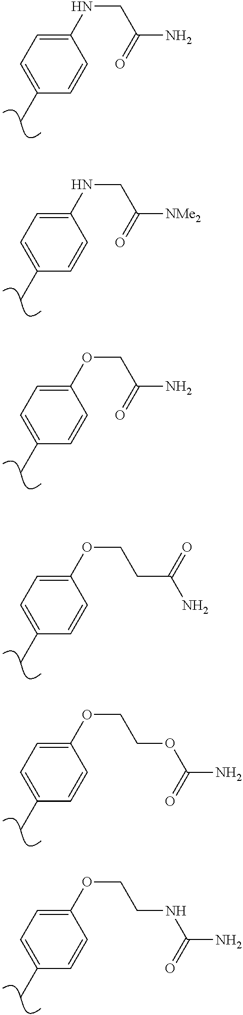

### Linking Groups and Linkers

The invention provides therapeutic compounds that are linked to one or more phosphonate groups either directly or through a linker. The nature of the linker is not critical provided it does not interfere with the ability of the phosphonate containing compound to function as a therapeutic agent. The phosphonate or the linker can be linked to the compound (e.g. a compound of Formula 200-247) at any synthetically feasible position on the compound by removing a hydrogen or any portion of the compound to provide an open valence for attachment of the phosphonate or the linker.

In one embodiment of the invention the linking group or linker (which can be designated “L”) can include all or a portions of the group A⁰, A¹, A², or W³ described herein.

In another embodiment of the invention the linking group or linker has a molecular weight of from about 20 daltons to about 400 daltons.

In another embodiment of the invention the linking group or linker has a length of about 5 angstroms to about 300 angstroms.

In another embodiment of the invention the linking group or linker separates the DRUG and a P(═Y¹) residue by about 5 angstroms to about 200 angstroms, inclusive, in length.

In another embodiment of the invention the linking group or linker is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the linking group or linker is of the formula W-A wherein A is (C₁-C₂₄)alkyl, (C₂-C₂₄)alkenyl, (C₂-C₂₄)alkynyl, (C₃-C₈)cycloalkyl, (C₆-C₁₀)aryl or a combination thereof, wherein W is —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

In another embodiment of the invention the linking group or linker is a divalent radical formed from a peptide.

In another embodiment of the invention the linking group or linker is a divalent radical formed from an amino acid.

In another embodiment of the invention the linking group or linker is a divalent radical formed from poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-lysine or poly-L-lysine-L-tyrosine.

In another embodiment of the invention the linking group or linker is of the formula W—(CH₂)ₙ wherein, n is between about 1 and about 10; and W is —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

In another embodiment of the invention the linking group or linker is methylene, ethylene, or propylene.

In another embodiment of the invention the linking group or linker is attached to the phosphonate group through a carbon atom of the linker.

### Nucleoside Analog Compounds

The conjugates of the invention include nucleoside analogs, e.g., compounds that inhibit DNA and/or RNA synthesis. For example, the conjugates of the invention include phosphonate containing analogs of LY-582563, L-Fd4C, L-FddC, telbivudine, clevudine, dOTCP, dOTC, DDL DDLP, ddcP, ddC, DADP, DAPD, d4TP, D4T, 3TC, 3TCP FTCP, ABCP, AZT, IsoddAP, FTC, ribavirin, viramidine, L-enantiomers of ribavirin and viramidine, levovirin, ISODD A, fosteabine, gemcitabine, cladribine, decitabine, entecavir, carbovir, abacavir, pentostatin, enocitabine, clofarabine, BCX-1777, ANA-245, and DADMe-IMMG. The conjugates of the inventions typically bare one or more (e.g. 1, 2, 3, or 4) phosphonate groups, which may be a prodrug moiety.

Typically, compounds of the invention have a molecular weight of from about 400 amu to about 10,000 amu; in a specific embodiment of the invention, compounds have a molecular weight of less than about 5000 amu; in another specific embodiment of the invention, compounds have a molecular weight of less than about 2500 amu; in another specific embodiment of the invention, compounds have a molecular weight of less than about 1000 amu; in another specific embodiment of the invention, compounds have a molecular weight of less than about 800 amu; in another specific embodiment of the invention, compounds have a molecular weight of less than about 600 amu; and in another specific embodiment of the invention, compounds have a molecular weight of less than about 600 amu and a molecular weight of greater than about 400 amu.

The compounds of the invention also typically have a log D (polarity) less than about 5. In one embodiment the invention provides compounds having a log D less than about 4; in another one embodiment the invention provides compounds having a log D less than about 3; in another one embodiment the invention provides compounds having a log D greater than about −5; in another one embodiment the invention provides compounds having a log D greater than about −3; and in another one embodiment the invention provides compounds having a log D greater than about 0 and less than about 3.

Selected substituents within the compounds of the invention are present to a recursive degree. In this context, “recursive substituent” means that a substituent may recite another instance of itself Because of the recursive nature of such substituents, theoretically, a large number may be present in any given claim. For example, R^{x} contains a R^{y} substituent. R^{y} can be R², which in turn can be R³. If R³ is selected to be R^{3c}, then a second instance of R^{x} can be selected. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

By way of example and not limitation, W³, R^{y} and R³ are all recursive substituents in certain claims. Typically, each of these may independently occur 20, 19, 18, 17, 16, 15, 14, 13, 12, 11, 10, 9, 8, 7, 6, 5, 4, 3, 2, 1, or 0, times in a given claim. More typically, each of these may independently occur 12 or fewer times in a given claim. More typically yet, W³ will occur 0 to 8 times, R^{y} will occur 0 to 6 times and R³ will occur 0 to 10 times in a given claim. Even more typically, W³ will occur 0 to 6 times, R^{y} will occur 0 to 4 times and R³ will occur 0 to 8 times in a given claim.

Recursive substituents are an intended aspect of the invention. One of ordinary skill in the art of medicinal chemistry understands the versatility of such substituents. To the degree that recursive substituents are present in an claim of the invention, the total number will be determined as set forth above.

Whenever a compound described herein is substituted with more than one of the same designated group, e.g., “R¹” or “R^{6a}”, then it will be understood that the groups may be the same or different, i.e., each group is independently selected. Wavy lines indicate the site of covalent bond attachments to the adjoining groups, moieties, or atoms.

The phosphonate group may be a phosphonate prodrug moiety. The prodrug moiety may be sensitive to hydrolysis, such as, but not limited to, a pivaloyloxymethyl carbonate (POC) or POM group. Alternatively, the prodrug moiety may be sensitive to enzymatic potentiated cleavage, such as a lactate ester or a phosphonamidate-ester group.

In one embodiment of the invention, the compound is in isolated and purified form. Generally, the term “isolated and purified” means that the compound is significantly free of biological materials (e.g. blood, cells, etc.). In one specific embodiment of the invention, the term means that the compound or conjugate of the invention is at least about 50% pure by weight in a mixture; in another specific embodiment, the term means that the compound or conjugate of the invention is at least about 75% pure by weight in a mixture; in another specific embodiment, the term means that the compound or conjugate of the invention is at least about 90% pure by weight in a mixture; in another specific embodiment, the term means that the compound or conjugate of the invention is at least about 98% pure by weight in a mixture; and in another embodiment, the term means that the compound or conjugate of the invention is at least about 99% pure by weight in a mixture. In another specific embodiment, the invention provides a compound or conjugate of the invention that has been synthetically prepared (e.g. prepared ex vivo).

In one embodiment the compound is not an anti-inflammatory compound; in another embodiment the compound is not an anti-infective; in another embodiment the compound is not a compound that is active against immune-mediated conditions; in another embodiment the compound is is not a compound that is active against metabolic diseases; in another embodiment the compound is not an antiviral agent; in another embodiment the compound is not a kinase inhibitor; in another embodiment the compound is not an IMPDH inhibitor; in another embodiment the compound is not an antimetabolite; in another embodiment the compound is not a PNP inhibitor; in another embodiment the compound is not an anti-cancer compound; in another embodiment the compound is not a substituted compound of formula 247; in another embodiment the compound is not a substituted compound of formula 242 or 246; in another embodiment the compound is not a substituted compound of any one of formulae 200, 237-242, and 246-247; in another embodiment the compound is not a substituted compound of any one of formulae 200, 236-238, 240-242, and 246.

### Intracellular Targeting

The phosphonate group of the compounds of the invention may cleave in vivo in stages after they have reached the desired site of action, i.e. inside a cell. One mechanism of action inside a cell may entail a first cleavage, e.g. by esterase, to provide a negatively-charged “locked-in” intermediate. Cleavage of a terminal ester grouping in a compound of the invention thus affords an unstable intermediate which releases a negatively charged “locked in” intermediate.

After passage inside a cell, intracellular enzymatic cleavage or modification of the phosphonate or prodrug compound may result in an intracellular accumulation of the cleaved or modified compound by a “trapping” mechanism. The cleaved or modified compound may then be “locked-in” the cell by a significant change in charge, polarity, or other physical property change which decreases the rate at which the cleaved or modified compound can exit the cell, relative to the rate at which it entered as the phosphonate prodrug. Other mechanisms by which a therapeutic effect are achieved may be operative as well. Enzymes which are capable of an enzymatic activation mechanism with the phosphonate prodrug compounds of the invention include, but are not limited to, amidases, esterases, microbial enzymes, phospholipases, cholinesterases, and phosphatases.

In selected instances in which the drug is of the nucleoside type, such as is the case of zidovudine and numerous other antiretroviral agents, it is known that the drug is activated in vivo by phosphorylation. Such activation may occur in the present system by enzymatic conversion of the “locked-in” intermediate with phosphokinase to the active phosphonate diphosphate and/or by phosphorylation of the drug itself after its release from the “locked-in” intermediate as described above. In either case, the original nucleoside-type drug will be convened, via the derivatives of this invention, to the active phosphorylated species.

From the foregoing, it will be apparent that many different drugs can be derivatized in accord with the present invention. Numerous such drugs are specifically mentioned herein. However, it should be understood that the discussion of drug families and their specific members for derivatization according to this invention is not intended to be exhaustive, but merely illustrative.

### Cellular Accumulation

In one embodiment, the invention is provides compounds capable of accumulating in human PBMC (peripheral blood mononuclear cells). PBMC refer to blood cells having round lymphocytes and monocytes. Physiologically, PBMC are critical components of the mechanism against infection. PBMC may be isolated from heparinized whole blood of normal healthy donors or buffy coats, by standard density gradient centrifugation and harvested from the interface, washed (e.g. phosphate-buffered saline) and stored in freezing medium. PBMC may be cultured in multi-well plates. At various times of culture, supernatant may be either removed for assessment, or cells may be harvested and analyzed (Smith R. et al (2003) Blood 102(7):2532-2540). The compounds of this claim may further comprise a phosphonate or phosphonate prodrug. More typically, the phosphonate or phosphonate prodrug can have the structure A³ as described herein.

Typically, compounds of the invention demonstrate improved intracellular half-life of the compounds or intracellular metabolites of the compounds in human PBMC when compared to analogs of the compounds not having the phosphonate or phosphonate prodrug. Typically, the half-life is improved by at least about 50%, more typically at least in the range 50-100%, still more typically at least about 100%, more typically yet greater than about 100%.

In one embodiment of the invention the intracellular half-life of a metabolite of the compound in human PBMCs is improved when compared to an analog of the compound not having the phosphonate or phosphonate prodrug. In such claims, the metabolite may be generated intracellularly, e.g. generated within human PBMC. The metabolite may be a product of the cleavage of a phosphonate prodrug within human PBMCs. The phosphonate prodrug may be cleaved to form a metabolite having at least one negative charge at physiological pH. The phosphonate prodrug may be enzymatically cleaved within human PBMC to form a phosphonate having at least one active hydrogen atom of the form P—OH.

### Stereoisomers

The compounds of the invention may have chiral centers, e.g., chiral carbon or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through well-known techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

The compounds of the invention can also exist as tautomeric isomers in certain cases. All though only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

### Salts and Hydrates

The compositions of this invention optionally comprise salts of the compounds herein, especially pharmaceutically acceptable non-toxic salts containing, for example, Na⁺, Li⁺, K⁺, Ca⁺² and Mg⁺². Such salts may include those derived by combination of appropriate cations such as alkali and alkaline earth metal ions or ammonium and quaternary amino ions with an acid anion moiety, typically a carboxylic acid. Monovalent salts are preferred if a water soluble salt is desired.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their un-ionized, as well as zwitterionic form, and combinations with stoichiometric amounts of water as in hydrates.

Also included within the scope of this invention are the salts of the parental compounds with one or more amino acids. Any of the amino acids described above are suitable, especially the naturally-occurring amino acids found as protein components, although the amino acid typically is one bearing a side chain with a basic or acidic group, e.g., lysine, arginine or glutamic acid, or a neutral group such as glycine, serine, threonine, alanine, isoleucine, or leucine.

Methods of Inhibition of DNA and/or RNA Synthesis

Another aspect of the invention relates to methods of inhibiting DNA and/or RNA synthesis, comprising the step of treating a sample with a composition of the invention.

Compositions of the invention may act as inhibitors of DNA and/or RNA synthesis, as intermediates for such inhibitors or have other utilities as described below. The inhibitors can bind to locations on the surface or in a cavity of a substrate, e.g., an enzyme, e.g., a reverse transcriptase or a viral polymerase. Compositions so binding may bind with varying degrees of reversibility. Those compounds binding substantially irreversibly are ideal candidates for use in this method of the invention. Once labeled, the substantially irreversibly binding compositions are useful as probes for the detection of DNA and/or RNA synthesis. Accordingly, the invention relates to methods of detecting DNA and/or RNA synthesis in a sample suspected of synthesizing DNA and/or RNA comprising the steps of: treating a sample suspected of synthesizing DNA and/or RNA with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl or amino.

Within the context of the invention samples include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of synthesizing DNA and/or RNA. Samples can be contained in any medium including water and organic solvent/water mixtures. Samples include living organisms such as humans, and man made materials such as cell cultures.

The treating step of the invention comprises adding the composition of the invention to the sample or it comprises adding a precursor of the composition to the sample. The addition step comprises any method of administration as described above.

If desired, the DNA and/or RNA synthesis after application of the composition can be observed by any method including direct and indirect methods of detecting such activity. Quantitative, qualitative, and semiquantitative methods of determining such activity are all contemplated. Typically one of the screening methods described above are applied, however, any other method such as observation of the physiological properties of a living organism are also applicable.

Many organisms suffer from cancer and viral infections. The compounds of this invention are useful in the treatment or prophylaxis of such conditions in animals or in man.

However, in screening compounds it should be kept in mind that the results of enzyme assays may not correlate with cell culture assays. Thus, a cell based assay should be the primary screening tool.

### Screens for Inhibitors

Compositions of the invention are screened for inhibitory activity, e.g., inhibition of DNA and/or RNA synthesis, by any of the conventional techniques for evaluating enzyme activity. Within the context of the invention, typically compositions are first screened for inhibition in vitro and compositions showing inhibitory activity are then screened for activity in vivo. Compositions having in vitro Ki (inhibitory constants) of less then about 5×10⁻⁶ M, typically less than about 1×10⁻⁷ M and preferably less than about 5×10⁻⁸ M are preferred for in vivo use.

Useful in vitro screens have been described in detail and will not be elaborated here.

### Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of cancer or viral infections.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Combination Therapy

Active ingredients of the invention are also used in combination with other active ingredients. Such combinations are selected based on the condition to be treated, cross-reactivities of ingredients and pharmaco-properties of the combination.

It is also possible to combine any compound of the invention with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

### Metabolites of the Compounds of the Invention

Also falling within the scope of this invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled (e.g., C¹⁴ or H³) compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no inhibitory activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The phosphonate prodrugs of the invention typically will be stable in the digestive system but are substantially hydrolyzed to the parental drug in the digestive lumen, liver or other metabolic organ, or within cells in general.

### Exemplary Methods of Making the Compounds of the Invention.

The invention also relates to methods of making the compositions of the invention. The compositions are prepared by any of the applicable techniques of organic synthesis. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985), Comprehensive Organic Synthesis. Selectivity, Strategy & Efficiency in Modern Organic Chemistry. In 9 Volumes, Barry M. Trost, Editor-in-Chief (Pergamon Press, New York, 1993 printing).

A number of exemplary methods for the preparation of the compositions of the invention are provided below. These methods are intended to illustrate the nature of such preparations are not intended to limit the scope of applicable methods.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g., inert gas environments) are common in the art and will be applied when applicable.

### Schemes and Examples

General aspects of these exemplary methods are described below and in the Examples. Each of the products of the following processes is optionally separated, isolated, and/or purified prior to its use in subsequent processes.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g., inert gas environments) are common in the art and will be applied when applicable.

The terms “treated”, “treating”, “treatment”, and the like, when used in connection with a chemical synthetic operation, mean contacting, mixing, reacting, allowing to react, bringing into contact, and other terms common in the art for indicating that one or more chemical entities is treated in such a manner as to convert it to one or more other chemical entities. This means that “treating compound one with compound two” is synonymous with “allowing compound one to react with compound two”, “contacting compound one with compound two”, “reacting compound one with compound two”, and other expressions common in the art of organic synthesis for reasonably indicating that compound one was “treated”, “reacted”, “allowed to react”, etc., with compound two. For example, treating indicates the reasonable and usual manner in which organic chemicals are allowed to react. Normal concentrations (0.01M to 10M, typically 0.1M to 1M), temperatures (−100° C. to 250° C., typically −78° C. to 150° C., more typically −78° C. to 100° C., still more typically 0° C. to 100° C.), reaction vessels (typically glass, plastic, metal), solvents, pressures, atmospheres (typically air for oxygen and water insensitive reactions or nitrogen or argon for oxygen or water sensitive), etc., are intended unless otherwise indicated. The knowledge of similar reactions known in the art of organic synthesis are used in selecting the conditions and apparatus for “treating” in a given process. In particular, one of ordinary skill in the art of organic synthesis selects conditions and apparatus reasonably expected to successfully carry out the chemical reactions of the described processes based on the knowledge in the art.

Modifications of each of the exemplary schemes and in the examples (hereafter “exemplary schemes”) leads to various analogs of the specific exemplary materials produce. The above-cited citations describing suitable methods of organic synthesis are applicable to such modifications.

In each of the exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

A single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller, C. H., (1975) J. Chromatogr., 113:(3) 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers of atropisomeric compounds can be separated and isolated by normal- and reverse-phase chromatography following methods for separation of atropisomeric naphthyl-isoquinolines (Hoye, T., WO 96/15111). By method (3), a racemic mixture of two enantiomers can be separated by chromatography using a chiral stationary phase (Chiral Liquid Chromatography (1989) W. J. Lough, Ed. Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

### Examples General Section

A number of exemplary methods for the preparation of compounds of the invention are provided herein, for example, in the Examples hereinbelow. These methods are intended to illustrate the nature of such preparations are not intended to limit the scope of applicable methods. Certain compounds of the invention can be used as intermediates for the preparation of other compounds of the invention. For example, the interconversion of various phosphonate compounds of the invention is illustrated below.

### Interconversions of the Phosphonates R-Link-P(O)(OR¹)₂, R-Link-P(O)(OR¹)(OH) and R-Link-P(O)(OH)₂.

The following schemes 32-38 described the preparation of phosphonate esters of the general structure R-link-P(O)(OR¹)₂, in which the groups R¹ may be the same or different. The R¹ groups attached to a phosphonate ester, or to precursors thereto, may be changed using established chemical transformations. The interconversion reactions of phosphonates are illustrated in Scheme S32. The group R in Scheme 32 represents the substructure, i.e. the drug “scaffold, to which the substituent link-P(O)(OR¹)₂ is attached, either in the compounds of the invention, or in precursors thereto. At the point in the synthetic route of conducting a phosphonate interconversion, certain functional groups in R may be protected. The methods employed for a given phosphonate transformation depend on the nature of the substituent R¹, and of the substrate to which the phosphonate group is attached. The preparation and hydrolysis of phosphonate esters is described in Organic Phosphorus Compounds, G. M. Kosolapoff, L. Maeir, eds, Wiley, 1976, p. 9ff.

In general, synthesis of phosphonate esters is achieved by coupling a nucleophile amine or alcohol with the corresponding activated phosphonate electrophilic precursor. For example, chlorophosphonate addition on to 5′-hydroxy of nucleoside is a well known method for preparation of nucleoside phosphate monoesters. The activated precursor can be prepared by several well known methods. Chlorophosphonates useful for synthesis of the prodrugs are prepared from the substituted-1,3-propanediol (Wissner, et al, (1992) J. Med Chem. 35:1650). Chlorophosphonates are made by oxidation of the corresponding chlorophospholanes (Anderson, et al, (1984) J. Org. Chem. 49:1304) which are obtained by reaction of the substituted diol with phosphorus trichloride. Alternatively, the chlorophosphonate agent is made by treating substituted-1,3-diols with phosphorusoxychloride (Patois, et al, (1990) J. Chem. Soc. Perkin Trans. I, 1577). Chlorophosphonate species may also be generated in situ from corresponding cyclic phosphites (Silverburg, et al., (1996) Tetrahedron lett., 37:771-774), which in turn can be either made from chlorophospholane or phosphoramidate intermediate. Phosphoroflouridate intermediate prepared either from pyrophosphate or phosphoric acid may also act as precursor in preparation of cyclic prodrugs (Watanabe et al., (1988) Tetrahedron lett., 29:5763-66).

Phosphonate prodrugs of the present invention may also be prepared from the free acid by Mitsunobu reactions (Mitsunobu, (1981) Synthesis, 1; Campbell, (1992) J. Org. Chem. 57:6331), and other acid coupling reagents including, but not limited to, carbodiimides (Alexander, et al, (1994) Collect. Czech. Chem. Commun. 59:1853; Casara et al, (1992) Bioorg. Med. Chem. Lett. 2:145; Ohashi et al, (1988) Tetrahedron Lett., 29:1189), and benzotriazolyloxytris-(dimethylamino)phosphonium salts (Campagne et al (1993) Tetrahedron Lett. 34:6743).

Aryl halides undergo Ni⁺² catalyzed reaction with phosphite derivatives to give aryl phosphonate containing compounds (Balthazar, et al (1980) J. Org. Chem. 45:5425). Phosphonates may also be prepared from the chlorophosphonate in the presence of a palladium catalyst using aromatic triflates (Petrakis et al (1987) J. Am. Chem. Soc. 109:2831; Lu et al (1987) Synthesis 726). In another method, aryl phosphonate esters are prepared from aryl phosphates under anionic rearrangement conditions (Melvin (1981) Tetrahedron Lett. 22:3375; Casteel et al (1991) Synthesis, 691). N-Alkoxy aryl salts with alkali met al derivatives of cyclic alkyl phosphonate provide general synthesis for heteroaryl-2-phosphonate linkers (Redmore (1970) J. Org. Chem. 35:4114). These above mentioned methods can also be extended to compounds where the W⁵ group is a heterocycle. Cyclic-1,3-propanyl prodrugs of phosphonates are also synthesized from phosphonic diacids and substituted propane-1,3-diols using a coupling reagent such as 1,3-dicyclohexylcarbodiimide (DCC) in presence of a base (e.g., pyridine). Other carbodiimide based coupling agents like 1,3-disopropylcarbodiimide or water soluble reagent, 1-(3-dimethylaminopropyl)-3-ethylcarbodiimide hydrochloride (EDCI) can also be utilized for the synthesis of cyclic phosphonate prodrugs.

The conversion of a phosphonate diester S32.1 into the corresponding phosphonate monoester S32.2 (Scheme 32, Reaction 1) is accomplished by a number of methods. For example, the ester S32.1 in which R¹ is an aralkyl group such as benzyl, is converted into the monoester compound S32.2 by reaction with a tertiary organic base such as diazabicyclooctane (DABCO) or quinuclidine, as described in J. Org. Chem. (1995) 60:2946. The reaction is performed in an inert hydrocarbon solvent such as toluene or xylene, at about 110° C. The conversion of the diester S32.1 in which R¹ is an aryl group such as phenyl, or an alkenyl group such as allyl, into the monoester S32.2 is effected by treatment of the ester S32.1 with a base such as aqueous sodium hydroxide in acetonitrile or lithium hydroxide in aqueous tetrahydrofuran. Phosphonate diesters S32.1 in which one of the groups R¹ is aralkyl, such as benzyl, and the other is alkyl, is converted into the monoesters S32.2 in which R¹ is alkyl by hydrogenation, for example using a palladium on carbon catalyst. Phosphonate diesters in which both of the groups R¹ are alkenyl, such as allyl, is converted into the monoester S32.2 in which R¹ is alkenyl, by treatment with chlorotris(triphenylphosphine)rhodium (Wilkinson's catalyst) in aqueous ethanol at reflux, optionally in the presence of diazabicyclooctane, for example by using the procedure described in J. Org. Chem. (1973) 38:3224, for the cleavage of allyl carboxylates.

The conversion of a phosphonate diester S32.1 or a phosphonate monoester S32.2 into the corresponding phosphonic acid S32.3 (Scheme 32, Reactions 2 and 3) can be effected by reaction of the diester or the monoester with trimethylsilyl bromide, as described in J. Chem. Soc., Chem. Comm., (1979) 739. The reaction is conducted in an inert solvent such as, for example, dichloromethane, optionally in the presence of a silylating agent such as bis(trimethylsilyl)trifluoroacetamide, at ambient temperature. A phosphonate monoester S32.2 in which R¹ is aralkyl such as benzyl, is converted into the corresponding phosphonic acid S32.3 by hydrogenation over a palladium catalyst, or by treatment with hydrogen chloride in an ethereal solvent such as dioxane. A phosphonate monoester S32.2 in which R¹ is alkenyl such as, for example, allyl, is converted into the phosphonic acid S32.3 by reaction with Wilkinson's catalyst in an aqueous organic solvent, for example in 15% aqueous acetonitrile, or in aqueous ethanol, for example using the procedure described in Helv. Chim. Acta. (1985) 68:618. Palladium catalyzed hydrogenolysis of phosphonate esters S32.1 in which R¹ is benzyl is described in J. Org. Chem. (1959) 24:434. Platinum-catalyzed hydrogenolysis of phosphonate esters S32.1 in which R¹ is phenyl is described in J. Am. Chem. Soc. (1956) 78:2336.

The conversion of a phosphonate monoester S32.2 into a phosphonate diester S32.1 (Scheme 32, Reaction 4) in which the newly introduced R¹ group is alkyl, aralkyl, haloalkyl such as chloroethyl, or aralkyl is effected by a number of reactions in which the substrate S32.2 is reacted with a hydroxy compound R¹OH, in the presence of a coupling agent. Typically, the second phosphonate ester group is different than the first introduced phosphonate ester group, i.e. R¹ is followed by the introduction of R² where each of R¹ and R² is alkyl, aralkyl, haloalkyl such as chloroethyl, or aralkyl (Scheme 32, Reaction 4a) whereby S32.2 is converted to S32.1a. Suitable coupling agents are those employed for the preparation of carboxylate esters, and include a carbodiimide such as dicyclohexylcarbodiimide, in which case the reaction is preferably conducted in a basic organic solvent such as pyridine, or (benzotriazol-1-yloxy)tripyrrolidinophosphonium hexafluorophosphate (PYBOP, Sigma), in which case the reaction is performed in a polar solvent such as dimethylformamide, in the presence of a tertiary organic base such as diisopropylethylamine, or Aldrithiol-2 (Aldrich) in which case the reaction is conducted in a basic solvent such as pyridine, in the presence of a triaryl phosphine such as triphenylphosphine. Alternatively, the conversion of the phosphonate monoester S32.2 to the diester S32.1 is effected by the use of the Mitsunobu reaction, as described above (Scheme 7). The substrate is reacted with the hydroxy compound R¹OH, in the presence of diethyl azodicarboxylate and a triarylphosphine such as triphenyl phosphine. Alternatively, the phosphonate monoester S32.2 is transformed into the phosphonate diester S32.1, in which the introduced R¹ group is alkenyl or aralkyl, by reaction of the monoester with the halide R¹Br, in which R¹ is as alkenyl or aralkyl. The alkylation reaction is conducted in a polar organic solvent such as dimethylformamide or acetonitrile, in the presence of a base such as cesium carbonate. Alternatively, the phosphonate monoester is transformed into the phosphonate diester in a two step procedure. In the first step, the phosphonate monoester S32.2 is transformed into the chloro analog RP(O)(OR¹)Cl by reaction with thionyl chloride or oxalyl chloride and the like, as described in Organic Phosphorus Compounds, G. M. Kosolapoff, L. Maeir, eds, Wiley, 1976, p. 17, and the thus-obtained product RP(O)(OR¹)Cl is then reacted with the hydroxy compound R¹OH, in the presence of a base such as triethylamine, to afford the phosphonate diester S32.1.

A phosphonic acid R-link-P(O)(OH)₂ is transformed into a phosphonate monoester RP(O)(OR¹)(OH) (Scheme 32, Reaction 5) by means of the methods described above of for the preparation of the phosphonate diester R-link-P(O)(OR¹)₂ S32.1, except that only one molar proportion of the component R¹OH or R¹Br is employed. Dialkyl phosphonates may be prepared according to the methods of: Quast et al (1974) Synthesis 490; Stowell et al (1990) Tetrahedron Lett. 3261; U.S. Pat. No. 5,663,159.

A phosphonic acid R-link-P(O)(OH)₂ S32.3 is transformed into a phosphonate diester R-link-P(O)(OR¹)₂ S32.1 (Scheme 32, Reaction 6) by a coupling reaction with the hydroxy compound R¹OH, in the presence of a coupling agent such as Aldrithiol-2 (Aldrich) and triphenylphosphine. The reaction is conducted in a basic solvent such as pyridine. Alternatively, phosphonic acids S32.3 are transformed into phosphonic esters S32.1 in which R¹ is aryl, by means of a coupling reaction employing, for example, dicyclohexylcarbodiimide in pyridine at ca 70° C. Alternatively, phosphonic acids S32.3 are transformed into phosphonic esters S32.1 in which R¹ is alkenyl, by means of an alkylation reaction. The phosphonic acid is reacted with the alkenyl bromide R¹Br in a polar organic solvent such as acetonitrile solution at reflux temperature, the presence of a base such as cesium carbonate, to afford the phosphonic ester S32.1.

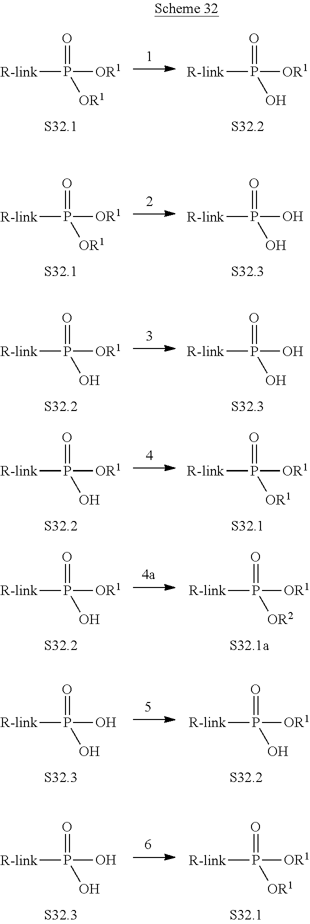

### Preparation of Phosphonate Carbamates.

Phosphonate esters may contain a carbamate linkage. The preparation of carbamates is described in Comprehensive Organic Functional Group Transformations, A. R. Katritzky, ed., Pergamon, 1995, Vol. 6, p. 416ff, and in Organic Functional Group Preparations, by S. R. Sandler and W. Karo, Academic Press, 1986, p. 260ff. The carbamoyl group may be formed by reaction of a hydroxy group according to the methods known in the art, including the teachings of Ellis, US 2002/0103378 A1 and Hajima, U.S. Pat. No. 6,018,049.

Scheme 33 illustrates various methods by which the carbamate linkage is synthesized. As shown in Scheme 33, in the general reaction generating carbamates, an alcohol S33.1, is converted into the activated derivative S33.2 in which Lv is a leaving group such as halo, imidazolyl, benztriazolyl and the like, as described herein. The activated derivative S33.2 is then reacted with an amine S33.3, to afford the carbamate product S33.4. Examples 1-7 in Scheme 33 depict methods by which the general reaction is effected. Examples 8-10 illustrate alternative methods for the preparation of carbamates.

Scheme 33, Example 1 illustrates the preparation of carbamates employing a chloroformyl derivative of the alcohol S33.5. In this procedure, the alcohol S33.5 is reacted with phosgene, in an inert solvent such as toluene, at about 0° C., as described in Org. Syn. Coll. Vol. 3, 167, 1965, or with an equivalent reagent such as trichloromethoxy chloroformate, as described in Org. Syn. Coll. Vol. 6, 715, 1988, to afford the chloroformate S33.6. The latter compound is then reacted with the amine component S33.3, in the presence of an organic or inorganic base, to afford the carbamate S33.7. For example, the chloroformyl compound S33.6 is reacted with the amine S33.3 in a water-miscible solvent such as tetrahydrofuran, in the presence of aqueous sodium hydroxide, as described in Org. Syn. Coll. Vol. 3, 167, 1965, to yield the carbamate S33.7. Alternatively, the reaction is performed in dichloromethane in the presence of an organic base such as diisopropylethylamine or dimethylaminopyridine.

Scheme 33, Example 2 depicts the reaction of the chloroformate compound S33.6 with imidazole to produce the imidazolide S33.8. The imidazolide product is then reacted with the amine S33.3 to yield the carbamate S33.7. The preparation of the imidazolide is performed in an aprotic solvent such as dichloromethane at 0°, and the preparation of the carbamate is conducted in a similar solvent at ambient temperature, optionally in the presence of a base such as dimethylaminopyridine, as described in J. Med. Chem., 1989, 32, 357.

Scheme 33 Example 3, depicts the reaction of the chloroformate S33.6 with an activated hydroxyl compound R″OH, to yield the mixed carbonate ester S33.10. The reaction is conducted in an inert organic solvent such as ether or dichloromethane, in the presence of a base such as dicyclohexylamine or triethylamine. The hydroxyl component R″OH is selected from the group of compounds S33.19-S33.24 shown in Scheme 33, and similar compounds. For example, if the component R″OH is hydroxybenztriazole S33.19, N-hydroxysuccinimide S33.20, or pentachlorophenol, S33.21, the mixed carbonate S33.10 is obtained by the reaction of the chloroformate with the hydroxyl compound in an ethereal solvent in the presence of dicyclohexylamine, as described in Can. J. Chem., 1982, 60, 976. A similar reaction in which the component R″OH is pentafluorophenol S33.22 or 2-hydroxypyridine S33.23 is performed in an ethereal solvent in the presence of triethylamine, as described in Syn., 1986, 303, and Chem. Ber. 118, 468, 1985.

Scheme 33 Example 4 illustrates the preparation of carbamates in which an alkyloxycarbonylimidazole S33.8 is employed. In this procedure, an alcohol S33.5 is reacted with an equimolar amount of carbonyl diimidazole S33.11 to prepare the intermediate S33.8. The reaction is conducted in an aprotic organic solvent such as dichloromethane or tetrahydrofuran. The acyloxyimidazole S33.8 is then reacted with an equimolar amount of the amine R′NH₂ to afford the carbamate S33.7. The reaction is performed in an aprotic organic solvent such as dichloromethane, as described in Tet. Lett., 42, 2001, 5227, to afford the carbamate S33.7.

Scheme 33, Example 5 illustrates the preparation of carbamates by means of an intermediate alkoxycarbonylbenztriazole S33.13. In this procedure, an alcohol ROH is reacted at ambient temperature with an equimolar amount of benzotriazole carbonyl chloride S33.12, to afford the alkoxycarbonyl product S33.13. The reaction is performed in an organic solvent such as benzene or toluene, in the presence of a tertiary organic amine such as triethylamine, as described in Synthesis., 1977, 704. The product is then reacted with the amine R′NH₂ to afford the carbamate S33.7. The reaction is conducted in toluene or ethanol, at from ambient temperature to about 80° C. as described in Synthesis., 1977, 704.

Scheme 33, Example 6 illustrates the preparation of carbamates in which a carbonate (R″O)₂CO, S33.14, is reacted with an alcohol S33.5 to afford the intermediate alkyloxycarbonyl intermediate S33.15. The latter reagent is then reacted with the amine R′NH₂ to afford the carbamate S33.7. The procedure in which the reagent S33.15 is derived from hydroxybenztriazole S33.19 is described in Synthesis, 1993, 908; the procedure in which the reagent S33.15 is derived from N-hydroxysuccinimide S33.20 is described in Tet. Lett., 1992, 2781; the procedure in which the reagent S33.15 is derived from 2-hydroxypyridine S33.23 is described in Tet. Lett., 1991, 4251; the procedure in which the reagent S33.15 is derived from 4-nitrophenol S33.24 is described in Synthesis. 1993, 103. The reaction between equimolar amounts of the alcohol ROH and the carbonate S33.14 is conducted in an inert organic solvent at ambient temperature.

Scheme 33, Example 7 illustrates the preparation of carbamates from alkoxycarbonyl azides S33.16. In this procedure, an alkyl chloroformate S33.6 is reacted with an azide, for example sodium azide, to afford the alkoxycarbonyl azide S33.16. The latter compound is then reacted with an equimolar amount of the amine R′NH₂ to afford the carbamate S33.7. The reaction is conducted at ambient temperature in a polar aprotic solvent such as dimethylsulfoxide, for example as described in Synthesis., 1982, 404.

Scheme 33, Example 8 illustrates the preparation of carbamates by means of the reaction between an alcohol ROH and the chloroformyl derivative of an amine S33.17. In this procedure, which is described in Synthetic Organic Chemistry, R. B. Wagner, H. D. Zook, Wiley, 1953, p. 647, the reactants are combined at ambient temperature in an aprotic solvent such as acetonitrile, in the presence of a base such as triethylamine, to afford the carbamate S33.7.

Scheme 33, Example 9 illustrates the preparation of carbamates by means of the reaction between an alcohol ROH and an isocyanate S33.18. In this procedure, which is described in Synthetic Organic Chemistry, R. B. Wagner, H. D. Zook, Wiley, 1953, p. 645, the reactants are combined at ambient temperature in an aprotic solvent such as ether or dichloromethane and the like, to afford the carbamate S33.7.

Scheme 33, Example 10 illustrates the preparation of carbamates by means of the reaction between an alcohol ROH and an amine R′NH₂. In this procedure, which is described in Chem. Lett. 1972, 373, the reactants are combined at ambient temperature in an aprotic organic solvent such as tetrahydrofuran, in the presence of a tertiary base such as triethylamine, and selenium. Carbon monoxide is passed through the solution and the reaction proceeds to afford the carbamate S33.7.

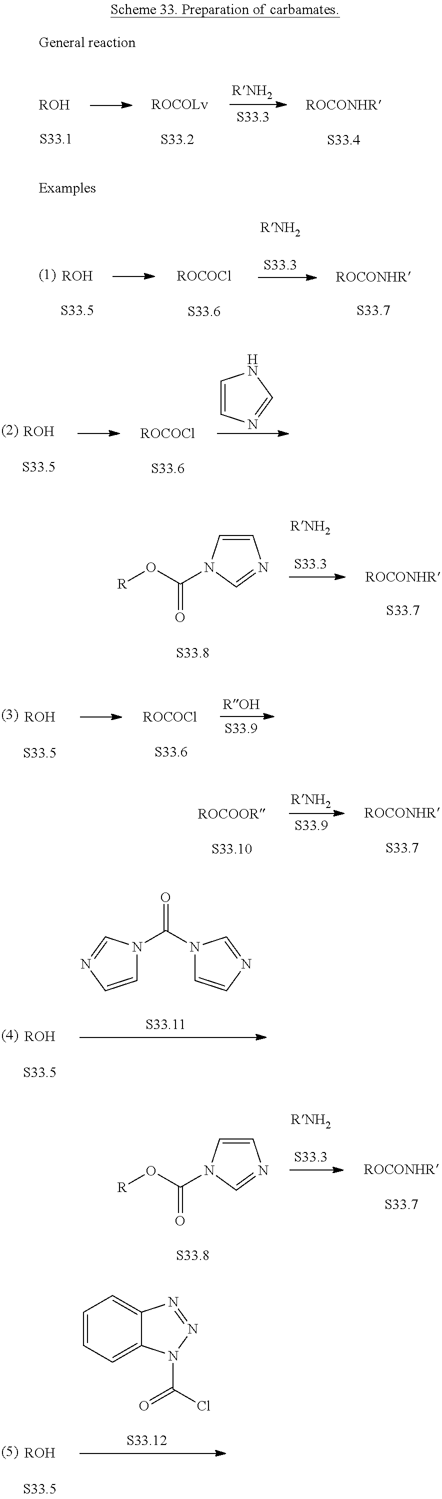

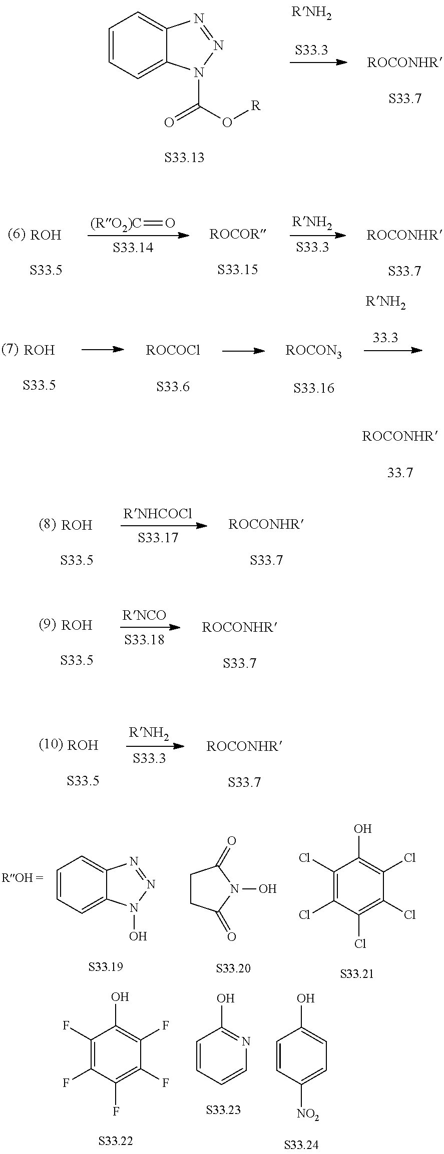

### Preparation of Carboalkoxy-Substituted Phosphonate Bisamidates, Monoamidates, Diesters and Monoesters.

A number of methods are available for the conversion of phosphonic acids into amidates and esters. In one group of methods, the phosphonic acid is either converted into an isolated activated intermediate such as a phosphoryl chloride, or the phosphonic acid is activated in situ for reaction with an amine or a hydroxy compound.

The conversion of phosphonic acids into phosphoryl chlorides is accomplished by reaction with thionyl chloride, for example as described in J. Gen. Chem. USSR, 1983, 53, 480, Zh. Obschei Khim., 1958, 28, 1063, or J. Org. Chem., 1994, 59, 6144, or by reaction with oxalyl chloride, as described in J. Am. Chem. Soc., 1994, 116, 3251, or J. Org. Chem., 1994, 59, 6144, or by reaction with phosphorus pentachloride, as described in J. Org. Chem., 2001, 66, 329, or in J. Med. Chem., 1995, 38, 1372. The resultant phosphoryl chlorides are then reacted with amines or hydroxy compounds in the presence of a base to afford the amidate or ester products.

Phosphonic acids are converted into activated imidazolyl derivatives by reaction with carbonyl diimidazole, as described in J. Chem. Soc., Chem. Comm. (1991) 312, or Nucleosides & Nucleotides (2000) 19:1885. Activated sulfonyloxy derivatives are obtained by the reaction of phosphonic acids with trichloromethylsulfonyl chloride or with triisopropylbenzenesulfonyl chloride, as described in Tet. Lett. (1996) 7857, or Bioorg. Med. Chem. Lett. (1998) 8:663. The activated sulfonyloxy derivatives are then reacted with amines or hydroxy compounds to afford amidates or esters.

Alternatively, the phosphonic acid and the amine or hydroxy reactant are combined in the presence of a diimide coupling agent. The preparation of phosphonic amidates and esters by means of coupling reactions in the presence of dicyclohexyl carbodiimide is described, for example, in J. Chem. Soc., Chem. Comm. (1991) 312 or Coll. Czech. Chem. Comm. (1987) 52:2792. The use of ethyl dimethylaminopropyl carbodiimide for activation and coupling of phosphonic acids is described in Tet. Lett., (2001) 42:8841, or Nucleosides & Nucleotides (2000) 19:1885.

A number of additional coupling reagents have been described for the preparation of amidates and esters from phosphonic acids. The agents include Aldrithiol-2, and PYBOP and BOP, as described in J. Org. Chem., 1995, 60, 5214, and J. Med. Chem. (1997) 40:3842, mesitylene-2-sulfonyl-3-nitro-1,2,4-triazole (MSNT), as described in J. Med. Chem. (1996) 39:4958, diphenylphosphoryl azide, as described in J. Org. Chem. (1984) 49:1158, 1-(2,4,6-triisopropylbenzenesulfonyl-3-nitro-1,2,4-triazole (TPSNT) as described in Bioorg. Med. Chem. Lett. (1998) 8:1013, bromotris(dimethylamino)phosphonium hexafluorophosphate (BroP), as described in Tet. Lett., (1996) 37:3997, 2-chloro-5,5-dimethyl-2-oxo-1,3,2-dioxaphosphinane, as described in Nucleosides Nucleotides 1995, 14, 871, and diphenyl chlorophosphate, as described in J. Med. Chem., 1988, 31, 1305.

Phosphonic acids are converted into amidates and esters by means of the Mitsunobu reaction, in which the phosphonic acid and the amine or hydroxy reactant are combined in the presence of a triaryl phosphine and a dialkyl azodicarboxylate. The procedure is described in Org. Lett., 2001, 3, 643, or J. Med. Chem., 1997, 40, 3842.

Phosphonic esters are also obtained by the reaction between phosphonic acids and halo compounds, in the presence of a suitable base. The method is described, for example, in Anal. Chem., 1987, 59, 1056, or J. Chem. Soc. Perkin Trans., I, 1993, 19, 2303, or J. Med. Chem., 1995, 38, 1372, or Tet. Lett., 2002, 43, 1161.

Schemes 34-37 illustrate the conversion of phosphonate esters and phosphonic acids into carboalkoxy-substituted phosphonbisamidates (Scheme 34), phosphonamidates (Scheme 35), phosphonate monoesters (Scheme 36) and phosphonate diesters, (Scheme 37). Scheme 38 illustrates synthesis of gem-dialkyl amino phosphonate reagents.

Scheme 34 illustrates various methods for the conversion of phosphonate diesters S34.1 into phosphonbisamidates S34.5. The diester S34.1, prepared as described previously, is hydrolyzed, either to the monoester S34.2 or to the phosphonic acid S34.6. The methods employed for these transformations are described above. The monoester S34.2 is converted into the monoamidate S34.3 by reaction with an aminoester S34.9, in which the group R² is H or alkyl; the group R^{4b} is a divalent alkylene moiety such as, for example, CHCH₃, CHCH₂CH₃, CH(CH(CH₃)₂), CH(CH₂Ph), and the like, or a side chain group present in natural or modified aminoacids; and the group R^{5b} is C₁-C₁₂ alkyl, such as methyl, ethyl, propyl, isopropyl, or isobutyl; C₆-C₂₀ aryl, such as phenyl or substituted phenyl; or C₆-C₂₀ arylalkyl, such as benzyl or benzyhydryl. The reactants are combined in the presence of a coupling agent such as a carbodiimide, for example dicyclohexyl carbodiimide, as described in J. Am. Chem. Soc., (1957) 79:3575, optionally in the presence of an activating agent such as hydroxybenztriazole, to yield the amidate product S34.3. The amidate-forming reaction is also effected in the presence of coupling agents such as BOP, as described in J. Org. Chem. (1995) 60:5214, Aldrithiol, PYBOP and similar coupling agents used for the preparation of amides and esters. Alternatively, the reactants S34.2 and S34.9 are transformed into the monoamidate S34.3 by means of a Mitsunobu reaction. The preparation of amidates by means of the Mitsunobu reaction is described in J. Med. Chem. (1995) 38:2742. Equimolar amounts of the reactants are combined in an inert solvent such as tetrahydrofuran in the presence of a triaryl phosphine and a dialkyl azodicarboxylate. The thus-obtained monoamidate ester S34.3 is then transformed into amidate phosphonic acid S34.4. The conditions used for the hydrolysis reaction depend on the nature of the R¹ group, as described previously. The phosphonic acid amidate S34.4 is then reacted with an aminoester S34.9, as described above, to yield the bisamidate product S34.5, in which the amino substituents are the same or different. Alternatively, the phosphonic acid S34.6 may be treated with two different amino ester reagents simultaneously, i.e. S34.9 where R², R^{4b} or R^{5b} are different. The resulting mixture of bisamidate products S34.5 may then be separable, e.g. by chromatography.

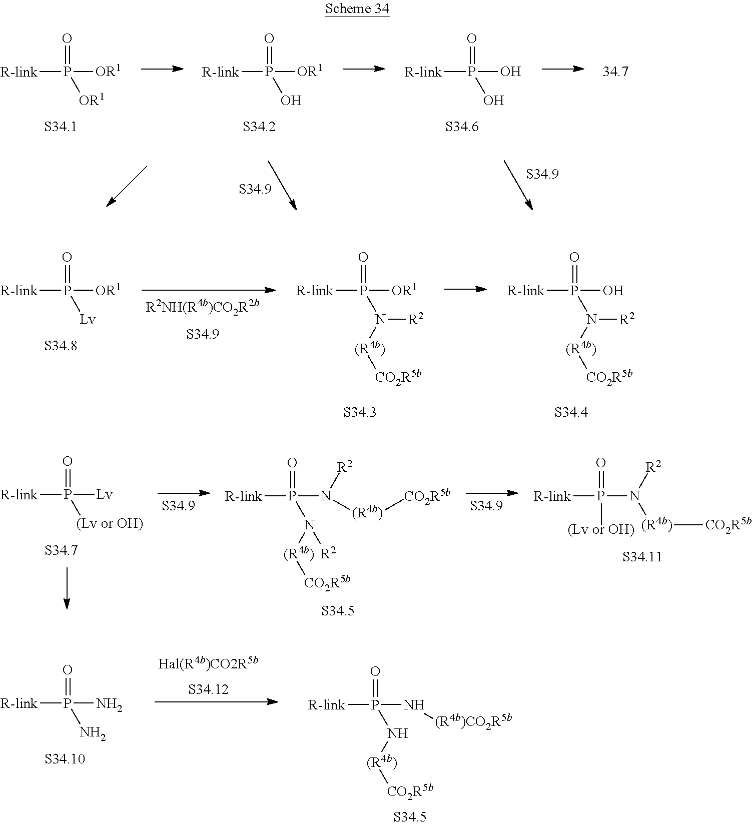

An example of this procedure is shown in Scheme 34, Example 1. In this procedure, a dibenzyl phosphonate S34.14 is reacted with diazabicyclooctane (DABCO) in toluene at reflux, as described J. Org. Chem., 1995, 60, 2946, to afford the monobenzyl phosphonate S34.15. The product is then reacted with equimolar amounts of ethyl alaninate S34.16 and dicyclohexyl carbodiimide in pyridine, to yield the amidate product S34.17. The benzyl group is then removed, for example by hydrogenolysis over a palladium catalyst, to give the monoacid product S34.18 which may be unstable according to J. Med. Chem. (1997) 40(23):3842. This compound S34.18 is then reacted in a Mitsunobu reaction with ethyl leucinate S34.19, triphenyl phosphine and diethylazodicarboxylate, as described in J. Med. Chem., 1995, 38, 2742, to produce the bisamidate product S34.20.

Using the above procedures, but employing in place of ethyl leucinate S34.19 or ethyl alaninate S34.16, different aminoesters S34.9, the corresponding products S34.5 are obtained.

Alternatively, the phosphonic acid S34.6 is converted into the bisamidate S34.5 by use of the coupling reactions described above. The reaction is performed in one step, in which case the nitrogen-related substituents present in the product S34.5 are the same, or in two steps, in which case the nitrogen-related substituents can be different.

An example of the method is shown in Scheme 34, Example 2. In this procedure, a phosphonic acid S34.6 is reacted in pyridine solution with excess ethyl phenylalaninate S34.21 and dicyclohexylcarbodiimide, for example as described in J. Chem. Soc., Chem. Comm., 1991, 1063, to give the bisamidate product S34.22.

Using the above procedures, but employing, in place of ethyl phenylalaninate, different aminoesters S34.9, the corresponding products S34.5 are obtained.

As a further alternative, the phosphonic acid S34.6 is converted into the mono or bis-activated derivative S34.7, in which Lv is a leaving group such as chloro, imidazolyl, triisopropylbenzenesulfonyloxy etc. The conversion of phosphonic acids into chlorides S34.7 (Lv=Cl) is effected by reaction with thionyl chloride or oxalyl chloride and the like, as described in Organic Phosphorus Compounds, G. M. Kosolapoff, L. Maeir, eds, Wiley, 1976, p. 17. The conversion of phosphonic acids into monoimidazolides S34.7 (Lv=imidazolyl) is described in J. Med. Chem., 2002, 45, 1284 and in J. Chem. Soc. Chem. Comm., 1991, 312. Alternatively, the phosphonic acid is activated by reaction with triisopropylbenzenesulfonyl chloride, as described in Nucleosides and Nucleotides, 2000, 10, 1885. The activated product is then reacted with the aminoester S34.9, in the presence of a base, to give the bisamidate S34.5. The reaction is performed in one step, in which case the nitrogen substituents present in the product S34.5 are the same, or in two steps, via the intermediate S34.11, in which case the nitrogen substituents can be different.

Examples of these methods are shown in Scheme 34, Examples 3 and 5. In the procedure illustrated in Scheme 34, Example 3, a phosphonic acid S34.6 is reacted with ten molar equivalents of thionyl chloride, as described in Zh. Obschei Khim., 1958, 28, 1063, to give the dichloro compound S34.23. The product is then reacted at reflux temperature in a polar aprotic solvent such as acetonitrile, and in the presence of a base such as triethylamine, with butyl serinate S34.24 to afford the bisamidate product S34.25.

Using the above procedures, but employing, in place of butyl serinate S34.24, different aminoesters S34.9, the corresponding products S34.5 are obtained.

In the procedure illustrated in Scheme 34, Example 5, the phosphonic acid S34.6 is reacted, as described in J. Chem. Soc. Chem. Comm., 1991, 312, with carbonyl diimidazole to give the imidazolide S34.S32. The product is then reacted in acetonitrile solution at ambient temperature, with one molar equivalent of ethyl alaninate S34.33 to yield the monodisplacement product S34.34. The latter compound is then reacted with carbonyl diimidazole to produce the activated intermediate S34.35, and the product is then reacted, under the same conditions, with ethyl N-methylalaninate S34.33a to give the bisamidate product S34.36.

Using the above procedures, but employing, in place of ethyl alaninate S34.33 or ethyl N-methylalaninate S34.33a, different aminoesters S34.9, the corresponding products S34.5 are obtained.

The intermediate monoamidate S34.3 is also prepared from the monoester S34.2 by first converting the monoester into the activated derivative S34.8 in which Lv is a leaving group such as halo, imidazolyl etc, using the procedures described above. The product S34.8 is then reacted with an aminoester S34.9 in the presence of a base such as pyridine, to give an intermediate monoamidate product S34.3. The latter compound is then converted, by removal of the R¹ group and coupling of the product with the aminoester S34.9, as described above, into the bisamidate S34.5.

An example of this procedure, in which the phosphonic acid is activated by conversion to the chloro derivative S34.26, is shown in Scheme 34, Example 4. In this procedure, the phosphonic monobenzyl ester S34.15 is reacted, in dichloromethane, with thionyl chloride, as described in Tet. Letters., 1994, 35, 4097, to afford the phosphoryl chloride S34.26. The product is then reacted in acetonitrile solution at ambient temperature with one molar equivalent of ethyl 3-amino-2-methylpropionate S34.27 to yield the monoamidate product S34.28. The latter compound is hydrogenated in ethylacetate over a 5% palladium on carbon catalyst to produce the monoacid product S34.29. The product is subjected to a Mitsunobu coupling procedure, with equimolar amounts of butyl alaninate S34.30, triphenyl phosphine, diethylazodicarboxylate and triethylamine in tetrahydrofuran, to give the bisamidate product S34.31.

Using the above procedures, but employing, in place of ethyl 3-amino-2-methylpropionate S34.27 or butyl alaninate S34.30, different aminoesters S34.9, the corresponding products S34.5 are obtained.

The activated phosphonic acid derivative S34.7 is also converted into the bisamidate S34.5 via the diamino compound S34.10. The conversion of activated phosphonic acid derivatives such as phosphoryl chlorides into the corresponding amino analogs S34.10, by reaction with ammonia, is described in Organic Phosphorus Compounds, G. M. Kosolapoff, L. Maeir, eds, Wiley, 1976. The bisamino compound S34.10 is then reacted at elevated temperature with a haloester S34.12 (Hal=halogen, i.e. F, Cl, Br, I), in a polar organic solvent such as dimethylformamide, in the presence of a base such as 4,4-dimethylaminopyridine (DMAP) or potassium carbonate, to yield the bisamidate S34.5. Alternatively, S34.6 may be treated with two different amino ester reagents simultaneously, i.e. S34.12 where R^{4b} or R^{5b} are different. The resulting mixture of bisamidate products S34.5 may then be separable, e.g. by chromatography.

An example of this procedure is shown in Scheme 34, Example 6. In this method, a dichlorophosphonate S34.23 is reacted with ammonia to afford the diamide S34.37. The reaction is performed in aqueous, aqueous alcoholic or alcoholic solution, at reflux temperature. The resulting diamino compound is then reacted with two molar equivalents of ethyl 2-bromo-3-methylbutyrate S34.38, in a polar organic solvent such as N-methylpyrrolidinone at ca. 150° C., in the presence of a base such as potassium carbonate, and optionally in the presence of a catalytic amount of potassium iodide, to afford the bisamidate product S34.39.

Using the above procedures, but employing, in place of ethyl 2-bromo-3-methylbutyrate S34.38, different haloesters S34.12 the corresponding products S34.5 are obtained.

The procedures shown in Scheme 34 are also applicable to the preparation of bisamidates in which the aminoester moiety incorporates different functional groups. Scheme 34, Example 7 illustrates the preparation of bisamidates derived from tyrosine. In this procedure, the monoimidazolide S34.32 is reacted with propyl tyrosinate S34.40, as described in Example 5, to yield the monoamidate S34.41. The product is reacted with carbonyl diimidazole to give the imidazolide S34.42, and this material is reacted with a further molar equivalent of propyl tyrosinate to produce the bisamidate product S34.43.

Using the above procedures, but employing, in place of propyl tyrosinate S34.40, different aminoesters S34.9, the corresponding products S34.5 are obtained. The aminoesters employed in the two stages of the above procedure can be the same or different, so that bisamidates with the same or different amino substituents are prepared.

Scheme 35 illustrates methods for the preparation of phosphonate monoamidates.

In one procedure, a phosphonate monoester S34.1 is converted, as described in Scheme 34, into the activated derivative S34.8. This compound is then reacted, as described above, with an aminoester S34.9, in the presence of a base, to afford the monoamidate product S35.1.

The procedure is illustrated in Scheme 35, Example 1. In this method, a monophenyl phosphonate S35.7 is reacted with, for example, thionyl chloride, as described in J. Gen. Chem. USSR., 1983, 32, 367, to give the chloro product S35.8. The product is then reacted, as described in Scheme 34, with ethyl alaninate S3, to yield the amidate S35.10.

Using the above procedures, but employing, in place of ethyl alaninate S35.9, different aminoesters S34.9, the corresponding products S35.1 are obtained.

Alternatively, the phosphonate monoester S34.1 is coupled, as described in Scheme 34, with an aminoester S34.9 to produce the amidate. If necessary, the R¹ substituent is then altered, by initial cleavage to afford the phosphonic acid S35.2. The procedures for this transformation depend on the nature of the R¹ group, and are described above. The phosphonic acid is then transformed into the ester amidate product S35.3, by reaction with the hydroxy compound R³OH, in which the group R³ is aryl, heterocycle, alkyl, cycloalkyl, haloalkyl etc, using the same coupling procedures (carbodiimide, Aldrithiol-2, PYBOP, Mitsunobu reaction etc) described in Scheme 34 for the coupling of amines and phosphonic acids.

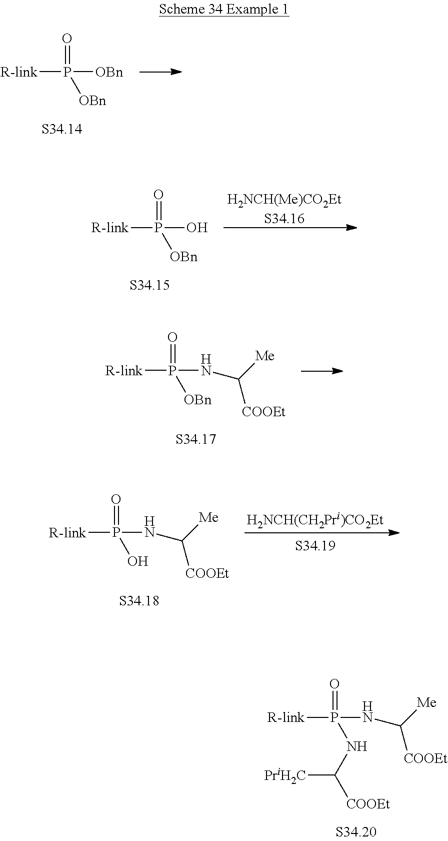

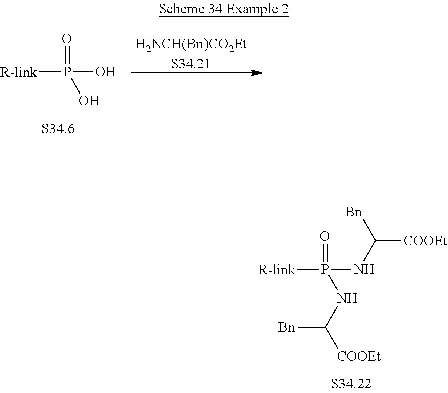

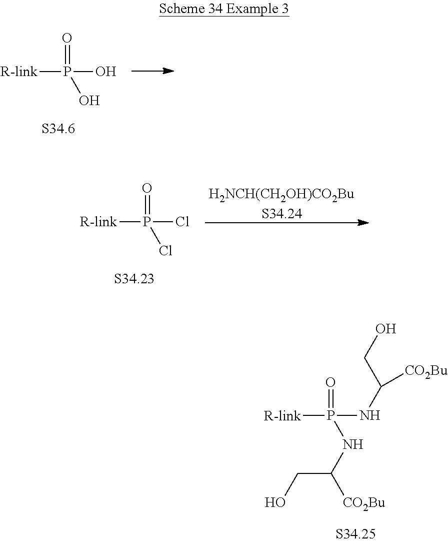

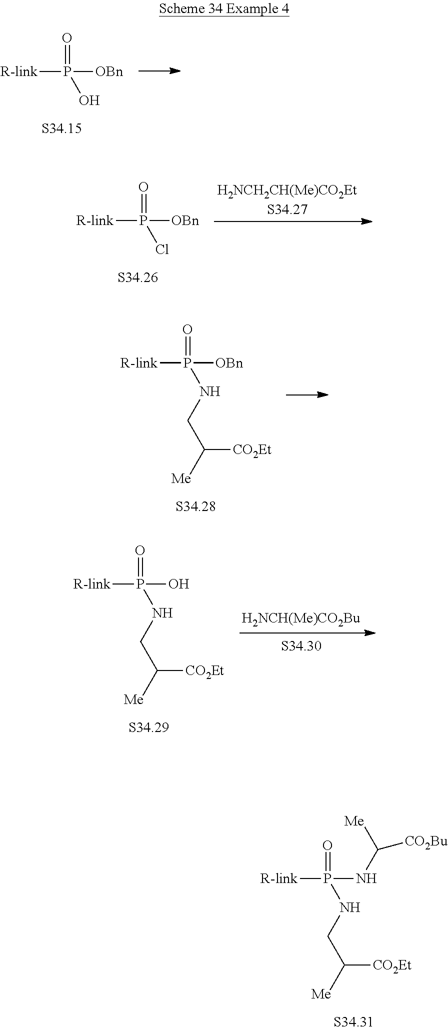

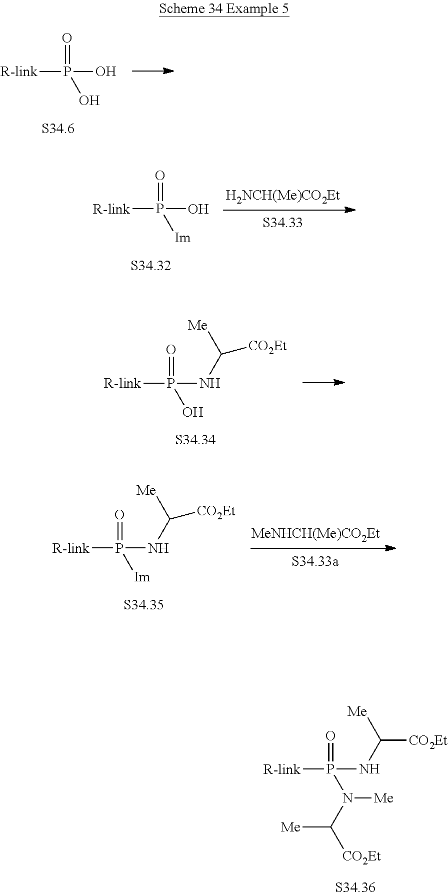

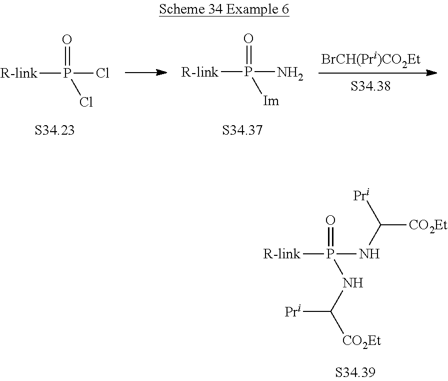

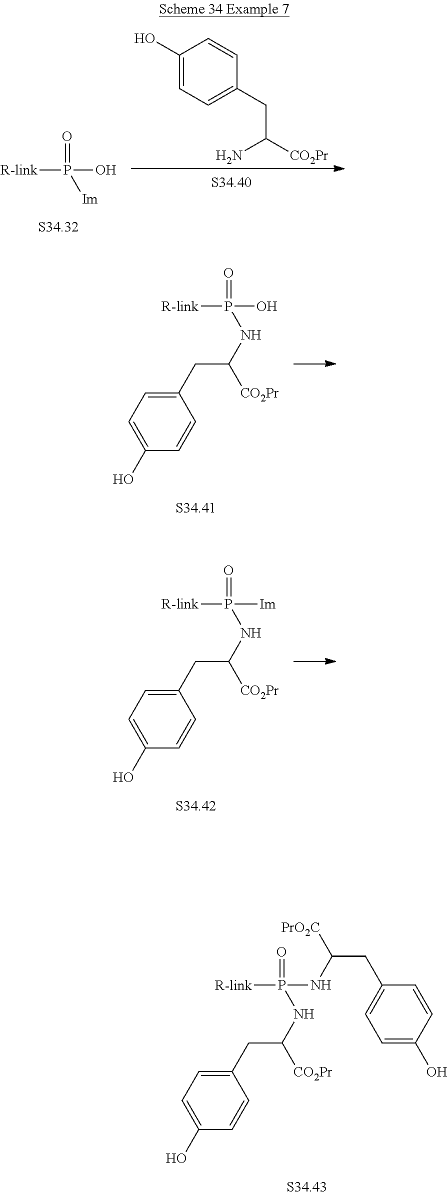

Examples of this method are shown in Scheme 35, Examples and 2 and 3. In the sequence shown in Example 2, a monobenzyl phosphonate S35.11 is transformed by reaction with ethyl alaninate, using one of the methods described above, into the monoamidate S35.12. The benzyl group is then removed by catalytic hydrogenation in ethylacetate solution over a 5% palladium on carbon catalyst, to afford the phosphonic acid amidate S35.13. The product is then reacted in dichloromethane solution at ambient temperature with equimolar amounts of 1-(dimethylaminopropyl)-3-ethylcarbodiimide and trifluoroethanol S35.14, for example as described in Tet. Lett., 2001, 42, 8841, to yield the amidate ester S35.15.

In the sequence shown in Scheme 35, Example 3, the monoamidate S35.13 is coupled, in tetrahydrofuran solution at ambient temperature, with equimolar amounts of dicyclohexyl carbodiimide and 4-hydroxy-N-methylpiperidine S35.16, to produce the amidate ester product S35.17.

Using the above procedures, but employing, in place of the ethyl alaninate product S35.12 different monoacids S35.2, and in place of trifluoroethanol S35.14 or 4-hydroxy-N-methylpiperidine S35.16, different hydroxy compounds R³OH, the corresponding products S35.3 are obtained.

Alternatively, the activated phosphonate ester S34.8 is reacted with ammonia to yield the amidate S35.4. The product is then reacted, as described in Scheme 34, with a haloester S35.5, in the presence of a base, to produce the amidate product S35.6. If appropriate, the nature of the R¹ group is changed, using the procedures described above, to give the product S35.3. The method is illustrated in Scheme 35, Example 4. In this sequence, the monophenyl phosphoryl chloride S35.18 is reacted, as described in Scheme 34, with ammonia, to yield the amino product S35.19. This material is then reacted in N-methylpyrrolidinone solution at 170° with butyl 2-bromo-3-phenylpropionate S35.20 and potassium carbonate, to afford the amidate product S35.21.

Using these procedures, but employing, in place of butyl 2-bromo-3-phenylpropionate S35.20, different haloesters S35.5, the corresponding products S35.6 are obtained.

The monoamidate products S35.3 are also prepared from the doubly activated phosphonate derivatives S34.7. In this procedure, examples of which are described in Synlett., 1998, 1, 73, the intermediate S34.7 is reacted with a limited amount of the aminoester S34.9 to give the mono-displacement product S34.11. The latter compound is then reacted with the hydroxy compound R³OH in a polar organic solvent such as dimethylformamide, in the presence of a base such as diisopropylethylamine, to yield the monoamidate ester S35.3.

The method is illustrated in Scheme 35, Example 5. In this method, the phosphoryl dichloride S35.22 is reacted in dichloromethane solution with one molar equivalent of ethyl N-methyl tyrosinate S35.23 and dimethylaminopyridine, to generate the monoamidate S35.24. The product is then reacted with phenol S35.25 in dimethylformamide containing potassium carbonate, to yield the ester amidate product S35.26.

Using these procedures, but employing, in place of ethyl N-methyl tyrosinate S35.23 or phenol S35.25, the aminoesters 34.9 and/or the hydroxy compounds R³OH, the corresponding products S35.3 are obtained.

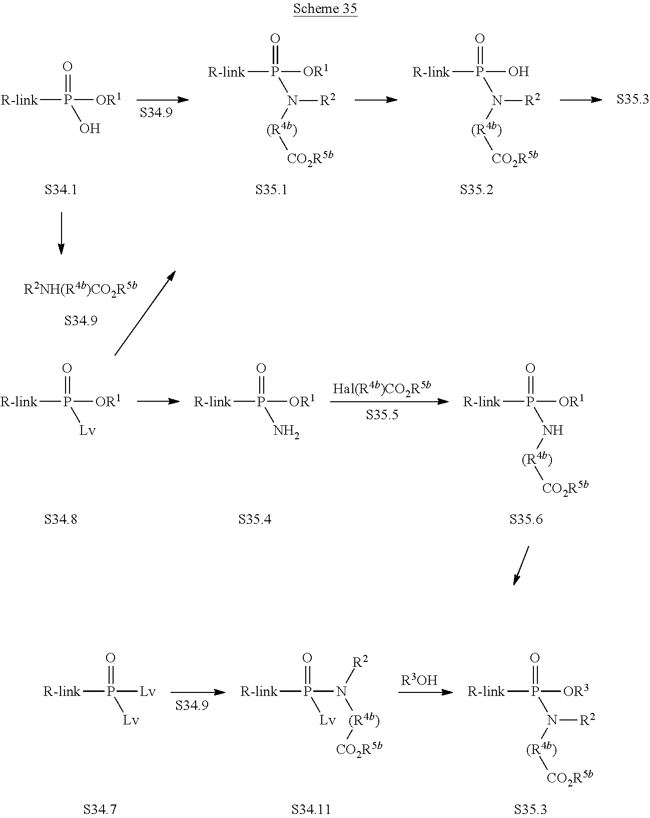

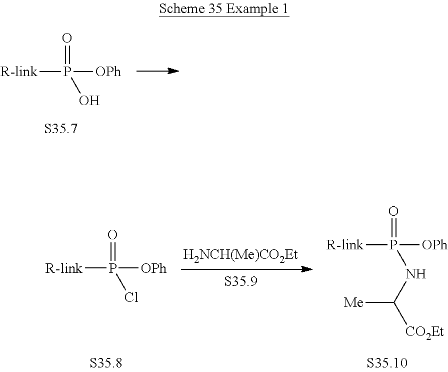

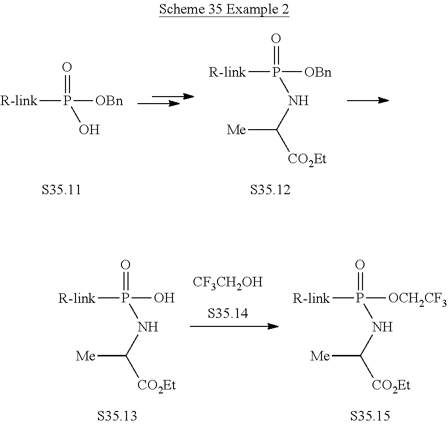

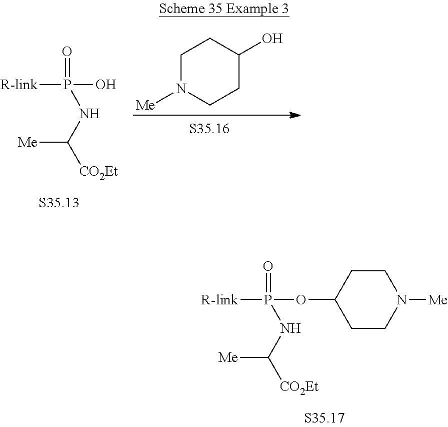

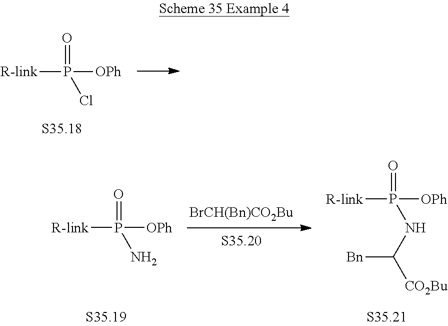

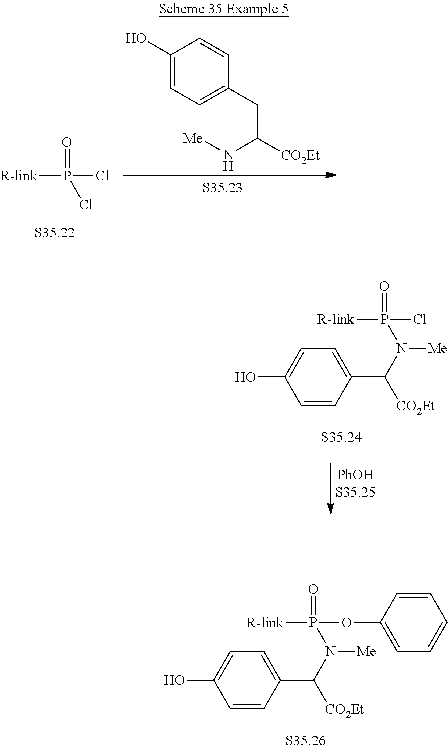

Scheme 36 illustrates methods for the preparation of carboalkoxy-substituted phosphonate diesters in which one of the ester groups incorporates a carboalkoxy substituent.

In one procedure, a phosphonate monoester S34.1, prepared as described above, is coupled, using one of the methods described above, with a hydroxyester S36.1, in which the groups R^{4b} and R^{5b} are as described in Scheme 34. For example, equimolar amounts of the reactants are coupled in the presence of a carbodiimide such as dicyclohexyl carbodiimide, as described in Aust. J. Chem., 1963, 609, optionally in the presence of dimethylaminopyridine, as described in Tet., 1999, 55, 12997. The reaction is conducted in an inert solvent at ambient temperature.

The procedure is illustrated in Scheme 36, Example 1. In this method, a monophenyl phosphonate S36.9 is coupled, in dichloromethane solution in the presence of dicyclohexyl carbodiimide, with ethyl 3-hydroxy-2-methylpropionate S36.10 to yield the phosphonate mixed diester S36.11.

Using this procedure, but employing, in place of ethyl 3-hydroxy-2-methylpropionate S36.10, different hydroxyesters S33.1, the corresponding products S33.2 are obtained.

The conversion of a phosphonate monoester S34.1 into a mixed diester S36.2 is also accomplished by means of a Mitsunobu coupling reaction with the hydroxyester S36.1, as described in Org. Lett., 2001, 643. In this method, the reactants 34.1 and S36.1 are combined in a polar solvent such as tetrahydrofuran, in the presence of a triarylphosphine and a dialkyl azodicarboxylate, to give the mixed diester S36.2. The R¹ substituent is varied by cleavage, using the methods described previously, to afford the monoacid product S36.3. The product is then coupled, for example using methods described above, with the hydroxy compound R³OH, to give the diester product S36.4.

The procedure is illustrated in Scheme 36, Example 2. In this method, a monoallyl phosphonate S36.12 is coupled in tetrahydrofuran solution, in the presence of triphenylphosphine and diethylazodicarboxylate, with ethyl lactate S36.13 to give the mixed diester S36.14. The product is reacted with tris(triphenylphosphine) rhodium chloride (Wilkinson catalyst) in acetonitrile, as described previously, to remove the allyl group and produce the monoacid product S36.15. The latter compound is then coupled, in pyridine solution at ambient temperature, in the presence of dicyclohexyl carbodiimide, with one molar equivalent of 3-hydroxypyridine S36.16 to yield the mixed diester S36.17.

Using the above procedures, but employing, in place of the ethyl lactate S36.13 or 3-hydroxypyridine, a different hydroxyester S36.1 and/or a different hydroxy compound R³OH, the corresponding products S36.4 are obtained.

The mixed diesters S36.2 are also obtained from the monoesters S34.1 via the intermediacy of the activated monoesters S36.5. In this procedure, the monoester S34.1 is converted into the activated compound S36.5 by reaction with, for example, phosphorus pentachloride, as described in J. Org. Chem., 2001, 66, 329, or with thionyl chloride or oxalyl chloride (Lv=Cl), or with triisopropylbenzenesulfonyl chloride in pyridine, as described in Nucleosides and Nucleotides, 2000, 19, 1885, or with carbonyl diimidazole, as described in J. Med. Chem., 2002, 45, 1284. The resultant activated monoester is then reacted with the hydroxyester S36.1, as described above, to yield the mixed diester S36.2.

The procedure is illustrated in Scheme 36, Example 3. In this sequence, a monophenyl phosphonate S36.9 is reacted, in acetonitrile solution at 70° C., with ten equivalents of thionyl chloride, so as to produce the phosphoryl chloride S36.19. The product is then reacted with ethyl 4-carbamoyl-2-hydroxybutyrate S36.20 in dichloromethane containing triethylamine, to give the mixed diester S36.21.

Using the above procedures, but employing, in place of ethyl 4-carbamoyl-2-hydroxybutyrate S36.20, different hydroxyesters S36.1, the corresponding products S36.2 are obtained.

The mixed phosphonate diesters are also obtained by an alternative route for incorporation of the R³O group into intermediates S36.3 in which the hydroxyester moiety is already incorporated. In this procedure, the monoacid intermediate S36.3 is converted into the activated derivative S36.6 in which Lv is a leaving group such as chloro, imidazole, and the like, as previously described. The activated intermediate is then reacted with the hydroxy compound R³OH, in the presence of a base, to yield the mixed diester product S36.4.

The method is illustrated in Scheme 36, Example 4. In this sequence, the phosphonate monoacid S36.22 is reacted with trichloromethanesulfonyl chloride in tetrahydrofuran containing collidine, as described in J. Med. Chem., 1995, 38, 4648, to produce the trichloromethanesulfonyloxy product S36.23. This compound is reacted with 3-(morpholinomethyl)phenol S36.24 in dichloromethane containing triethylamine, to yield the mixed diester product S36.25.

Using the above procedures, but employing, in place of with 3-(morpholinomethyl)phenol S36.24, different alcohols R³OH, the corresponding products S36.4 are obtained.

The phosphonate esters S36.4 are also obtained by means of alkylation reactions performed on the monoesters S34.1. The reaction between the monoacid S34.1 and the haloester S36.7 is performed in a polar solvent in the presence of a base such as diisopropylethylamine, as described in Anal. Chem., 1987, 59, 1056, or triethylamine, as described in J. Med. Chem., 1995, 38, 1372, or in a non-polar solvent such as benzene, in the presence of 18-crown-6, as described in Syn. Comm., 1995, 25, 3565.

The method is illustrated in Scheme 36, Example 5. In this procedure, the monoacid S36.26 is reacted with ethyl 2-bromo-3-phenylpropionate S36.27 and diisopropylethylamine in dimethylformamide at 80° C. to afford the mixed diester product S36.28.

Using the above procedure, but employing, in place of ethyl 2-bromo-3-phenylpropionate S36.27, different haloesters S36.7, the corresponding products S36.4 are obtained.

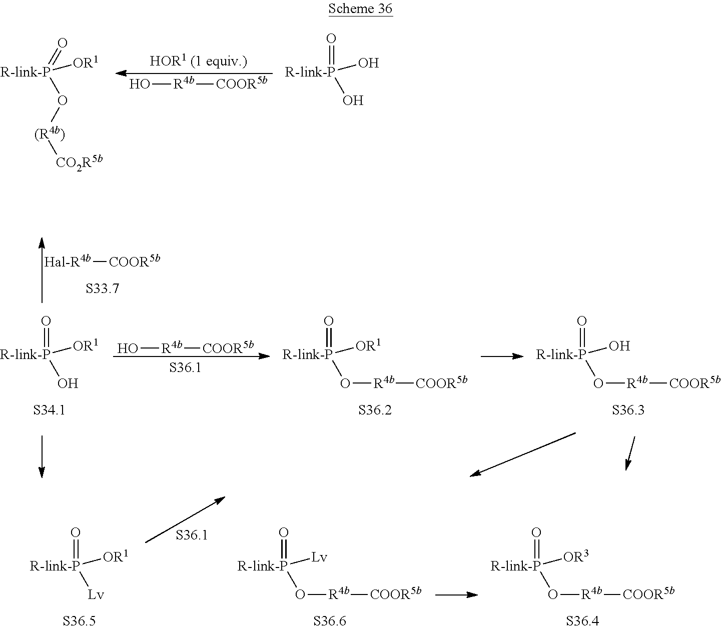

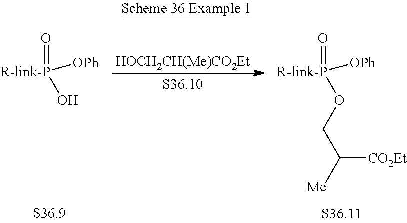

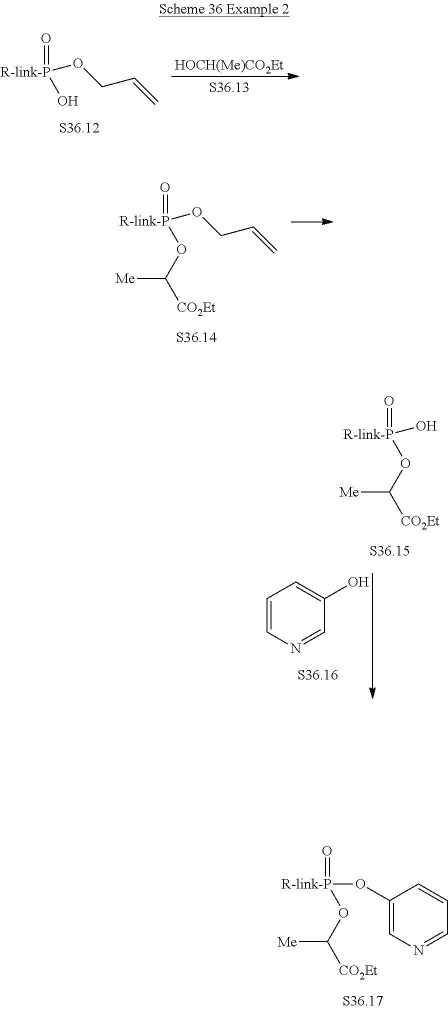

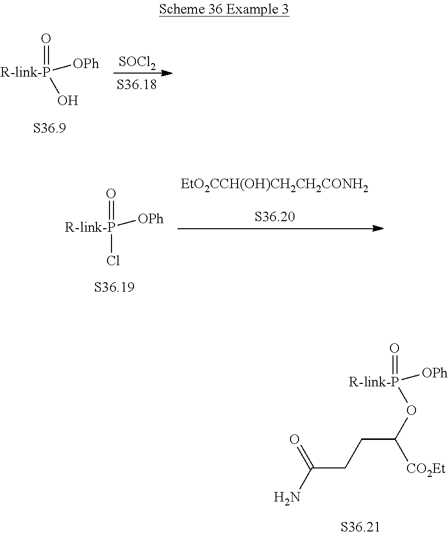

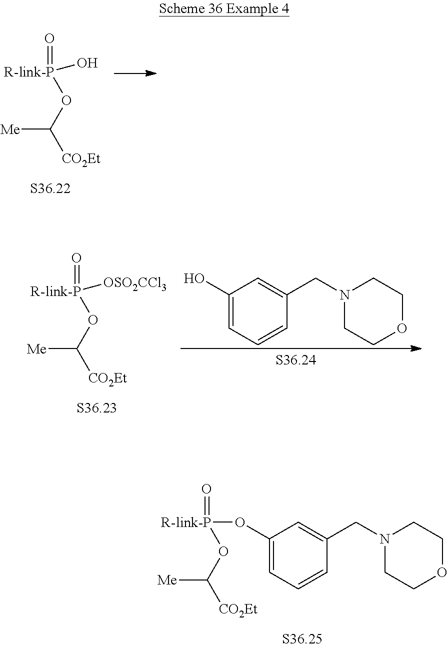

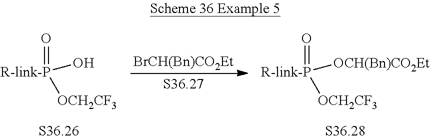

Scheme 37 illustrates methods for the preparation of phosphonate diesters in which both the ester substituents incorporate carboalkoxy groups.

The compounds are prepared directly or indirectly from the phosphonic acids S34.6. In one alternative, the phosphonic acid is coupled with the hydroxyester S37.2, using the conditions described previously in Schemes 34-36, such as coupling reactions using dicyclohexyl carbodiimide or similar reagents, or under the conditions of the Mitsunobu reaction, to afford the diester product S37.3 in which the ester substituents are identical.

This method is illustrated in Scheme 37, Example 1. In this procedure, the phosphonic acid S34.6 is reacted with three molar equivalents of butyl lactate S37.5 in the presence of Aldrithiol-2 and triphenyl phosphine in pyridine at ca. 70° C., to afford the diester S37.6.

Using the above procedure, but employing, in place of butyl lactate S37.5, different hydroxyesters S37.2, the corresponding products S37.3 are obtained.

Alternatively, the diesters S37.3 are obtained by alkylation of the phosphonic acid S34.6 with a haloester S37.1. The alkylation reaction is performed as described in Scheme 36 for the preparation of the esters S36.4.

This method is illustrated in Scheme 37, Example 2. In this procedure, the phosphonic acid S34.6 is reacted with excess ethyl 3-bromo-2-methylpropionate S37.7 and diisopropylethylamine in dimethylformamide at ca. 80° C., as described in Anal. Chem., 1987, 59, 1056, to produce the diester S37.8.

Using the above procedure, but employing, in place of ethyl 3-bromo-2-methylpropionate S37.7, different haloesters S37.1, the corresponding products S37.3 are obtained.

The diesters S37.3 are also obtained by displacement reactions of activated derivatives S34.7 of the phosphonic acid with the hydroxyesters S37.2. The displacement reaction is performed in a polar solvent in the presence of a suitable base, as described in Scheme 36. The displacement reaction is performed in the presence of an excess of the hydroxyester, to afford the diester product S37.3 in which the ester substituents are identical, or sequentially with limited amounts of different hydroxyesters, to prepare diesters S37.3 in which the ester substituents are different.

The methods are illustrated in Scheme 37, Examples 3 and 4. As shown in Example 3, the phosphoryl dichloride S35.22 is reacted with three molar equivalents of ethyl 3-hydroxy-2-(hydroxymethyl)propionate S37.9 in tetrahydrofuran containing potassium carbonate, to obtain the diester product S37.10.

Using the above procedure, but employing, in place of ethyl 3-hydroxy-2-(hydroxymethyl)propionate S37.9, different hydroxyesters S37.2, the corresponding products S37.3 are obtained.

Scheme 37, Example 4 depicts the displacement reaction between equimolar amounts of the phosphoryl dichloride S35.22 and ethyl 2-methyl-3-hydroxypropionate S37.11, to yield the monoester product S37.12. The reaction is conducted in acetonitrile at 70° in the presence of diisopropylethylamine. The product S37.12 is then reacted, under the same conditions, with one molar equivalent of ethyl lactate S37.13, to give the diester product S37.14.

Using the above procedures, but employing, in place of ethyl 2-methyl-3-hydroxypropionate S37.11 and ethyl lactate S37.13, sequential reactions with different hydroxyesters S37.2, the corresponding products S37.3 are obtained.

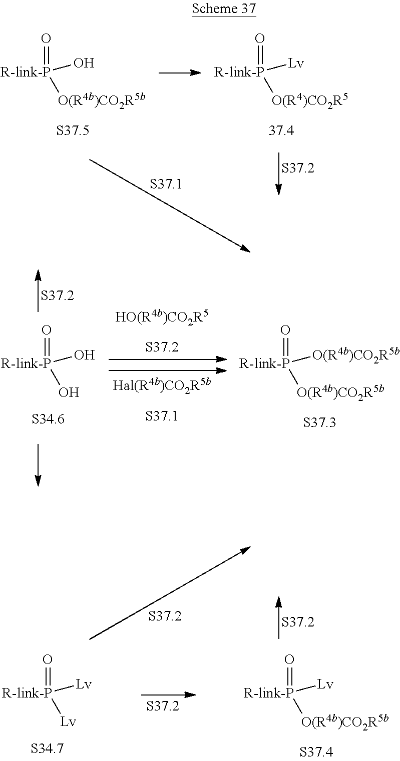

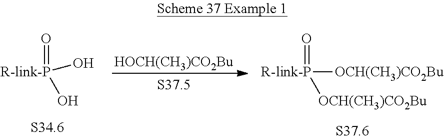

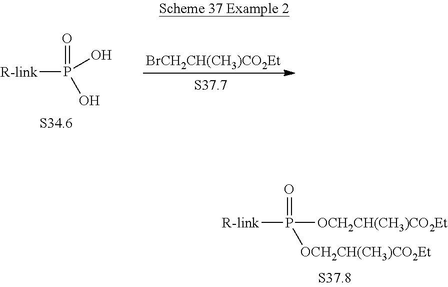

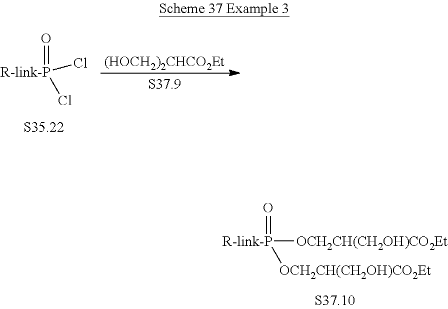

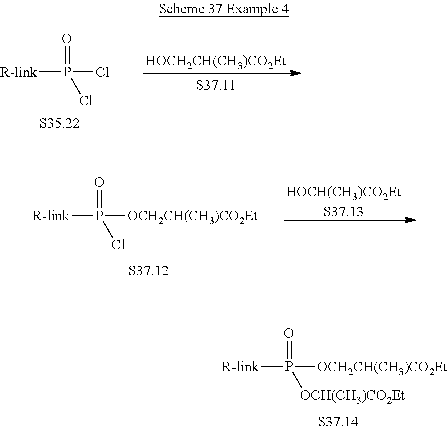

2,2-Dimethyl-2-aminoethylphosphonic acid intermediates can be prepared by the route in Scheme 5. Condensation of 2-methyl-2-propanesulfinamide with acetone give sulfinyl imine S38.11 (J. Org. Chem. 1999, 64, 12). Addition of dimethyl methylphosphonate lithium to S38.11 afford S38.12. Acidic methanolysis of S38.12 provide amine S38.13. Protection of amine with Cbz group and removal of methyl groups yield phosphonic acid S38.14, which can be converted to desired S38.15 (Scheme 38a) using methods reported earlier on. An alternative synthesis of compound S38.14 is also shown in Scheme 38b. Commercially available 2-amino-2-methyl-1-propanol is converted to aziridines S38.16 according to literature methods (J. Org. Chem. 1992, 57, 5813; Syn. Lett. 1997, 8, 893). Aziridine opening with phosphite give S38.17 (Tetrahedron Lett. 1980, 21, 1623). Reprotection) of S38.17 affords S38.14.

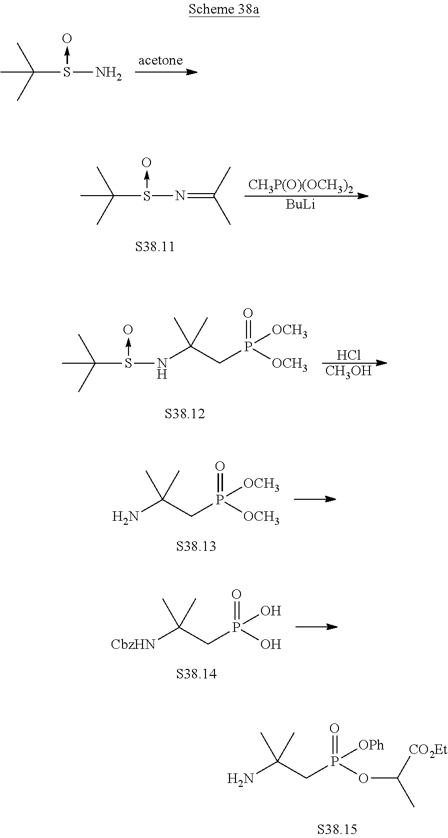

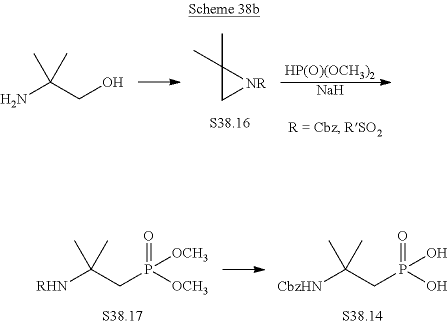

The invention will now be illustrated by the following non-limiting Examples.

### EXAMPLE 1.

### Synthesis of Representative Compounds of Formula 1

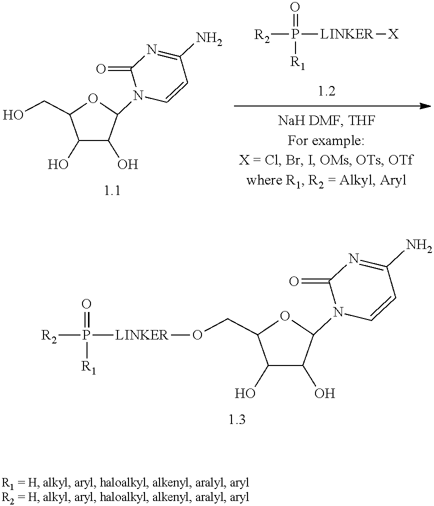

Representative compounds of the invention can be prepared as illustrated above. The desired phosphonate substituted analogs are prepared by reaction of arabinofuranosylcytosine 1.1 (obtained as described in U.S. Pat. No. 3,116,282, col. 26 line 65 to col. 28 line 25) with the respective alkylating reagents 1.2. Illustrated above is the preparation of phosphonate linkage to 1.1 through the 5′-hydroxyl group. Compound 1.1 is dissolved in a solvent such as DMF, THF and is treated with a phosphonate reagent bearing a leaving group, for example, bromine, mesyl, tosyl, or trifluoromethanesulfonyl in the presence of a suitable organic or inorganic base.

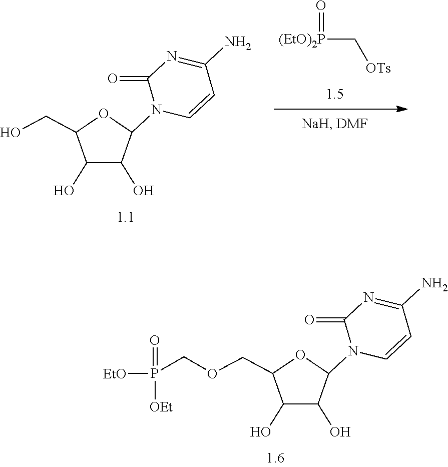

For instance, 1.1 dissolved in DMF, is treated with 8 equivalents of sodium hydride and two equivalents of (toluene-4-sulfonylmethyl)-phosphonic acid diethyl ester 1.5, prepared according to the procedures in JOC, 1996, 61, 7697, to give phosphonate 1.6 in which the linkage is a methylene group. Using the above procedure but employing different phosphonate reagents 1.2 in place of 1.5, the corresponding products 1.3 bearing different linking groups are obtained.

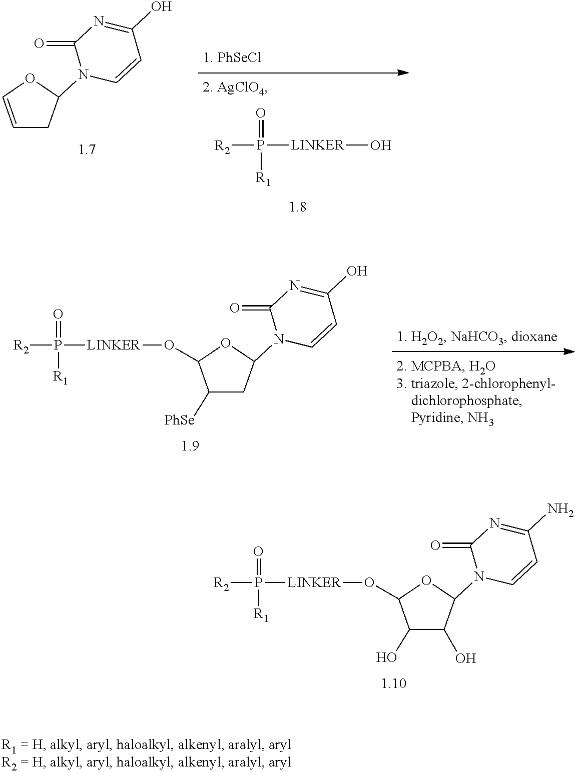

Representative compounds of the invention can be prepared as illustrated above. The desired phosphonate substituted analogs can be prepared by first reacting glycal 1.7 (obtained as described in J. Am. Chem. Soc. 1972, 94, 3213) with phenylselenyl chloride followed by treatment with the respective phosphonate alcohols 1.8 in the presence of silver perchlorate (J. Org. Chem. 1991, 56, 2642-2647). Oxidation of the resulting chloride using hydrogen peroxide followed by dihydroxylation of the resulting double bond with MCPBA and water generates the anti-diol (Synth. Commun. 1989, 19, 1939), which upon aminolysis of uracil using triazole, 2-chlorophenyldichlorophosphate, pyridine and ammonia (Bioorg. Med. Chem Lett. 1997, 7, 2567) provides the desired product 1.10. Alternatively, the anti-diol can be accessed through an osmium tetroxide oxidation followed by selective protection and inversion using Mitsunobu conditions.

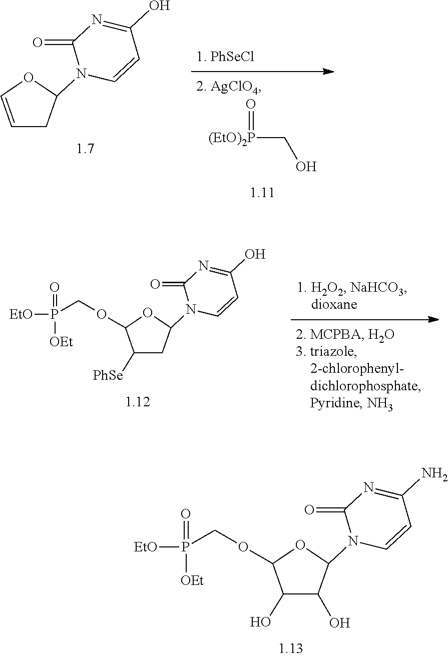

A specific compound of the invention can be prepared as follows. Compound 1.7 dissolved in CH₂Cl₂, is treated with one equivalent of phenyl selenyl chloride at −70° C. followed by silver perchlorate in the presence of diethyl(hydroxymethyl) phosphonate to generate 1.12. The phosphonate is transformed into the desired analog by first oxidation with hydrogen peroxide, followed by an MCPBA oxidation and finally conversion of uracil to cytosine to the desired product 1.13. Using the above procedure, but employing different phosphonate reagents 1.8 in place of 1.11, the corresponding products 1.10 bearing different linking groups are obtained.

In some cases, conversions to the desired phosphonates may require the use of suitable protecting groups for the amino group of cytosine as well as the diol. Other bases could also be used to generate similar analogs of both 1.3 and 1.10.

### EXAMPLE 2

### Synthesis of Representative Compounds of Formula 2

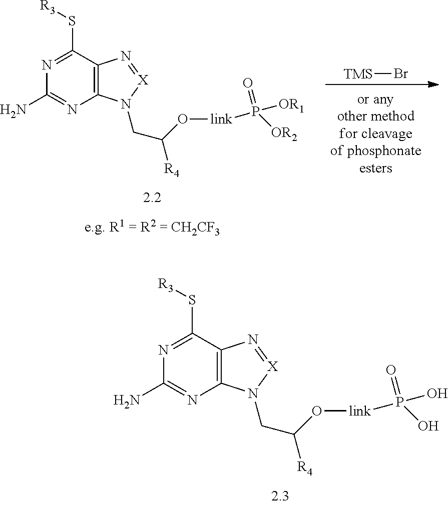

Representative compounds of the invention can be prepared as illustrated above. Intermediates 2.2 are prepared according to the methods described in U.S. Pat. No. 6,194,398 and any literature cited therein. The phosphonate ester of 2.2 may be converted to the final desired phosphonic acid functionality. Alternatively, phosphonic acids 2.3 may be formed by cleavage of esters 2.2 by treatment with a reagent such as, but not limited to, TMS-bromide in a solvent such as MeCN. Phosphonic acid 2.3 may then be converted to the final desired phosphonic acid functionality.

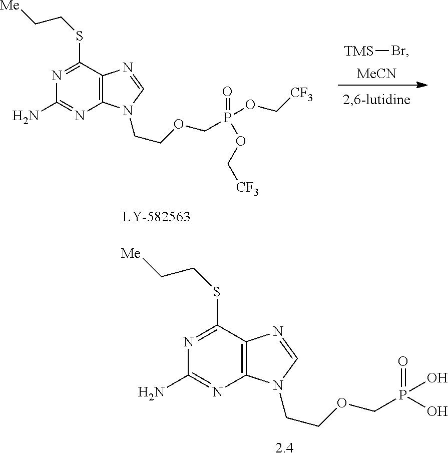

For instance, LY-582563, prepared as described in U.S. Pat. No. 6,194,398 is treated with TMS-Br and 2,6-lutidine in MeCN to provide phosphonic acid 2.4. Either LY-582563 or 2.4 may then be converted to the final desired phosphonate derivative.

### EXAMPLE 3

### Synthesis of Representative Compounds of Formulae 3 and 4

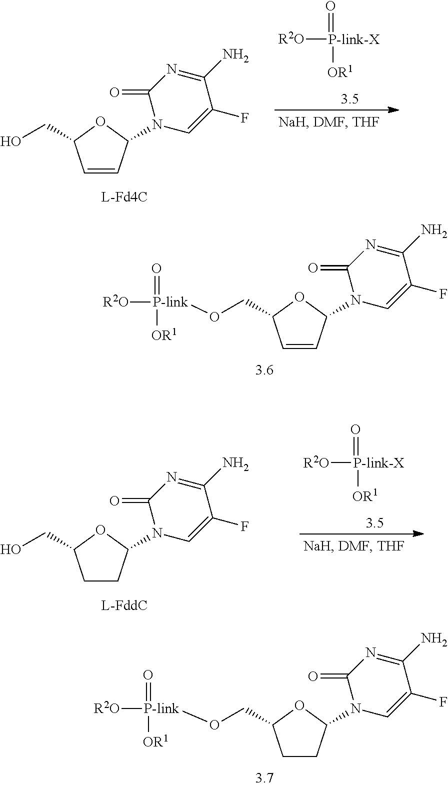

Representative compounds of the invention can be prepared as illustrated above. L-Fd4C and L-FddC are prepared according to methods in U.S. Pat. No. 5,561,120, U.S. Pat. No. 5,627,160, and U.S. Pat. No. 5,631,239 and any literature references cited therein. Either can be treated with a base such as, but not limited to, NaH or Cs₂CO₃, in a solvent such as, but not limited to, THF or DMF, and an alkylating agent of structure 3.5. In compounds 3.5, X is a leaving group such as, but not limited to, bromide, chloride, iodide, p-toluenesulfonate, trifluoromethanesulfonate, or methanesulfonate. It should be noted that cytosine-containing compounds sometimes require protection of the amino group at the 4-position of the base. If necessary, a protecting group may be introduced onto this position before these alkylation reactions are carried out. Introduction of such protecting groups (and their subsequent removal at the end of a synthetic scheme) are processes well known to those skilled in the art of nucleoside and nucleotide synthesis.

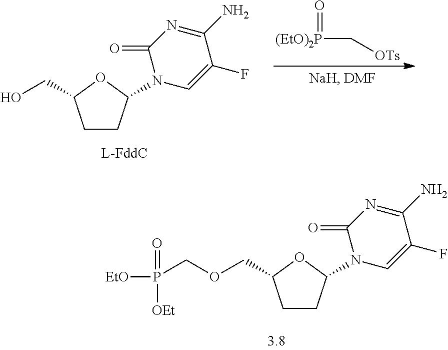

For instance, L-FddC is treated with NaH in DMF at 0° C. When bubbling has ceased, diethyl phosphonomethyltriflate (prepared according to Tetrahedron Lett. 1986, 27, 1477) is added. The resulting product 3.8 is isolated by standard chromatographic means. It may be necessary to protect the amino group at the 4-position of the base before this alkylation is carried out. See the note above regarding such protecting groups.

### EXAMPLE 4

### Synthesis of Representative Compounds of Formulae 5 and 6

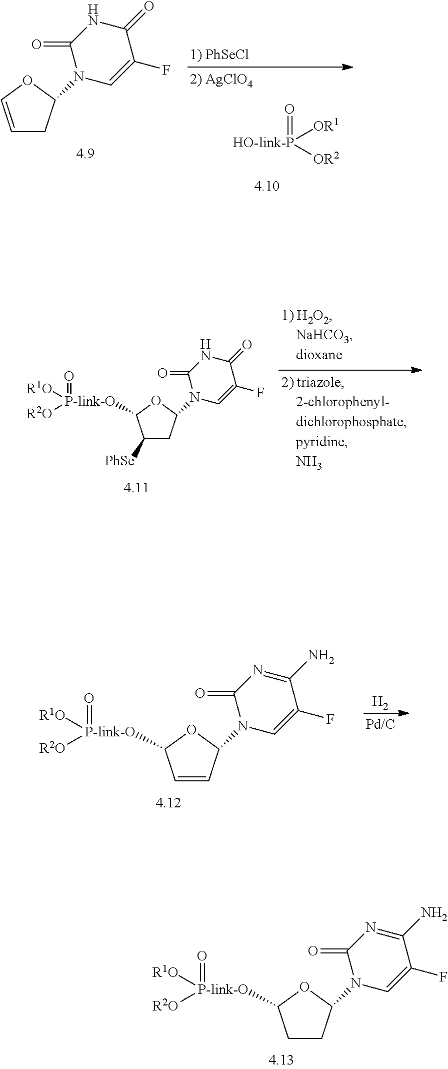

In Example 4, glycal 4.9 (obtained as described in J. Am. Chem. Soc. 1972, 94, 3213) is reacted with phenylselenyl chloride followed by treatment with the respective phosphonate alcohols 4.10 in the presence of silver perchlorate (J. Org. Chem. 1991, 56, 2642-2647). Oxidation of the resulting chloride using hydrogen peroxide followed by aminolysis of uracil using triazole, 2-chlorophenyldichlorophosphate, pyridine and ammonia (Bioorg. Med. Chem. Lett. 1997, 7, 2567) provides the L-Fd4C phosphonate derivative 4.12. Hydrogenation over 10% Pd/C provides the L-FddC derivative 4.13.

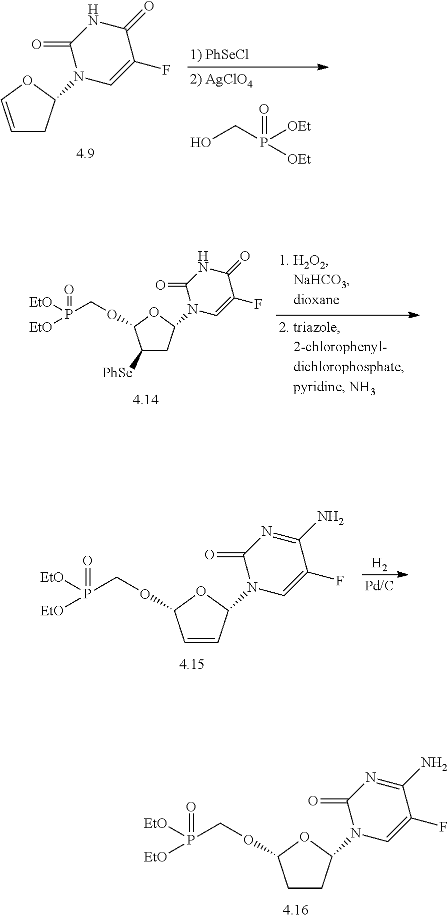

For instance, glycal 4.9 is reacted with phenylselenyl chloride and then treated with AgClO₄ and diethyl phosphonomethanol (available from Aldrich) providing compound 4.14. Treatment of 4.14 with H₂O₂ and NaHCO₃ in 1,4-dioxane followed by triazole, 2-chlorophenyldichlorophospate, in pyridine with ammonia yields the fluorocytosine derivative 4.15. Hydrogenation at 1 atm, over 10% Pd/C yields derivative 4.16.

### EXAMPLE 5

### Synthesis of Representative Compounds of Formula 9

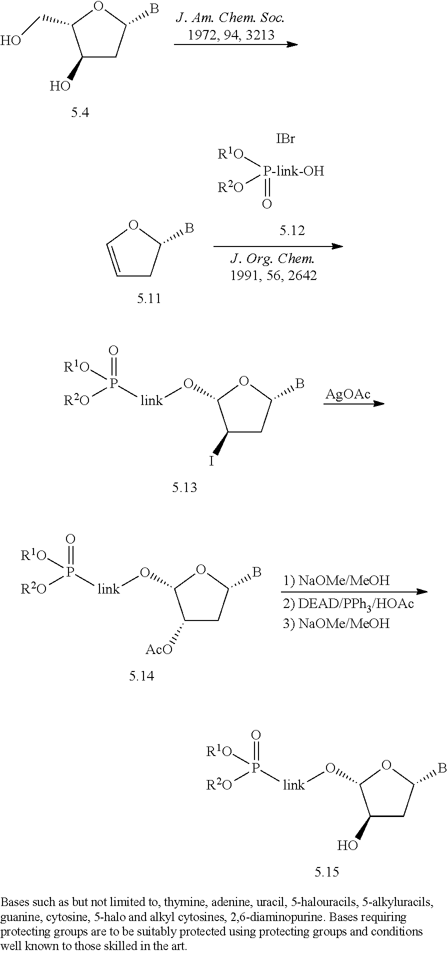

Representative compounds of the invention can be prepared as illustrated above. Compounds 5.4, prepared as described in WO 00/09531, U.S. Pat. No. 6,395,716, and U.S. Pat. No. 6,444,652, can be converted to glycal 5.11 according to the process reported in J. Am. Chem. Soc. 1972, 94, 3213. Glycal 5.11 is then treated with IBr in the presence of alcohol 5.12 to provide intermediate 5.13 (see J. Org. Chem. 1991, 56, 2642). The iodide of intermediate 5.13 can be treated with AgOAc to provide acetate 5.14, which can be deacetylated in the presence of catalytic sodium methoxide in methanol. Treatment of this product with DEAD and PPh₃ in the presence of acetic acid, followed by another deprotection with catalytic sodium methoxide in methanol will provide intermediate 5.15, which is representative of Formula 9. The phosphonates of intermediates 5.15 can be converted into other embodiments of the invention according to procedures know to those of skill in the art.

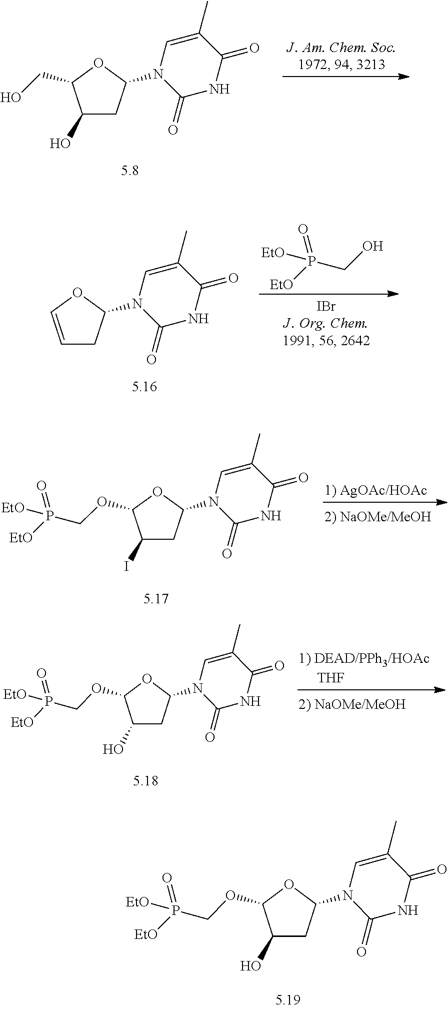

For instance, compound 5.8 is converted into glycal 5.16 according to the procedures reported in J. Am. Chem. Soc. 1972, 94, 3213. Glycal 5.16 is then treated with IBr in the presence of diethyl phosphonomethanol to provide intermediate 5.17 (see J. Org. Chem. 1991, 56, 2642). Intermediate 5.17 is then treated with AgOAc followed by deprotection with catalytic NaOMe in MeOH to provide 5.18. This compound is then converted into epimer 5.19 by a Mitsunobu reaction with DEAD/PPh₃ and HOAc in THF, followed by a second catalytic NaOMe/MeOH deprotection. At any point in the synthetic sequence where it is appropriate, the phosphonate group may be converted into a phosphonate with the desired substitution.

### EXAMPLE 6

### Synthesis of Representative Compounds of Formulae 10 and 11

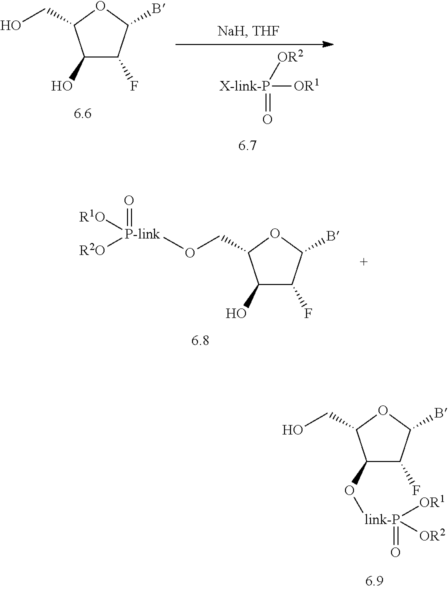

Representative compounds of the invention can be prepared as illustrated above. The preparation of compounds of structural type 6.6 are described in U.S. Pat. No. 5,565,438, U.S. Pat. No. 5,567,688, and U.S. Pat. No. 5,587,362, and the references cited therein. The compounds are then treated with a limiting amount of NaH in an appropriate solvent such as, but not limited to THF or DMF, and are then treated with an alkylating agent of type 6.7 (X=leaving group such as, but not limited to bromide, chloride, iodide, methanesulfonate, trifluoromethanesulfonate, and p-toluenesulfonate). Intermediates 6.8 and 6.9 result as a mixture and can be separated by chromatographic means that are well known to those skilled in the art. It should be noted that if a base requires a protecting group during this alkylation reaction, suitable protecting groups either will have already been installed throughout the synthetic schemes that provided starting materials 6.6 described in the cited patents, or can be installed prior to the alkylation reaction according to methods well known to chemists skilled in the art. If a protecting group had been added, it may be cleaved at this time according to the methods described in the patents cited above or according to any appropriate method known to those skilled in the art. At this point, the phosphonate esters may be converted to the desired final phosphonate functionality.

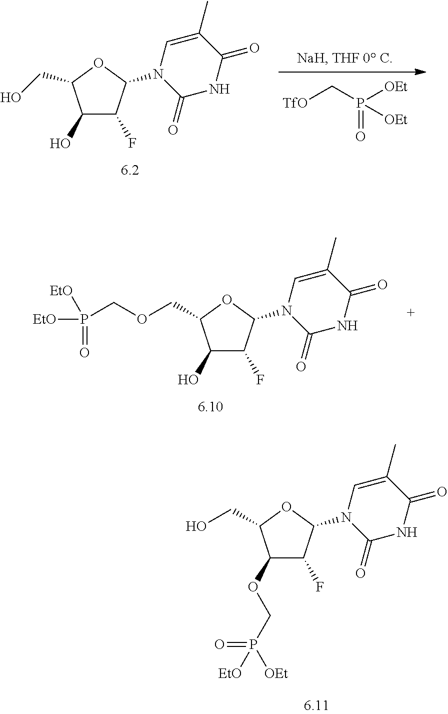

Clevudine, prepared as described in the patents cited above, is treated in anhydrous THF with NaH at 0° C. When bubbling ceases, diethyl phosphonomethyltriflate (prepared as in Tetrahedron Lett. 1986, 27, 1477) is added. The resulting alkylation products 6.10 and 6.11 are isolated after work-up either using silica gel or reversed-phase chromatography. The phosphonates may then be converted to the final desired products.

### EXAMPLE 7

### Synthesis of Representative Compounds of Formula 12

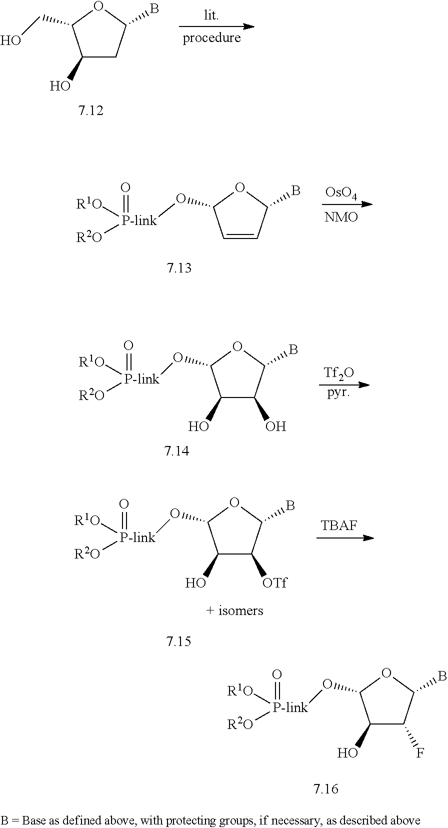

Representative compounds of the invention can be prepared as illustrated above. L-Deoxynucleoside 7.12 is synthesized according to literature procedure (see the methods reported by Holy, Collect. Czech. Chem. Commun. 1972, 37, 4072). L-Deoxynucleoside 7.12 is then converted into 7.13 through the procedures reported in J. Am. Chem. Soc. 1972, 94, 3213 and J. Org. Chem. 1991, 56, 2642. Dimethyl phosphonomethanol may be replaced with any alcohol linked to a phosphonate. The double bond of compound 7.13 is then treated with OsO₄ and N-methylmorpholine N-oxide to provide the dihydroxylated derivatives 7.14. Triflation of 7.14 results in a mixture of triflates, the desired of which, 7.15, is isolated by the appropriate chromatographic method. The fluoride is installed by treatment of 7.15 with tetra-n-butylammonium fluoride (TBAF) in an appropriate solvent, such as THF, yielding the desired intermediate 7.16.

A specific compound of Formula 12 can be prepared as follows.

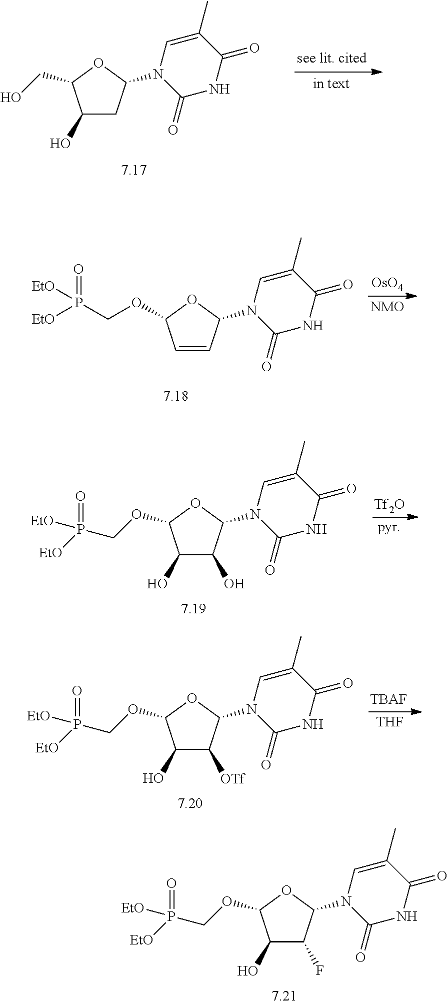

L-Thymidine 7.17, synthesized by Holy's method, is converted according to the literature procedures cited above to d4 nucleoside derivative 7.18. Compound 7.18 is then treated with OsO₄ and NMO to give dihydroxylated product 7.19, which is triflated to provide 7.20 (separated by silica gel chromatography from a mixture of its regioisomers and di-triflated material). Compound 7.20 is then treated with TBAF to convert it to the desired compound 7.21. The diethyl phosphonate may now be converted into any group that is desired according to methods well known to chemists skilled in the art.

### EXAMPLE 8

### Synthesis of Representative Compounds of the Invention

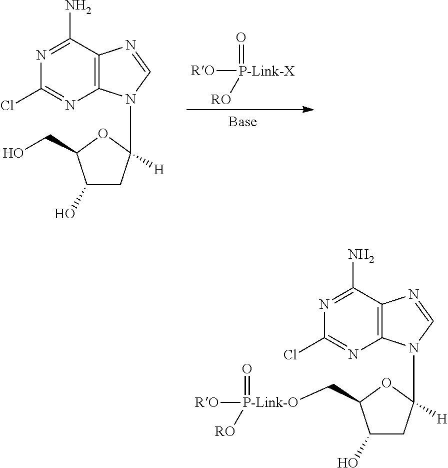

### EXAMPLE 9

### Synthesis of Representative Compounds of the Invention

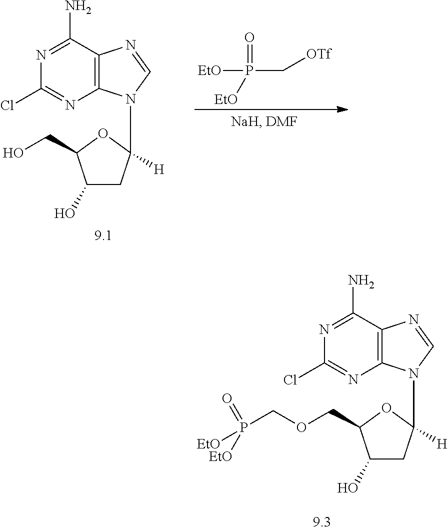

Representative compounds of the invention can be prepared as illustrated above. The 2-chloro-2′-deoxyadenosine 9.1 (prepared according to the procedure of Ikehara, M. et al., J. Am. Chem. Soc., (1963), 85, 2344, also see Ikehara, M. et al., J. Am. Chem. Soc., (1965), 87, 3, 606) can be treated in a solvent such as tetrahydrofuran or dimethylformamide with a base such as sodium hydride. When bubbling ceases, diethyl phosphonomethyltriflate (prepared according to Tetrahedron Lett., (1986), 27, 1477) is added, yielding the desired phosphonate diester 9.3.

### EXAMPLE 10

### Synthesis of Representative Compounds of the Invention

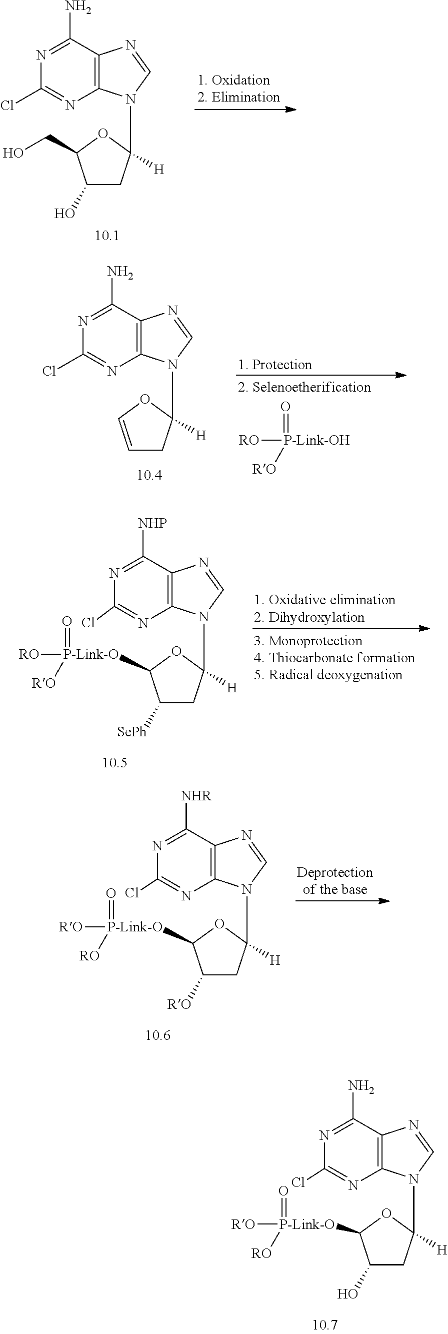

Representative compounds of the invention can be prepared as illustrated above. The preparation of compound 10.7 is illustrated above. Compound 10.1 (2-chloro-2′-deoxyadenosine) can be prepared as described in Ikehara, M. et al., J. Am. Chem. Soc., (1963), 85, 2344; see also Ikehara, M. et al., J. Am. Chem. Soc., (1965), 87, 3, 606. Oxidation of the 5′-OH followed by elimination provides glycal 10.4 (see the procedure of Zemlicka J. et al., J. Am. Chem. Soc., (1972), 94, 9, 3213). Protection of the chloroadenine at the 6 position followed by selenoetherification provides the protected phosphonate 10.5 (Kim, C. et al., J. Org. Chem., (1991), 56, 2642). Oxidative elimination of the phenylselenide (as described in Kim, C. et al., J. Org. Chem., (1991), 56, 2642) followed by stereoselective dihydroxylation provides the diol which can then be converted to the 3′ monoprotected sugar. Acylation of the 2′ alcohol with phenyl chlorothionoformate provides the precursor for Robins deoxygenation. Subsequent deoxygenation provides compound 10.6 (Metteucci, M. D. et al., Tetrahedron Lett., (1987), 28, 22, 2469, see also Robins, M. J. et al., J. Org. Chem., (1995), 60, 7902). Finally, the protecting groups are removed.

### EXAMPLE 11

### Synthesis of Representative Compounds of the Invention

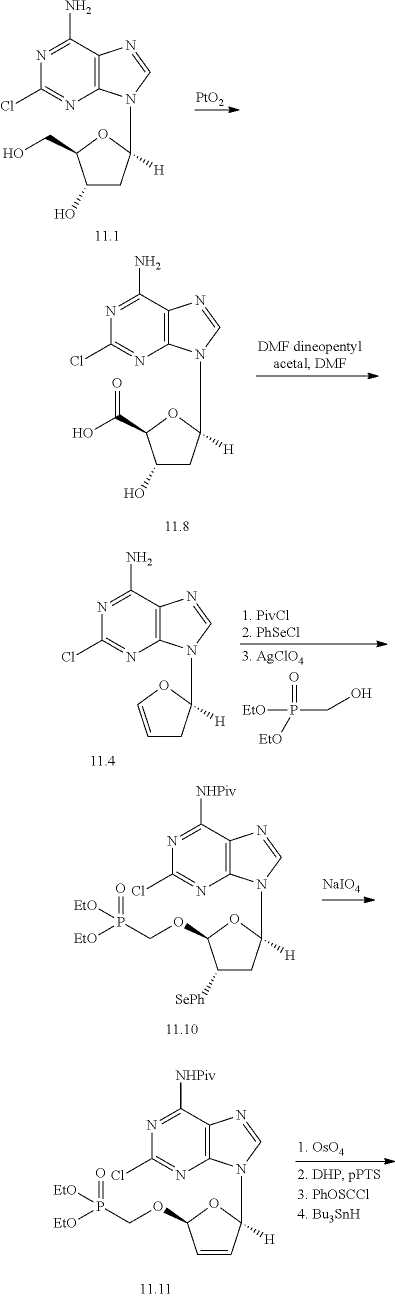

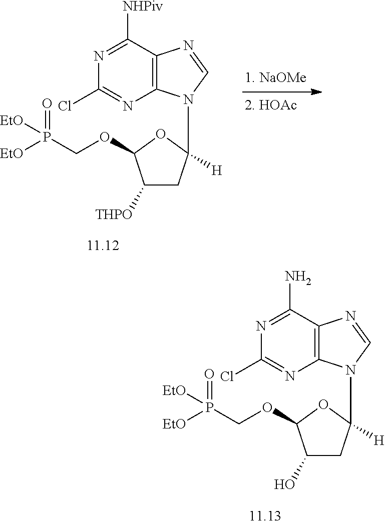

Representative compounds of the invention can be prepared as illustrated above. Specifically, 2-chloro-2′-deoxyadenosine, compound 11.1 can be oxidized with PtO₂ to provide carboxylic acid 11.8. Decarboxylative elimination is achieved using dimethylformamide dineopentyl acetal in DMF at high temperature (Zemlicka J. et al., J. Am. Chem. Soc., (1972), 94, 9, 3213). Once the furanoid glycal 11.4 is in hand, it is first protected at the 6-position of the 2-chloroadenosine using PivCl conditions as described in Greene, T., Protective groups in organic synthesis, Wiley-Interscience, 1999. Treatment of the protected glycal with silver perchlorate in the presence of diethyl(hydroxylmethyl)phosphonate (Phillion, D. et al., Tetrahedron Lett., 1986, 27, 1477) provides the phosphonate 11.10 (Kim, C. et al., J. Org. Chem., (1991), 56, 2642). Oxidative elimination of the selenide followed by dihydroxylation using osmium tetraoxide provides a diol which can be monoprotected at the 3′ position using a THP group. Further acylation of the 2′ alcohol with phenyl chlorothionoformate provides the precursor for Robins deoxygenation, performed with tributyltin hydride, to give compound 11.12 (Metteucci, M. D. et al., Tetrahedron Lett., (1987), 28, 22, 2469, also see Robins, M. J. et al., J. Org. Chem., (1995), 60, 7902). Deprotection of the pivaloyl group by treatment with sodium methoxide (Greene, T., Protective groups in organic synthesis, Wiley-Interscience, (1999)) is followed by a final deprotection of the THP group in acetic acid.

### EXAMPLE 12

### Synthesis of Representative Compounds of the Invention

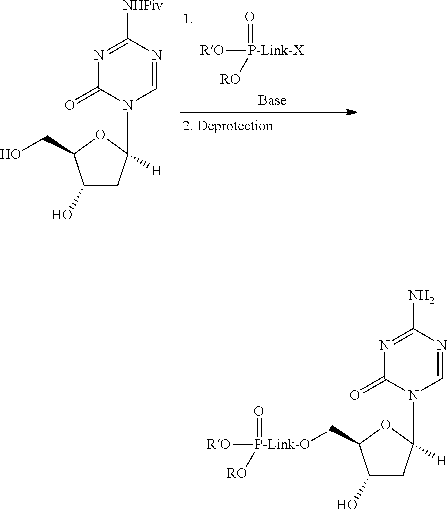

### EXAMPLE 13

### Synthesis of Representative Compounds of the Invention

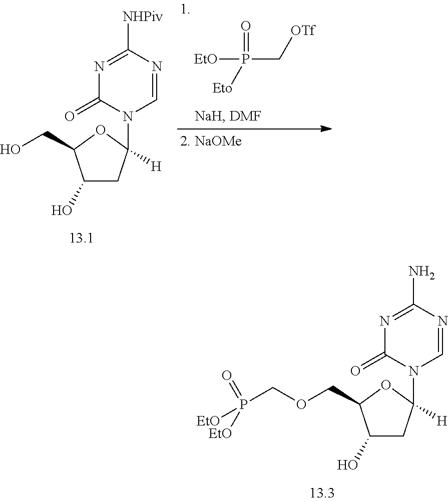

Representative compounds of the invention can be prepared as illustrated above. The appropriately protected 5-aza-2′-deoxycytidine, prepared according to the procedure of Winkley, M. W., Robins, R. K., J. Org. Chem., (1970), 35, 2, 491 (see also Ben-Hattar J., Jiricny, J. J. Org. Chem., (1986), 51, 3211), can be treated in a solvent such as tetrahydrofuran or dimethylformamide with a base such as sodium hydride. Formation of the pivaloyl compound 13.1 can be accomplished by protecting 5-aza-2′-deoxycytidine with a pivaloyl group (Greene, T., Protective Groups in Organic Synthesis, Wiley-Interscience, (1999)). When bubbling ceases, diethyl phosphonomethyltriflate (prepared according to Tetrahedron Lett., (1986), 27, 1477) is added, yielding the protected product 13.3. The pivaloyl group can be removed with sodium ethoxide to provide the desired phosphonate diester 13.3.

### EXAMPLE 14

### Synthesis of Representative Compounds of the Invention

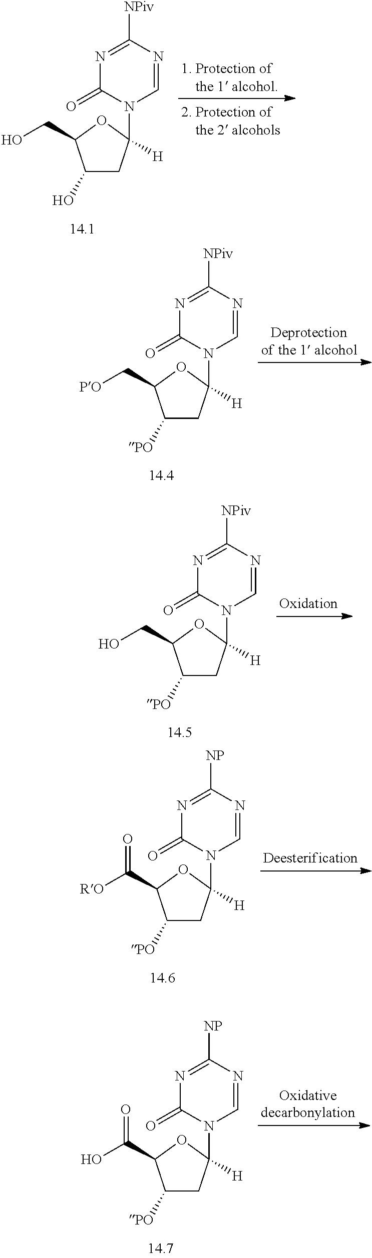

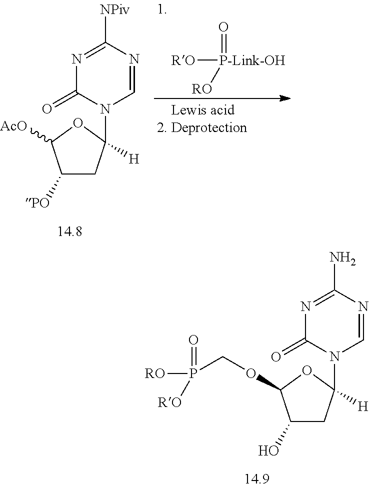

Representative compounds of the invention can be prepared as illustrated above. Compound 14.1 may be the pivaloyl protected 5-aza-2′-deoxycytidine that is described in Winkley, M. W., Robins, R. K., J. Org. Chem., (1970), 35, 2, 491 and Ben-Hattar J., Jiricny, J., J. Org. Chem., (1986), 51, 3211. Protection of the 5′ hydroxyl group followed by protection of 2′ alcohol provides compound 14.4. Removal of the 5′ protecting group provides the free primary alcohol. Corey's one-step oxidation procedure (Corey, E. J. et al., J. Org. Chem., (1984), 49, 4735) can be utilized to transform the primary alcohol to the ester 14.6. Deesterification, followed by oxidative decarbonylation using a modified Hunsdiecker reaction (Chu, C. K. et al., Tetrahedron Lett., (1991), 32, 3791) converts 14.7 to the acetate 14.8. The stereochemistry of the Vorbruggen glycosylation under Lewis acid conditions is controlled by protecting group participation at the 4′ position. A final deprotection provides the desired pro-drug 14.9.

### EXAMPLE 15

### Synthesis of Representative Compounds of the Invention

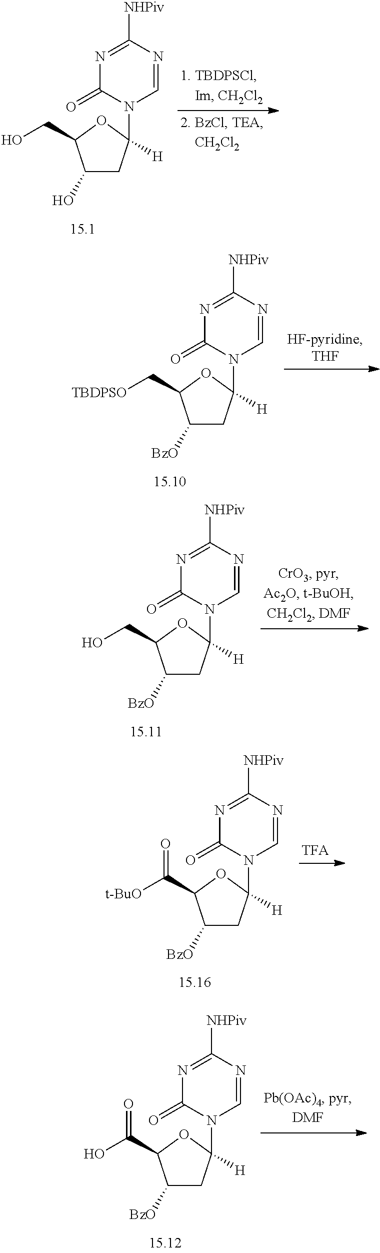

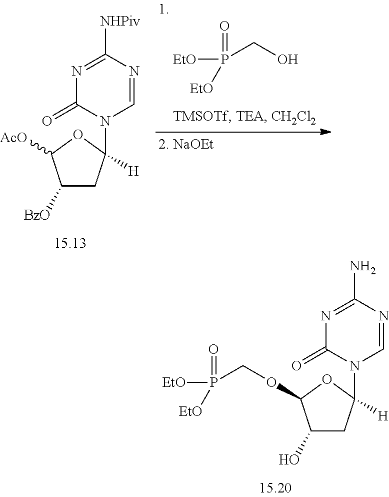

Representative compounds of the invention can be prepared as illustrated above. Specifically, compound 15.1, prepared by protection of 5-aza-2′-deoxycytidine (prepared as in Winkley, M. W., Robins, R. K., J. Org. Chem., (1970), 35, 2, 491 and Ben-Hattar J., Jiricny, J. J. Org. Chem., (1986), 51, 3211 using pivaloyl chloride, can be protected with a tert-butyldiphenylsilyl (TBDPS) group to provide the 5′-O-TBDPS analog. Further protection of the 3′ alcohol with the benzoyl group provides compound 15.10 (Teng, K., Cook, D. J. Org. Chem. (1994), 59, 278). Exposure of the fully protected compound 15.10 to HF-pyridine reagent selectively deprotects the 5′ hydroxyl group, which is then oxidized to the t-butyl ester using the Corey-Samuelsson oxidation (Corey, E. J., Samuelsson, B. J. Org. Chem., (1984), 49, 4735). Deesterification of the oxidized product using trifluoroacetic acid (TFA) provides compound 15.12. Oxidative decarboxylation using a modified Hunsdiecker reaction (Chu, C. K. et al., Tetrahedron Lett., (1991), 32, 3791) converts the free acid to the acetate 15.13 which may be a mixture of anomers at 5′. While separation of the anomers may be achieved by column chromatography, it is not necessary to do so. The stereochemical outcome of a Vorbruggen glycosylation is controlled by the stereochemistry of the 4′-benzoyl group due to anchimeric assistance, rendering separation of the isomers is unnecessary. Vorbruggen glycosylation using hydroxymethylphosphonic acid diethyl ester proceeds to provide the protected phosphonate. Final saponification to remove the pivaloate and the benzoate groups completes the synthesis of compound 15.20 (Greene, T., Protective Groups in Organic Synthesis, Wiley-Interscience, (1999)).

### EXAMPLE 16

### Synthesis of Representative Compounds of the Invention

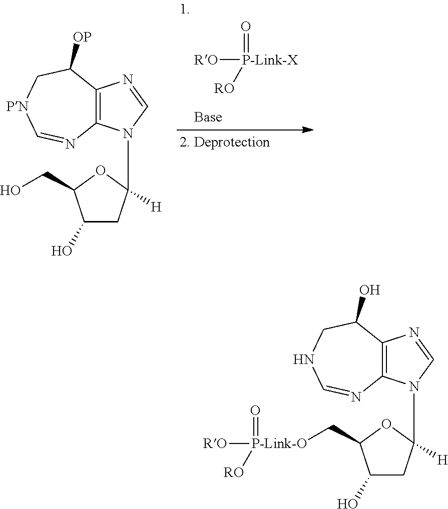

### EXAMPLE 17

### Synthesis of Representative Compounds of the Invention

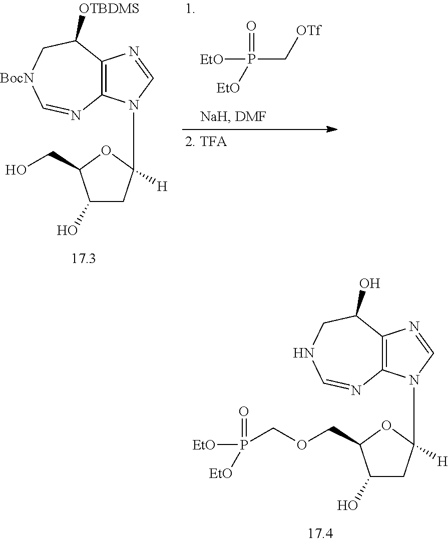

Representative compounds of the invention can be prepared as illustrated above. The appropriately protected 2′-deoxycoformycin prepared according to U.S. Pat. No. 3,923,785 (also reported in Chan, E. et al., J. Org. Chem., (1982), 47, 3457) can be treated in a solvent such as tetrahydrofuran or dimethylformamide with a base such as sodium hydride. Formation of the fully protected compound 17.3 can be accomplished utilizing (8R)-6-(t-butoxycarbonyl)-8-[(t-butyldimethylsilyl)oxy]-3,6,7,8-tetrahedroimidazo[4,5-d]-[1,3]diazapine, prepared by Truong, T. V. et al. J. Org. Chem. (1993), 58, 6090, through the Vorbruggen glycosylation reaction as described in Chan, E. et al., J. Org. Chem., (1982), 47, 3457. When bubbling ceases, diethyl phosphonomethyltriflate (prepared according to Tetrahedron Lett., (1986), 27, 1477) is added, yielding the desired phosphonate diester 17.4.

### EXAMPLE 18

### Synthesis of Representative Compounds of the Invention

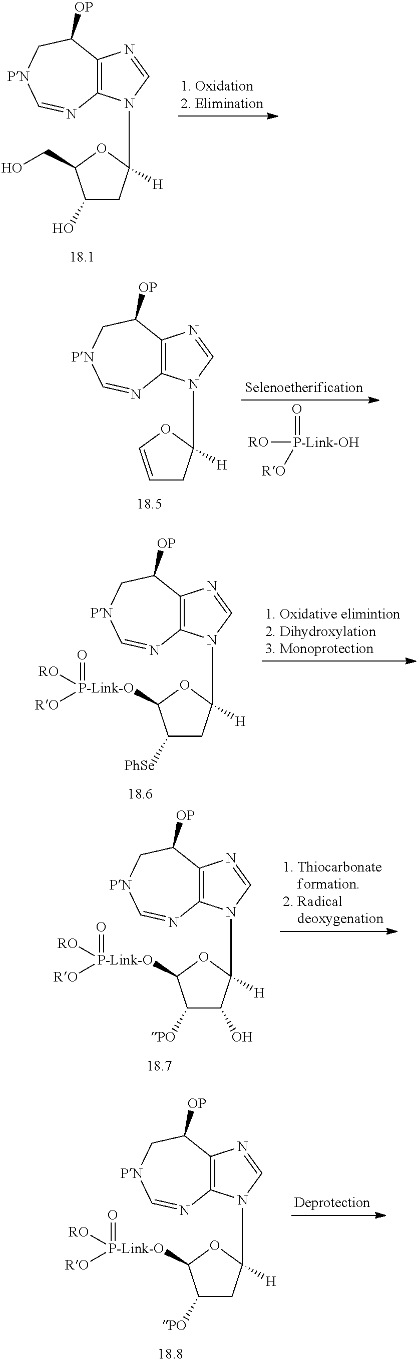

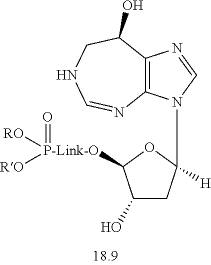

Representative compounds of the invention can be prepared as illustrated above. Compound 18.1 (8-(tert-butyl-dimethyl-silanyloxy)-3-(4-hydroxy-5-hydroxymethyl-tetrahydro-furan-2-yl)-7,8-dihydro-3H-imidazo[4,5-d][1,3]diazepine-6-carboxylic acid tert-butyl ester) can be prepared as described in Truong, T. V. et al. J. Org. Chem., (1993), 58, 6090 and Chan, E. et al., J. Org. Chem., (1982), 47, 3457. Oxidation of the 5′-OH followed by elimination of the carboxylic acid provides glycal 18.5 (see the procedure of Zemlicka J. et al., J. Am. Chem. Soc., (1972), 94, 9, 3213). Selenoetherification provides the protected phosphonate 18.6 (Kim, C. et al., J. Org. Chem., (1991), 56, 2642). Oxidative elimination of the phenylselenide (as described in Kim, C. et al., J. Org. Chem., (1991), 56, 2642) followed by stereoselective dihydroxylation provides the diol, which can then be converted to a monotetrahydropyran protected compound 18.7. Acylation of the 2′ alcohol with phenyl chlorothionoformate provides the precursor for Robins deoxygenation. Subsequent deoxygenation provides compound 18.8 (Metteuci, M. D. et al. Tetrahedron Lett., (1987), 28, 22, 2459, also see Robins, M. J. et al. J. Org. Chem., (1995), 60, 7902). The order of formation of the 3′ protected alcohol and thiocarbonate formation can also be reversed if the first protection proceeds exclusively at the 2′ position. In that case, the 2′ thiocarbonate is formed first, followed by protection of the 3′ hydroxyl group and a final Robins deoxygenation. Trifluoroacetic acid (TFA)-mediated deprotection removed all three protecting groups to provide compound 18.9.

### EXAMPLE 19

### Synthesis of Representative Compounds of the Invention

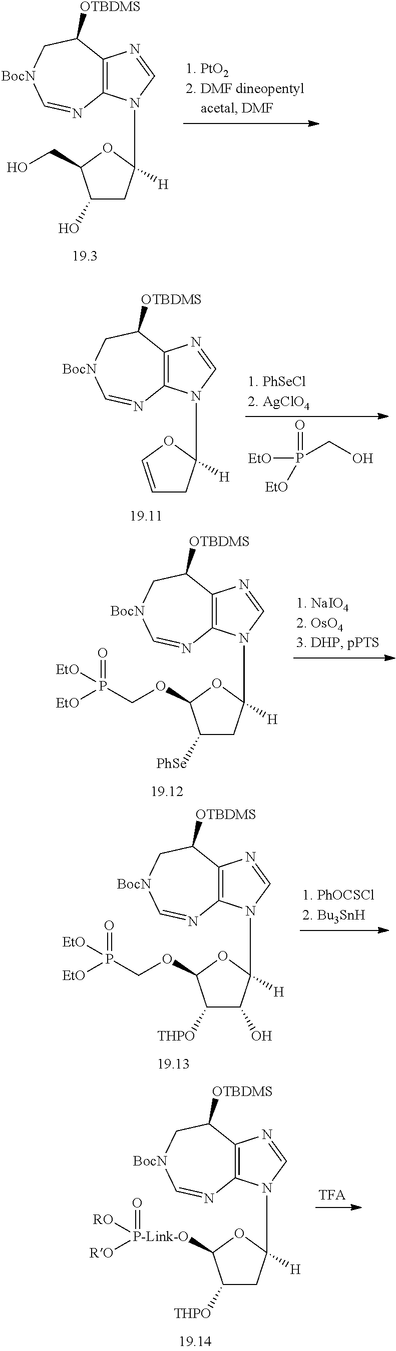

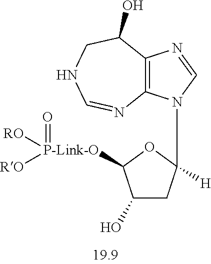

Representative compounds of the invention can be prepared as illustrated above. Specifically, compound 19.3 (Truong, T. V. et al., J. Org. Chem., (1993), 58, 6090 and Chan, E. et al., J. Org. Chem., (1982), 47, 3457) can be oxidized with PtO₂ to provide a carboxylic acid. Decarboxylative elimination is achieved using dimethylformamide dineopentyl acetal in DMF at high temperature (Zemlicka J. et al., J. Am. Chem. Soc., (1972), 94, 9, 3213). Once the furanoid glycal 19.11 is in hand, it is treated with phenylselenyl chloride to perform the selenoetherification followed by treatment with silver perchlorate in the presence of diethyl(hydroxylmethyl)phosphonate (Phillion, D. et al., Tetrahedron Lett., (1986), 27, 1477) to give phosphonate 19.12 (Kim, C. et al., J. Org. Chem., (1991), 56, 2642). Oxidative elimination of the selenide followed by dihydroxylation using osmium tetraoxide provides a diol, which is converted to the mono-protected tetrahydropyranyl ether compound 19.13. Acylation of the 2′ alcohol with phenyl chlorothionoformate provides the precursor for Robins deoxygenation, which is performed with tributyltin hydride to give compound 19.14 (Metteuci, M. D. et al., Tetrahedron Lett., (1987), 28, 22, 2459, also see Robins, M. J. et al., J. Org. Chem., (1995), 60, 7902). Removal of all the protecting groups is achieved using TFA to give compound 19.9 (Greene, T., Protective Groups in Organic Synthesis, Wiley-Interscience, (1999)).

### EXAMPLE 20

### Synthesis of Representative Compounds of the Invention

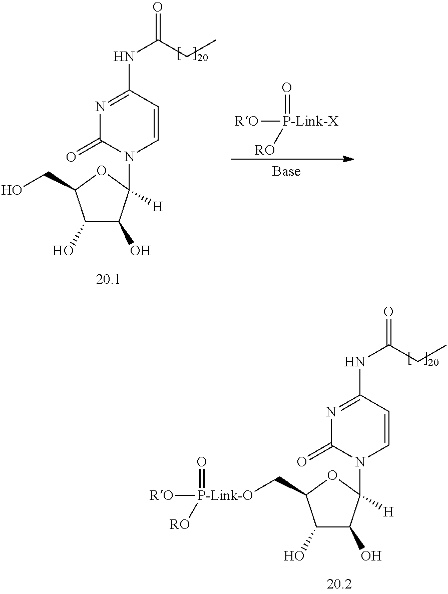

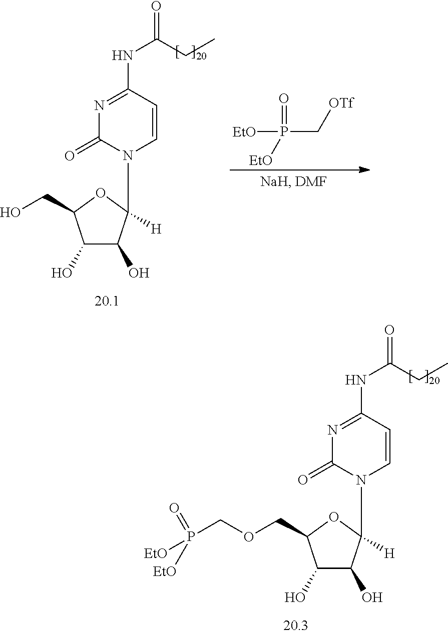

Representative compounds of the invention can be prepared as illustrated above. The N-(1-β-D-arabinofuranosyl-1,2-dihydro-2-oxo-4-pyrimidinyl)docosanamide (U.S. Pat. No. 3,991,045, also see Akiyama, M. et al., Chem. Pharm. Bull., (1978), 26, 3, 981) can be treated in a solvent such as tetrahydrofuran or dimethylformamide with a base such as sodium hydride. When bubbling ceases, diethyl phosphonomethyltriflate (prepared according to Tetrahedron Lett., (1986), 27, 1477) is added, yielding the desired phosphonate diesters 20.2 and 20.3.

### EXAMPLE 21

### Synthesis of Representative Compounds of the Invention

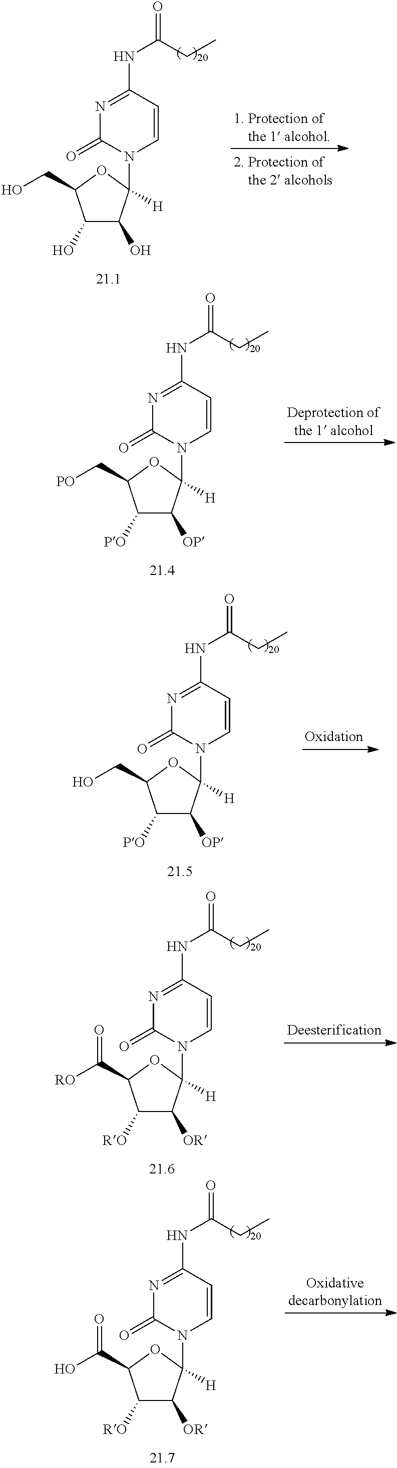

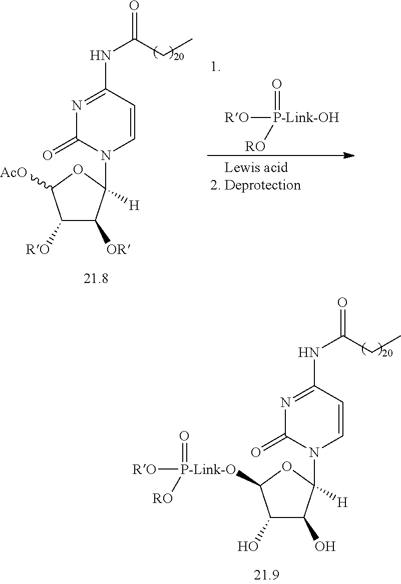

Representative compounds of the invention can be prepared as illustrated above. Compound 21.1 can be prepared according to U.S. Pat. No. 3,991,045. Protection of the 5′ hydroxyl group followed by protection of 2′ and 3′ alcohols provides compound 21.4. Removal of the 5′ protecting group provides the free primary alcohol precursor to the oxidation. Corey's one-step oxidation procedure (Corey, E. J. et al., J. Org. Chem., (1984), 49, 4735) can be utilized to transform the primary alcohol to the ester 21.6. Deesterification followed by oxidative decarbonylation using a modified Hunsdiecker reaction (Chu, C. K. et al., Tetrahedron Lett., (1991), 32, 3791) converts 21.7 to the acetate 21.8. A Vorbruggen glycosylation using Lewis acid conditions is controlled by the protecting group participation at the 4′ position. A final deprotection provides the desired prodrug 21.9.

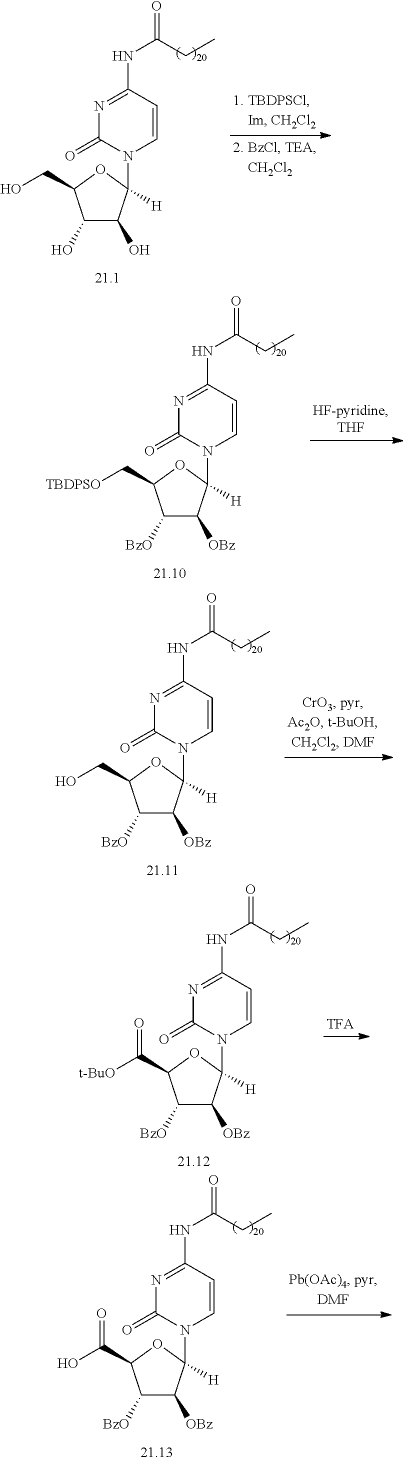

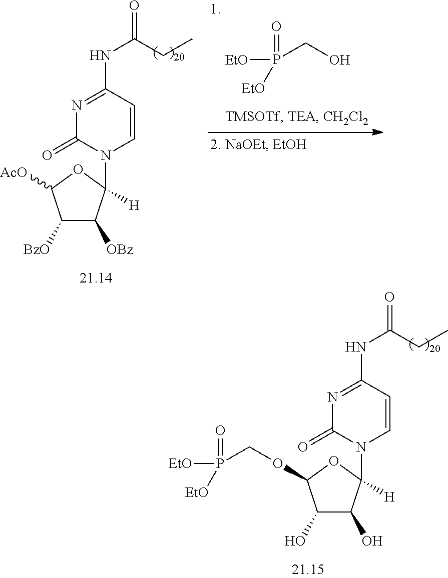

Specifically, compound 21.1, N-(1-β-D-arabinofuranosyl-1,2-dihydro-2-oxo-4-pyrimidinyl)docosanamide (U.S. Pat. No. 3,991,045) can be selectively protected with a tert-butyldiphenylsilyl (TBDPS) group to provide the 5′-O-TBDPS analog. Further protection of the 3′ and 4′ alcohols as benzoate esters provides compound 21.10 (Teng, K., Cook, D. J. Org. Chem., (1994), 59, 278). Exposure of the fully protected compound 21.10 to HF-pyridine reagent selectively deprotects the 5′ hydroxyl group that can then be oxidized to the t-butyl ester using the Corey-Samuelsson oxidation (Corey, E. J., Samuelsson, B. J. Org. Chem., (1984), 49, 4735). Deesterification of the oxidized product using trifluoroacetic acid provides compound 21.13. Oxidative decarboxylation using a modified Hunsdiecker reaction (Chu, C. K. et al., Tetrahedron Lett., (1991), 32, 3791) converts the free acid to the acetate 21.14, which may be a mixture of anomers at 5′. While separation of the anomers may be achieved by column chromatography, it is not necessary to do so. The stereochemical outcome of a Vorbruggen glycosylation is controlled by the stereochemistry of the 4′-benzoyl group due to anchimeric assistance, rendering separation of the isomers is unnecessary. Vorbruggen glycosylation using hydroxymethylphosphonic acid diethyl ester proceeds to provide the protected phosphonate. A final deprotection using hydrolysis conditions completes the synthesis of compound 21.15.

### EXAMPLE 22

### Synthesis of Representative Compounds of Formula 23

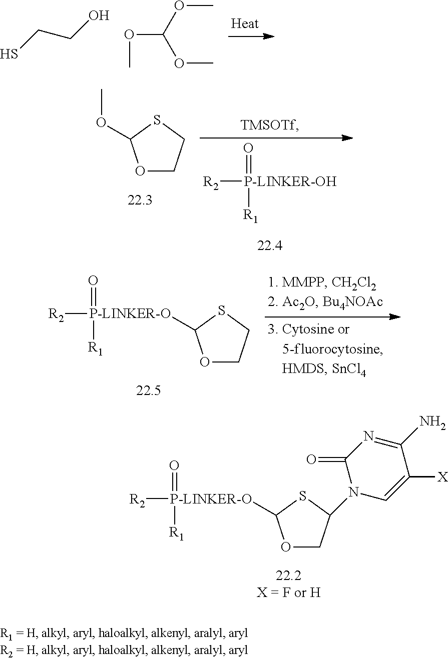

Representative compounds of the invention can be prepared as illustrated above. Condensation of commercially available 2-mercapto-ethanol and trimethoxymethane (J. Org. Chem. USSR (Engl. Transl) 1981, 1369-1371) generates heterocycle 22.3. Glycosidation using, for example, trimethylsilyl triflate and the phosphonate substituted alcohol 22.4, provides intermediate 22.5. Oxidation of sulfur to the sulfoxide using monoperoxyphthalic acid, magnesium salt (see U.S. Pat. No. 6,228,860 col. 15 ln. 45-60) followed by a Pummerer rearrangement (see U.S. Pat. No. 6,228,860 col. 16 ln. 25-40) and base introduction (cytosine or 5′-fluoro-cytosine) using conditions as outlined in U.S. Pat. No. 6,228,860 (col. 17 ln. 15-42) provides the desired phosphonate substituted analogs 22.2.

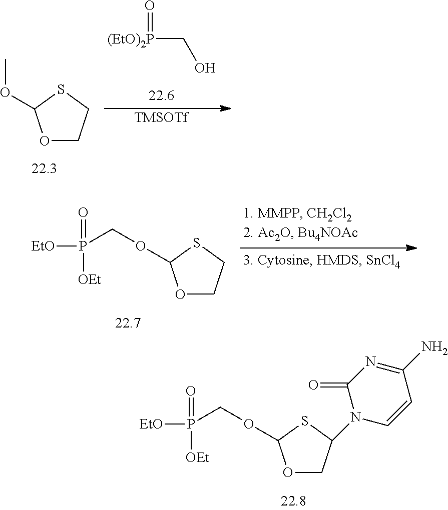

Specifically, as shown above, starting with heterocycle 22.3 and using diethyl(hydroxymethyl) phosphonate 22.6 generates 22.7. Introduction of cytosine as outlined above provides the desired product 22.8. Using the above procedure, but employing different phosphonate reagents 22.4 in place of 22.6, the corresponding products 22.2 bearing different linking groups are obtained.

### EXAMPLE 23

### Synthesis of Representative Compounds of Formula 24

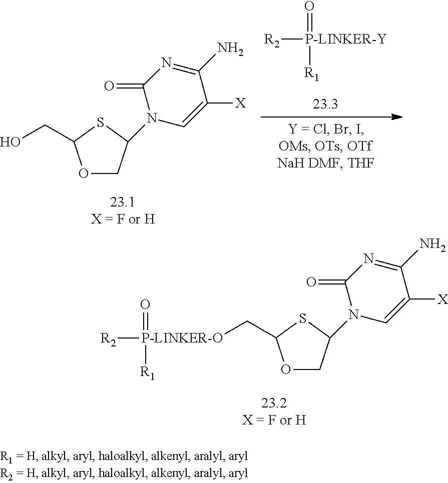

Representative compounds of the invention can be prepared by reaction of dOTC analogs of type 23.1 (obtained as described in U.S. Pat. No. 6,228,860 (col. 14 line 45 to col. 30 line 50 and references cited therein)) with the respective alkylating reagents 23.3. The above scheme shows the preparation of phosphonate linkage to dOTC through the 5′ hydroxyl group. Substrate 23.1 (dOTC) is dissolved in a solvent such as, but not limited to, DMF or THF, and is treated with a phosphonate reagent bearing a leaving group in the presence of a suitable organic or inorganic base. In compounds 23.3, Y is a leaving group including, but not limited to, bromide, chloride, iodide, p-toluenesulfonate, trifluoromethanesulfonate or methanesulfonate.

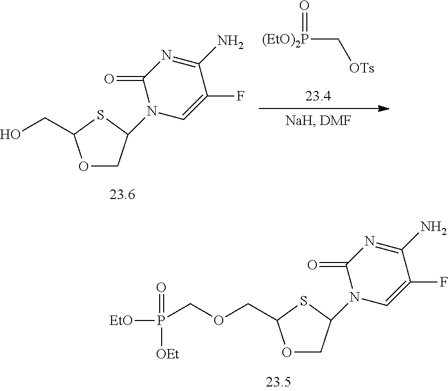

For instance, 23.6 is dissolved in DMF and treated with one equivalent of sodium hydride and one equivalent of (toluene-4-sulfonylmethyl)-phosphonic acid diethyl ester 23.4, prepared according to the procedures in J. Org. Che.m 1996, 61, 7697, to give fluoro-cytosine phosphonate derivative 23.5, in which the linkage is a methylene group.

Using the above procedure, but employing different phosphonate reagents 23.3 in place of 23.4, the corresponding products 23.2 bearing different linking groups are obtained.

### EXAMPLE 24

### Synthesis of Representative Compounds of Formula 25

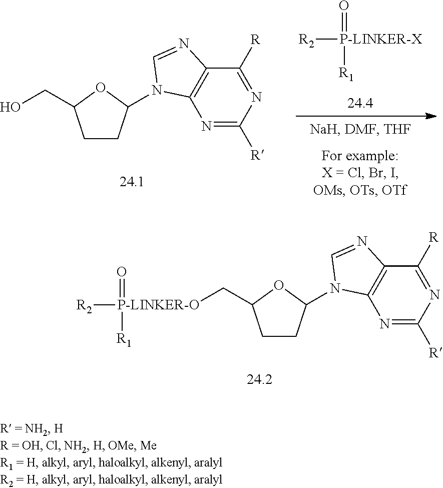

Representative compounds of the invention can be prepared as illustrated above. Phosphonate substituted analogs are prepared by reaction of furanoside purine nucleosides, structure 24.1 (obtained as described in U.S. Pat. No. 5,185,437 (col. 9 ln. 16 to col. 35 ln. 19, and references cited therein)) with the respective alkylating reagents 24.4. Illustrated above is the preparation of the phosphonate linkage to furanoside nucleoside cores through the 5′-hydroxyl group. The parent analog 24.1 is dissolved in a solvent such as, but not limited to, DMF or THF, and is treated with a phosphonate reagent bearing a leaving group in the presence of a suitable organic or inorganic base. In compounds 24.4, X is a leaving group such as, but not limited to, bromide, chloride, iodide, p-toluenesulfonate, trifluoromethanesulfonate, or methanesulfonate.

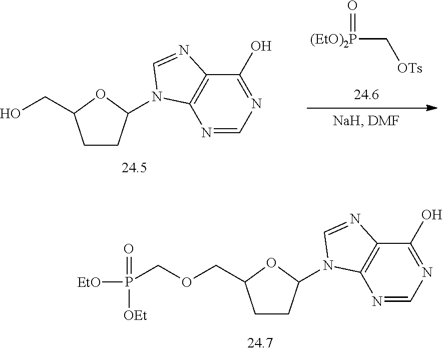

For instance, 24.5 (obtained as described in U.S. Pat. No. 5,185,437; col. 9 ln. 16 to col. 35 ln. 19, and references cited therein) is dissolved in DMF, is treated with three equivalents of sodium hydride and two equivalents of (toluene-4-sulfonylmethyl)-phosphonic acid diethyl ester 24.6, prepared according to the procedures in J. Org. Chem. 1996, 61, 7697, to give the corresponding phosphonate 24.7, in which the linkage is a methylene group.

Using the above procedure, but employing different phosphonate reagents 24.4 in place of 24.6, the corresponding products 24.2 bearing different linking groups are obtained.

### EXAMPLE 25

### Synthesis of Representative Compounds of Formula 26

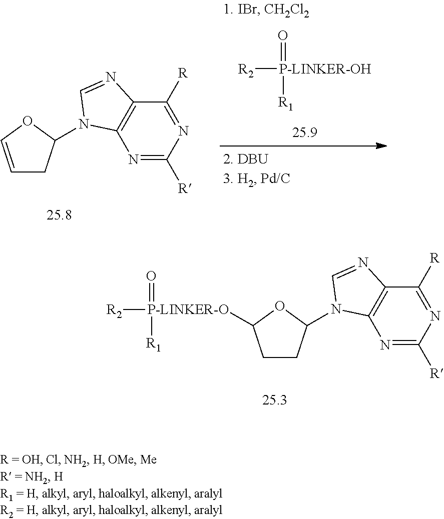

Representative compounds of the invention can be prepared as illustrated above. Phosphonate substituted analogs 25.3 are prepared by reacting glycal 25.8 (obtained as described in J. Am. Chem. Soc. 1972, 94, 3213; in some cases the nucleoside bases may need prior protection) with the respective phosphonate alcohols 25.9, followed by treatment with iodine monobromide (J. Org. Chem. 1991, 56, 2642-2647). Elimination of the resulting iodide followed by reduction with palladium on carbon provides the desired product 25.3.

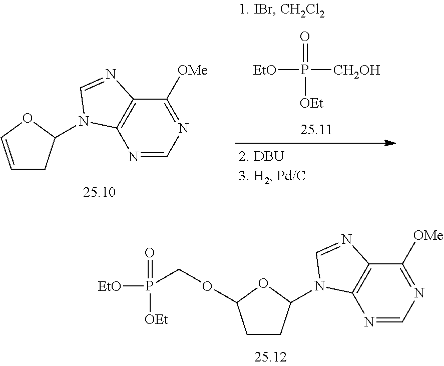

For instance, dihydrofuran 25.10 is dissolved in CH₂Cl₂ and is combined with 3.5 equivalents of diethyl(hydroxymethyl) phosphonate. The resulting solution is treated with two equivalents of iodine monobromide at −25° C. The resulting phosphonate-iodide is treated with DBU and reduced under hydrogenation conditions to afford the desired product 25.12. Using the above procedure, but employing different phosphonate reagents 25.9 in place of 25.11, the corresponding products 25.3 bearing different linking groups are obtained.

### EXAMPLE 26

### Synthesis of Representative Compounds of Formula 27

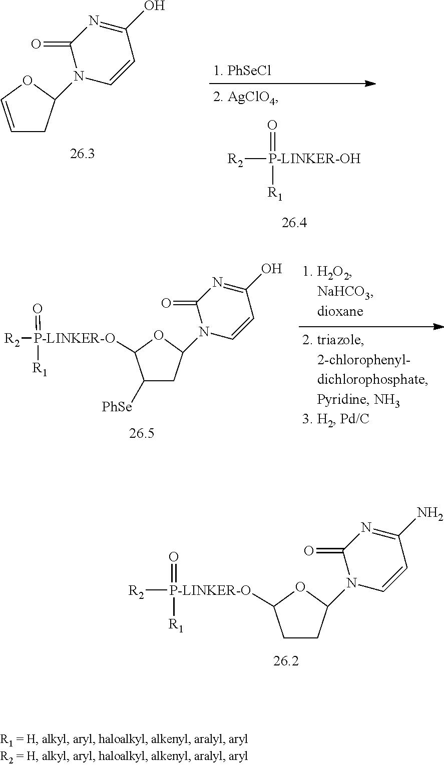

Representative compounds of the invention can be prepared as illustrated above. Phosphonate substituted analogs 26.2 are prepared by reacting glycal 26.3 (obtained as described in J. Am. Chem. Soc. 1972, 94, 3213) with phenylselenyl chloride followed by treatment with the respective phosphonate alcohols 26.4 in the presence of silver perchlorate (J. Org. Chem. 1991, 56, 2642-2647). Oxidation of the resulting chloride using hydrogen peroxide, followed by aminolysis treatment of uracil using triazole, 2-chlorophenyldichlorophosphate, pyridine and ammonia (Bioorg. Med. Chem. Lett. 1997, 7, 2567) and a palladium on carbon reduction provides the desired product 26.2.

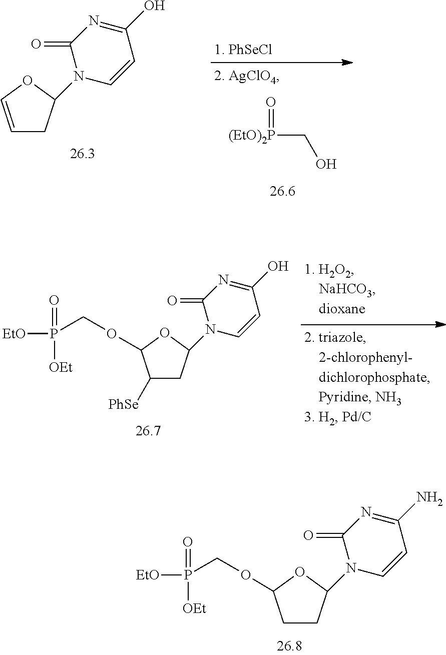

For instance, 26.3 dissolved in CH₂Cl₂, is treated with one equivalent of phenyl selenyl chloride at −70° C., followed by treatment with silver perchlorate in the presence of diethyl(hydroxymethyl) phosphonate to generate selenide 26.7. The phosphonate is transformed into the d4CP analog by first oxidation with hydrogen peroxide, followed by conversion of the uracil moiety to a cytosine, and finally hydrogenation to the desired product 26.8. Using the above procedure, but employing different phosphonate reagents 26.4 in place of 26.6, the corresponding products 26.2 bearing different linking groups are obtained.

In some cases conversions to desired compounds may require the use of suitable protecting groups for the amino group of cytosine. Similarly, using different natural and unnatural bases with appropriate protecting groups, other analogs containing a variety of bases can be prepared.

### EXAMPLE 27

### Synthesis of Representative Compounds of Formula 28

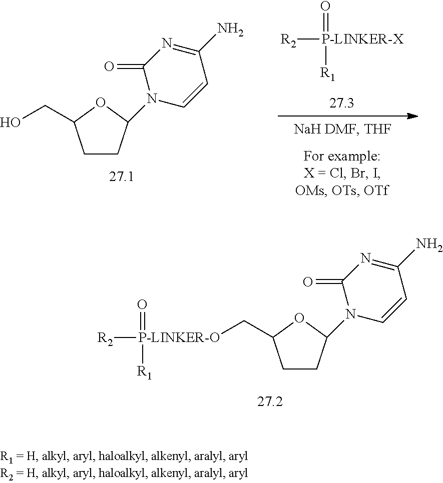

Representative compounds of the invention can be prepared as illustrated above. Phosphonate substituted analogs 27.2 are prepared by reaction of ddC 27.1 (D5782 Sigma-Aldrich, or prepares as described in J. Org. Chem. 1967, 32, 817) with the respective alkylating reagents 27.3. The scheme shown above illustrates the preparation of phosphonate linkage to ddC through the 5′-hydroxyl group. Substrate 27.1 (ddC or an analog) is dissolved in a solvent such as, but not limited to, DMF or THF, and is treated with a phosphonate reagent bearing a leaving group, in the presence of a suitable organic or inorganic base. In compounds 27.3, X is a leaving group such as, but not limited to, bromide, chloride, iodide, p-toluenesulfonate, trifluoromethanesulfonate, or methanesulfonate.

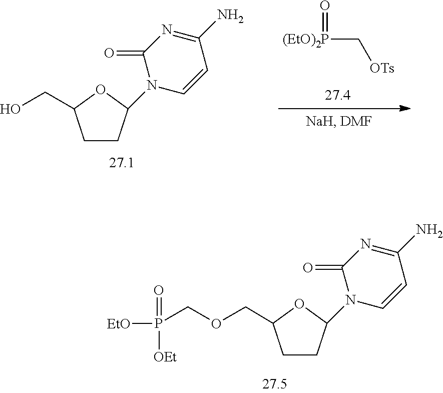

For instance, 27.1 dissolved in DMF, is treated with two equivalent of sodium hydride and two equivalent of (toluene-4-sulfonylmethyl)-phosphonic acid diethyl ester 27.4, prepared according to the procedures in J. Org. Chem. 1996, 61, 7697, to give ddC phosphonate 27.5 in which the linkage is a methylene group. Using the above procedure, but employing different phosphonate reagents 27.3 in place of 27.4, the corresponding products 27.2 bearing different linking groups are obtained.

### EXAMPLE 28

### Synthesis of Representative Compounds of Formula 29

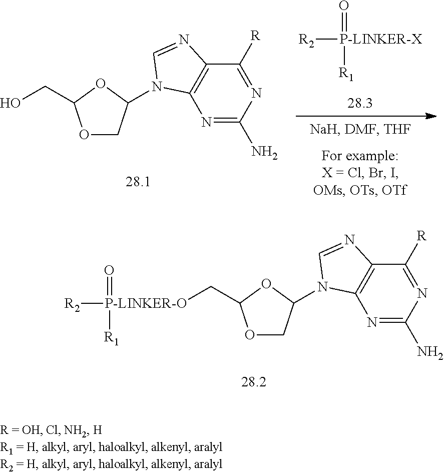

Representative compounds of the invention can be prepared as illustrated above. Phosphonate substituted analogs 28.2 are prepared by reaction of dioxolanyl purine nucleosides, structure 28.1 (obtained as described in U.S. Pat. No. 5,925,643; col. 4 ln. 47 to col. 12 ln. 20, and references therein) with the respective alkylating reagents 28.3. Illustrated above is the preparation of phosphonate linkage to dioxalane nucleoside cores through the 5′-hydroxyl group. The parent analog 28.1 is dissolved in a solvent such as, but not limited to, DMF and/or THF, and is treated with a phosphonate reagent bearing a leaving group, in the presence of a suitable organic or inorganic base. In compounds 28.3, X is a leaving group such as, but not limited to, bromide, chloride, iodide, p-toluenesulfonate, trifluoromethanesulfonate, or methanesulfonate.

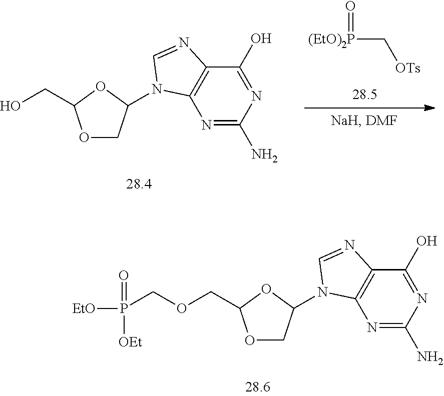

For instance, 28.4 is dissolved in DMF and treated with five equivalents of sodium hydride and one equivalent of (toluene-4-sulfonylmethyl)-phosphonic acid diethyl ester 28.5, prepared according to the procedures in J. Org. Chem. 1996, 61, 7697, to give the corresponding phosphonate 28.6, in which the linkage is a methylene group. Using the above procedure, but employing different phosphonate reagents 28.3 in place of 28.5, the corresponding products 28.2 bearing different linking groups are obtained.

### EXAMPLE 29

### Synthesis of Representative Compounds of Formula 30

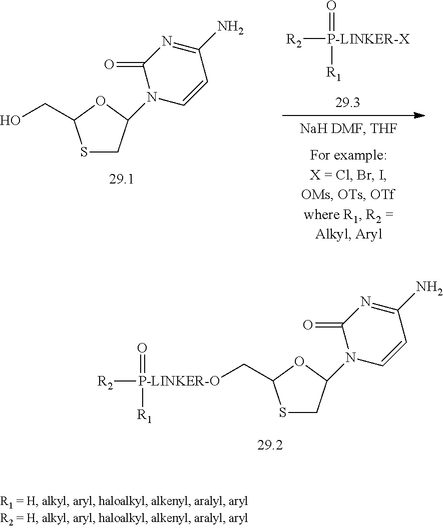

Representative compounds of the invention can be prepared as illustrated above. Phosphonate substituted analogs 29.2 are prepared by reaction of 3TC (29.1) (obtained as described in U.S. Pat. No. 5,047,407; col. 9 line 7 to col. 12 line 30, and references cited therein) with the respective alkylating reagents 29.3. Illustrated above is the preparation of phosphonate linkage to 3TC through the 5′-hydroxyl group. 3TC is dissolved in a solvent such as, but not limited to, DMF and/or THF, and is treated with a phosphonate reagent bearing a leaving group, in the presence of a suitable organic or inorganic base. In compounds 29.3, X is a leaving group such as, but not limited to, bromide, chloride, iodide, p-toluenesulfonate, trifluoromethanesulfonate, or methanesulfonate.

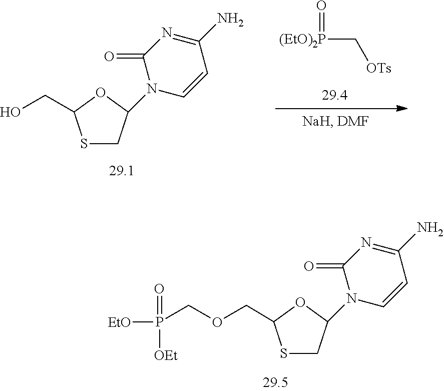

For instance, 29.1 dissolved in DMF, is treated with one equivalent of sodium hydride and one equivalent of (toluene-4-sulfonylmethyl)-phosphonic acid diethyl ester 29.4 (prepared according to the procedure in J. Org. Chem. 1996, 61, 7697) to give 3TC phosphonate 29.5, in which the linkage is a methylene group. Using the above procedure, but employing different phosphonate reagents 29.3 in place of 29.4, the corresponding products 29.2 bearing different linking groups are obtained.

### EXAMPLE 30

### Synthesis of Representative Compounds of Formula 31

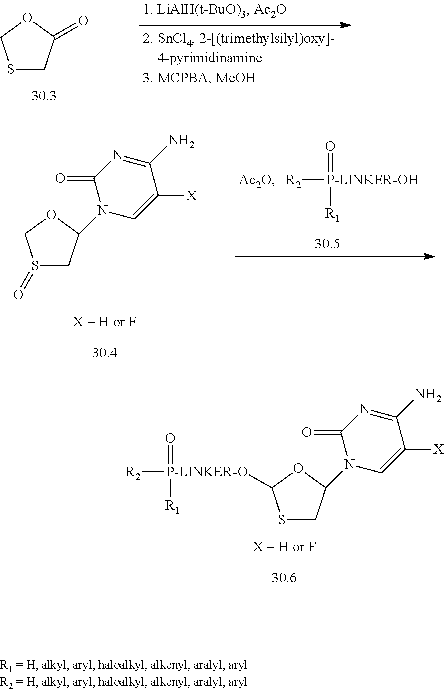

Representative compounds of the invention can be prepared as illustrated above. Starting with the known oxathiolan-5-one (30.3) (Acta Chem. Scand., Ser. A 1976, 30, 457), reduction followed by base introduction using the conditions outlined in U.S. Pat. No. 5,914,331 (col. 11 ln. 62 to col. 12 ln. 54) provides the substrate for the Pummerer reaction. Oxidation using m-chloroperbenzoic acid in methanol (U.S. Pat. No. 5,047,407 col. 12 ln. 35 to col. 12 ln. 50) generates sulfoxide 30.4. The Pummerer reaction in the presence of the phosphonate linked alcohol 30.5 and acetic anhydride provides phosphonate 30.6.

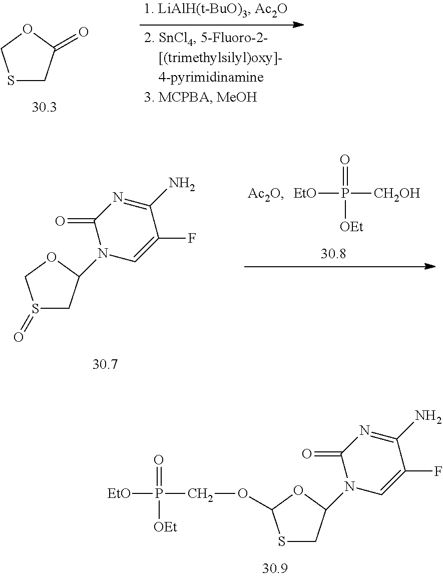

As an example, subjecting oxathiolan-5-one to conditions above but using 5-fluoro-2-[(trimethylsilyl)oxy]-4-pyrimidinamine followed by oxidation provides intermediate 30.7. Introduction of phosphonate moiety 30.8, using Pummerer conditions (Org. React. 1991, 40, 157) provides the diethyl phosphonate product 30.9.

### EXAMPLE 31

### Synthesis of Representative Compounds of Formula 32

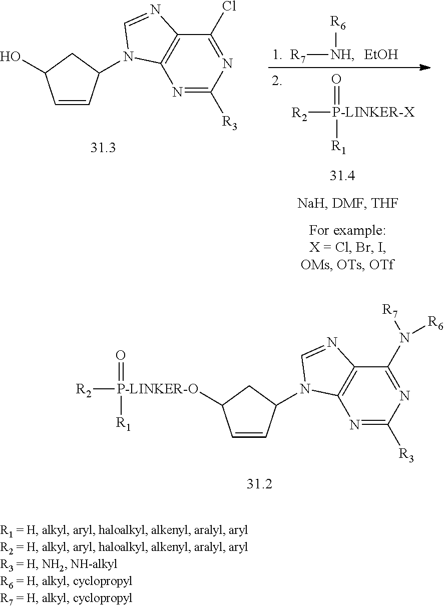

Representative compounds of the invention can be prepared as illustrated above. Alcohol 31.3 can be prepared as described in J. Chem. Soc., Perkin Trans. 1 1994, 1477. Note that other base derivatives can be prepared in a similar manner starting with their respective bases. Displacement of the chloride of 31.3 with an amine in ethanol under reflux conditions (U.S. Pat. No. 5,034,394, col. 9, ln. 60 to col. 10 ln. 21) provides the key intermediate alcohol. Treatment of this alcohol with the respective alkylating reagents 31.4, provides the desired phosphonate substituted analogs 31.2. In the above compounds, R₆ is H, R₇ is cyclopropyl, R₃ is NH₂.

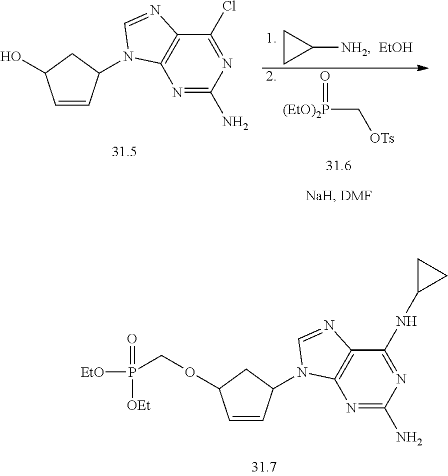

As an example, treatment of the key intermediate alcohol, as described above (J. Chem. Soc., Perkin Trans. 1994, 1, 1477), with one equivalent of sodium hydride and one equivalent of (toluene-4-sulfonylmethyl)-phosphonic acid diethyl ester 31.6 (prepared according to the procedures in J. Org. Chem. 1996, 61, 7697) affords ABC phosphonate 31.7, in which the linkage is a methylene group. Using the above procedure, but employing different R₃, R₆, R₇ and phosphonate reagents 31.4 in place of 31.6, the corresponding products 31.2 bearing different linking groups are obtained.

### EXAMPLE 32

### Synthesis of Representative Compounds of Formula 33

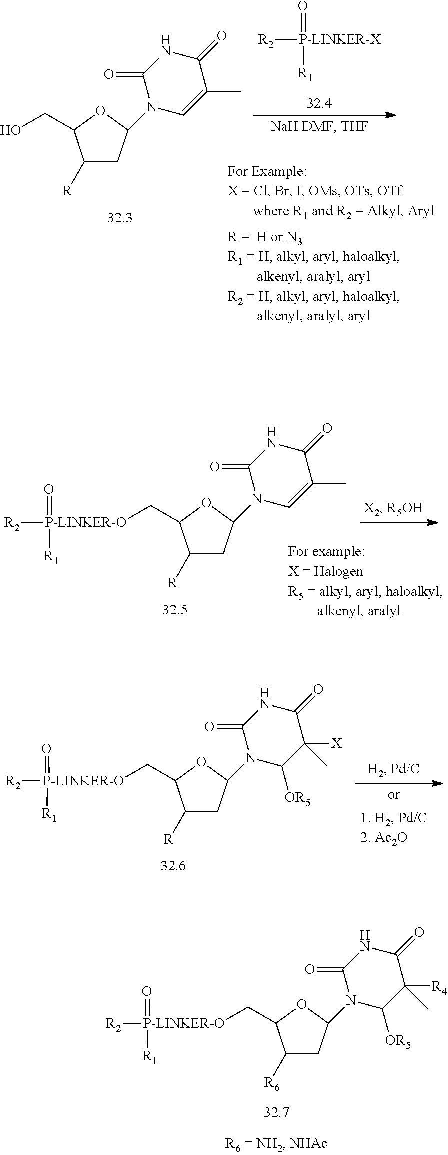

Representative compounds of the invention can be prepared as illustrated above. Phosphonate substituted analogs 32.5 are prepared by reaction of 32.3 (for example, AZT (A 2169, Sigma Aldrich or obtained as described in U.S. Pat. No. 4,724,232) or 3′-deoxythymidine (D 1138 Sigma Aldrich)) with the respective alkylating reagents 32.4. Further modification of either the base or the 3′-substituent can be carried out as illustrated above. AZT is dissolved in a solvent such as, but not limited to, DMF and/or THF, and is treated with a phosphonate reagent bearing a leaving group, in the presence of a suitable organic or inorganic base. In compounds 32.4, X is a leaving group such as, but not limited to, bromide, chloride, iodide, p-toluenesulfonate, trifluoromethanesulfonate, or methanesulfonate.

Treatment of compound 32.5 with methyl hypobromite provides the 5-bromo-6-alkoxy analog 32.6 (J. Med. Chem. 1994, 37, 4297 and U.S. Patent Publication 00/22600). Compound 32.6 can be elaborated by reducing the 3′-azide to the amine and converting the amine to the corresponding acetyl to provide compounds 32.7.

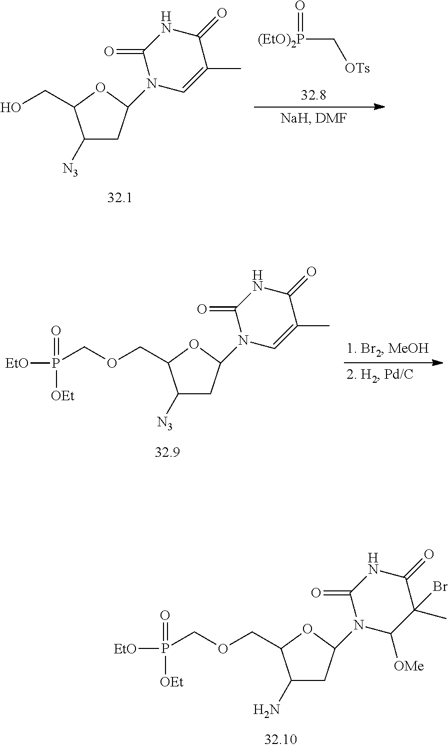

For instance, 32.1 dissolved in DMF, is treated with one equivalent of sodium hydride and one equivalent of (toluene-4-sulfonylmethyl)-phosphonic acid diethyl ester 32.8 (prepared according to the procedures in J. Org. Chem. 1996, 61, 7697) to give AZT phosphonate 32.9, in which the linkage is a methylene group. Treatment with methyl hypobromite followed by hydrogenation provides analog 32.10. Using the above procedure, but employing different phosphonate reagents 32.4 in place of 32.8, the corresponding products 32.2 bearing different linking groups are obtained. Additionally, the R₃-R₅ groups can be varied to generate other compounds.

### EXAMPLE 33

### Synthesis of Representative Compounds of Formula 34

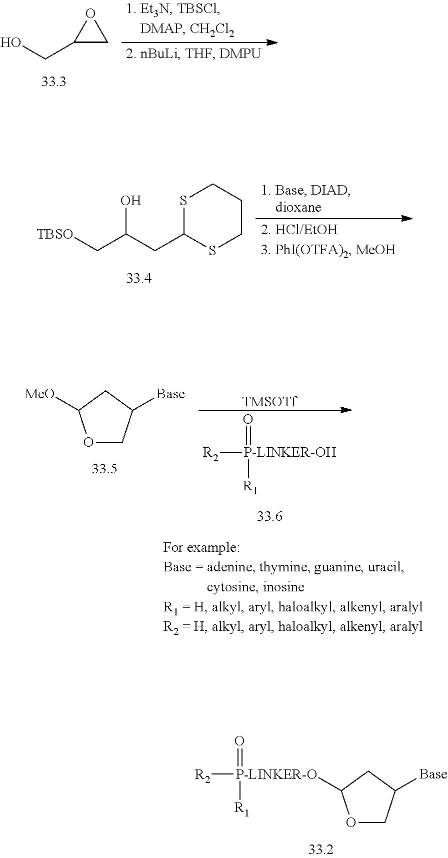

Representative compounds of the invention can be prepared as illustrated above. Starting with commercially available glycidol, silyl protection of the alcohol followed by a lithium-mediated opening of the epoxide generates alcohol 33.4 (see Angew. Chem., Int. Ed. Engl. 1998, 37, 187-192). Introduction of the appropriately protected bases using Mitsunobu reaction conditions (Tetrahedron Lett. 1997, 38, 4037-4038; Tetrahedron 1996, 52, 13655) followed by acid mediated removal of the silyl protecting group (J. Org. Chem. 1980, 45, 4797) and dithiane removal and in situ cyclization (J. Am. Chem. Soc. 1990, 112, 5583) produces furanoside 33.5. Introduction of phosphonate linkage using the appropriate alcohol in the presence of TMSOTf (Synlett 1998, 177) generates analog 33.2.

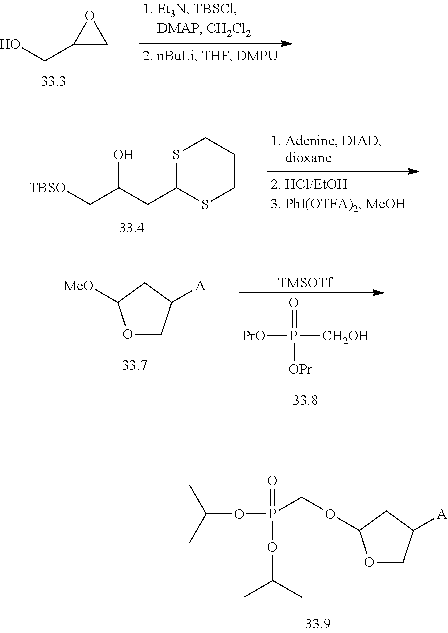

For instance, 3 equivalents of DIAD (in 3 portions) is added dropwise to a stirred solution of alcohol 33.4 and adenine (3 equivalents) in dioxane. The reaction is stirred for 20 hours. The resulting product is treated with hydrochloric acid in ethanol for 15 hours and filtered. The residue is stirred with [bis(trifluoroacetoxy)iodo]benzene (1.5 equivalents) in methanol to generate 33.7. Lewis acid-mediated reaction (Synlett 1998, 177) of diisopropyl hydroxymethylphosphonate 33.8 (Tetrahedron Lett. 1986, 27, 1477) produces a diastereomeric mixture of phosphonates 33.9, in which the linkage is a methylene group. Using the above procedure, but employing different appropriately protected bases and phosphonate reagents 33.6 in place of 33.8, the corresponding products 33.2 bearing different linking groups are obtained.

### EXAMPLE 34

### Synthesis of Representative Compounds of Formula 35

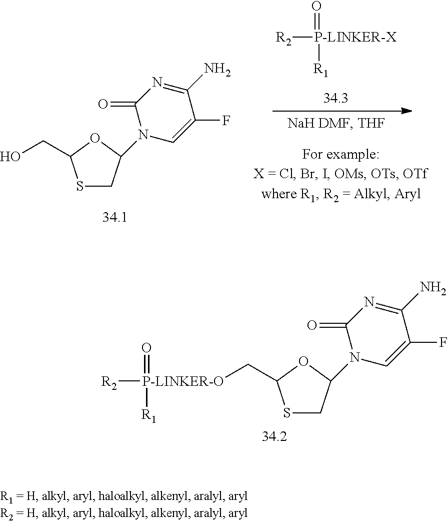

Representative compounds of the invention can be prepared as illustrated above. Phosphonate substituted analogs 34.2 are prepared by reaction of FTC (34.1) (obtained as described in U.S. Pat. No. 5,914,331; col. 10 line 40 to col. 18 line 15 and references cited therein) with the respective alkylating reagents 34.3. Illustrated above is the preparation of phosphonate linkage to FTC through the 5′-hydroxyl group. FTC is dissolved in a solvent such as, but not limited to, DMF and/or THF, and is treated with a phosphonate reagent bearing a leaving group, in the presence of a suitable organic or inorganic base. In compounds 34.3, X is a leaving group such as, but not limited to, bromide, chloride, iodide, p-toluenesulfonate, tri fluoromethanesulfonate, or methanesulfonate.

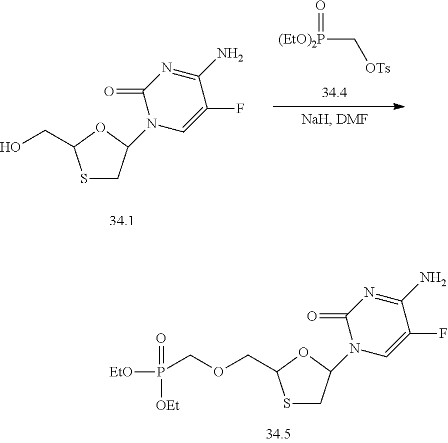

For instance, 34.1 dissolved in DMF, is treated with one equivalent of sodium hydride and one equivalent of (toluene-4-sulfonylmethyl)-phosphonic acid diethyl ester 34.4 (prepared according to the procedures in J. Org. Chem. 1996, 61, 7697) to give FTC phosphonate 34.5 in which the linkage is a methylene group. Using the above procedure but employing different phosphonate reagents 34.3 in place of 34.4, the corresponding products 34.2 bearing different linking groups can be obtained.

### EXAMPLE 35

### Synthesis of Representative Compounds of Formulae 36-37

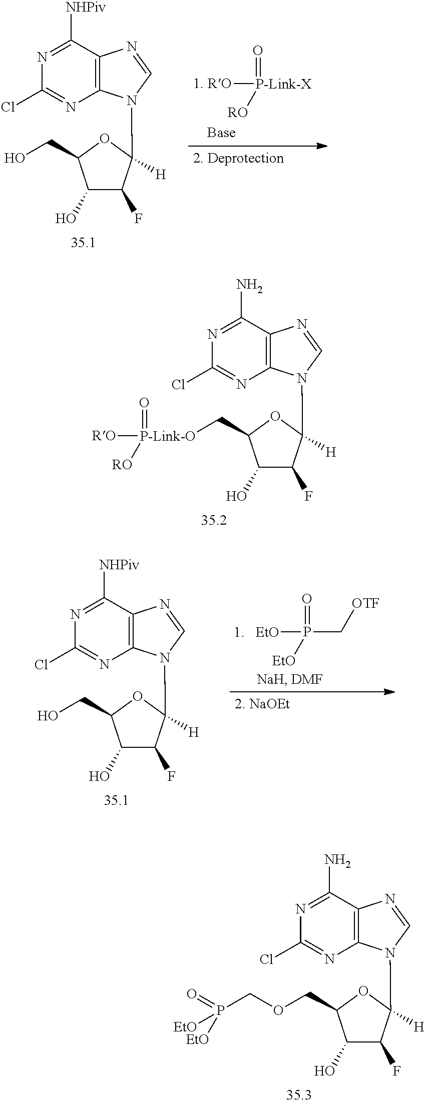

Representative compounds of the invention can be prepared as illustrated above. The appropriately protected 2-chloro-9-(2-deoxy-2-fluoro-β-D-arabinofuranosyl)-9H-purin-6-amine 35.1, prepared according to U.S. Pat. No. 5,034,518 (also described in WO 03011877) can be treated in a solvent such as tetrahydrofuran or dimethylformamide with a base such as sodium hydride. Formation of the pivaloyl compound 35.1 can be accomplished by protecting 2-chloro-9-(2-deoxy-2-fluoro-β-D-arabinofuranosyl)-9H-purin-6-amine with a pivaloyl group (Greene, T., “Protective Groups in Organic Gynthesis,” Wiley-Interscience, (1999)). When bubbling ceases, diethyl phosphonomethyltriflate (prepared according to Tetrahedron Lett., (1986), 27, 1477) is added, yielding the protected product 35.2 or 35.3. The pivaloyl group is removed with sodium ethoxide to provide the desired phosphonate diester 35.2 or 35.3.

### EXAMPLE 36

### Synthesis of Representative Compounds of Formulae 36-37

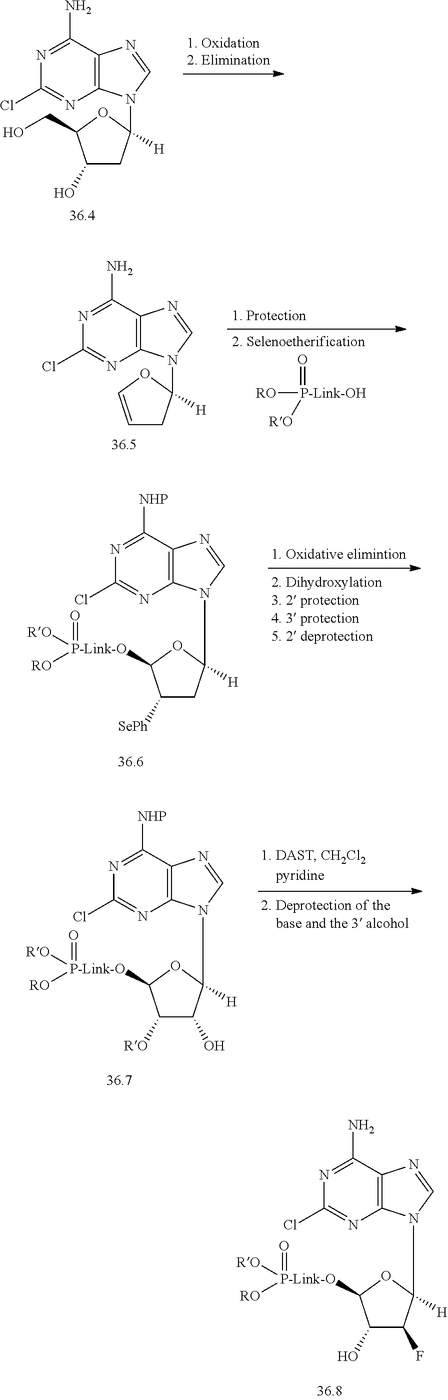

Representative compounds of the invention can be prepared as illustrated above. Compound 36.4, 9-(2-deoxy-α-D-ribofuranosyl)-2-fluoroadenine, can be prepared as described in Montgomery, J. et al., J. Med. Chem., (1969), 12, 3, 498. Oxidation of the 5′-OH followed by elimination provides glycal 36.5 (see the_procedure of Zemlicka J. et al., J. Am. Chem. Soc., (1972), 94, 9, 3213). Protection of the chloroadenine at the 6 position followed by selenoetherification provides the protected phosphonate 36.6 (Kim, C. et al., J. Org. Chem., (1991), 56, 2642). Oxidative elimination of the phenylselenide (as described in Kim, C. et al., J. Org. Chem., (1991), 56, 2642) followed by stereoselective dihydroxylation provides the diol that can then be converted to the 2′ protected alcohol. Protection of the 3′ alcohol followed by removal of the protecting group at the 2′ hydroxyl group provides compound 36.7. Fluorination and inversion of the stereochemistry at the 2′ position can be simultaneously achieved by exposing the compound to dimethylaminosulfur trifluoroide (DAST) and pyridine (Pankiewicz, K. W. et al., J. Org. Chem., (1992), 57, 553, also see Pankiewicz, K. W. et al., J. Org. Chem., (1992), 57, 7315). Finally, the protecting groups are removed to provide compound 36.8.

### EXAMPLE 37

### Synthesis of Representative Compounds of Formulae 36-37

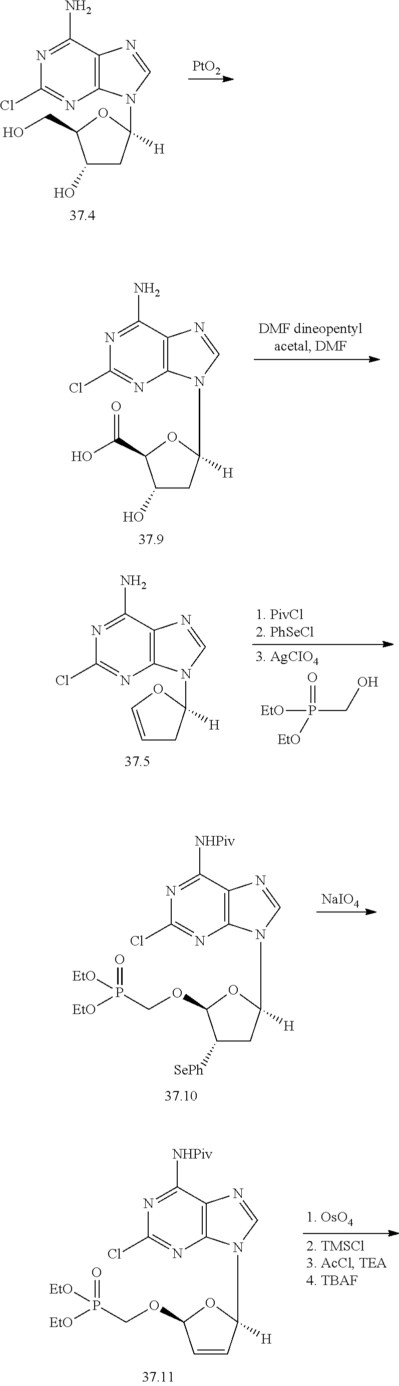

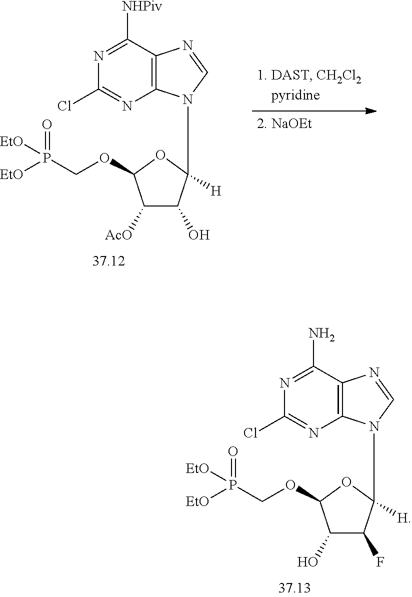

Representative compounds of the invention can be prepared as illustrated above. Specifically, 9-(2-deoxy-α-D-ribofuranosyl)2-fluoroadenine, compound 37.4 (Montgomery, J. et. al., J. Med. Chem., (1969), 12, 3, 498), can be oxidized with PtO₂ to provide carboxylic acid 37.9. Decarboxylative elimination is achieved using dimethylformamide dineopentyl acetal in dimethylformamide at high temperature (Zemlicka J. et al., J. Am. Chem. Soc., (1972), 94, 9, 3213). The furanoid glycal 37.5 is first protected at the 6-position of the 2-chloroadenosine with pivaloyl chloride, using conditions as described in Greene, T., “Protective Groups in Organic Synthesis,” Wiley-Interscience, (1999). Treatment of the protected glycal with silver perchlorate in the presence of diethyl(hydroxylmethyl)phosphonate (Phillion, D. et al., Tetrahedron Lett., (1986), 27, 1477) provides the phosphonate 37.10 (Kim, C. et al., J. Org. Chem., (1991), 56, 2642). Oxidative elimination of the selenide followed by dihydroxylation using osmium tetraoxide provides a diol which can be turned into a mono protected acetate 37.12 by first silylating at the 2′-OH group, followed by protection of the 3′ alcohol with an acetate group and subsequent deprotection of the silyl group. Conversion of the 2′ alcohol to the 2′ fluoride with the opposite stereochemistry can be performed with DAST (Pankiewicz, K. W. et al., J. Org. Chem., (1992), 57, 553, also see Pankiewicz, K. W. et al., J. Org. Chem., (1992), 57, 7315). Conditions that deprotect the pivaloyl group (Greene, T., Protective groups in organic synthesis, Wiley-Interscience, (1999)) also remove the 3′ acetate to provide compound 37.13.

### EXAMPLE 38

### Synthesis of Representative Compounds of Formulae 38-39

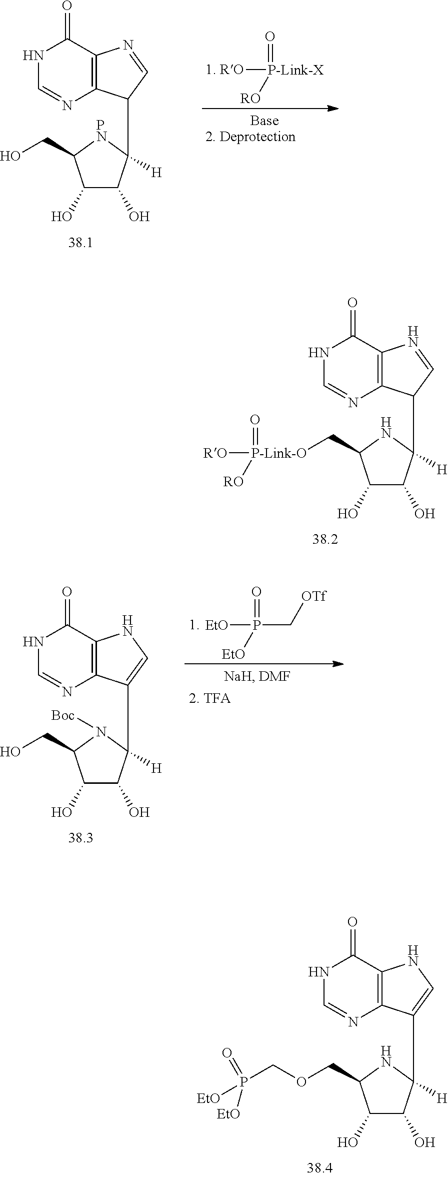

Representative compounds of the invention can be prepared as illustrated above. The Boc-protected (1S)-1-(9-deazaguanin-9-yl)-1,4-dideoxy-1,4-imino-D-ribitol, compound 38.3, is prepared by stirring the (1S)-1-(9-deazaguanin-9-yl)-1,4-dideoxy-1,4-imino-D-ribitol (WO 99/19338 and Evans, G. B. et al., Tetrahedron, 2000, 56, 3053, also reported in Evans, G. B. et al., J. Med. Chem. 2003, 46, 3412) with BOC anhydride as described in Greene, T., “Protective Groups in Organic Synthesis,” Wiley-Interscience, 1999. Compound 38.3, is then treated in a solvent such as tetrahydrofuran or dimethylformamide with a base such as sodium hydride. When bubbling ceases, diethyl phosphonomethyl-triflate (prepared according to Tetrahedron Lett., 1986, 27, 1477) is added yielding the desired phosphonate diester, 38.4, after deprotection of the BOC group using trifluoroacetic acid (TFA).

### EXAMPLE 39

### Synthesis of Representative Compounds of Formulae 38-39

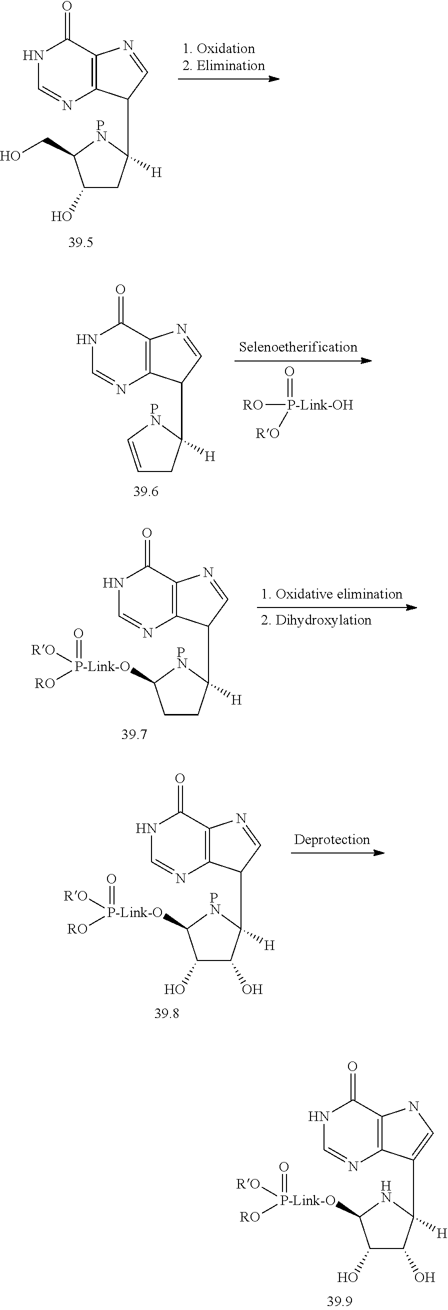

Representative compounds of the invention can be prepared as illustrated above. The deprotected version of compound 39.5 ((1R)-1-(9-deazahypoxanthin-9-yl)-1,2,4-trideoxy-1,4-imino-D-erythro-pentitol, as the hydrochloride salt) is prepared as described in Evans, G. B. et al., Tetrahedron, 2000, 56, 3053, using di-t-butyl dicarbonate in dichloromethane. Oxidation of the 5′-OH followed by elimination provides glycal 39.6 (see the procedure of Zemlicka J. et al., J. Am. Chem. Soc., 1972, 94, 9, 3213). Selenoetherification provides the protected phosphonate 39.7 (Kim, C. et al., J. Org. Chem., 1991, 56, 2642). Oxidative elimination of the phenylselenide (as described in Kim, C. et al., J. Org. Chem., 1991, 56, 2642) followed by stereoselective dihydroxylation provides the desired diol 39.8. Finally, the protecting group is removed to provide compound 39.9.

### EXAMPLE 40

### Synthesis of Representative Compounds of Formulae 38-39

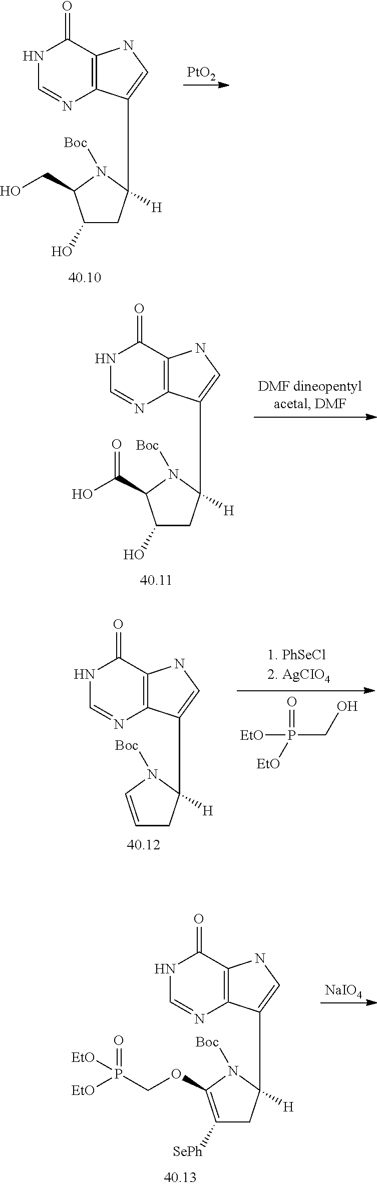

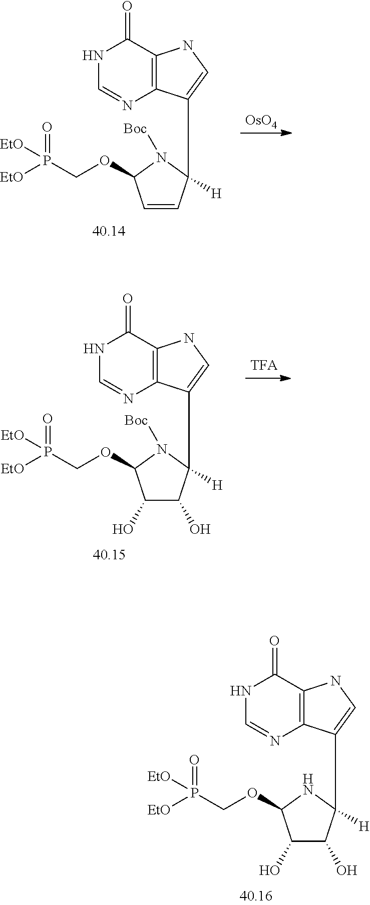

Representative compounds of the invention can be prepared as illustrated above. Specifically, (1R)-1-(9-deazahypoxanthin-9-yl)-1,2,4-trideoxy-1,4-imino-D-erythro-pentitol, prepared as the HCl salt as described in Evans, G. B. et al., Tetrahedron, (2000), 56, 3053, is first protected and then oxidized with PtO₂ to provide carboxylic acid 40.11. Decarboxylative elimination is achieved using dimethylformamide dineopentyl acetal in dimethylformamide at high temperature (Zemlicka J. et al., J. Am. Chem. Soc., (1972), 94, 9, 3213). Selenoetherification followed by treatment of the protected glycal with silver perchlorate in the presence of diethyl(hydroxylmethyl)phosphonate (Phillion, D. et al., Tetrahedron Lett., 1986, 27, 1477) provides the phosphonate 40.13 (Kim, C. et al., J. Org. Chem., (1991), 56, 2642). Oxidative elimination of the selenide followed by dihydroxylation using osmium tetraoxide provides diol 40.15. Removal of the amine protecting group, according to the procedure of Greene, T., “Protective Groups in Organic Synthesis,” Wiley-Interscience, (1999), provides compound 40.16.

### EXAMPLE 41

### Synthesis of Representative Compounds of Formula 46

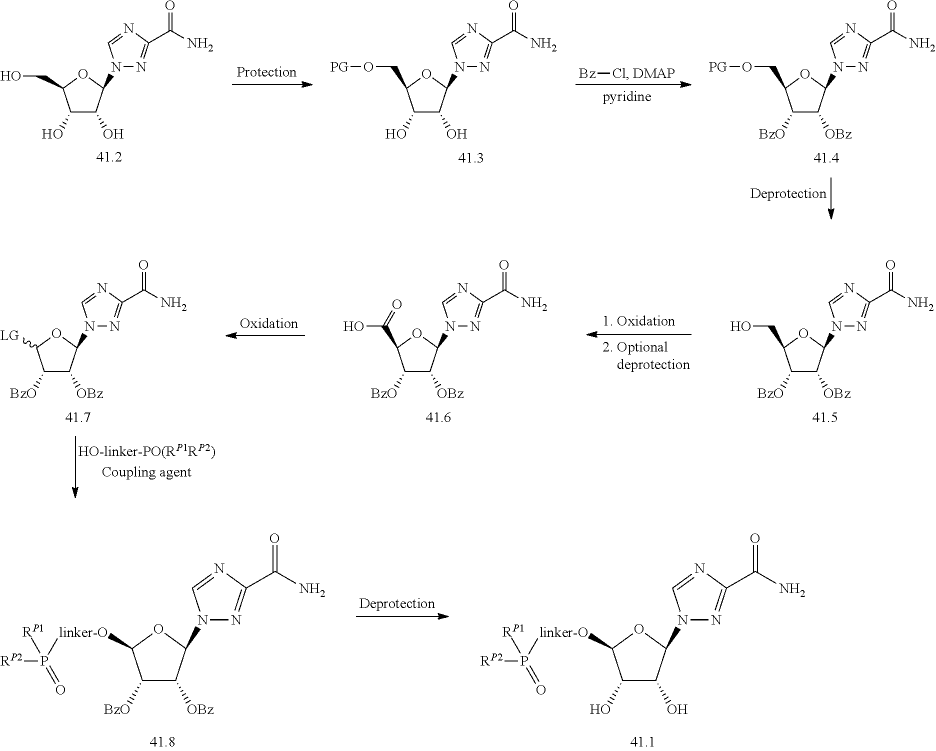

Representative compounds of the invention can be prepared as illustrated above. The 5′-hydroxyl group of ribavirin (41.2) can be selectively protected with an appropriate protecting group. The product, 41.3, can be treated with benzoyl chloride, an appropriate base, in the presence of catalytic amount of 4-dimethylaminopyridine, to convert 2′- and 3′-hydroxyl groups to their corresponding benzoyl esters, producing dibenzoate 41.4. The 5′-hydroxyl group can be selectively deprotected to afford alcohol 41.5. Following procedure described for the analogous compound in U.S. Pat. No. 6,087,482, FIG. 2, dibenzoate 41.4 can be converted to 41.7 in a three-step sequence. Treating electrophile 41.7 with a coupling agent, such as trimethylsilyl trifluoromethanesulfonate, in the presence of an appropriate alcohol containing a phosphonate group can produce phosphonate 41.8. Treating 41.8 with aqueous sodium hydroxide can deprotect the 2′- and 3′-hydroxyl groups to provide diol 41.1. Note that R^{P1} and R^{P2} in 41.8 and 41.1 can be the same or different protecting groups.

### EXAMPLE 42

### Synthesis of Representative Compounds of Formula 47

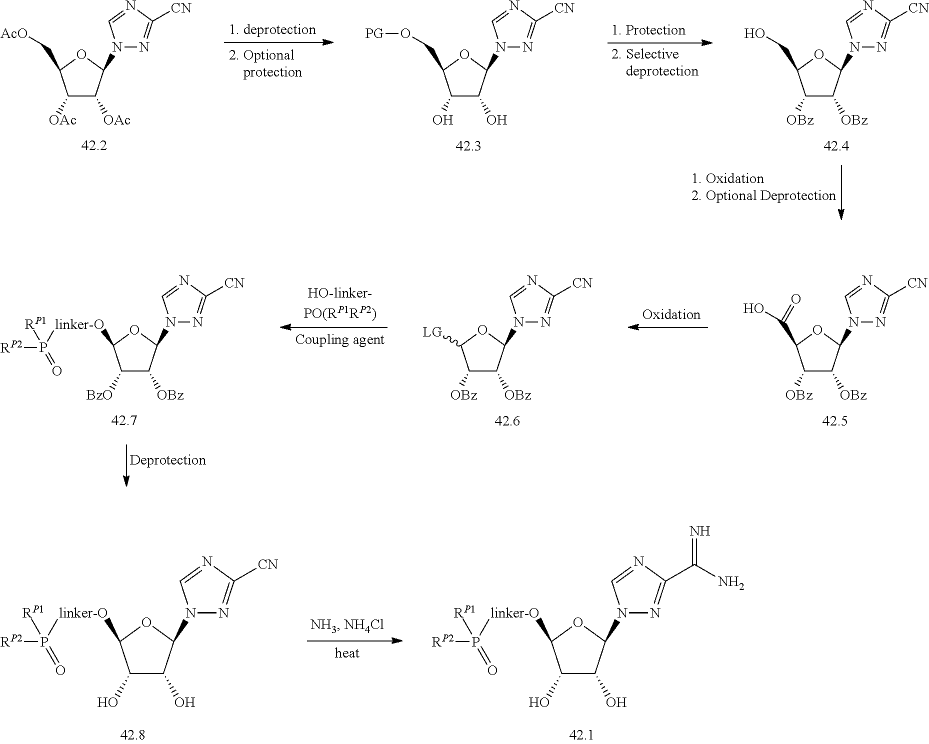

Representative compounds of the invention can be prepared as illustrated above. The synthesis of 3-cyano-1-(2,3,5-tri-O-acetyl-β-D-ribofuranosyl)-1,2,4-triazole (42.2) is described in US 2002/0156030 A1, page 6, paragraph 0078 to paragraph 0079. Using this starting material, one can synthesize compound 42.1 using the sequence of chemical transformations outlined above.

Appropriate protection and deportection procedures (see Greene and Wuts, “Protective Groups in Organic Synthesis,” 1999) can be employed to prepare 42.3, in which the 5′-hydroxyl group is protected, while the 2′-, and 3′-hydroxyl groups are not. Subsequent protection, deprotection procedures can introduce protecting groups such as benzoyl group to the 2′- and 3′-hydroxyls, leaving the 5′-hydroxyl group unprotected as in alcohol 42.4. Oxidation can convert the primary alcohol in 42.4 to the corresponding carboxylic acid or its ester. An optional deprotection of the ester can give the acid 42.5 as product. Further oxidation using oxidant such as lead tetraacetate can convert acid 42.5 to electrophile 42.6, in which the leaving group is an acetate. Treating 42.6 with an alcohol containing a phosphonate moiety in the presence of appropriate coupling agent, such as trimethylsilyl trifluoromethanesulfonate, affords phosphonate 42.8. Finally, treating 42.8 with the procedure described in US 2002/0156030 A1, page 6, paragraph 0081, provides phosphonate 42.1. Note that R^{P1} and R^{P2} in 42.7, 42.8 and 42.1 do not need to be the same.

### EXAMPLE 43

### Synthesis of Representative Compounds of Formula 48

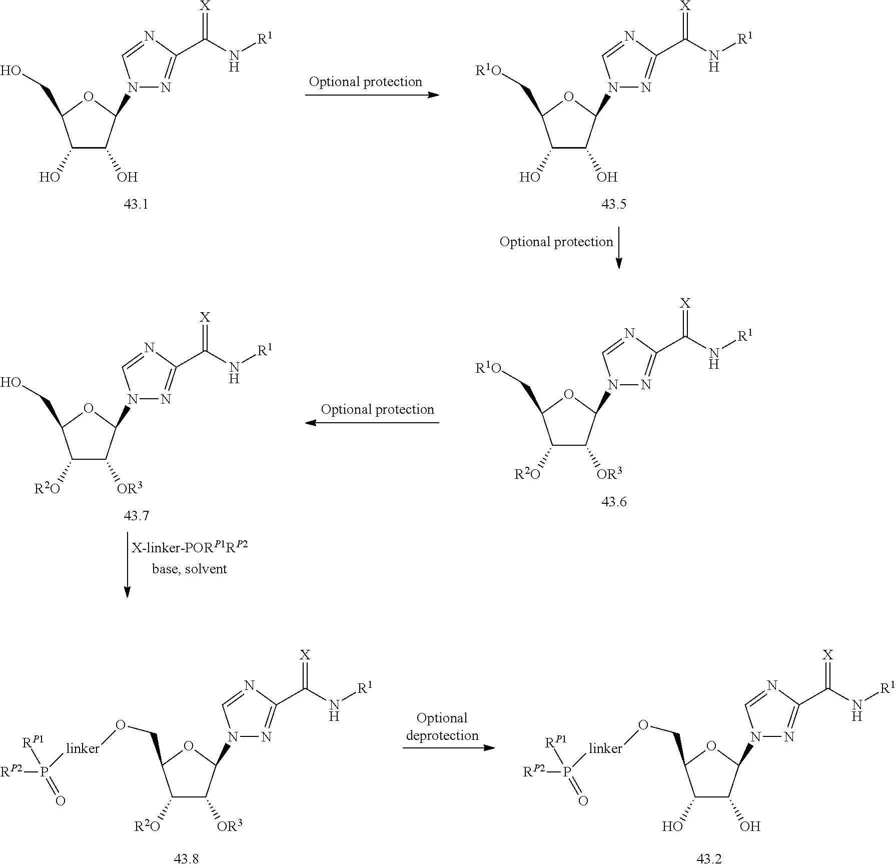

Representative compounds of the invention can be prepared as illustrated above. Compound 43.2 can be prepared from 43.1 by a series of selective protections of the 2′-, 3′-, and 5′-hydroxyl groups to give 43.6. The 5′-hydroxyl can then be selectively deprotected to give alcohol 43.7. Compound 43.7 in an appropriate aprotic solvent can be treated with at least two equivalence of an appropriate organic or inorganic base, and an appropriate electrophile bearing a leaving group, as in the structure X-linker-POR^{P1}R^{P2}, where X is a leaving group, to produce phosphonate 43.8. Appropriate deprotection procedures can be employed to convert 43.8 to diol 43.2. Note that R^{P1} and R^{P2} in 43.8 and 43.2 do not need to be the same.

Suitable aprotic solvents include, but are not limited to dimethyl formamide, dimethyl sulfoxide, and N-methylpyrrolidinone. Suitable organic or inorganic base include, but are not limited to sodium hydride, potassium t-butoxide, and triethylamine. Suitable leaving groups include, but are not limited to, chlorine, bromine, iodine, p-toluenesulfonate, methanesulfonate, and trifluoromethanesulfonate.

### EXAMPLE 44

### Synthesis of Representative Compounds of Formulae 49 and 50

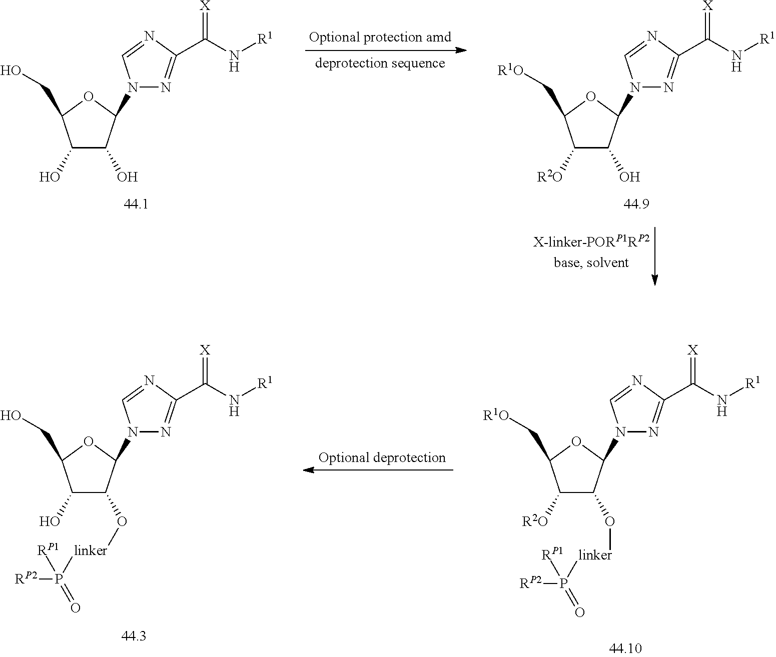

Representative compounds of the invention can be prepared as illustrated above. Triol 44.1 can be converted to alcohol 44.9, having an unprotected 2′-hydroxyl, by selecting appropriate protecting groups for the 3′- and 5′-hydroxyl groups through a series of protection and deprotection sequences. Alkylation of 44.9 by an electrophile containing a phosphonate as described in Example 43 can produce 44.10. After appropriate deprotection, 44.10 can be converted to phosphonate 44.3.

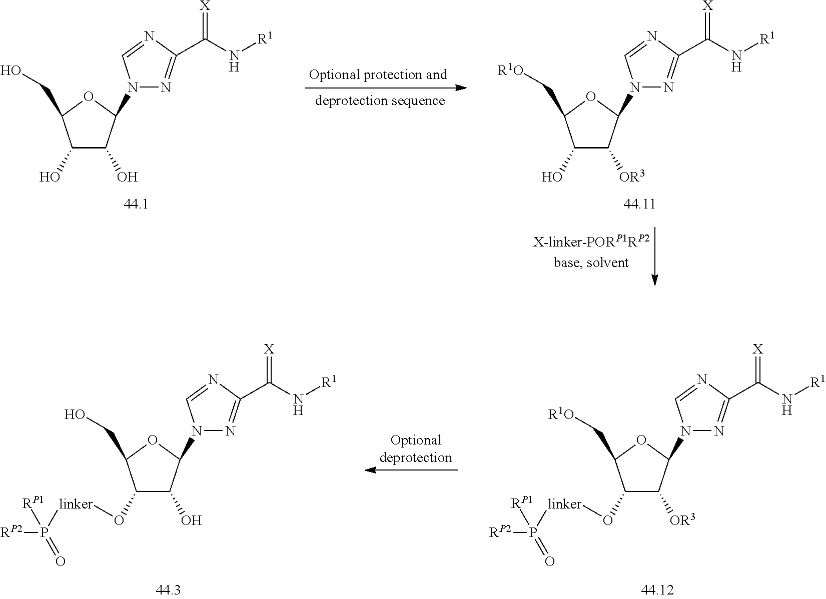

The preparation of 44.4 is illustrated above. The reaction sequence and conditions are similar to that of 44.2 and 44.3 described above.

### EXAMPLE 45

### Synthesis of Representative Compounds of the Invention

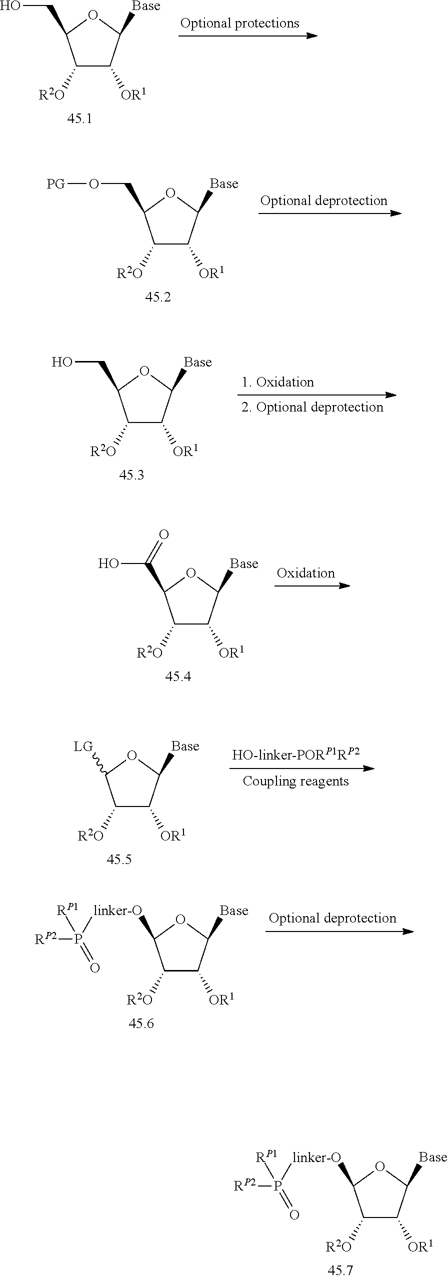

Representative compounds of the invention can be prepared as illustrated above. Compounds having general formula 45.1 can be prepared using procedures described in the literature (see, for example, Townsend, “Chemistry of Nucleosides and Nucleotides,” Plenum Press, 1994; and Vorbruggen and Ruh-Pohlenz, “Handbook of Nucleoside Synthesis,” John Wiley & Sons, Inc., 2001) or be purchased from commercial sources. More specifically, the preparation of generic structure 45.1 is described in Nagahara et al., J. Med. Chem. 33, 1990; 407-415. The structure 45.2 is described in Kini et al., J. Med. Chem. 34, 1991; 3006-3010.

The core components of this reaction sequence are the transformation of compound from 45.3 to 45.6. Appropriate oxidant(s) can convert the primary alcohol (5′-hydroxy) shown in 45.3 to a carboxylic acid or its corresponding ester. In the case of an ester, an additional deprotection step will give the carboxylic acid, 45.4. A variety of oxidation procedures exist in the literature and can be utilized here. These include, but are not limited to, the following methods: (i) pyridinium dichromate in Ac₂O, t-BuOH, and dichloromethane producing the t-butyl ester, followed by a depretection using reagent such as trifluoroacetic acid to convert the ester to the corresponding carboxylic acid (see Classon et al., Acta Chem. Scand. Ser. B, 39 1985; 501-504; Cristalli et al., J. Med. Chem.; 31; 1988; 1179-1183.); (ii) iodobenzene diacetate and 2,2,6,6-tetramethyl-1-piperidinyloxy, free radical (TEMPO) in acetonitrile, producing the carboxylic acid (see Epp et al., J. Org. Chem. 64; 1999; 293-295; Jung et al., J. Org. Chem.; 66; 2001; 2624-2635); (iii) sodium periodate, ruthenium(III) chloride in chloroform producing the carboxylic acid (see Kim et al., J Med. Chem. 37; 1994; 4020-4030; Homma et al., J. Med. Chem.; 35; 1992; 2881-2890); (iv) chromium trioxide in acetic acid producing the carboxylic acid (see Olsson et al., J. Med. Chem.; 29; 1986; 1683-1689; Gallo-Rodriguez et al., J. Med. Chem.; 37; 1994; 636-646); (v) potassium permanganate in aqueous potassium hydroxide producing the carboxylic acid (see Ha et al., J. Med. Chem.; 29; 1986; 1683-1689; Franchetti et al., J. Med. Chem., 41; 1998; 1708-1715); (vi) nucleoside oxidase from S. maltophilia to give the carboxylic acid (see Mahmoudian et al., Tetrahedron; 54; 1998; 8171-8182).

The preparation of compound 45.5 starting with compound 45.4 using lead(IV) tetraacetate (Lv=OAc) is described by Teng et al., J. Org. Chem.; 59; 1994; 278-280 and Schultz et al., J. Org. Chem.; 48; 1983; 3408-3412. When lead(IV) tetraacetate is used together with lithium chloride (see Kochi et al., J. Am. Chem. Soc.; 87; 1965; 2052), the corresponding chloride is obtained (45.5, LG=Cl). Lead(IV) tetraacetate in combination with N-chlorosuccinimide can produce the same product (45.5, LG=Cl) (see Wang et al., Tet. Asym.; 1; 1990; 527 and Wilson et al., Tet. Asym.; 1990; 1;525). Alternatively, the acetate leaving group (LG) can also be converted to other leaving group such as bromide by treatment of trimethylsilyl bromide to give 45.5 (see Spencer, et al; J. Org. Chem.; 64; 1999; 3987-3995).

The coupling of 45.5 (Lv=OAc) with a variety of nucleophiles is described by Teng et al., Synlett; 1996; 346-348 and in U.S. Pat. No. 6,087,482 (column 54, line 64 to column 55, line 20). Specifically, the coupling between 45.5 and diethyl hydroxymethylphosphonate in the presence of trimethylsilyl trifluoromethanesulfonate (TMS-OTf) is described. It can be envisioned that other compounds with the general structure of HO-linker-POR^{P1}R^{P2} can also be used so long as the functional groups in these compounds are compatible with the coupling reaction conditions. There are many examples in the published literature describing the coupling of 45.5 (Lv=halogen) with a variety of alcohols. The reactions can be facilitated with a number of reagents, such as silver(I) salts (see Kim et al., J. Org. Chem.; 56; 1991; 2642-2647; Toikka et al., J. Chem. Soc. Perkins Trans. 1; 13; 1999; 1877-1884), mercury(II) salts (see Veeneman et al., Recl. Tray. Chim. Pays-Bas; 106; 1987; 129-131), boron trifluoride diethyl etherate (see Kunz et al., Hel. Chim Acta; 68; 1985; 283-287), Tin(II) chloride (see O'Leary et al., J. Org. Chem.; 59; 1994; 6629-6636), alkoxide (see Shortnacy-Fowler et al., Nucleosides Nucleotides; 20; 2001; 1583-1598), and iodine (see Kartha et al., J. Chem. Soc. Perkins Trans. 1; 2001; 770-772). These methods can be selectively used in conjunction with different methods in forming 45.5 with various leaving groups (Lv) to produce 45.6.

The transformations from 45.1 to 45.2, from 45.2 to 45.3, and from 45.6 to 45.7 are intended to allow the core components of the transformations (from 45.3 to 45.6) to occur while preserving the functional groups already existing in the compound structures. Thus, the syntheses may require the introduction and removal of protecting groups from a compound. This is a commonly practiced art in organic synthesis. It should be understood that in the transformation 45.6 to 45.7, R^{P1} and R^{P2} do not need to remain unchanged. The final form of R^{P1} and R^{P2} can be selected from a variety of possible structures.

### EXAMPLE 46

### Synthesis of Representative Compounds of the Invention

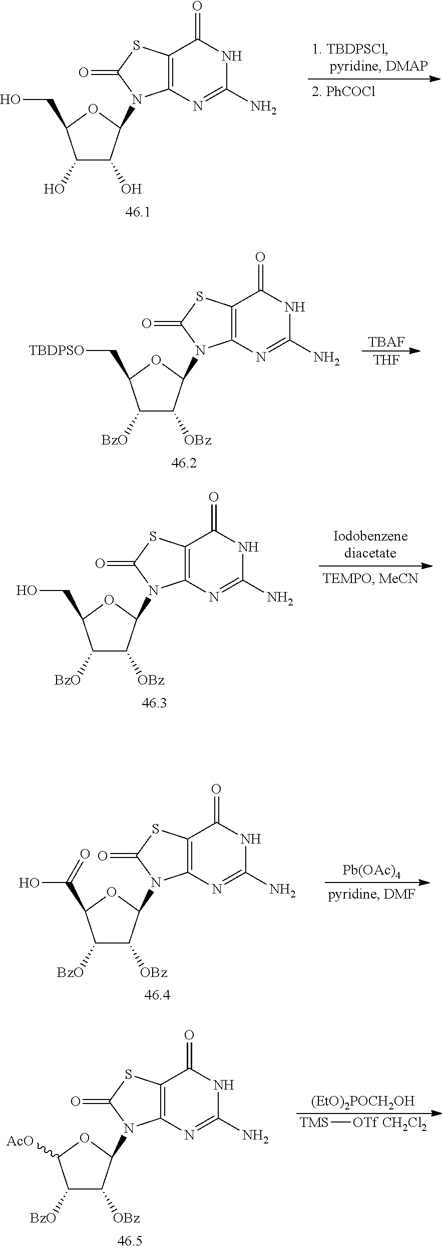

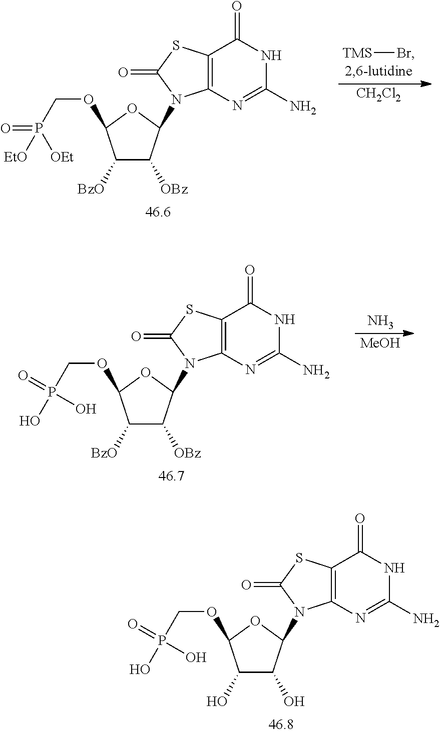

Representative compounds of the invention can be prepared as illustrated above. Compound 46.1 is prepared using the method described in the patent application WO01/90121 (table at page 115). The 5′-hydroxyl in 46.1 is protected as a tert-butyldimethylsilyl (TBDMS) ether. The 2′- and 3′-hydroxyl groups can be protected as bezoyl (Bz) esters to give 46.2. The 5′-hydroxyl can then be deprotected to give 46.3. Oxidation using iodobenzene diacetate and 2,2,6,6-tetramethyl-1-piperidinyloxy, free radical (TEMPO) convert the primary alcohol to the corresponding acid 46.4. Further oxidation of 46.4 using lead tetraacetate can produce 46.5. Coupling between 46.5 and diethyl hydroxy-methyl-phosphonate (available from Sigma-Aldrich, Cat. No. 39,262-6) effected by TMS-OTf can afford 46.6. Treating 46.6 with TMS-Br converts the phosphodiester to the corresponding phosphonic acid 46.7. Deprotection of the 2′- and 3′-hydroxyl gives 46.8, where Base is an 7-thia-8-oxo-guanosine, R¹, R², R^{P1} and R^{P2} are hydrogen, linker is a methylene group.

The phosphonic acids in 46.7 and 46.8 are used as examples for illustration purpose. Other forms of phosphonates can be acquired via the phosphonic acid, or other forms, such as the corresponding diesters as described herein.

### EXAMPLE 47

### Synthesis of Representative Compounds of the Invention

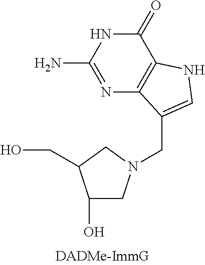

A highly potent analog inhibiting the PNP enzyme from Mycobacterium tuberculosis (MtPNP) called DADMe-ImmG, structure above, was prepared recently (Lewandowics A. et al., Biochemistry, (2003), 42, 6057).

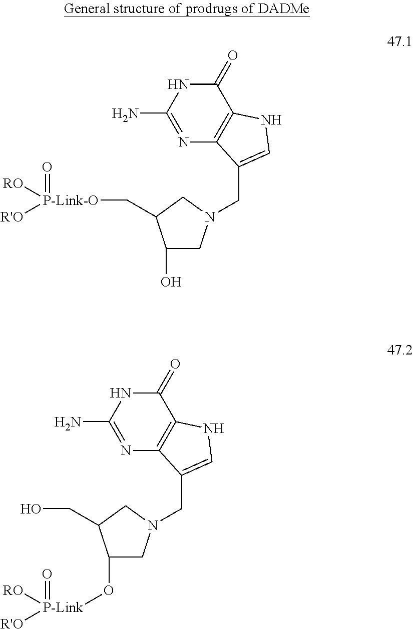

Reduction of the dose and/or improvement of efficacy might be achieved by the use of pro-drugs DADMe-ImmG that, upon cleavage inside the target cell, give rise to agents with increased intracellular half-lives. Such phosphonates pro-drug compounds are shown above.

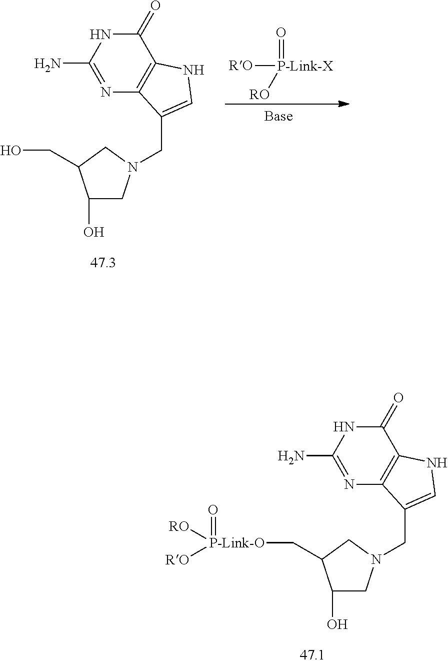

Representative compounds of the invention, such as 47.1, can be made according to the general route illustrated above.

### EXAMPLE 48

### Synthesis of Representative Compounds of the Invention

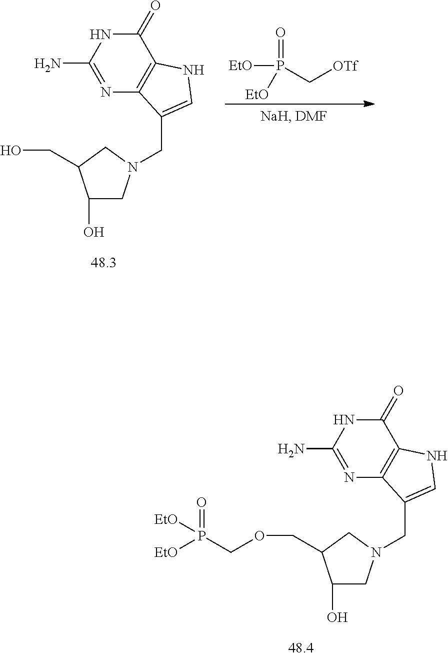

Representative compounds of the invention can be prepared as illustrated above. Preparation of DADMe-ImmG is reported in Lewandowics A. et al., Biochemistry, (2003), 42, 6057. The tertiary nitrogen of the ring may not interfere with the alkylation of the secondary alcohol and in that case does not need to be protected, although standard protection and deprotection protocols as described in Greene, T. “Protective Groups in Organic Synthesis,” Wiley-Interscience, (1999) may be used if necessary. Reaction of the primary alcohol 48.3 with base followed by addition of the appropriately activated phosphonate yields the protected product. Global deprotection yields the desired phosphonate 48.4.

### EXAMPLE 49

### Synthesis of Representative Compounds of the Invention

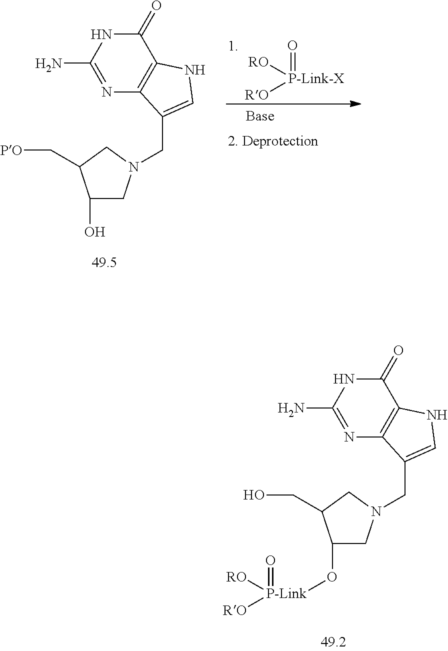

Representative compounds of the invention can be prepared as illustrated above. Preparation of DADMe-ImmG is reported in Lewandowics A. et al., Biochemistry, (2003), 42, 6057. Blocking of the primary alcohol can be achieved by methods described in Greene, T., “Protective groups in organic synthesis,” Wiley-Interscience, (1999). Reaction of the secondary alcohol in base followed by addition of the appropriately activated phosphonate yields the protected desired product. Deprotection yields the desired phosphonate.

### EXAMPLE 50

### Synthesis of Representative Compounds of the Invention

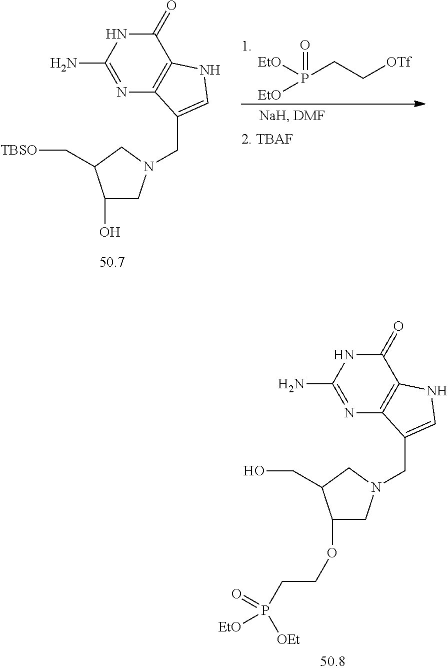

Specifically, the protected DADMe derivative can be treated with treated in a solvent such as tetrahydrofuran or dimethylformamide with a base such as sodium hydride. When bubbling ceases, diethyl phosphonoethylltrifiate (prepared according to Tetrahedron Lett. 1986, 27, 1477) is added, yielding the desired phosphonate ester. Removal of the protecting group can be performed as described in Greene, T., “Protective groups in organic synthesis,” Wiley-Interscience, (1999) to provide the desired phosphonate ester.

### EXAMPLE 51

### Synthesis of Representative Compounds of Formulae 51 and 52

Representative compounds of the invention can also be prepared by following the sequences illustrated in Examples 41-44 using enantiomeric starting materials corresponding to, for example, compounds 41.2 and 42.2 to provide compounds of Formulae 51 and 52, respectively.

### EXAMPLE 52

### Preparation of Representative Compounds of the Invention

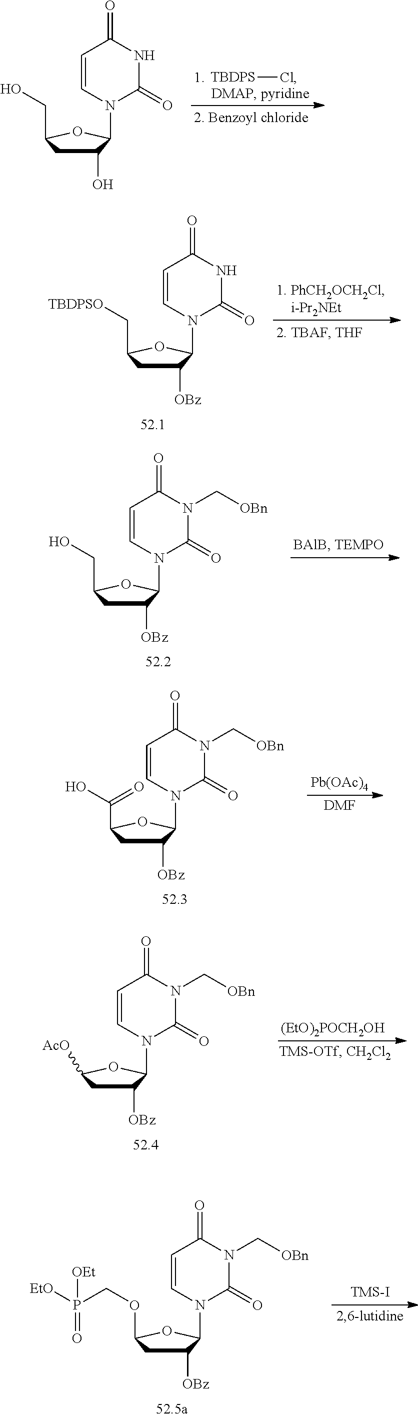

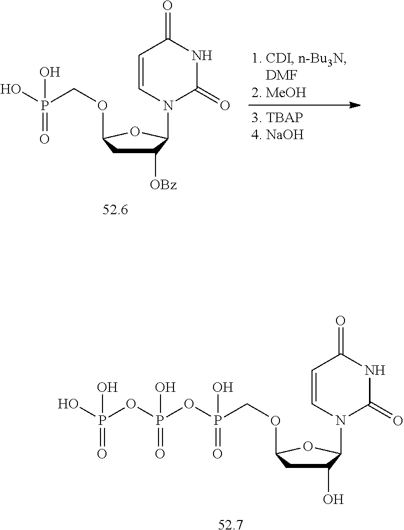

### EXAMPLE 53

### Preparation of Representative Compounds of the Invention

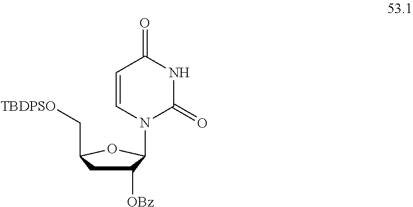

Synthesis of 53.1: To a solution of 3′-deoxyuridine (995 mg, 4.36 mmol) in 8 mL of anhydrous pyridine was added t-butyldiphenylsilyl chloride (TBDPS-Cl, 1.38 g, 5.01 mmol), and 4-dimethylaminopyridine (DMAP, 27 mg, 0.22 mmol). The mixture was stirred at 23° C. for 14 hours and then cooled to 0° C. in a ice-water bath. To this mixture was added benzoyl chloride (735 mg, 0.61 mL, 5.2 mmol). The mixture was warmed to 23° C. and stirred for another 2 hours. The mixture was concentrated in vacuo to give a paste, which was partitioned between water and ethyl acetate. The aqueous later was extracted once with ethyl acetate. The combined ethyl acetate layer was washed sequentially with 1 M aqueous citric acid, saturated sodium bicarbonate, and brine. It was dried over anhydrous sodium sulfate and concentrated in vacuo to give a crude product as a yellow oil. Purification by silica gel chromatography (15-65% ethyl acetate in hexane) gave a colorless oil. Yield 1.35 g (54%). ¹H NMR (DMSO-d6): δ 11.38 (s, 1H), 8.01 (d, J=7.9 Hz, 2H), 7.77 (d, J=8.2 Hz, 1H), 7.70-7.40 (m, 13 H), 5.99 (s, 1H), 5.58 (m, 1H), 7.34 (d, J=8.2 Hz, 1H), 4.47 (m ,1H), 4.03 (m, 1H), 3.84 (m, 1H), 2.43 (m, 1H), 2.21 (m, 1H), 1.03 (s, 9H) ppm. MS (m/z) 571.1 (M+H⁺), 593.3 (M+Na⁺).

### EXAMPLE 54

### Preparation of Representative Compounds of the Invention

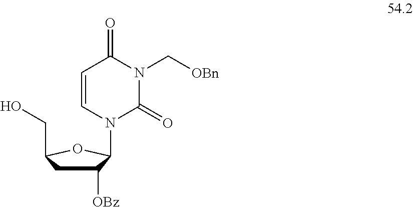

### Synthesis of 54.2

To a solution of 53.1 (1.31 g, 2.3 mmol) in 5 mL of anhydrous N,N-dimethylformamide was added benzyl chloromethyl ether (0.54 g, 3.45 mmol), N,N-diisopropylethylamine (446 mg, 0.60 mL, 3.45 mmol). The mixture was stirred at 23° C. for 4 hours. Water was added. The mixture was extracted with ethyl acetate. The organic layer was washed sequentially with 1 M aqueous citric acid, saturated sodium bicarbonate, and brine. It was dried over anhydrous sodium sulfate and concentrated in vacuo to give a crude product as a yellow oil, which was used in the next step without further purification.

The crude product obtained above was dissolved in 9 mL of THF. The solution was cooled to 0° C. A 1 M solution of TBAF (4.6 mL, 4.6 mmol) was added via syringe. The mixture was warmed to 23° C. and stirred for another 2 hours. An additional 2.3 mL of 1 M TBAF was added. The mixture was stirred for another 2 hours at 23° C. Saturated aqueous ammonium chloride was added to the solution. The mixture was evaporated in vacuo to remove most of THF. The aqueous phase was extracted with ethyl acetate. The aqueous layer was washed with brine. It was then dried over anhydrous sodium sulfate and concentrated in vacuo to give a crude product as a yellow oil. Purification by silica gel chromatography (30-80% ethyl acetate in hexane) gave a white solid. Yield of 54.2: 805 mg (77% for two steps). ¹H NMR (DMSO-d6): δ 8.04 (m, 3H), 7.67 (t, J=7.3 Hz, 1H), 7.55 (t, J=7.6 Hz, 2H), 7.30 (m, 5H), 5.98 (s, 1H), 5.78 (d, J=7.9 Hz, 1H), 5.55 (m, 1H), 5.31 (s, 2H), 5.22 (m, 1H), 4.57 (s, 2H), 4.41 (m ,1H), 3.80 (m, 1H), 3.60 (m, 1H), 2.31 (m, 1H), 2.15 (m, 1H) ppm. MS (m/z) 453.1 (M+H⁺), 475.3 (M+Na⁺).

### EXAMPLE 55

### Preparation of Representative Compounds of the Invention

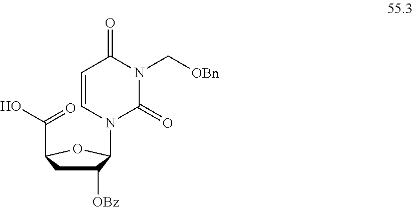

### Synthesis of 55.3

To a solution of 54.2 (800 mg, 1.77 mmol) in 3.5 mL of a 1:1 mixture of acetonitrile/water was added iodobenzene diacetate (1.25 g, 3.89 mmol), and TEMPO (55 mg, 0.35 mmol). The mixture was stirred at 23° C. for 14 hours. The mixture was then froze in a −78° C. bath and lyophilized to give a solid residue. This residue was purified by silica gel chromatography (0-15% methanol in dichloromethane). Product 55.3 was obtained as a white solid. Yield: 735 mg (89%). ¹H NMR (DMSO-d6): δ 8.13 (d, J=7.6 Hz, 1H), 8.03 (d, J=7.7 Hz, 2H), 7.68 (m, 1H), 7.58 (t, J=7.0 Hz, 2H), 7.29 (m, 5H), 6.04 (s, 1H), 5.85 (d, J=8.3 Hz, 1H), 5.62 (m, 1H), 5.31 (s, 2H), 4.87 (m, 1H), 4.58 (s, 2H), 2.40-2.20 (m, 2H) ppm. MS (m/z) 467.1 (M+H⁺), 489.3 (M+Na⁺).

### EXAMPLE 56

### Preparation of Representative Compounds of the Invention

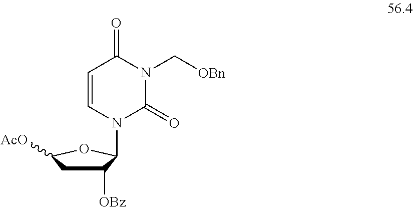

### Synthesis of 56.4

To a deoxygenated solution of 55.3 (730 mg, 1.57 mmol) and pyridine (0.51 mL, 6.26 mmol) in 7 mL of anhydrous DMF, was added lead tetraacetate (3.47 g, 7.83 mmol). The mixture was stirred at 23° C. for 14 hours shielded from light. The mixture was diluted with 15 mL of ethyl acetate and 10 mL of water. This mixture filtered through a pad of Celite and separated. The aqueous phase was extracted with another 10 mL of ethyl acetate. The combined ethyl acetate extract was washed with brine, dried over sodium sulfate, and evaporated in vacuo to give the crude product as an oil. The crude product 56.4 was purified by silica gel chromatography (10-50% ethyl acetate in hexane). Products of two diastereomers were obtained as a white foam. Yield: 400 mg (53%). ¹H NMR (DMSO-d6): δ 8.01 (m, 2H), 7.82-7.63 (m, 2H), 7.57 (m, 2H), 7.31 (m, 5H), 6.58 (m, 1H), 6.17 (m, 1H), 5.83 (m, 1H), 5.65 (m, 1H), 5.31 (s, 2H), 4.59 (s, 2H), 2.76 and 2.28 (m, 1H), 2.10 (m, 1H), 2.07 (s, 3H) ppm. MS (m/z) 481.0 (M+H⁺), 503.3 (M+Na⁺).

### EXAMPLE 57

### Preparation of Representative Compounds of the Invention

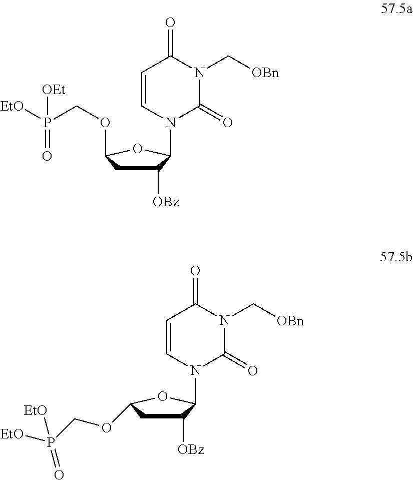

### Synthesis of 57.5a

To a solution of 56.4 (300 mg, 0.63 mmol) in 6 mL of anhydrous dichloromethane was added diethyl hydroxymethyl-phosphonate (0.37 mL, 2.5 mmol), followed by trimethylsilyl trifluoromethanesulfonate (0.34 mL, 1.88 mmol). The mixture was stirred at 23° C. for 6 hours. Triethylamine (0.44 mL, 3.15 mmol) was added, followed by water. The mixture was extracted with ethyl acetate. The organic layer was washed with 1 M aqueous citric acid, saturated sodium bicarbonate, and brine. It was then dried over anhydrous sodium sulfate, and evaporated in vacuo to give a residue. This crude product was purified by silica gel chromatography (75-95% ethyl acetate in hexane) to give two products, which were diastereomers of each other shown above (57.5a and 57.5b). Yield of 57.5a: 53 mg (14%). Yield of 57.5b: 129 mg (35%).

Analytical data for 57.5a: ¹H NMR (Acetonitrile-d3): δ 8.04 (d, J=7.0 Hz, 2H), 7.77 (d, J=7.9 Hz, 1H), 7.69 (t, J=7.5 Hz, 1H), 7.53 (m, 2H), 7.33 (m, 5H), 6.38 (d, J=4.0 Hz, 1H), 5.80 (d, J=8.2 Hz, 1H), 5.63 (m, 1H), 5.52 (m, 1H), 5.41 (s, 2H), 4.64 (s, 2H), 4.17 (m, 4H), 4.08 (dd, J=13.8, 10.1 Hz, 1H), 3.92 (dd, J=13.7, 9.5 Hz, 1H), 2.66-2.42 (m, 2H), 1.35 (t, J=7.0 Hz, 6H) ppm. MS (m/z) 589.2 (M+H⁺), 611.3 (M+Na⁺). Stereochemistry of 57.5a was confirmed by additional 2D NMR experiments.

Analytical data for 57.5b: ¹H NMR (Acetonitrile-d3): δ 8.08 (d, J=7.3 Hz, 2H), 7.69 (t, J=7.5 Hz, 1H), 7.55 (m, 2H), 7.43 (d, J=8.2 Hz, 1H), 7.36 (m, 5H), 6.11 (d, J=2.4 Hz, 1H), 5.77 (d, J=8.3 Hz, 1H), 5.57 (m, 2H), 5.41 (s, 2H), 4.66 (s, 2H), 4.12 (m, 5H), 3.88 (dd, J=14.0, 5.2 Hz, 1H), 2.82 (m, 1H), 2.25 (m, 1H), 1.27 (t, J=7.0 Hz, 6H) ppm. MS (m/z) 589.0 (M+H⁺), 611.2 (M+Na⁺).

### EXAMPLE 58

### Preparation of Representative Compounds of the Invention

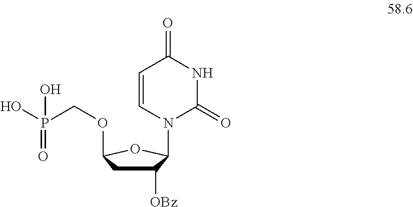

### Synthesis of 58.6

To a solution of 57.5a (110 mg, 0.19 mmol) in 3 mL of acetonitrile was added 2,6-lutidine (0.43 mL, 3.74 mmol), followed by iodotrimethylsilane (0.53 mL, 3.74 mmol). After stirring at 23° C. for 30 minutes, the mixture was heated to 40° C. and stirred at that temperature for another 4 hours. The reaction mixture was cooled to 23° C. Triethylamine (0.52 mL, 3.74 mmol) was added, followed by water (10 mL). The aqueous mixture was extracted twice with 5 mL of diethyl ether. The resulting aqueous solution was frozen in a −78° C. bath and was lyophilized to give a yellow solid. This crude product was purified by reversed phase HPLC to give 58.6 as a light yellow solid. Yield 26 mg (34%). MS (m/z) 411.3 (M−H⁻).

### EXAMPLE 59

### Preparation of Representative Compounds of the Invention

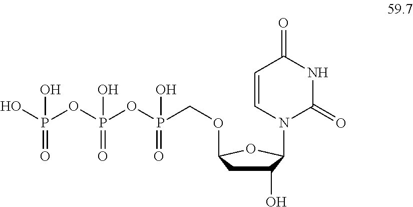

### Synthesis of 59.7

Phosphonate 58.6 (12 mg, 0.029 mmol), carbonyldiimidazole (47 mg, 0.29 mmol), and tri-n-butylamine (5.4 mg, 0.029 mmol) were dissolved in 0.3 mL of anhydrous dimethylformamide (DMF). The mixture was stirred at 23° C. for 4 hours. MeOH (0.020 mL) was added and the mixture was stirred for another 30 minutes. A solution of tributylammonium pyrophosphate (159 mg, 0.29 mmol) in 0.63 mL of anhydrous DMF was added. The resulting mixture was stirred at 23° C. for 14 hours. The mixture was evaporated in vacuo to remove most of the DMF. The residue was dissolved in 5 mL of water and was purified by ion-exchange chromatography (DEAE-cellulose resin, 0-50% triethylammonium bicarbonate in water) to give a white solid, which was used directly in the next reaction.

The product obtained above was dissolved in 2 mL of water. A 0.3 mL of a 1 M solution of sodium hydroxide in water was added. The mixture was stirred at 23° C. for 40 minutes. Acetic acid was added to adjust the pH of the solution to 5. The solution was diluted with water and purified with an ion-exchange column (DEAE-cellulose resin, 0-50% triethylammonium bicarbonate in water) to give diphosphophosphonate 59.7 as a white solid, which is the triethylammonium salt of the structure shown above. Yield 10 mg (45% for two steps). ¹H NMR (D₂O): δ 7.79 (d, J=7.6 Hz, 1H), 5.89 (m, 1H), 5.85 (d, J=7.6 Hz, 1H), 5.41 (m, 1H), 4.49 (m, 1H), 4.02-3.65 (m, 2H), 3.06 (m, 18H), 2.20 (m, 2H), 1.14 (m, 27H) ppm. ³¹P NMR (D₂O): δ 7.46 (d, 1P), -9.45 (d, 1P), −23.11 (t, 1P) ppm. MS (m/z) 467.0 (M−H⁻).

### EXAMPLE 60

### Preparation of Representative Compounds of the Invention

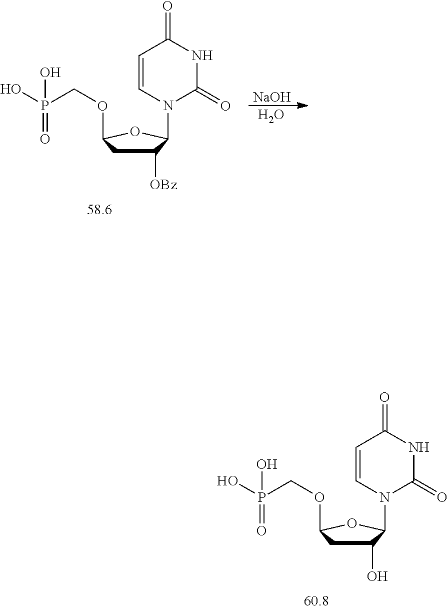

### Synthesis of 60.8

To a solution of 58.6 (16 mg, 0.039 mmol) in 0.4 mL of water was added NaOH (7.8 mg, 0.19 mmol). The solution was stirred at 23° C. for 1 hour. Acetic acid (0.012 mL) was added to the solution. The mixture was then purified by reversed phase HPLC (eluted with 100% water) to give 4.6 mg of 60.8 as a white solid (38% yield). ¹H NMR (D₂O): δ 7.83 (d, J=8.3 Hz, 1H), 5.86 (d, J=3.4 Hz, 1H), 5.82 (d, J=7.9 Hz, 1H), 4.48 (m, 1H), 3.68 (m, 1H), 3.37 (m, 1H), 2.16 (m, 2H) ppm. ³¹P NMR (D₂O): δ 12.60 (s, 1P) ppm. MS (m/z) 615.1 (2M−H⁻).

### EXAMPLES 61-63

The analogs described in Examples 61-63 show a methylene group as the linker. The linker may, however, be any other group described in this specification.

### EXAMPLE 61

### Preparation of Representative Compounds of Formula 66

### Cyclobutanes:

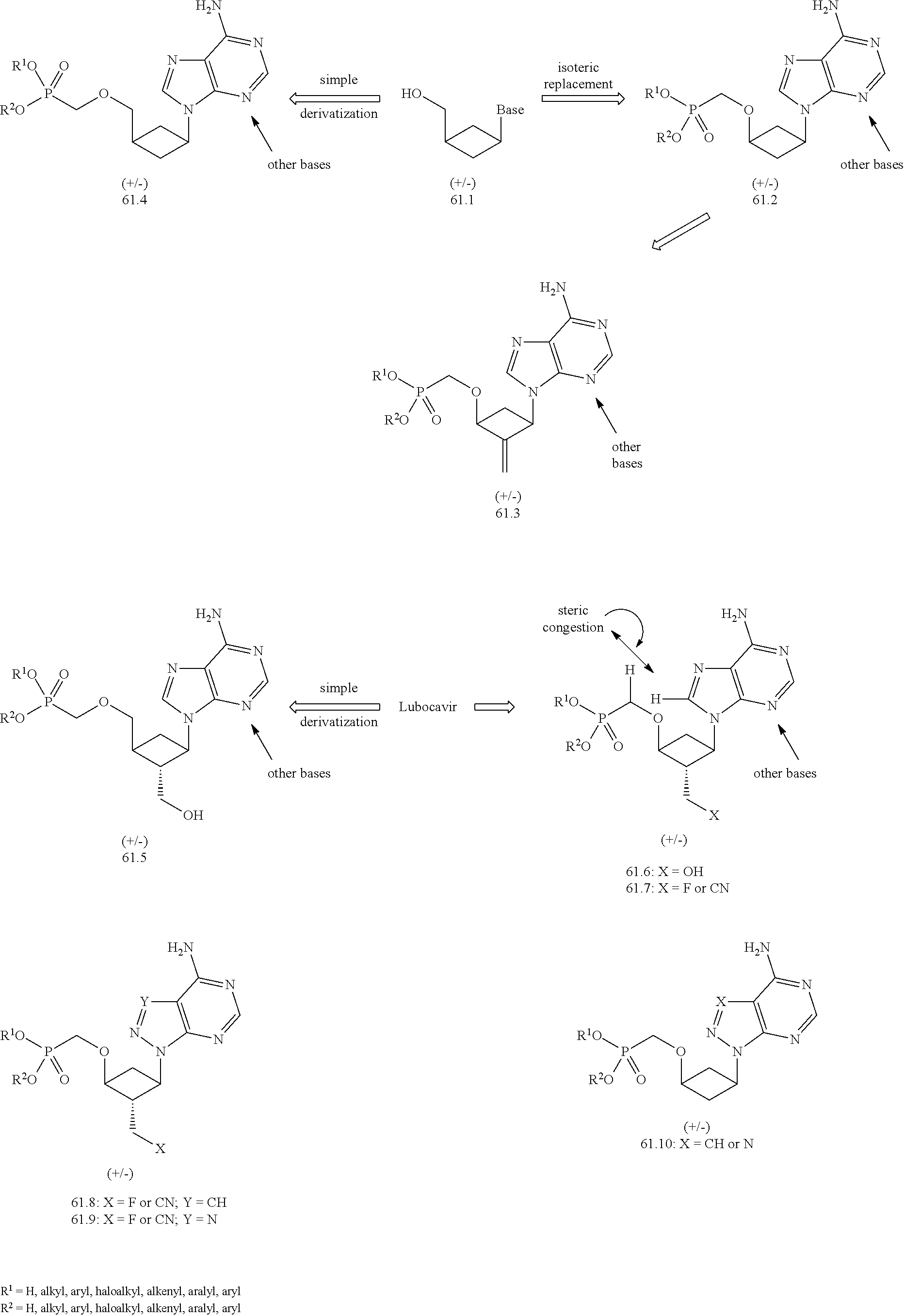

### 4-Membered Ring Nucleoside Series:

Representative compounds of the invention can be prepared as illustrated above. Compound 61.1 (Base=G), described in the literature, was shown to have reasonable anti-HIV activity (50-100 μM, cf. lubocavir 30 μM). Therefore, one compound of the invention is its isosteric phosphonate derivative 61.2. Also, compound 61.1 can be derivatized to its phosphonate 61.4. In a similar manner, a phosphonate group can be added onto lubocavir to prepare carbocyclic 61.5, or alternatively carbocyclic isostere 61.6. Compounds 61.6 have a hydroxyl group analogous to the 3’-hydroxyl group of natural nucleosides. Such compounds may be incorporated into elongating strands by host DNA polymerases, a phenomenon that may be associated with both carcinogenicity and mitochondrial toxicity. Replacement of hydroxyl groups with fluorine atoms is established in medicinal chemistry. A well-known example is the replacement of the terminal hydroxyl group of the antibiotic chloramphenicol with a fluorine atom, providing florfenicol. In the case of 61.6 (or 61.8 and 61.9), the fluorine maintains many beneficial H-bonding interactions with RT that the pseudo-3′-hydroxyl of 61.5 provided, but will not provide a handle for incorporation into nucleic acids.

### The Chemistry of Derivatives 61.8/61.9

A protected version of A maybe required (e.g., a pivalate) or a masked version of A (e.g., in lieu of aniline). If a masked version of A is required, synthesis of the base will be required. Fluorination reactions in the presence of unprotected A yield ˜90% when pyridine is used as a solvent. Acidic deprotection should not cause de-glycosidation of base since there is no formal glycosidic linkage (O—C—N). All reactions proceed with useful kinetic diastereoselectivity. In base introduction reactions, equilibration conditions may be used to improve the kinetic diastereoselection ratio. All compound made by this route are racemic. Enantiomerically pure compounds may be prepared by known methods.

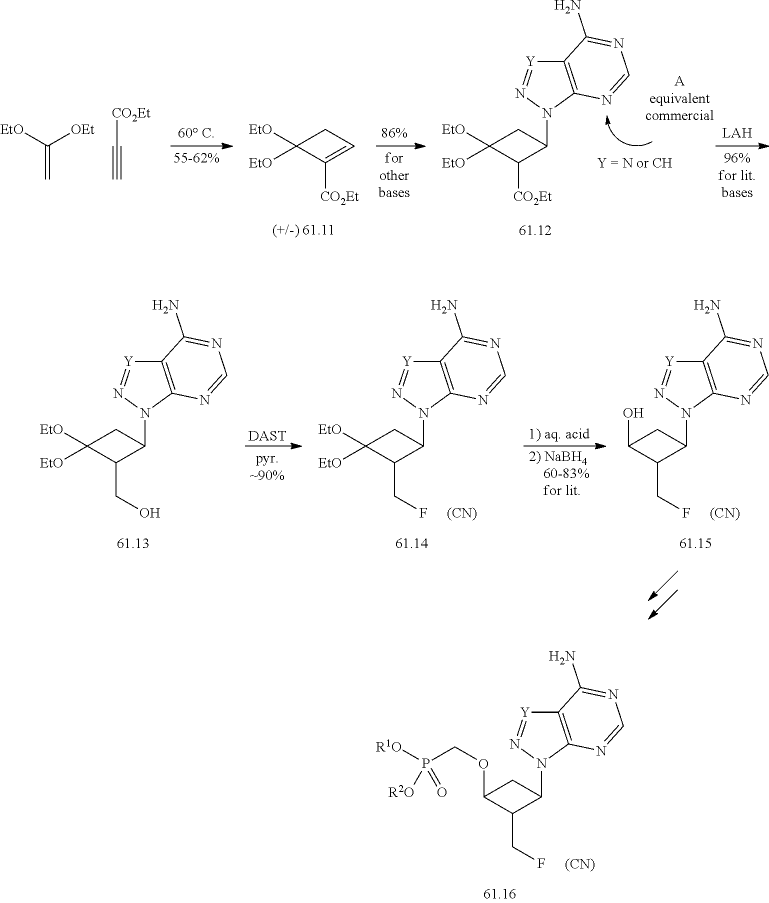

Illustrated above is a synthetic sequence for the preparation of compound 61.16. Other nucleotide bases may optionally be used in this synthetic sequence.

### EXAMPLE 62

### Preparation of Representative Compounds of Formula 67

### Cyclopropyl Nucleoside Series

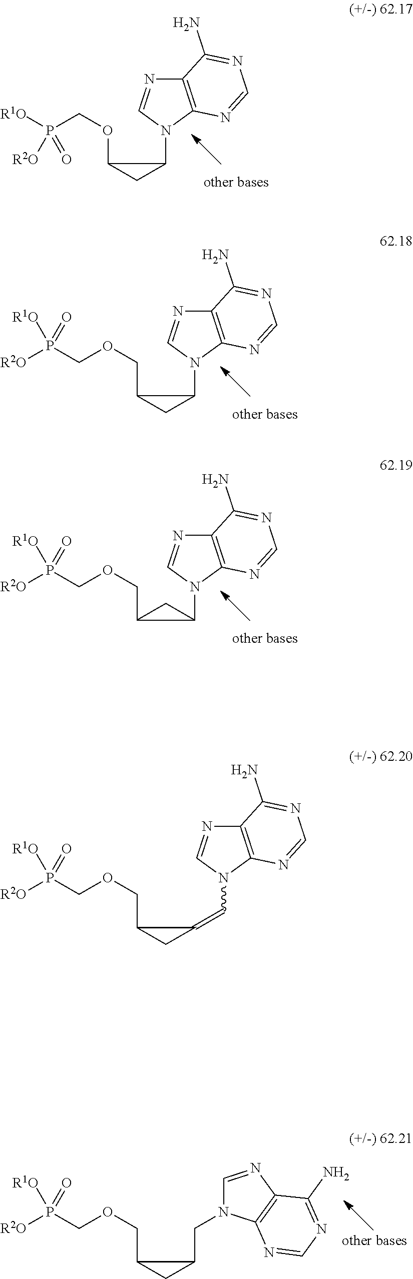

Representative compounds of the invention can be prepared as illustrated above. The synthesis of cyclopropyl nucleosides of type 62.18 and 62.19 is well documented. Synthetic methods allow for the homochiral production of 62.18 and 62.19. Syntheses of compound types 62.17, 62.20, and 62.21 are also reported. These provide for racemic material.

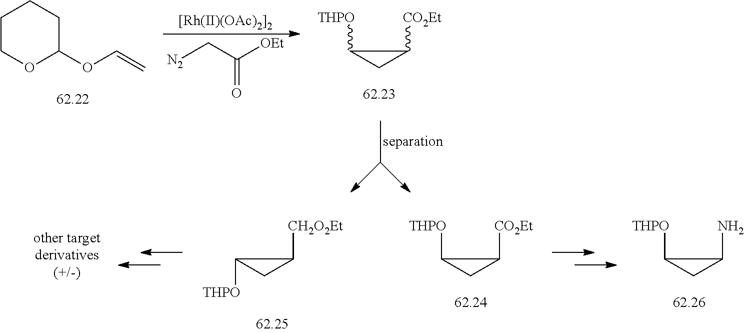

### Considerations in the Synthesis of 62.17

Literature reports of this synthesis are directed to industrial processes. Diastereomers are produced by a non-stereoselective cyclopropanation reactions and separation of the desired isomer with cis cyclopropyl substituents from that with trans may require rigorous separation techniques or alternate synthetic preparations because of the presence of an additional stereocenter at the THP anomeric position, as shown above in 62.23.

### Considerations in the Synthesis of 62.18 and 62.19

For the D series (compound 62.18), synthesis of key intermediate 62.29 (see below) can be preformed in 10 steps in 6 pots (24% overall yield from an abundant starting material, 1,2:5,6-di-O-isopropylidine-D-mannitol. Purine bases can be constructed from free amine 62.29. Phosphonate synthesis proceeds well according to known methodology. For the L series, the starting material is vitamin C.

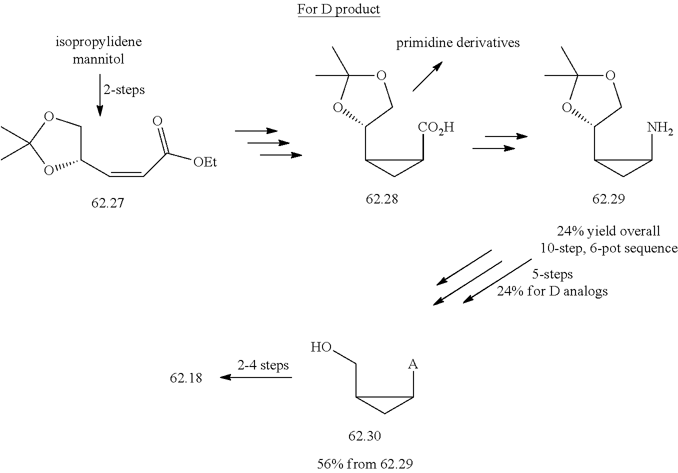

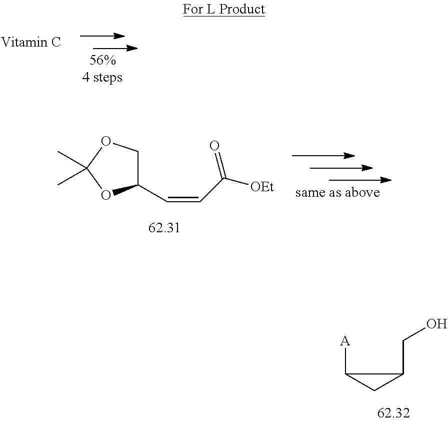

### EXAMPLE 63

### Synthesis of Representative Compounds of Formula 68

### Vinylic Nucleoside Series

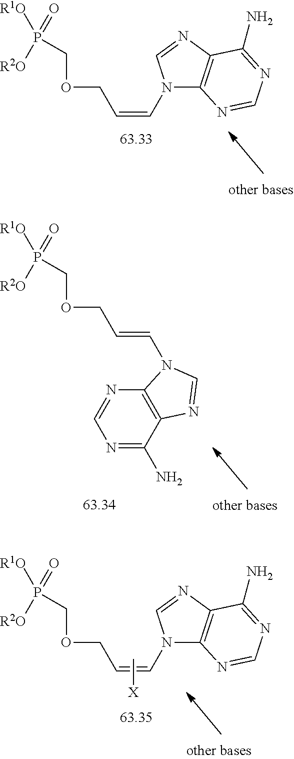

Representative compounds of the invention can be prepared as illustrated above. There are only a few reports of compound types 63.33 and 63.34 in the literature. Most reports provide for the syntheses of trans isomers 63.34. The one report that discusses cis isomers 63.33 does not state clear separation conditions from the mixture of cis and trans compounds formed. The cis isomers best resembles the geometry of the nucleoside antiviral agents and are therefore important compounds. Modeling studies indicate that 63.33 will be accommodated by the RT active site. However, when minimized in the RT active site side-by-side with tenofovir, some of the base stacking interactions that provide binding energy between the inhibitor and the template strand may be lost.

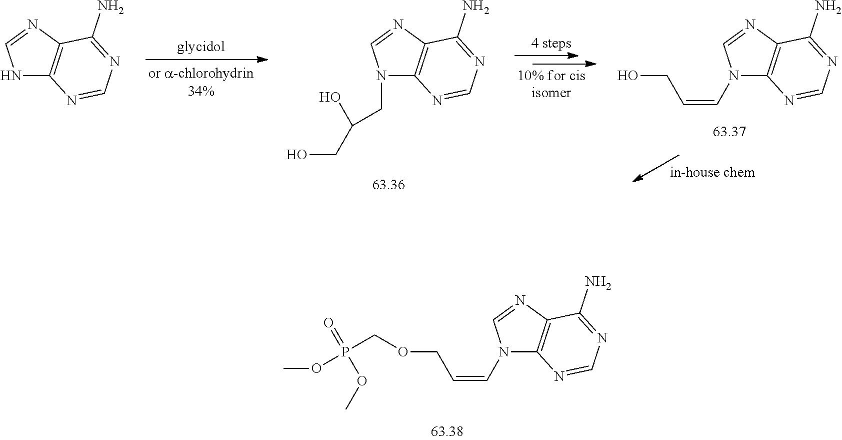

### EXAMPLE 64

### Preparation of Representative Compounds of the Invention

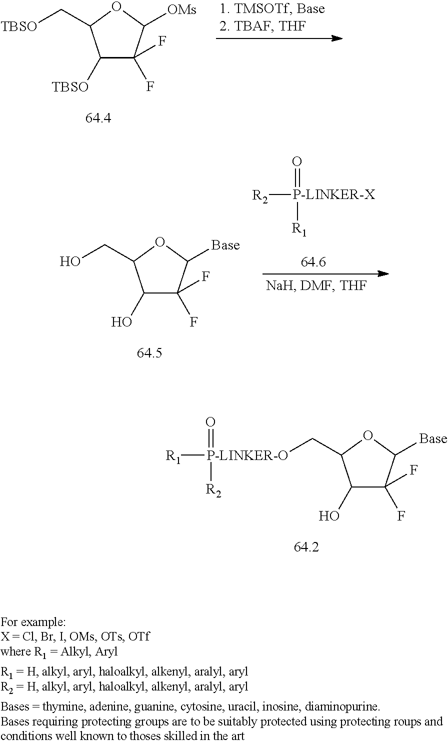

Representative compounds of the invention can be prepared as illustrated above. The desired phosphonate substituted analogs are prepared by reaction of intermediate 64.5 (obtained as described in U.S. Pat. No. 5,464,826) with the respective alkylating reagents 64.6. Illustrated above is the preparation of phosphonate linkage to 2′2′-difluoronucleosides through the 5′-hydroxyl group. The appropriately protected base as described in U.S. Pat. No. 5,464,826 is dissolved in a solvent such as DMF, THF and is treated with a phosphonate reagent bearing a leaving group, for example, bromine, mesyl, tosyl, or trifluoromethanesulfonyl in the presence of a suitable organic or inorganic base.

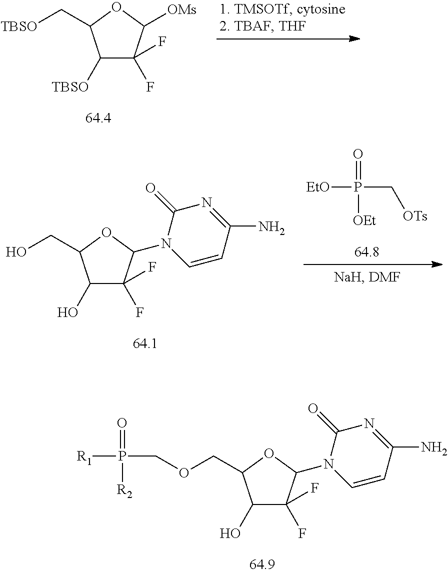

For instance, 64.1 (obtained as described in U.S. Pat. No. 5,464,826) dissolved in DMF, is treated with two equivalents of sodium hydride and one equivalent of (toluene-4-sulfonylmethyl)-phosphonic acid diethyl ester 64.8, prepared according to the procedures in J. Org. Chem. 1996, 61, 7697, to give the corresponding phosphonate 64.9 in which the linkage is a methylene group.

Using the above procedure but employing different phosphonate reagents 64.6 in place of 64.8, the corresponding products 64.2 bearing different linking groups are obtained.

### EXAMPLE 65

### Preparation of Representative Compounds of the Invention

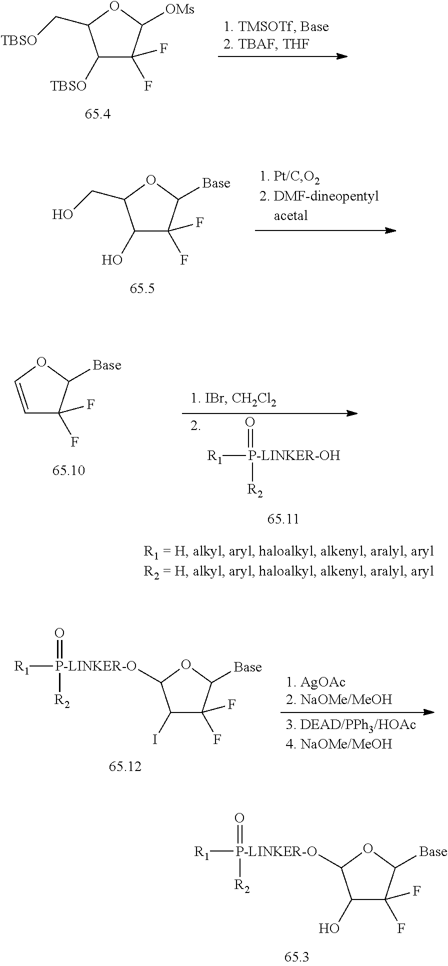

Representative compounds of the invention can be prepared as illustrated above. Compounds 65.5 containing a variety of suitably protected bases as cited and prepared according to U.S. Pat. No. 5,464,826 can be converted to glycal 65.10 according to the process reported in J. Am. Chem. Soc. 1972, 94, 3213. Glycal 65.10 is then treated with IBr in the presence of alcohol 65.11 to provide intermediate 65.12 (see J. Org. Chem. 1991, 56, 2642). The iodide of intermediate 65.12 can be treated with AgOAc to provide the corresponding acetate, which is deacetylated in the presence of catalytic sodium methoxide in methanol. Treatment of the resulting alcohol with DEAD and PPh₃ in the presence of acetic acid, followed by another deprotection with catalytic sodium methoxide in methanol will provide intermediate 65.3. The phosphonates of intermediates 65.3 can then be converted into their final desired forms.

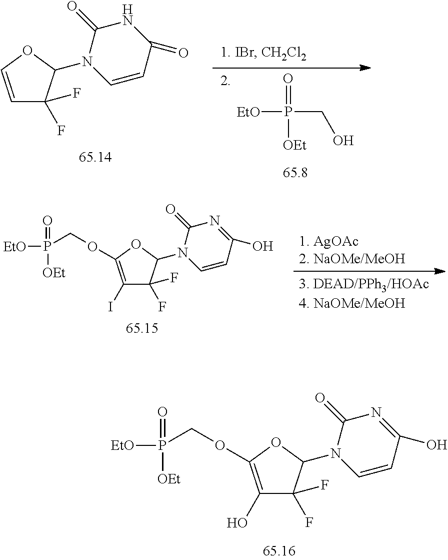

For instance, glycal 65.14 is prepared according to the procedures cited above (U.S. Pat. No. 5,464,826; J. Am. Chem. Soc. 1972, 94, 3213). Glycal 65.14 is then treated with IBr in the presence of diethyl phosphonomethanol, 65.8, to provide intermediate 65.15 (see J. Org. Chem. 1991, 56, 2642). Intermediate 65.15 is then treated with AgOAc followed by deprotection with catalytic NaOMe in MeOH. This compound is then converted into 65.16 by a Mitsunobu reaction with DEAD/PPh₃ and HOAc in THF, followed by a second catalytic NaOMe/MeOH deprotection. Base conversion of uracil to cytosine is carried out prior to the acetyl deprotection using the procedures in Bioorg. Med. Lett. 1997, 7, 2567. At any point in the synthesis sequence where it is appropriate the phosphonate group may be converted into the phosphonate with the desired substitution.

Using the above procedure but employing different phosphonate reagents 65.11 in place of 65.8, the corresponding products 65.3 bearing different linking groups are obtained.

### EXAMPLE 66

### Synthesis of Representative Compounds of Formula 72

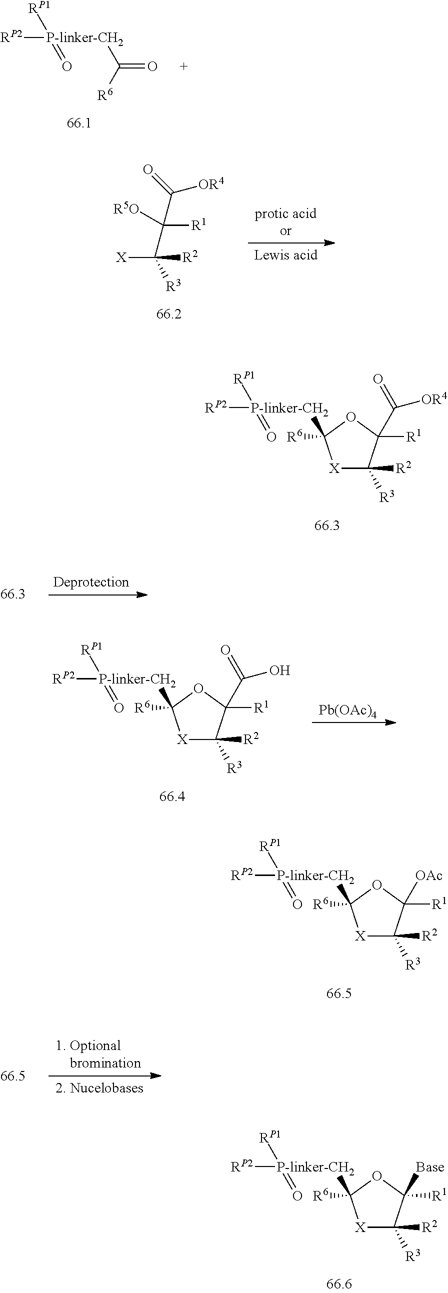

Representative compounds of the invention can be prepared as illustrated above. In the above scheme, R⁴ and R⁵ are appropriate protective groups. Group X is either hydroxyl (or oxygen) or thiol (or sulfur), or appropriately protected hydroxyl or thiol. Additionally, R⁵ can be a cyclic protecting group for both the hydroxyl and X. The general method for the preparation of intermediates 66.3, 66.4, 66.5, and final product 66.6, are described in WO 03/020222 A2 page 28 line 10 to page 53 line 22, as well as the references cited therein. Additional description is provided in WO 01/32153 A2 page 41 line 3 to page 56 line 29 and references cited therein. Other good sources of information for transformation from 66.5 to 66.6 are Townsend, Chemistry of Nucleosides and Nucleotides, Plenum Press, 1994; and Vorbruggen and Ruh-Pohlenz, Handbook of Nucleoside Synthesis, John Wiley & Sons, Inc., 2001.

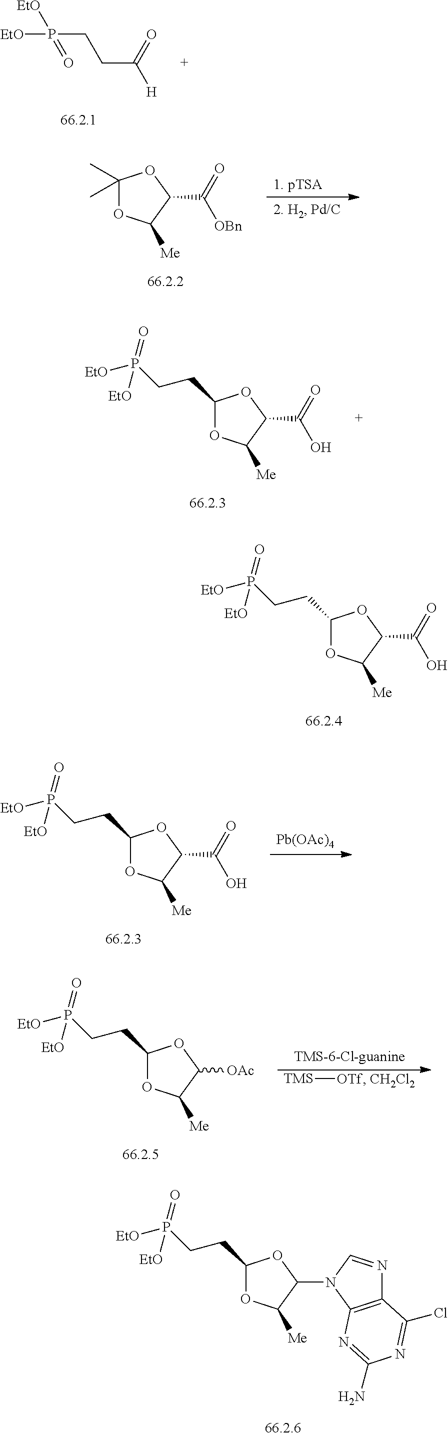

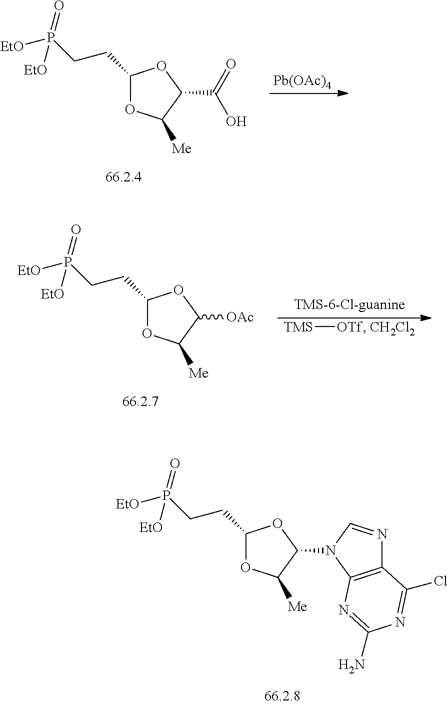

A specific example for the synthesis of a dioxolane nucleoside analog is illustrated above. Treating the mixture of 66.2.1 and 66.2.2 with p-toluenesulfonic acid, followed by removal of the benzyl protecting group on the carboxylic acid produces a mixture of carboxylic acids 66.2.3 and 66.2.4. Treating acid 66.2.3 with lead(IV) tetraacetate gives acetate 66.2.5, which can be converted to nucleoside 66.2.6 under the reaction conditional described above. Treating acid 66.2.4 with same reaction procedures for 66.2.3 to 66.2.6 can generate a different diastereomer 66.2.8, which is an L-nucleoside analog.

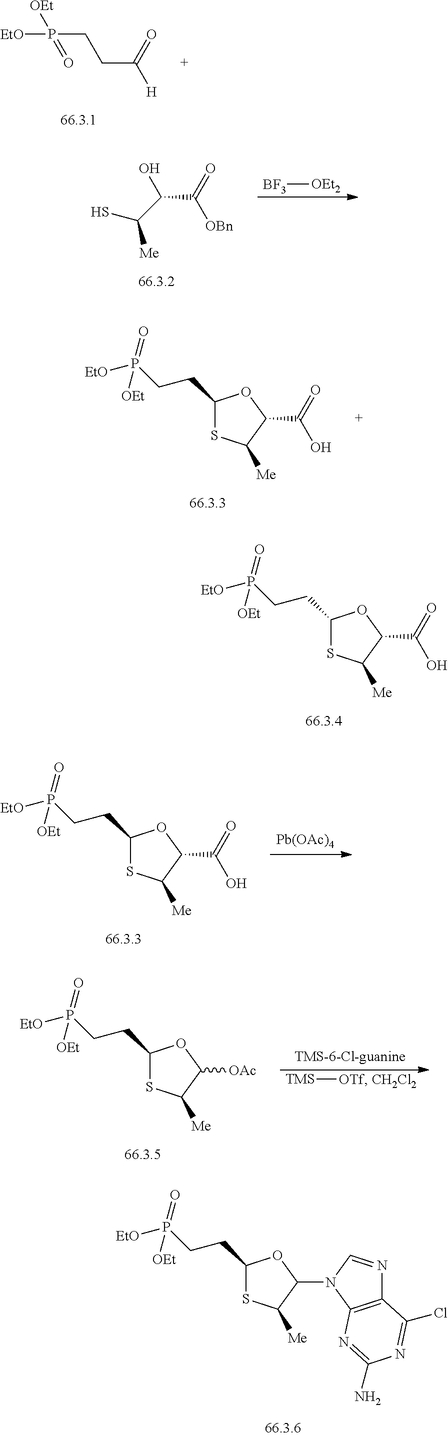

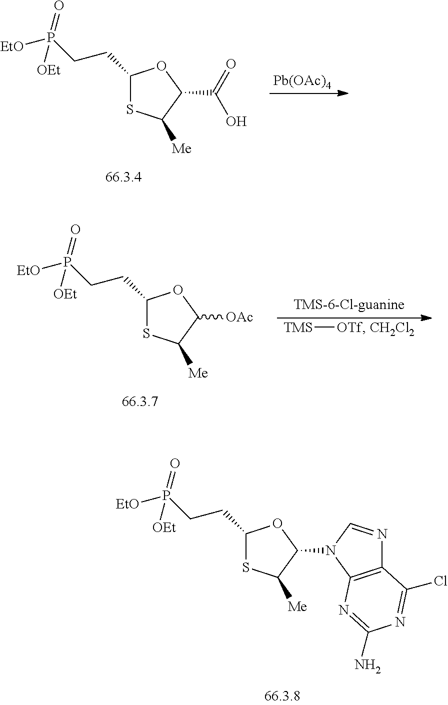

A specific example for the synthesis of a oxathiolane nucleoside analog is illustrated above. The syntheses of 66.3.6 and 66.3.8 are analogous to that of 66.2.6 and 66.2.8 described above.

### Preparation and Availability of Starting Materials

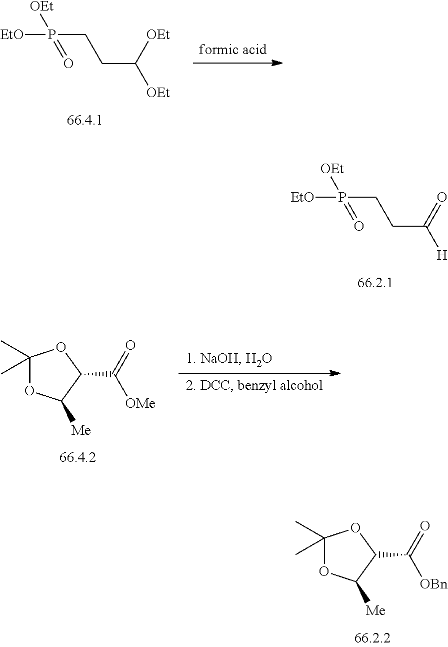

Compound 66.2.1 can be prepared from commercially available starting material 66.4.1 (available from Acros, catalog number 34693-0050 or 34693-0250, or from Epsilon, catalog number 95040) following the method illustrated above. Compound 66.2.2 can be prepared from commercially available starting material 66.4.2 (Fluka, catalog number 59437) following the method illustrated above. The preparation of 66.3.2 was described in WO 03/020222 A2 page 34 line 7 to page 36 line 5, and references cited therein.

### EXAMPLE 67

### Synthesis of Representative Compounds of Formula 73

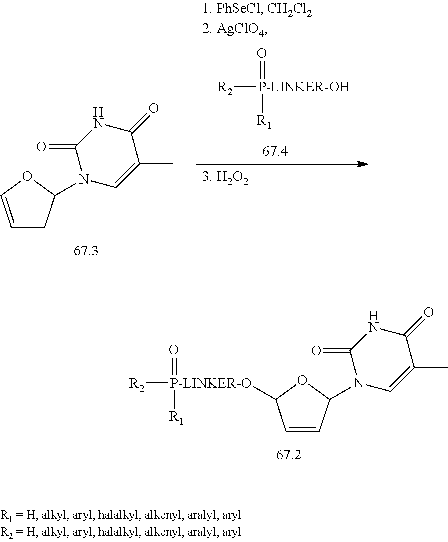

Representative compounds of the invention can be prepared as illustrated above. The desired phosphonate substituted analogs are prepared by first reacting glycal 67.3 (obtained as described in J. Am. Chem. Soc. 1972, 94, 3213) with phenylselenyl chloride followed by treatment with the respective phosphonate alcohols 67.4 in the presence of silver perchlorate (J. Org. Chem. 1991, 56, 2642-2647). Oxidation of the resulting chloride using hydrogen peroxide provides the desired phosphonate 67.2.

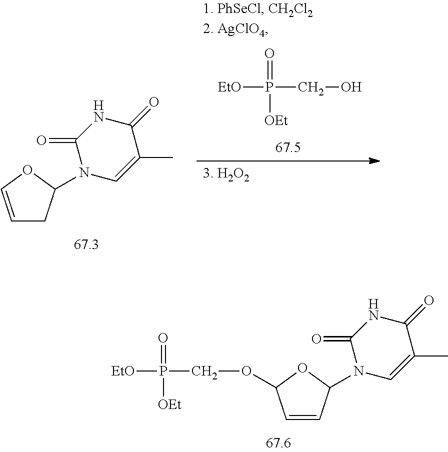

For instance, 67.3 dissolved in CH₂Cl₂, is treated with one equivalent of phenyl selenyl chloride at −70° C. followed by silver perchlorate in the presence of diethyl(hydroxymethyl) phosphonate (67.5). The phosphonate is transformed into the d4T analog 67.6 by oxidation with hydrogen peroxide. Using the above procedure, but employing different phosphonate reagents 67.4 in place of 67.5, the corresponding products 67.2 bearing different linking groups are obtained. Additionally, analogs containing a variety of bases can be prepared by starting with the appropriately protected glycals (see examples in: J. Am. Chem. Soc. 1972, 94, 3213).

### EXAMPLE 68

### Synthesis of Representative Compounds of Formula 74

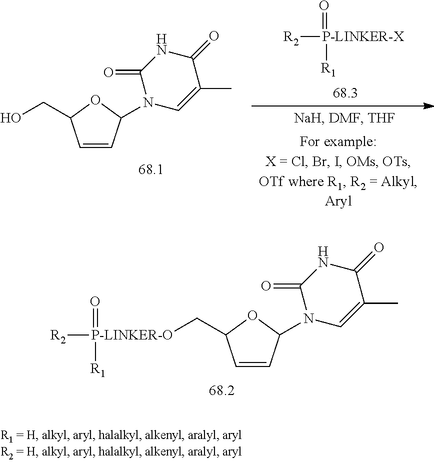

Representative compounds of the invention can be prepared as illustrated above. The desired phosphonate substituted analogs are prepared by reaction of d4T (68.1) (as described in U.S. Pat. No. 4,978,655 (col. 2 ln. 46 to col. 3 ln. 47)) with the respective alkylating reagents 68.3. Illustrated above is the preparation of phosphonate linkage to d4T through the 5′-hydroxyl group. D4T is dissolved in a solvent such as, but not limited to, DMF or THF, and is treated with a phosphonate reagent bearing a leaving group in the presence of a suitable organic or inorganic base. In compounds 68.3, X is a leaving group such as, but not limited to, bromide, chloride, iodide, p-toluenesulfonate, trifluoromethanesulfonate, or methanesulfonate.

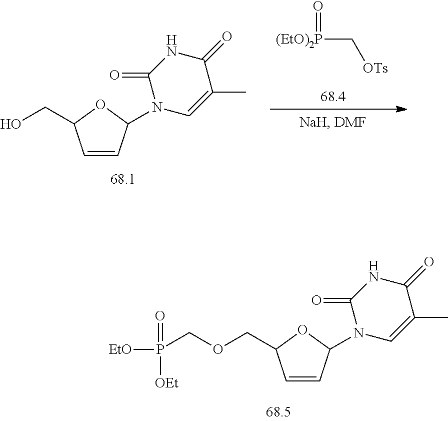

For instance, 68.1 dissolved in DMF, is treated with one equivalent of sodium hydride and one equivalent of (toluene-4-sulfonylmethyl)-phosphonic acid diethyl ester 68.4 (prepared according to the procedures in J. Org. Chem. 1996, 61, 7697) to give d4T phosphonate 68.5 in which the linkage is a methylene group. Using the above procedure, but employing different phosphonate reagents 68.3 in place of 68.4, the corresponding products 68.2 bearing different linking groups are obtained. In a similar manner, using a variety of d4Ts possessing different natural and non-natural nucleoside bases with the appropriate protecting groups, numerous other analogs can be obtained.

### EXAMPLE 70

### Synthesis of Representative Compounds of Formula 76

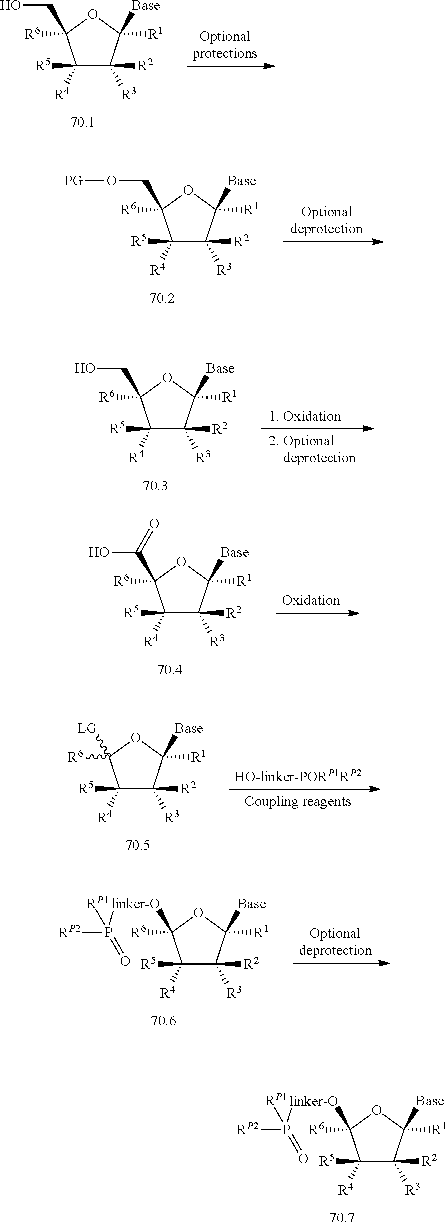

Representative compounds of the invention can be prepared as illustrated above. The core components of this reaction sequence are the transformations from primary alcohol 70.3 to phosphonate 70.6. Appropriate oxidant(s) can convert the primary alcohol (5′-hydroxy) in 70.3 to a carboxylic acid or its corresponding ester. In the case of an ester, an additional deprotection step will give the carboxylic acid 70.4. A variety of oxidation procedures can be found in the literature and can be utilized here. These include, but are not limited to, the following methods: (i) pyridinium dichromate in Ac₂O, t-BuOH, and dichloromethane producing the t-butyl ester, followed by deprotection using a reagent such as trifluoroacetic acid to convert the ester to the corresponding carboxylic acid (see Classon, et al., Acta Chem. Scand. Ser. B 1985, 39, 501-504; Cristalli, et al., J. Med. Chem. 1988, 31, 1179-1183); (ii) iodobenzene diacetate and 2,2,6,6-tetramethyl-1-piperidinyloxy, free radical (TEMPO) in acetonitrile, producing the carboxylic acid (see Epp, et al., J. Org. Chem. 1999, 64, 293-295; Jung et al., J. Org. Chem. 2001, 66, 2624-2635); (iii) sodium periodate, ruthenium(III) chloride in chloroform producing the carboxylic acid (see Kim, et al., J. Med. Chem. 1994, 37, 4020-4030; Homma, et al., J. Med. Chem. 1992, 35, 2881-2890); (iv) chromium trioxide in acetic acid producing the carboxylic acid (see Olsson et al.; J. Med. Chem. 1986, 29, 1683-1689; Gallo-Rodriguez et al.; J. Med. Chem. 1994, 37, 636-646); (v) potassium permanganate in aqueous potassium hydroxide producing the carboxylic acid (see Ha, et al., J. Med. Chem. 1986, 29, 1683-1689; Franchetti, et al., J Med. Chem. 1998, 41, 1708-1715); and (vi) nucleoside oxidase from S. maltophilia to give the carboxylic acid (see Mahmoudian, et al., Tetrahedron 1998, 54, 8171-8182).

The preparation of 70.5 from 70.4 using lead(IV) tetraacetate (LG=OAc) is described by Teng et al., J. Org. Chem. 1994, 59, 278-280 and Schultz, et al; J. Org. Chem. 1983, 48; 3408-3412. When lead(IV) tetraacetate is used together with lithium chloride (see Kochi, et al., J. Am. Chem. Soc. 1965, 87, 2052), the corresponding chloride is obtained (70.5, LG=Cl). Lead(IV) tetraacetate in combination with N-chlorosuccinimide can produce the same product (70.5, LG=Cl) (see Wang, et al., Tetrahedron: Asymmetry 1990, 1, 527; and Wilson et al., Tetrahedron: Asymmetry 1990, 1, 525). Alternatively, the acetate leaving group (LG) can also be converted to other leaving group such as bromide by treatment of trimethylsilyl bromide to give 70.5 (see Spencer, et al., J. Org. Chem. 1999, 64, 3987-3995).

The coupling of 70.5 (LG=OAc) with a variety of nucleophiles was described by Teng et al., Synlett 1996; 346-348 and U.S. Pat. No. 6,087,482; column 54 line 64 to column 55 line 20. Specifically, the coupling between 70.5 and diethyl hydroxymethylphosphonate in the presence of trimethylsilyl trifluoromethanesulfonate (TMS-OTf) is described. Other compounds with the general structure of HO-linker-POR^{P1}R^{P2} can also be used so long as the functional groups in these compounds are compatible with the coupling reaction conditions. There are many examples in the published literature describing the coupling of 70.5 (LG=halogen) with a variety of alcohols. The reactions can be facilitated with a number of reagents, such as silver(I) salts (see Kim et al.; J. Org. Chem. 1991, 56, 2642-2647; Toikka et al., J. Chem. Soc. Perkins Trans. 1, 1999, 13, 1877-1884), mercury(II) salts (see Veeneman et al.; Recl. Tray. Chim. Pays-Bas. 1987, 106, 129-131), boron trifluoride diethyl etherate (see Kunz et al., Hel. Chim Acta 1985, 68, 283-287), tin(II) chloride (see O'Leary et al., J. Org. Chem. 1994, 59, 6629-6636), alkoxide (see Shortnacy-Fowler et al., Nucleosides Nucleotides 2001, 20, 1583-1598), and iodine (see Kartha et al., J. Chem. Soc. Perkins Trans. 1, 2001, 770-772). These methods can be selectively used in conjunction with different methods in forming 70.5 with various leaving groups (LG) to produce 70.6.

The introduction and removal of protecting groups is commonly practiced in the art of organic synthesis. Many sources of information on transformations involving protecting groups are available in the published literature, e.g. Greene and Wuts, Protecting Groups in Organic Synthesis, 3^{rd} Ed., John Wiley & Sons, Inc., 1999. The main purpose is to temporarily transform a functional group so that it will survive a set of subsequent reaction procedures. Afterward, the original functional group can be restored by a preconceived deprotection procedure. Therefore, the transformations from 70.1 to 70.2, from 70.2 to 70.3, and from 70.6 to 70.7 are intended to allow important transformations (e.g., from 70.3 to 70.6) to occur while preserving the functional groups that already exist in the core structures.

It should be understood that in the transformation 70.6 to 70.7, R^{P1} and R^{P2} need not remain unchanged. The final form of R^{P1} and R^{P2} can be selected from a variety of possible structures.

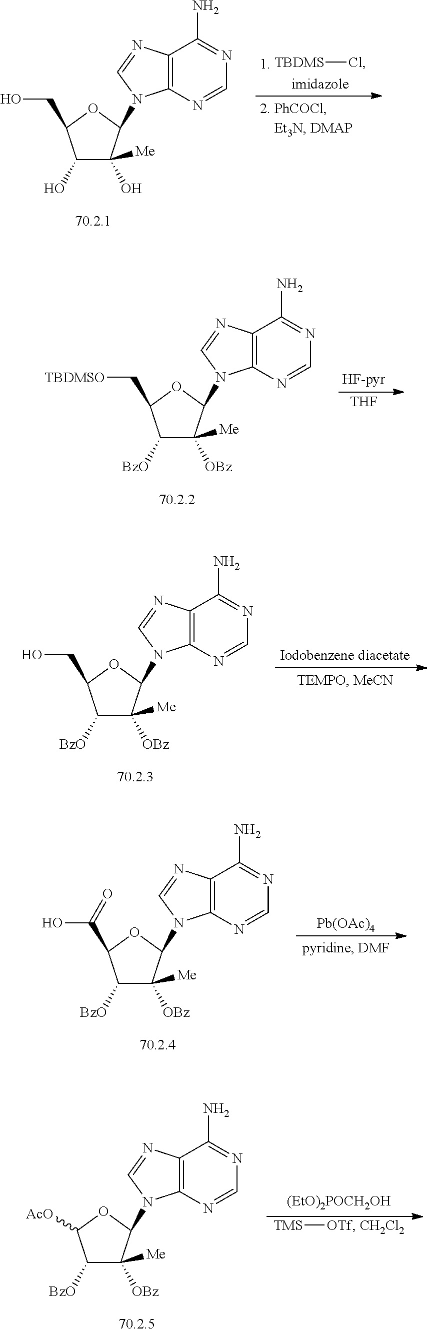

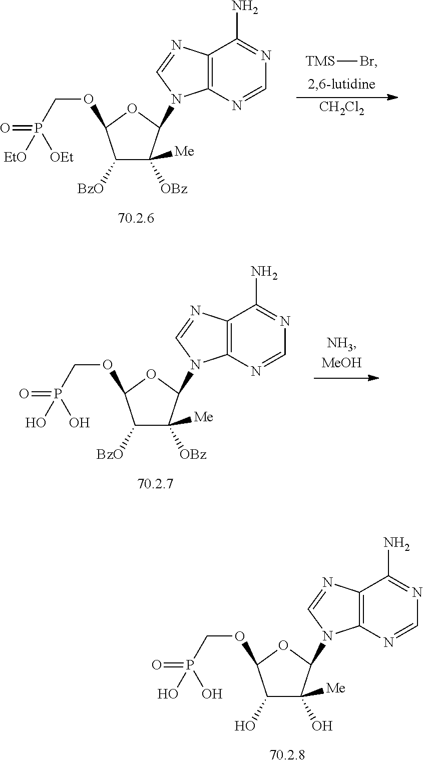

The scheme shown above provides a specific example for the general scheme discussed above. Compound 70.2.1 is prepared using method described in WO 01/90121 (page 115). The 5′-hydroxyl in 70.2.1 is protected as a t-butyldimethylsilyl (TBDMS) ether. The 2′- and 3′-hydroxyl groups can be protected as benzoyl (Bz) esters to give 70.2.2. The 5′-hydroxyl can then be deprotected to give 70.2.3. Oxidation using iodobenzene diacetate and 2,2,6,6-tetramethyl-1-piperidinyloxy, free radical (TEMPO) converts the primary alcohol to the corresponding acid 70.2.4. Further oxidation of 70.2.4 using lead tetraacetate can produce 70.2.5. Coupling between 70.2.5 and diethyl hydroxymethylphosphonate (available from Sigma-Aldrich, Cat. No. 39,262-6) effected by TMS-OTf can afford 70.2.6. Treating 70.2.6 with TMS-Br converts the phosphodiester to the corresponding phosphonic acid 70.2.7. Deprotection of the 2′- and 3′-hydroxyl gives 70.2.8 as an example of the generic structure 76, where Base is an adenine, R¹, R⁵, and R⁶ are hydrogen, R² is methyl group, R³ and R⁴ are hydroxyl groups, linker is a methylene group, and R^{P1} and R^{P2} are both hydroxyl groups.

The phosphonic acids in 70.2.7 and 70.2.8 are illustrative examples. Other forms of phosphonates can be accessed via the phosphonic acid, or other forms, such as the corresponding diesters.

### Preparation and Availability of Starting Materials for Examples 70-153

A variety of compounds of the general structure 70.1 can either be prepared using procedures described in the literature, or be purchased from commercial sources. The following are good sources for information on the art of preparing a variety of compounds of the general structure 70.1: Townsend, Chemistry of Nucleosides and Nucleotides, Plenum Press, 1994; and Vorbruggen and Ruh-Pohlenz, Handbook of Nucleoside Synthesis, John Wiley & Sons, Inc., 2001.

There are limited number of common precursors that were used to prepare the structures 70.1 in the examples that follow. Many of them are described in the various patents listed at the beginning of this document and the references cited therein. The following is a list of these common precursors and their commercial sources or method of preparation.

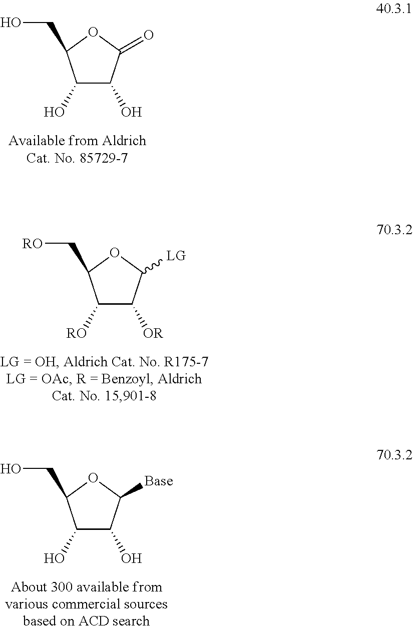

### EXAMPLES 71-153

Examples 71-153 employ the reaction conditions described above in Example 70. It should be understood that specific reagents, solvents, and reaction conditions used can be substituted by one skilled in the art to accommodate the structure and reactivity requirements of the starting materials. Alternative methods including, but not limited to, those discussed in Example 70 can be applied as needed. Alternative protection and deprotection procedures are also likely to be devised and adapted as needed.

### EXAMPLE 71

### Synthesis of Representative Compounds of Formula 76

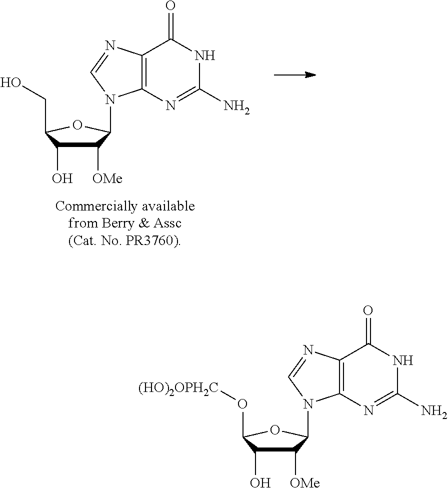

### EXAMPLE 72

### Synthesis of Representative Compounds of Formula 76

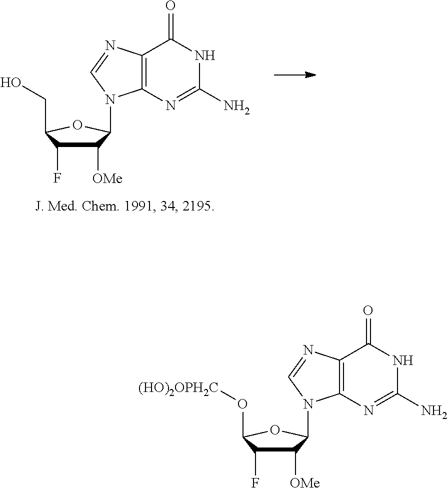

### EXAMPLE 73

### Synthesis of Representative Compounds of Formula 76

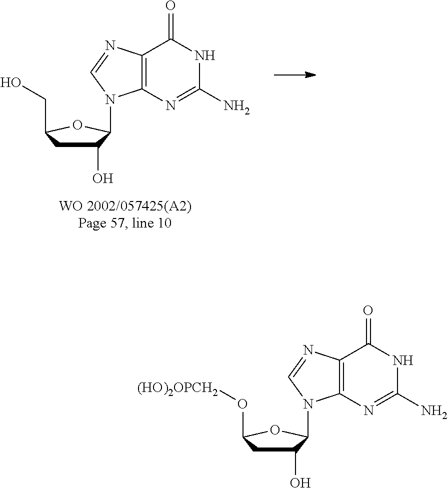

### EXAMPLE 74

### Synthesis of Representative Compounds of Formula 76

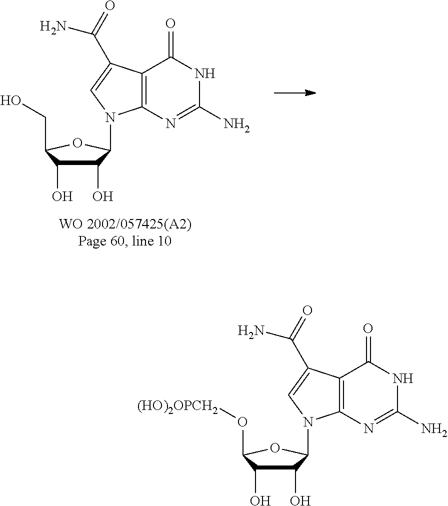

### EXAMPLE 75

### Synthesis of Representative Compounds of Formula 76

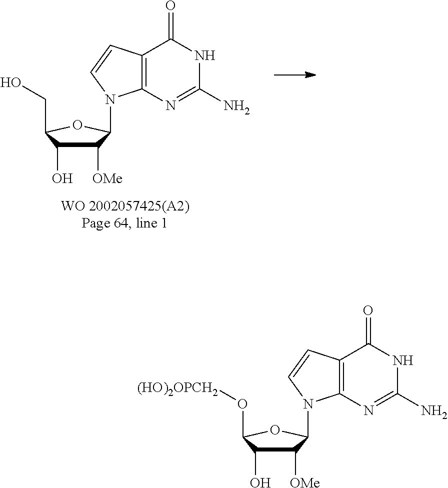

### EXAMPLE 76

### Synthesis of Representative Compounds of Formula 76

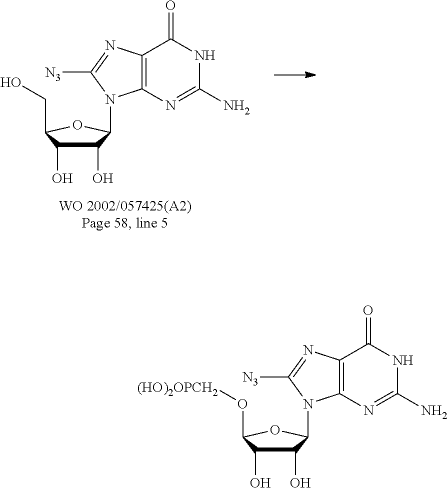

### EXAMPLE 77

### Synthesis of Representative Compounds of Formula 76

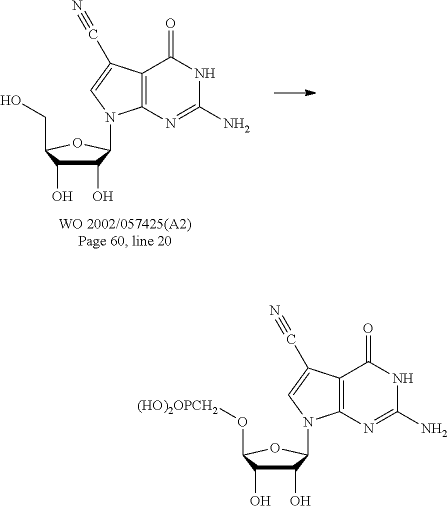

### EXAMPLE 78

### Synthesis of Representative Compounds of Formula 76

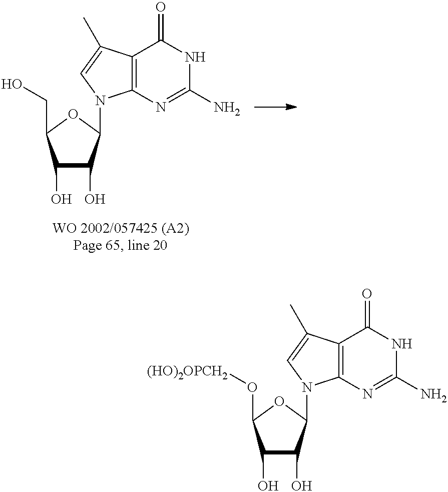

### EXAMPLE 79

### Synthesis of Representative Compounds of Formula 76

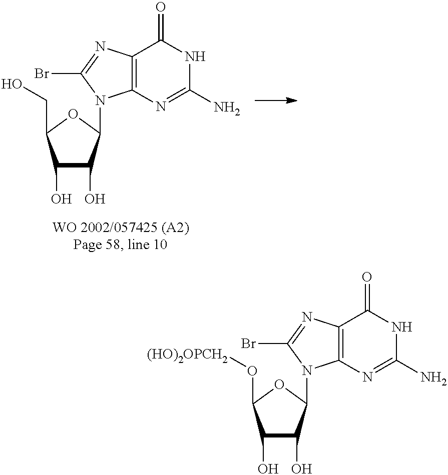

### EXAMPLE 80

### Synthesis of Representative Compounds of Formula 76

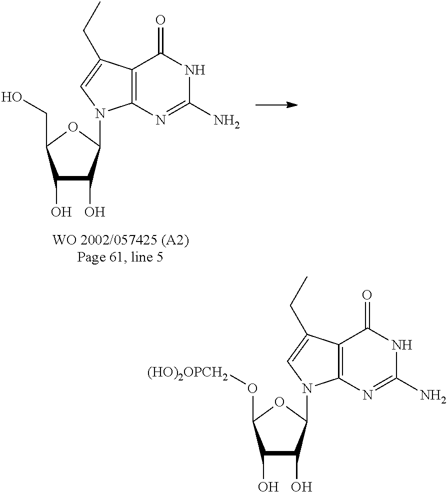

### EXAMPLE 81

### Synthesis of Representative Compounds of Formula 76

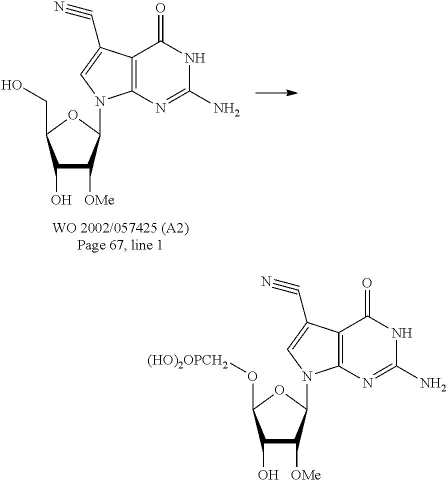

### EXAMPLE 82

### Synthesis of Representative Compounds of Formula 76

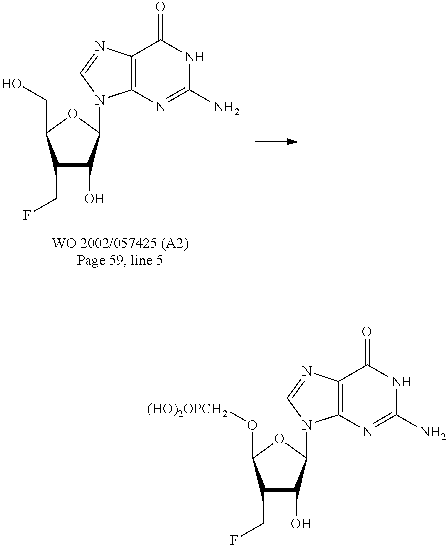

### EXAMPLE 83

### Synthesis of Representative Compounds of Formula 76

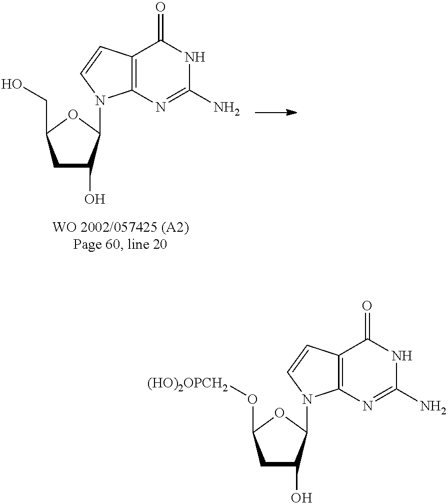

### EXAMPLE 84

### Synthesis of Representative Compounds of Formula 76

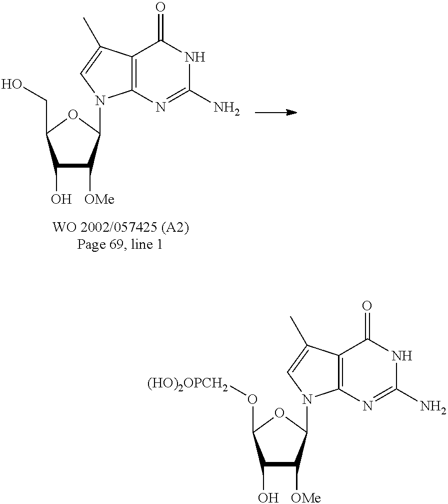

### EXAMPLE 85

### Synthesis of Representative Compounds of Formula 76

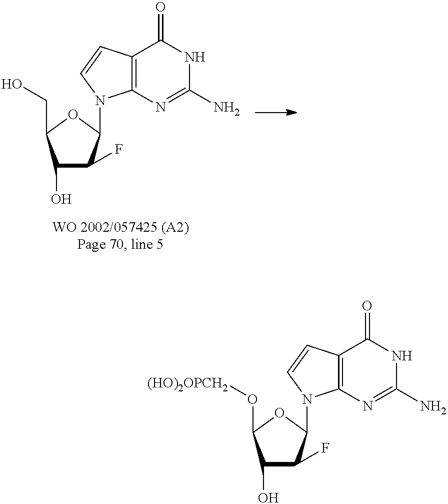

### EXAMPLE 86

### Synthesis of Representative Compounds of Formula 76

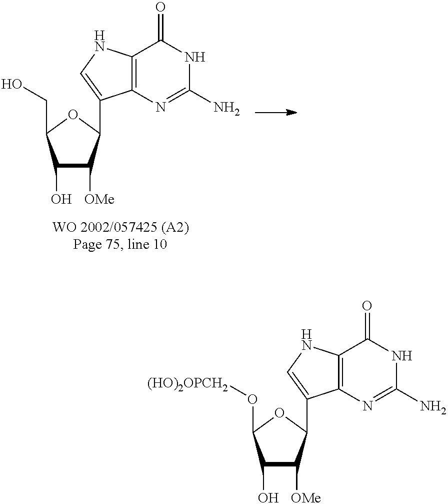

### EXAMPLE 87

### Synthesis of Representative Compounds of Formula 76

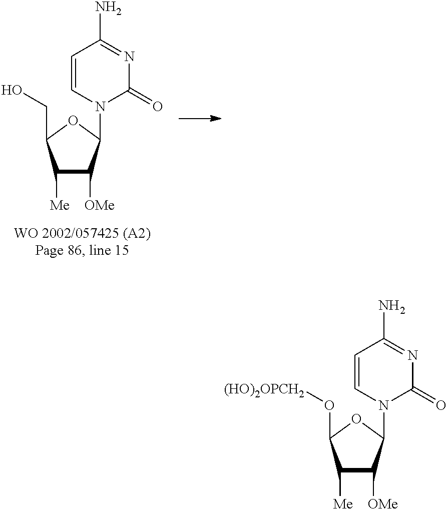

### EXAMPLE 88

### Synthesis of Representative Compounds of Formula 76

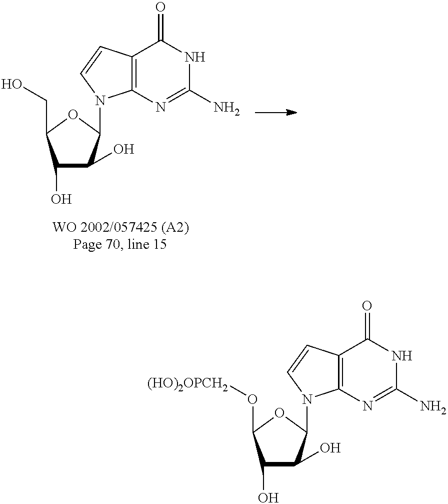

### EXAMPLE 89

### Synthesis of Representative Compounds of Formula 76

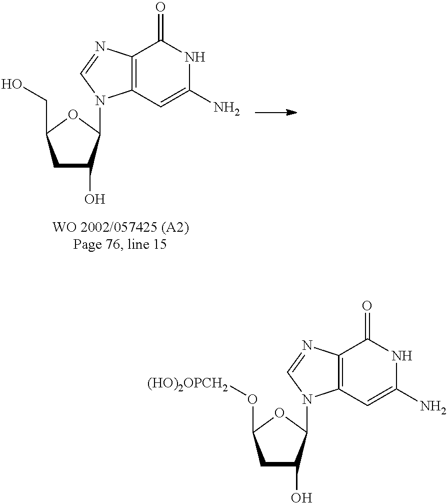

### EXAMPLE 90

### Synthesis of Representative Compounds of Formula 76

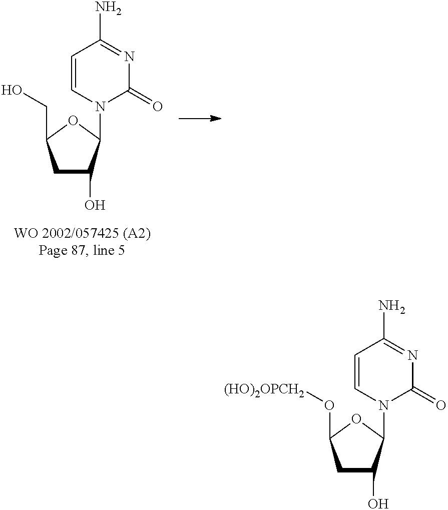

### EXAMPLE 91

### Synthesis of Representative Compounds of Formula 76

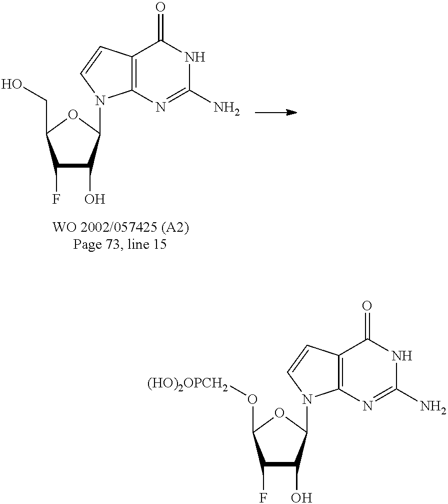

### EXAMPLE 92

### Synthesis of Representative Compounds of Formula 76

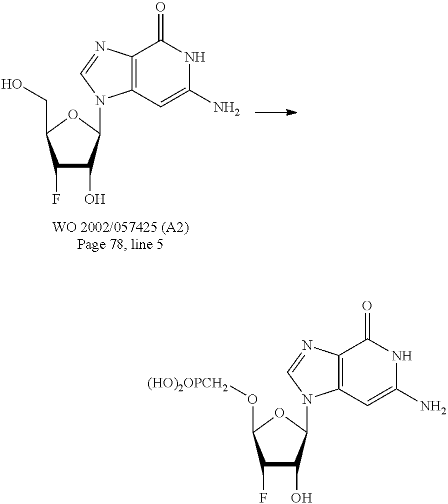

### EXAMPLE 93

### Synthesis of Representative Compounds of Formula 76

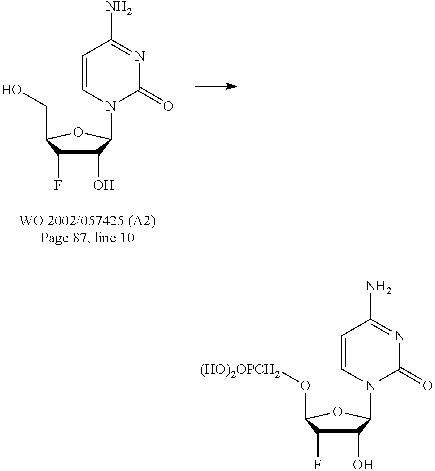

### EXAMPLE 94

### Synthesis of Representative Compounds of Formula 76

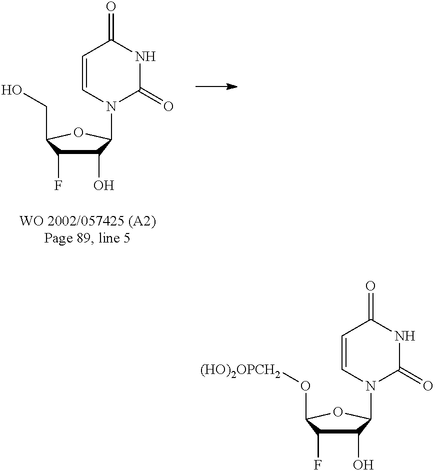

### EXAMPLE 95

### Synthesis of Representative Compounds of Formula 76

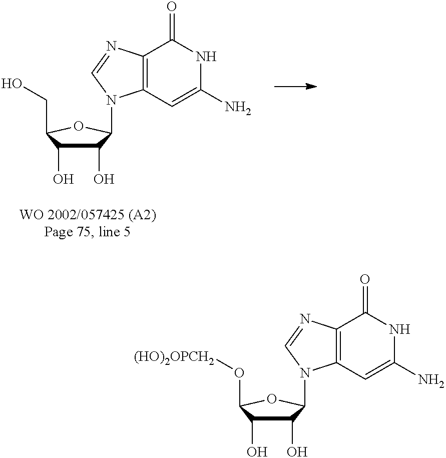

### EXAMPLE 96

### Synthesis of Representative Compounds of Formula 76

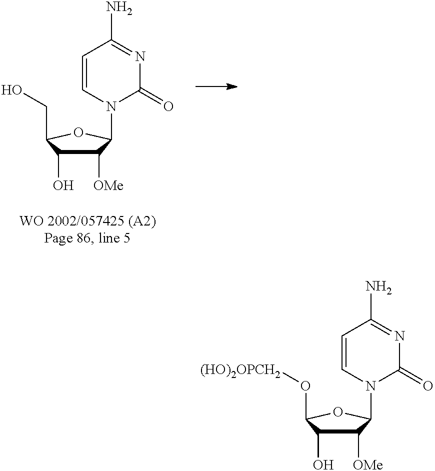

### EXAMPLE 97

### Synthesis of Representative Compounds of Formula 76

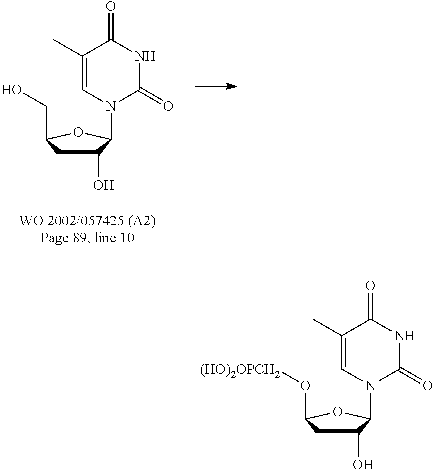

### EXAMPLE 98

### Synthesis of Representative Compounds of Formula 76

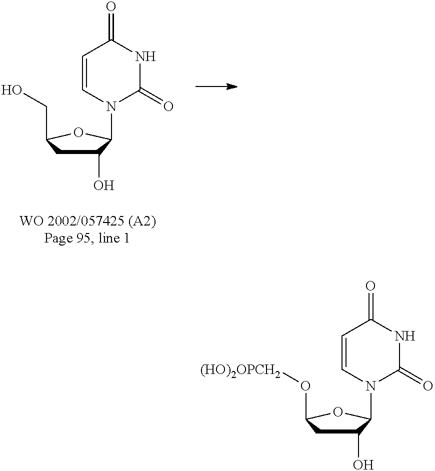

### EXAMPLE 99

### Synthesis of Representative Compounds of Formula 76

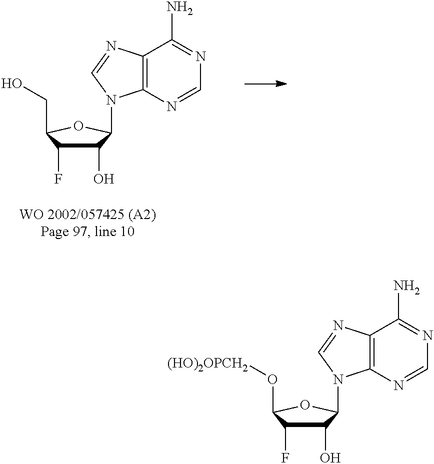

### EXAMPLE 100

### Synthesis of Representative Compounds of Formula 76

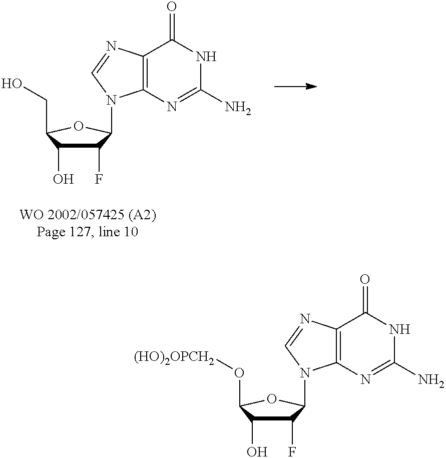

### EXAMPLE 101

### Synthesis of Representative Compounds of Formula 76

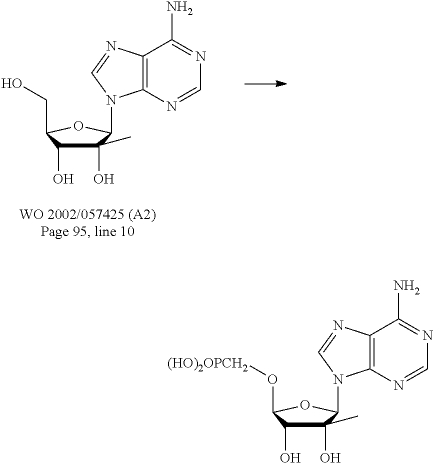

### EXAMPLE 102

### Synthesis of Representative Compounds of Formula 76

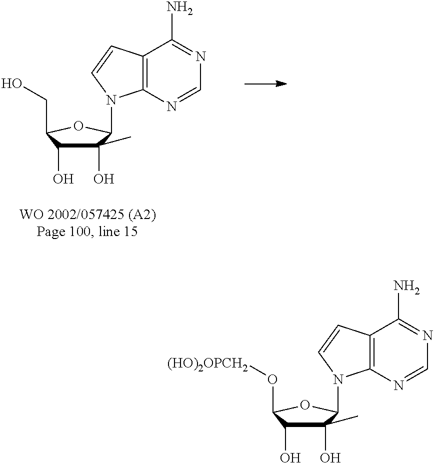

### EXAMPLE 103

### Synthesis of Representative Compounds of Formula 76

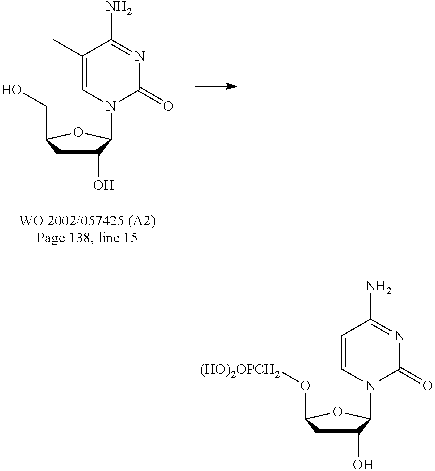

### EXAMPLE 104

### Synthesis of Representative Compounds of Formula 76

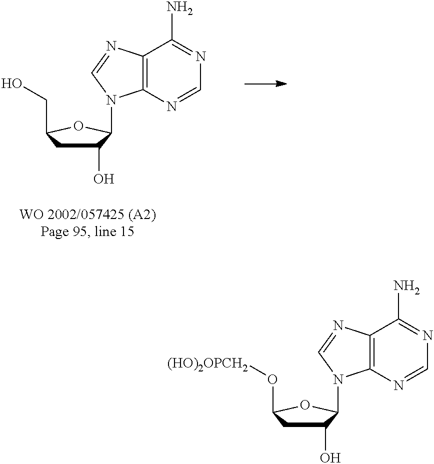

### EXAMPLE 105

### Synthesis of Representative Compounds of Formula 76

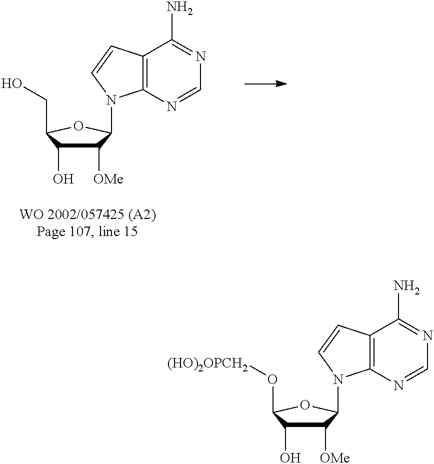

### EXAMPLE 106

### Synthesis of Representative Compounds of Formula 76

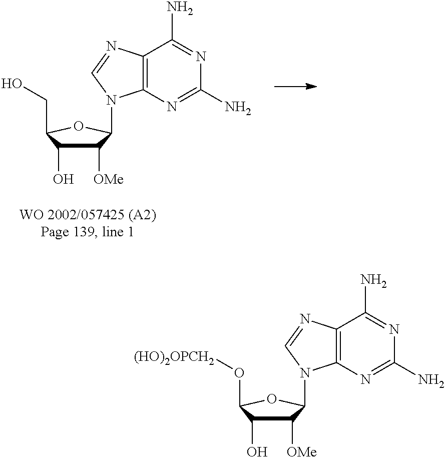

### EXAMPLE 107

### Synthesis of Representative Compounds of Formula 76

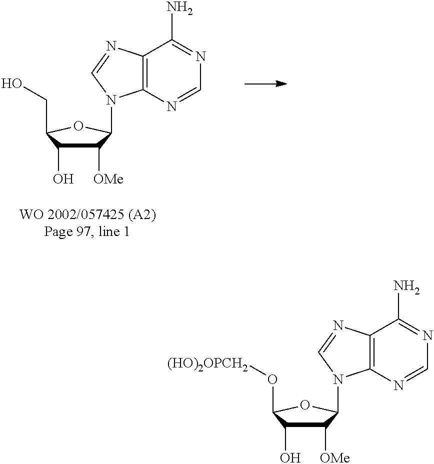

### EXAMPLE 108

### Synthesis of Representative Compounds of Formula 76

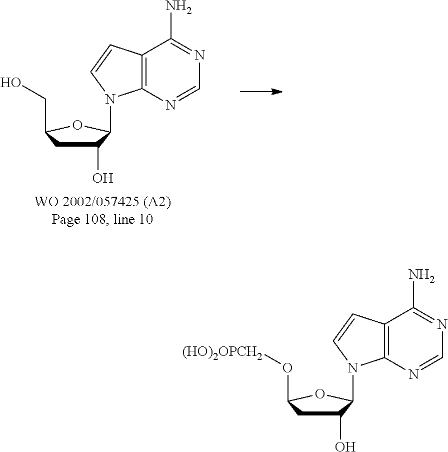

### EXAMPLE 109

### Synthesis of Representative Compounds of Formula 76

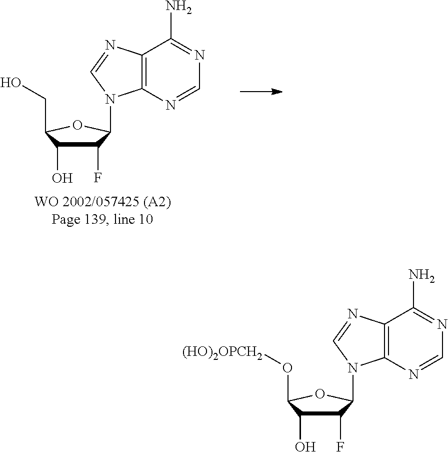

### EXAMPLE 110

### Synthesis of Representative Compounds of Formula 76

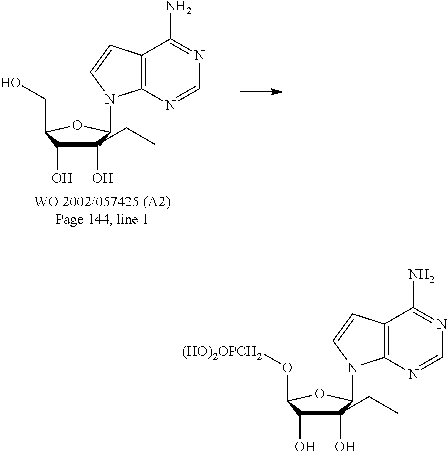

### EXAMPLE 111

### Synthesis of Representative Compounds of Formula 76

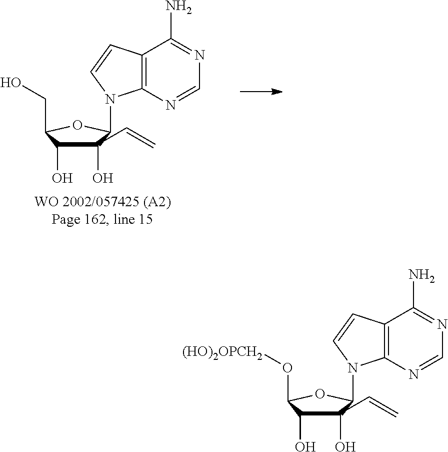

### EXAMPLE 112

### Synthesis of Representative Compounds of Formula 76

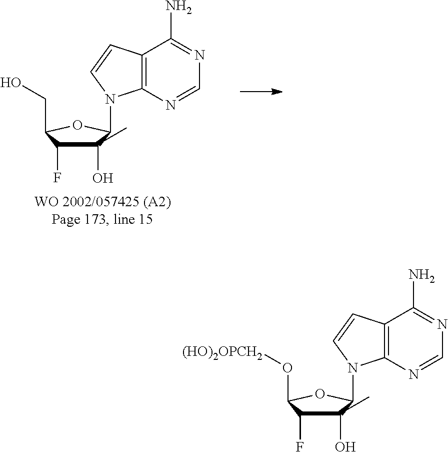

### EXAMPLE 113

### Synthesis of Representative Compounds of Formula 76

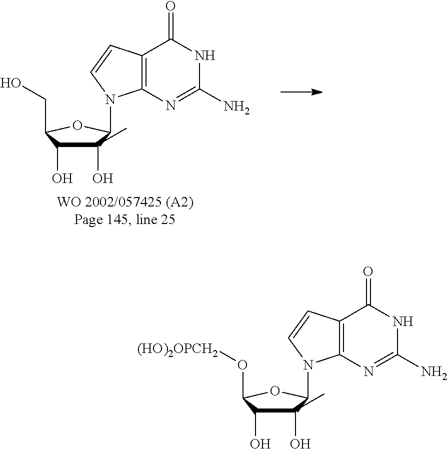

### EXAMPLE 114

### Synthesis of Representative Compounds of Formula 76

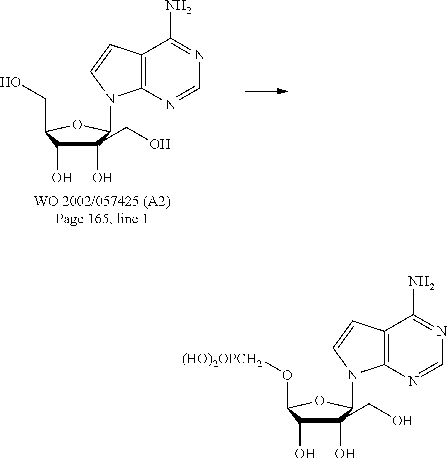

Note: one equivalent of TBDMS-Cl can be used in the protection of the 5′-hydroxyl. The mixture of two TBDMS ethers of the two primary alcohols can be separated, and the 5′-hydroxyl protected ether can be used in subsequent reactions.

### EXAMPLE 115

### Synthesis of Representative Compounds of Formula 76

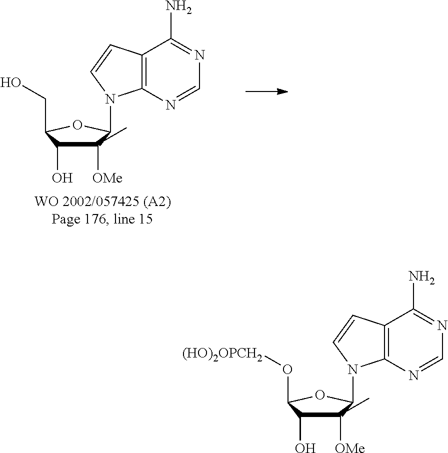

### EXAMPLE 116

### Synthesis of Representative Compounds of Formula 76

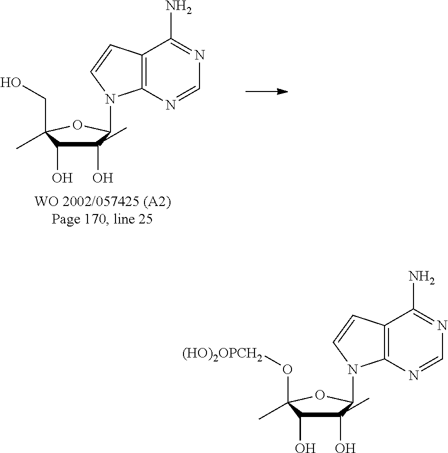

### EXAMPLE 117

### Synthesis of Representative Compounds of Formula 76

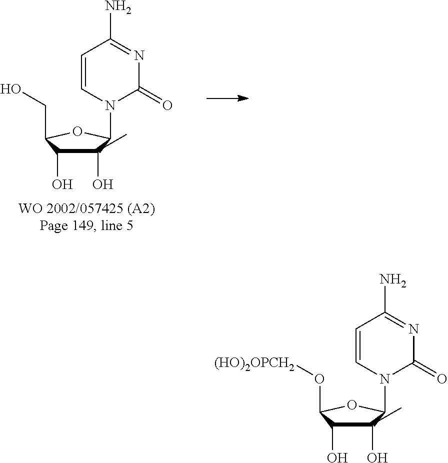

### EXAMPLE 118

### Synthesis of Representative Compounds of Formula 76

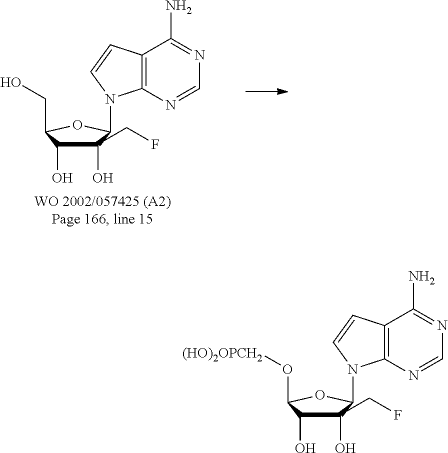

### EXAMPLE 119

### Synthesis of Representative Compounds of Formula 76

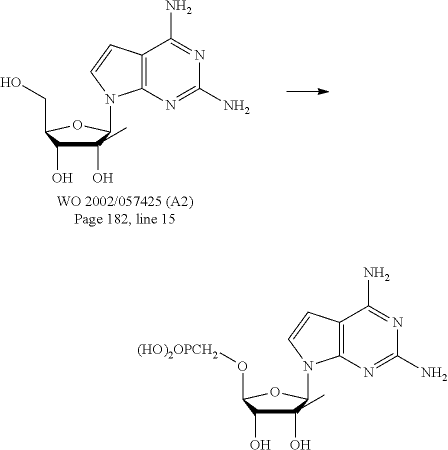

### EXAMPLE 120

### Synthesis of Representative Compounds of Formula 76

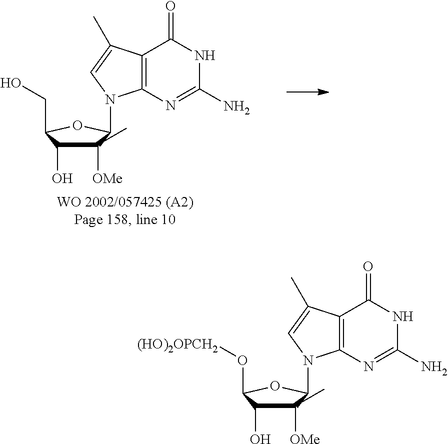

### EXAMPLE 121

### Synthesis of Representative Compounds of Formula 76

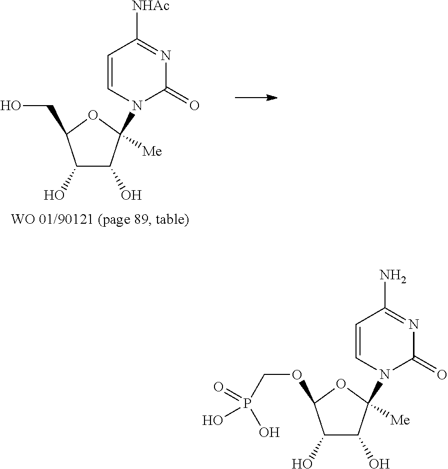

### EXAMPLE 122

### Synthesis of Representative Compounds of Formula 76

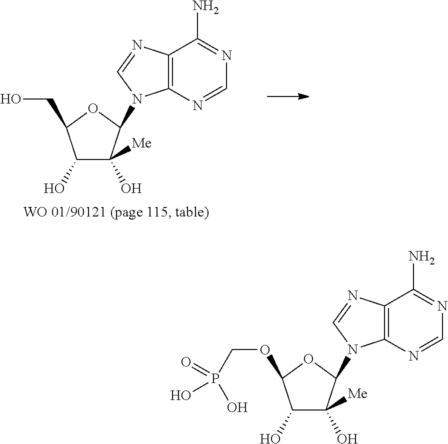

### EXAMPLE 123

### Synthesis of Representative Compounds of Formula 76

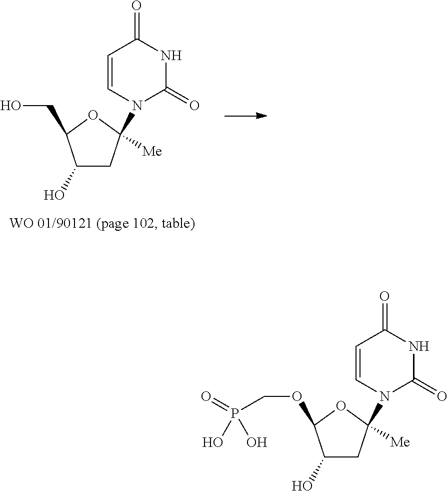

### EXAMPLE 124

### Synthesis of Representative Compounds of Formula 76

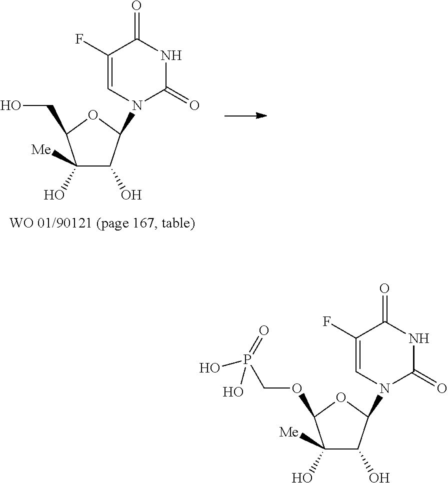

### EXAMPLE 125

### Synthesis of Representative Compounds of Formula 76

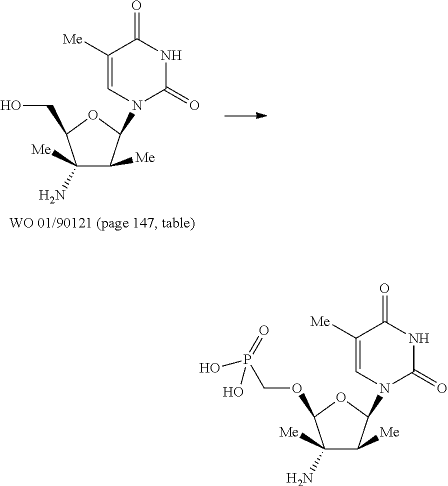

Several options exist for the protection of the amine in the starting material. It can be protected as its corresponding benzyl carbamate, allyl carbamate, trifluoroacetamide, or N-diphenylmethyleneamine derivative.

### EXAMPLE 126

### Synthesis of Representative Compounds of Formula 76

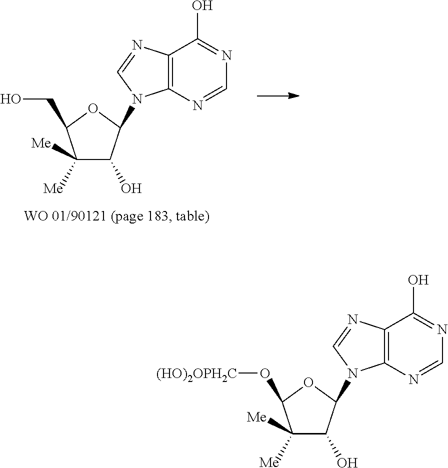

### EXAMPLE 127

### Synthesis of Representative Compounds of Formula 76

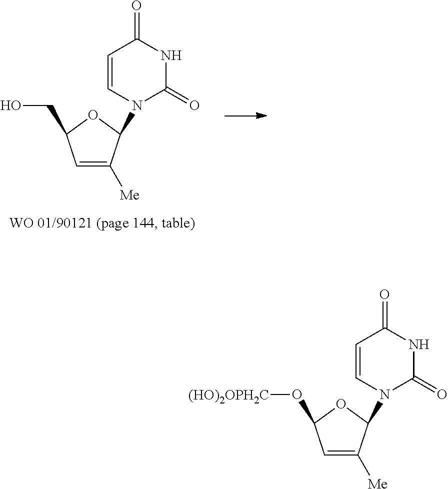

### EXAMPLE 128

### Synthesis of Representative Compounds of Formula 76

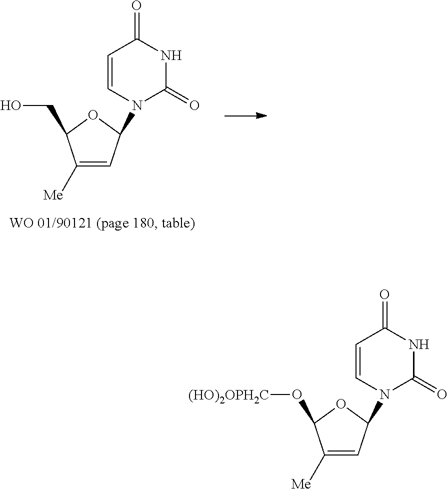

### EXAMPLE 129

### Synthesis of Representative Compounds of Formula 76

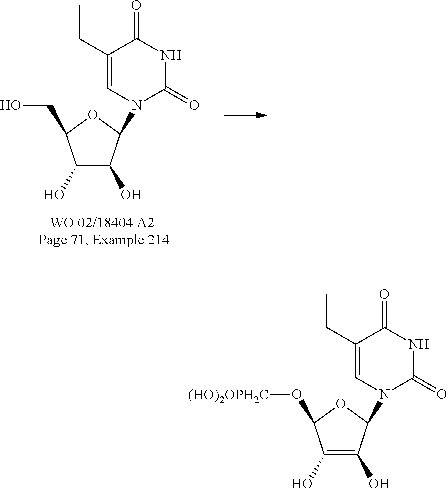

### EXAMPLE 130

### Synthesis of Representative Compounds of Formula 76

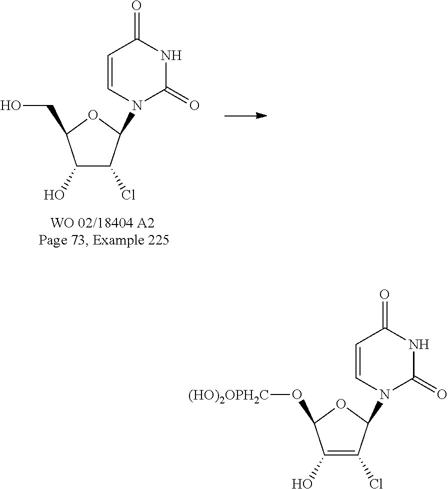

### EXAMPLE 131

### Synthesis of Representative Compounds of Formula 76

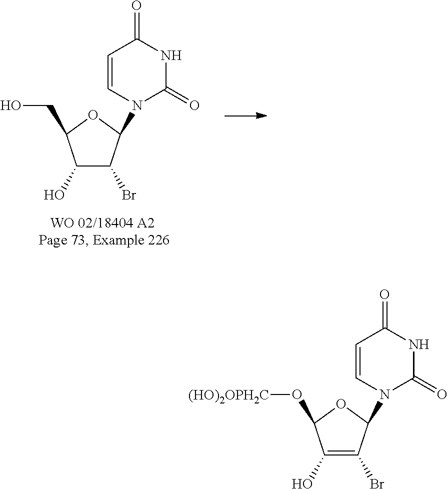

### EXAMPLE 132

### Synthesis of Representative Compounds of Formula 76

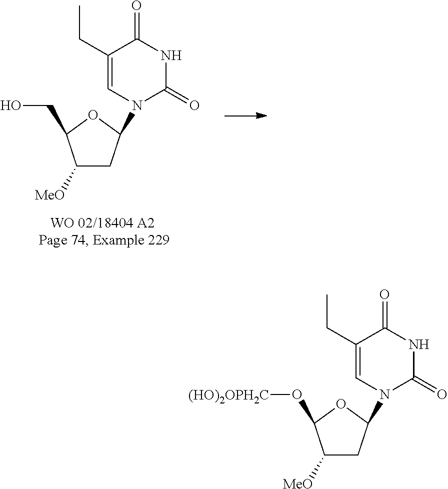

### EXAMPLE 133

### Synthesis of Representative Compounds of Formula 76

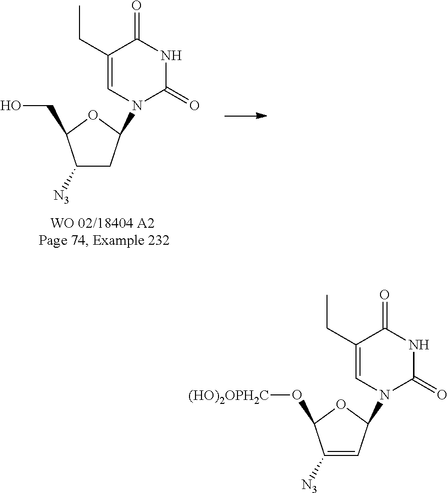

### EXAMPLE 134

### Synthesis of Representative Compounds of Formula 76

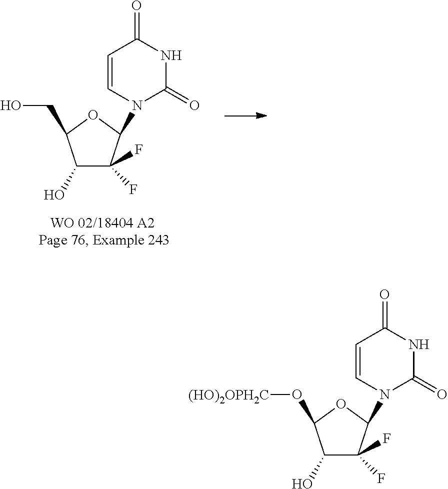

### EXAMPLE 135

### Synthesis of Representative Compounds of Formula 76

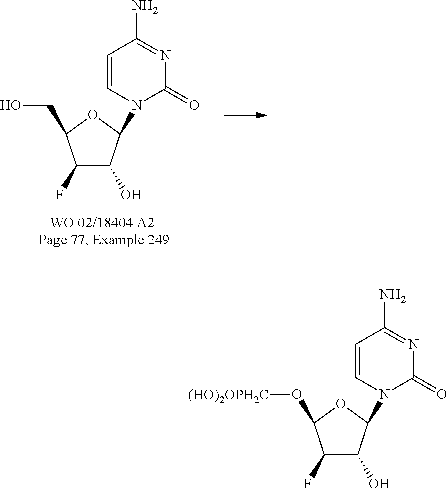

### EXAMPLE 136

### Synthesis of Representative Compounds of Formula 76

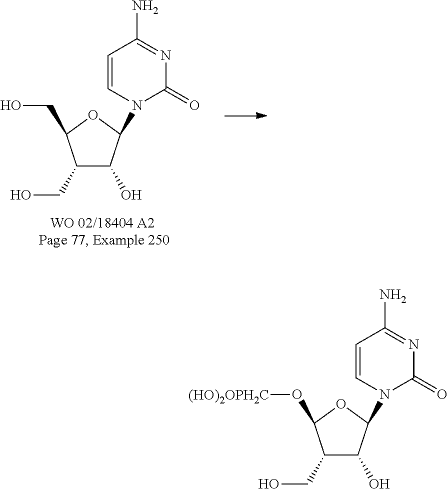

### EXAMPLE 137

### Synthesis of Representative Compounds of Formula 76

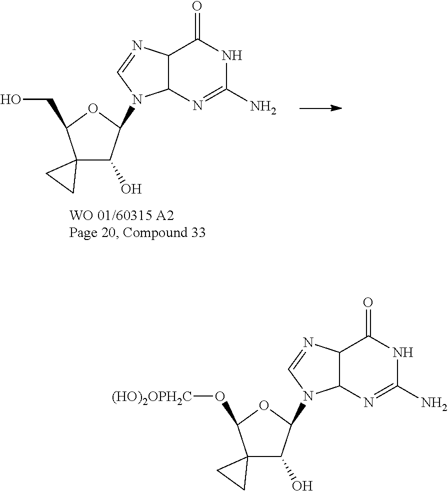

### EXAMPLE 138

### Synthesis of Representative Compounds of Formula 76

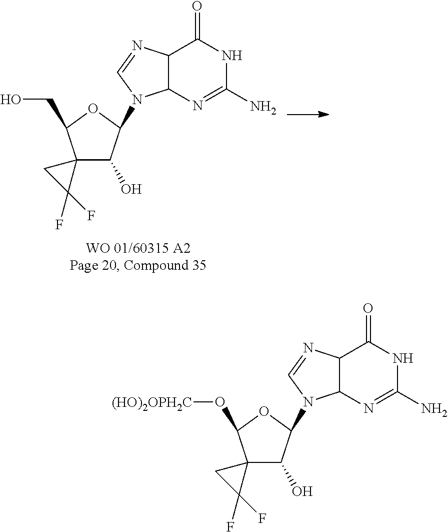

### EXAMPLE 139

### Synthesis of Representative Compounds of Formula 76

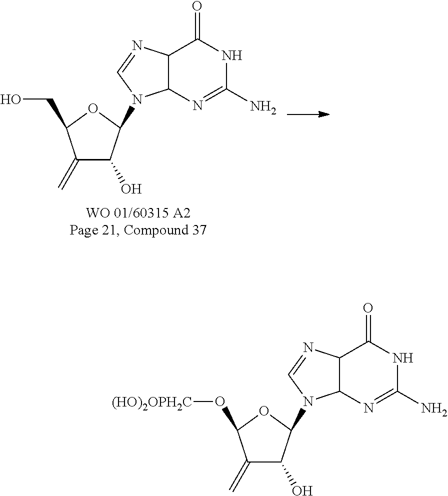

### EXAMPLE 140

### Synthesis of Representative Compounds of Formula 76

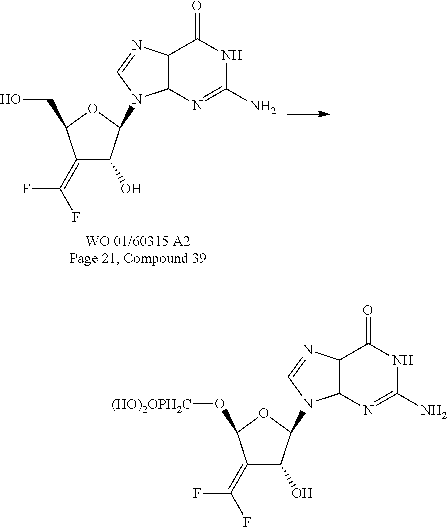

### EXAMPLE 141

### Synthesis of Representative Compounds of Formula 76

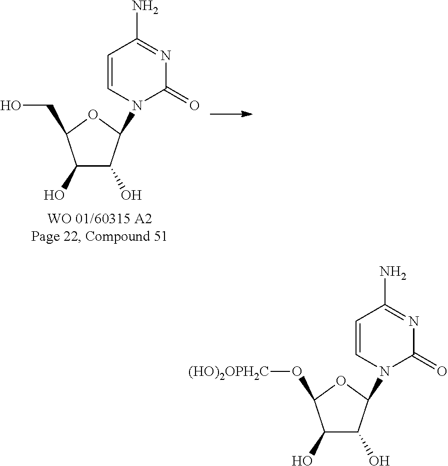

### EXAMPLE 142

### Synthesis of Representative Compounds of Formula 76

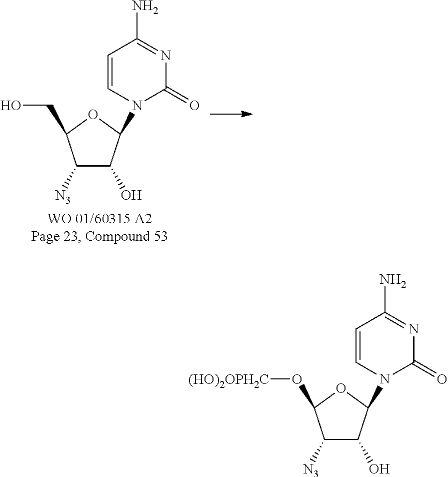

### EXAMPLE 143

### Synthesis of Representative Compounds of Formula 76

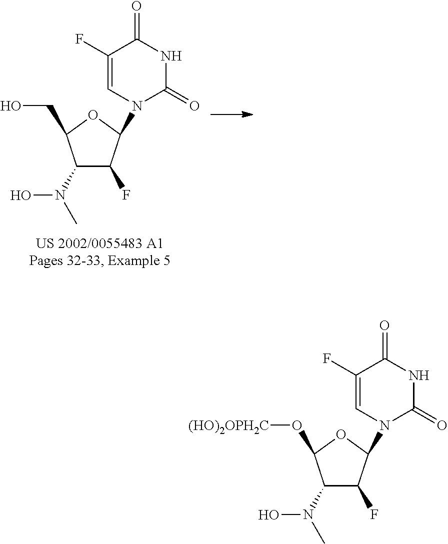

### EXAMPLE 144

### Synthesis of Representative Compounds of Formula 76

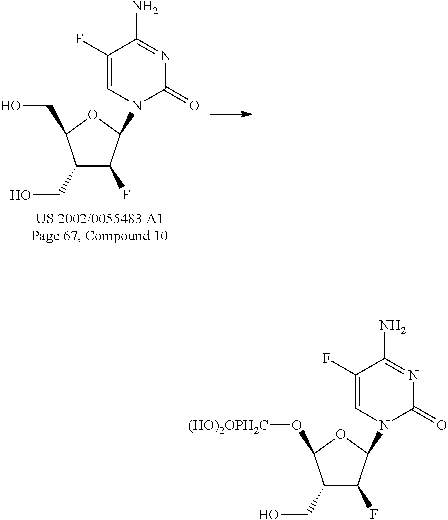

One equivalent of TBDMS-Cl can be used in the protection of the 5′-hydroxyl. The mixture of two TBDMS ethers of the two primary alcohols can be separated, and the 5′-hydroxyl protected ether can be used in subsequent reactions.

### EXAMPLE 145

### Synthesis of Representative Compounds of Formula 76

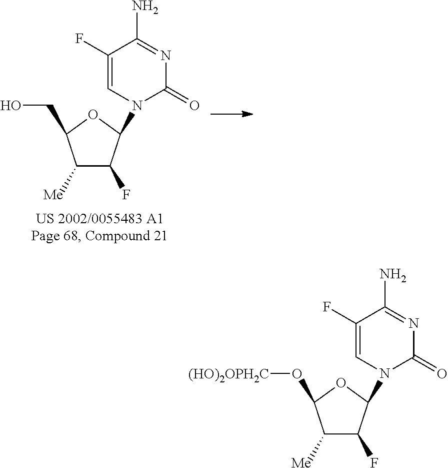

### EXAMPLE 146

### Synthesis of Representative Compounds of Formula 76

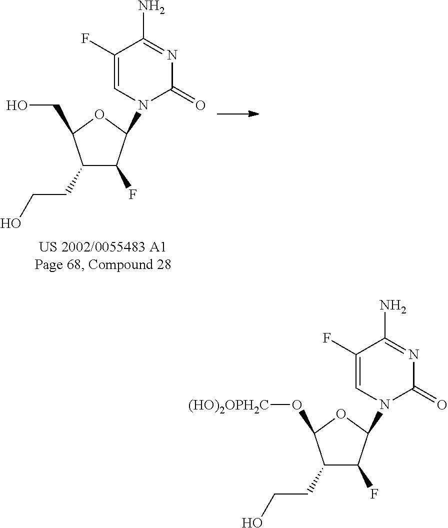

One equivalent of TBDMS-Cl can be used in the protection of the 5′-hydroxyl. The mixture of two TBDMS ethers of the two primary alcohols can be separated, and the 5′-hydroxyl protected ether can be used in subsequent reactions.

### EXAMPLE 147

### Synthesis of Representative Compounds of Formula 76

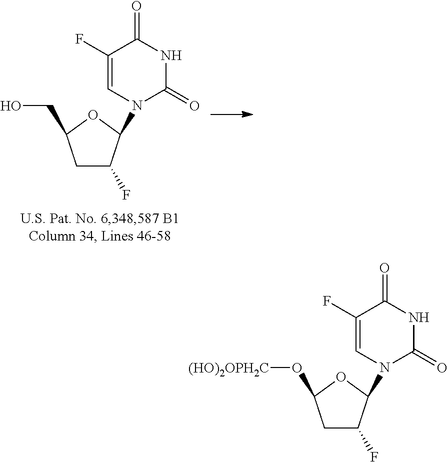

### EXAMPLE 148

### Synthesis of Representative Compounds of Formula 76

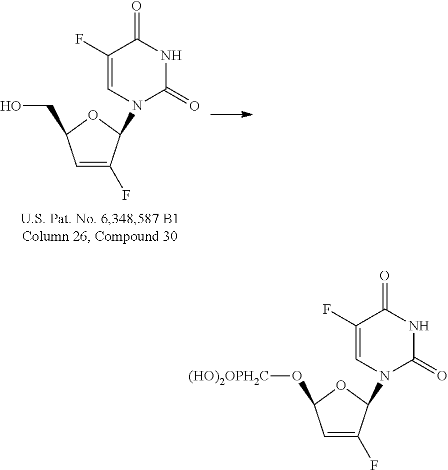

### EXAMPLE 149

### Synthesis of Representative Compounds of Formula 76

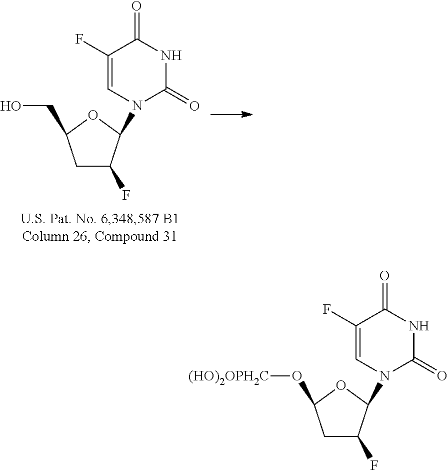

### EXAMPLE 150

### Synthesis of Representative Compounds of Formula 76

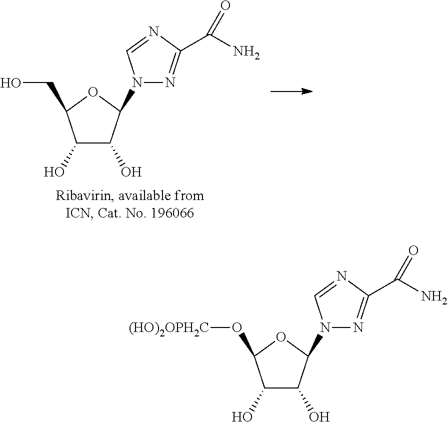

### EXAMPLE 151

### Synthesis of Representative Compounds of Formula 76

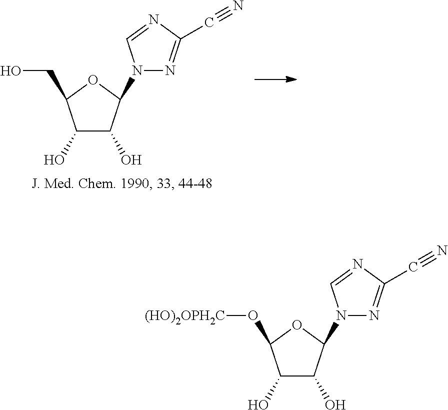

### EXAMPLE 152

### Synthesis of Representative Compounds of Formula 76

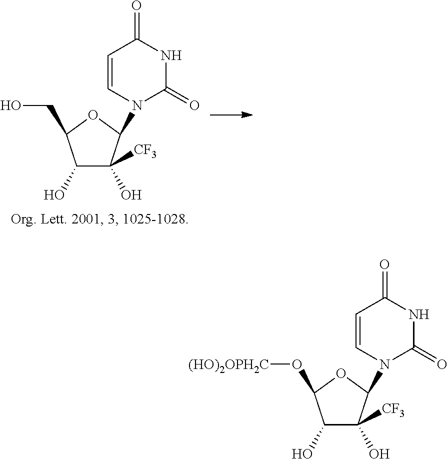

### EXAMPLE 153

### Synthesis of Representative Compounds of Formula 76

### L-Nucleoside Analogs

Many compounds of Formula 76 with a sugar moiety in its L-configuration are either commercially available or can be prepared by procedures described in the published literature. Many of the compounds illustrated in Examples 71-153 can be prepared from one of the precursors described in Example 70 (see Preparation And Availability Of Starting Materials). The enantiomers of other nucleoside analogs (the L-nucleosides) can be prepared from enantiomers of the precursors of 70.3.1, 70.3.2, and 70.3.3. This Example describes a preparation of the enantiomers of 70.3.1, 70.3.2, and 70.3.3.

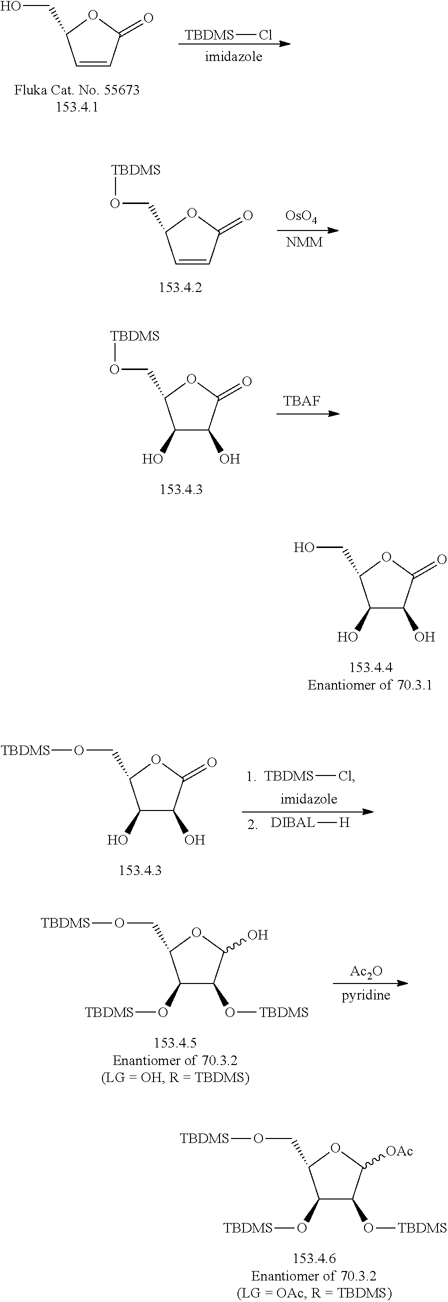

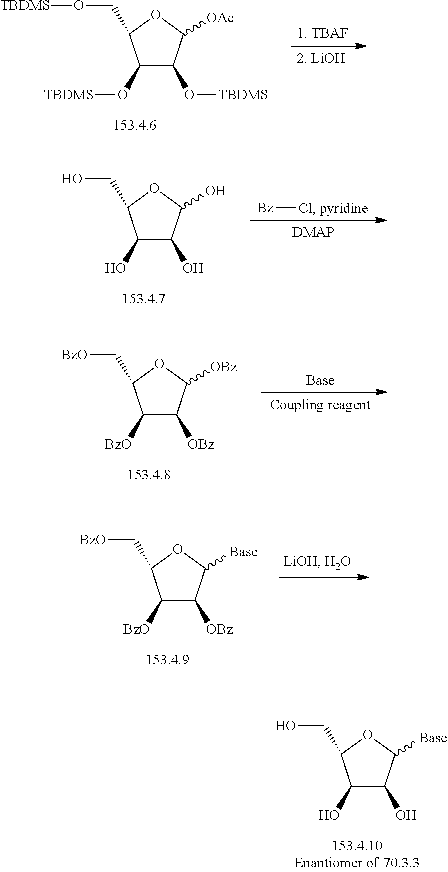

The commercially available starting material 153.4.1 can be converted to 153.4.4, which is the enantiomer of 70.3.1, using the sequence of reactions outlined above. The osmium tetroxide catalyzed dihydroxylation introduces the diol selectively to the face opposite of the tert-butyldimethylsilyl (TBDMS) ether of the hydroxymethyl group.

The diol in intermediate 153.4.3 can be protected as its TBDMS ether. Diisobutylaluminum hydride reduction of the lactone at low temperature produces 153.4.5, which can be converted to 153.4.6 by acetylation.

Deprotection of 153.4.6 produces L-ribose (153.4.7). Acylation converts all hydroxyl groups in 153.4.7 to the corresponding benzoyl esters. Standard coupling reactions with a variety of nucleobases produces 153.4.10, which is the enantiomer of 70.3.3.

From 70.3.1, 70.3.2, and 70.3.3, L-nucleosides can be prepared using known procedures. Many compounds in Examples 70-153 have their corresponding L-analog starting materials described in the literature. These L-nucleosides can then be used in the same reaction sequences to produce the phosphonate analogs of the L-nucleosides.

### EXAMPLE 154

### Synthesis of Representative Compounds of Formulae 84 and 85

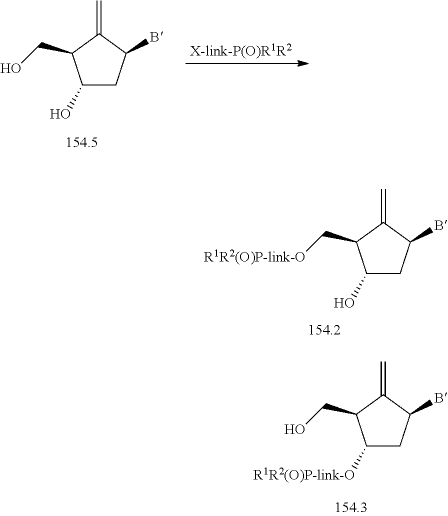

Representative compounds of the invention can be prepared as illustrated above. Direct alkylation of entecavir derivative 154.5 with a phosphonate attached to a leaving group can be performed in the presence of a suitable organic or inorganic base to obtain analogs of the types 154.2 and 154.3. Compound 154.5 is prepared from protected or deprotected intermediates described in U.S. Pat. No. 5,206,244 and U.S. Pat. No. 5,340,816. After reaction, a mixture of compounds 154.2 and 154.3 is furnished, which are separated by the appropriate chromatographic method.

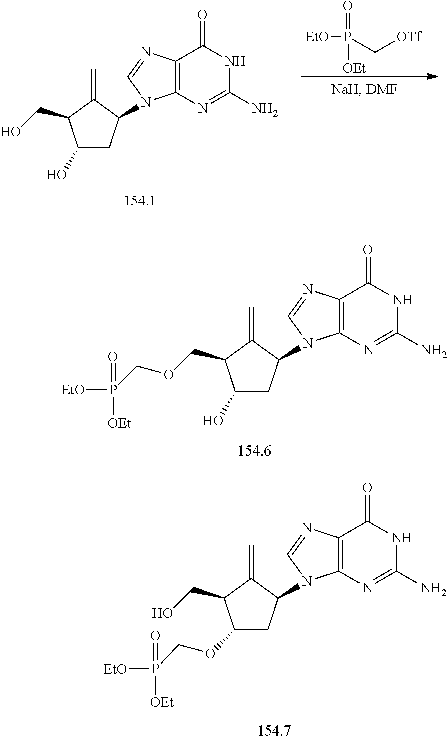

For instance, entecavir (154.1) is treated with sodium hydroxide and reacted with diethyl phosphomethyltriflate to afford a mixture of 154.6 and 154.7 as illustrated above. Silica gel chromatography is employed to give pure samples of the separated products.

### EXAMPLE 155

### Synthesis of Representative Compounds of Formulae 84 and 85

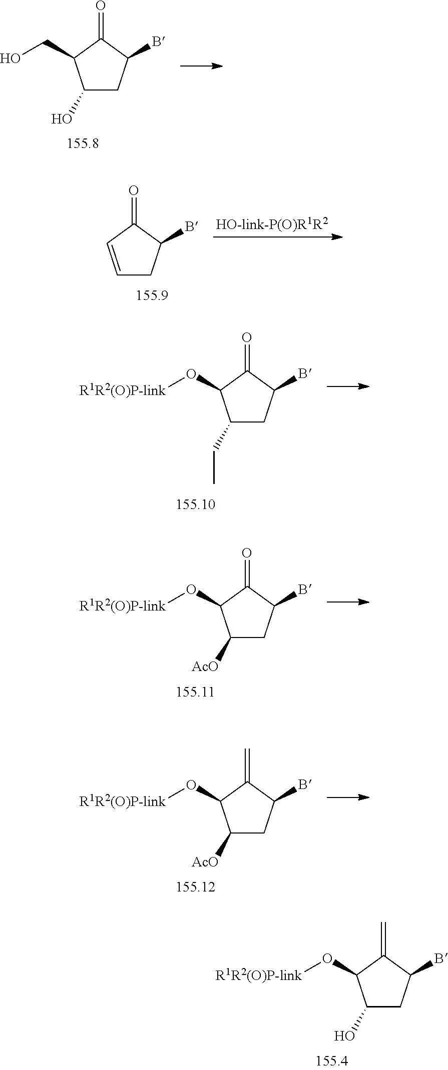

Representative compounds of the invention can be prepared as illustrated above. Compounds having the structure 155.4 are prepared from intermediate 155.8, which is derived from deprotected intermediates described in U.S. Pat. Nos. 5,206,244 and 5,340,816. Diol 155.8 is converted to glycal 155.9 through published procedures. Upon treatment with IBr in the presence of the appropriate phosphonate alcohol, glycal 155.9 is converted to iodide 155.10. Nysted methylenation provides alkene 155.12, whose hydroxy stereocenter is then inverted to give the final compound 155.4.

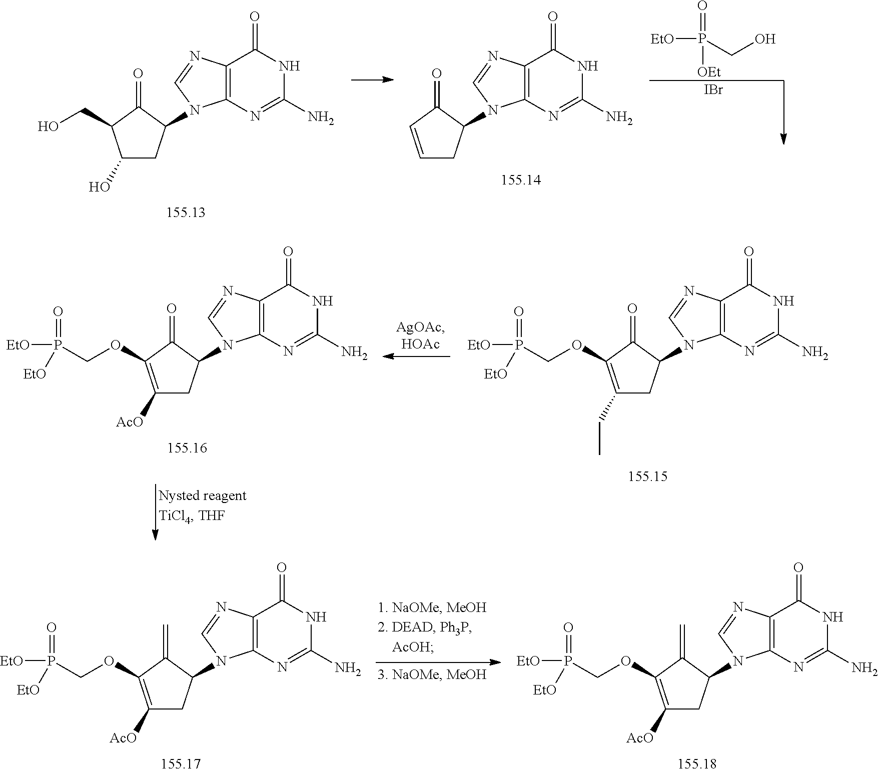

For instance, intermediate 155.13 is converted to glycal 155.14 (see J. Am. Chem. Soc. 1972, 94, 3213) and then treated with IBr and diethyl phosphomethanol to furnish iodide 155.15 (see J. Org. Chem. 1991, 56, 2642). Nucleophilic substitution of the iodide using AgOAc affords acetate 155.16. After methylenation using the procedure of Nysted (U.S. Pat. No. 3,865,848; Aldrichim. Acta 1993, 26, 14), the acetate group is removed using sodium methoxide in methanol. The resulting alcohol is inverted by the Mitsunobo protocol, and a second acetate deprotection produces the desired compound 155.18.

### EXAMPLE 156

By way of example and not limitation, embodiments of the invention are named below in tabular format (Table 100). These embodiments are of the general formula “MBF”:

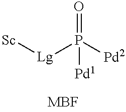

Each embodiment of MBF is depicted as a substituted nucleus (Sc). Sc is described in formula 1-71 herein, wherein A⁰ is the point of covalent attachment of Sc to Lg, as well as in Tables 1.1 to 1.5 below. For those embodiments described in Table 100, Sc is a nucleus designated by a number and each substituent is designated in order by letter or number. Tables 1.1 to 1.5 are a schedule of nuclei used in forming the embodiments of Table 100. Each nucleus (Sc) is given a number designation from Tables 1.1 to 1.5, and this designation appears first in each embodiment name. Similarly, Tables 10.1 to 10.19 and 20.1 to 20.36 list the selected linking groups (Lg) and prodrug (Pd¹ and Pd²) substituents, again by letter or number designation, respectively. Accordingly, a compound of the formula MBF includes compounds having Sc groups based on formula 1-71 herein as well as compounds according to Table 100 below. In all cases, compounds of the formula MBF have groups Lg, Pd¹ and Pd² setforth in the Tables below.

Accordingly, each named embodiment of Table 100 is depicted by a number designating the nucleus from Table 1.1-1.5, followed by a letter designating the linking group (Lg) from Table 10.1-10.19, and two numbers designating the two prodrug groups (Pd¹ and Pd²) from Table 20.1-20.36. In graphical tabular form, each embodiment of Table 100 appears as a name having the syntax:

Sc.Lg.Pd¹.Pd²

Each Sc group is shown having a tilda (“˜”). The tilda is the point of covalent attachment of Sc to Lg. Q¹ and Q² of the linking groups (Lg), it should be understood, do not represent groups or atoms but are simply connectivity designations. Q¹ is the site of the covalent bond to the nucleus (Sc) and Q² is the site of the covalent bond to the phosphorous atom of formula MBF. Each prodrug group (Pd¹ and Pd²) are covalently bonded to the phosphorous atom of MBF at the tilda symbol (“˜”). Some embodiments of Tables 10.1-10.19 and 20.1-20.36 may be designated as a combination of letters and numbers (Table 10.1-10.19) or number and letter (Table 20.1-20.36). For example⁻there are Table 10 entries for BJ1 and BJ2. In any event, entries of Table 10.1-10.19 always begin with a letter and those of Table 20.1-20.36 always begin with a number. When a nucleus (Sc) is shown enclosed within square brackets (“[ ]”) and a covalent bond extends outside the brackets, the point of covalent attachment of Sc to Lg may be at any substitutable site on SC. Selection of the point of attachment is described herein. By way of example and not limitation, the point of attachment is selected from those depicted in the schemes and examples.

Lengthy table referenced here
US20110071101A1-20110324-T00001
Please refer to the end of the specification for access instructions.

Lengthy table referenced here
US20110071101A1-20110324-T00002
Please refer to the end of the specification for access instructions.

Lengthy table referenced here
US20110071101A1-20110324-T00003
Please refer to the end of the specification for access instructions.

Lengthy table referenced here
US20110071101A1-20110324-T00004
Please refer to the end of the specification for access instructions.

Lengthy table referenced here
US20110071101A1-20110324-T00005
Please refer to the end of the specification for access instructions.

Lengthy table referenced here
US20110071101A1-20110324-T00006
Please refer to the end of the specification for access instructions.

Lengthy table referenced here
US20110071101A1-20110324-T00007
Please refer to the end of the specification for access instructions.

Lengthy table referenced here
US20110071101A1-20110324-T00008
Please refer to the end of the specification for access instructions.

Lengthy table referenced here
US20110071101A1-20110324-T00009
Please refer to the end of the specification for access instructions.

Lengthy table referenced here
US20110071101A1-20110324-T00010
Please refer to the end of the specification for access instructions.

Lengthy table referenced here
US20110071101A1-20110324-T00011
Please refer to the end of the specification for access instructions.

Lengthy table referenced here
US20110071101A1-20110324-T00012
Please refer to the end of the specification for access instructions.

Lengthy table referenced here
US20110071101A1-20110324-T00013
Please refer to the end of the specification for access instructions.

Lengthy table referenced here
US20110071101A1-20110324-T00014
Please refer to the end of the specification for access instructions.

Lengthy table referenced here
US20110071101A1-20110324-T00015
Please refer to the end of the specification for access instructions.

Lengthy table referenced here
US20110071101A1-20110324-T00016
Please refer to the end of the specification for access instructions.

Lengthy table referenced here
US20110071101A1-20110324-T00017
Please refer to the end of the specification for access instructions.

Lengthy table referenced here
US20110071101A1-20110324-T00018
Please refer to the end of the specification for access instructions.

Lengthy table referenced here
US20110071101A1-20110324-T00019
Please refer to the end of the specification for access instructions.

Lengthy table referenced here
US20110071101A1-20110324-T00020
Please refer to the end of the specification for access instructions.

Lengthy table referenced here
US20110071101A1-20110324-T00021
Please refer to the end of the specification for access instructions.

Lengthy table referenced here
US20110071101A1-20110324-T00022
Please refer to the end of the specification for access instructions.

Lengthy table referenced here
US20110071101A1-20110324-T00023
Please refer to the end of the specification for access instructions.

Lengthy table referenced here
US20110071101A1-20110324-T00024
Please refer to the end of the specification for access instructions.

Lengthy table referenced here
US20110071101A1-20110324-T00025
Please refer to the end of the specification for access instructions.

Lengthy table referenced here
US20110071101A1-20110324-T00026
Please refer to the end of the specification for access instructions.

Lengthy table referenced here
US20110071101A1-20110324-T00027
Please refer to the end of the specification for access instructions.

Lengthy table referenced here
US20110071101A1-20110324-T00028
Please refer to the end of the specification for access instructions.

Lengthy table referenced here
US20110071101A1-20110324-T00029
Please refer to the end of the specification for access instructions.

Lengthy table referenced here
US20110071101A1-20110324-T00030
Please refer to the end of the specification for access instructions.

Lengthy table referenced here
US20110071101A1-20110324-T00031
Please refer to the end of the specification for access instructions.

Lengthy table referenced here
US20110071101A1-20110324-T00032
Please refer to the end of the specification for access instructions.

Lengthy table referenced here
US20110071101A1-20110324-T00033
Please refer to the end of the specification for access instructions.

Lengthy table referenced here
US20110071101A1-20110324-T00034
Please refer to the end of the specification for access instructions.

Lengthy table referenced here
US20110071101A1-20110324-T00035
Please refer to the end of the specification for access instructions.

Lengthy table referenced here
US20110071101A1-20110324-T00036
Please refer to the end of the specification for access instructions.

Lengthy table referenced here
US20110071101A1-20110324-T00037
Please refer to the end of the specification for access instructions.

Lengthy table referenced here
US20110071101A1-20110324-T00038
Please refer to the end of the specification for access instructions.

Lengthy table referenced here
US20110071101A1-20110324-T00039
Please refer to the end of the specification for access instructions.

Lengthy table referenced here
US20110071101A1-20110324-T00040
Please refer to the end of the specification for access instructions.

Lengthy table referenced here
US20110071101A1-20110324-T00041
Please refer to the end of the specification for access instructions.

Lengthy table referenced here
US20110071101A1-20110324-T00042
Please refer to the end of the specification for access instructions.

Lengthy table referenced here
US20110071101A1-20110324-T00043
Please refer to the end of the specification for access instructions.

Lengthy table referenced here
US20110071101A1-20110324-T00044
Please refer to the end of the specification for access instructions.

Lengthy table referenced here
US20110071101A1-20110324-T00045
Please refer to the end of the specification for access instructions.

Lengthy table referenced here
US20110071101A1-20110324-T00046
Please refer to the end of the specification for access instructions.

Lengthy table referenced here
US20110071101A1-20110324-T00047
Please refer to the end of the specification for access instructions.

Lengthy table referenced here
US20110071101A1-20110324-T00048
Please refer to the end of the specification for access instructions.

Lengthy table referenced here
US20110071101A1-20110324-T00049
Please refer to the end of the specification for access instructions.

Lengthy table referenced here
US20110071101A1-20110324-T00050
Please refer to the end of the specification for access instructions.

Lengthy table referenced here
US20110071101A1-20110324-T00051
Please refer to the end of the specification for access instructions.

Lengthy table referenced here
US20110071101A1-20110324-T00052
Please refer to the end of the specification for access instructions.

Lengthy table referenced here
US20110071101A1-20110324-T00053
Please refer to the end of the specification for access instructions.

Lengthy table referenced here
US20110071101A1-20110324-T00054
Please refer to the end of the specification for access instructions.

Lengthy table referenced here
US20110071101A1-20110324-T00055
Please refer to the end of the specification for access instructions.

Lengthy table referenced here
US20110071101A1-20110324-T00056
Please refer to the end of the specification for access instructions.

Lengthy table referenced here
US20110071101A1-20110324-T00057
Please refer to the end of the specification for access instructions.

Lengthy table referenced here
US20110071101A1-20110324-T00058
Please refer to the end of the specification for access instructions.

Lengthy table referenced here
US20110071101A1-20110324-T00059
Please refer to the end of the specification for access instructions.

Lengthy table referenced here
US20110071101A1-20110324-T00060
Please refer to the end of the specification for access instructions.

Lengthy table referenced here
US20110071101A1-20110324-T00061
Please refer to the end of the specification for access instructions.

Lengthy table referenced here
US20110071101A1-20110324-T00062
Please refer to the end of the specification for access instructions.

All literature and patent citations above are hereby expressly incorporated by reference at the locations of their citation. Specifically cited sections or pages of the above cited works are incorporated by reference with specificity. The invention has been described in detail sufficient to allow one of ordinary skill in the art to make and use the subject matter of the following Claims. It is apparent that certain modifications of the methods and compositions of the following Claims can be made within the scope and spirit of the invention.

In the claims hereinbelow, the subscript and superscripts of a given variable are distinct. For example, R₁ is distinct from R¹.

LENGTHY TABLES
The patent application contains a lengthy table section. A copy of the table is available in electronic form from the USPTO web site (http://seqdata.uspto.gov/?pageRequest=docDetail&DocID=US20110071101A1). An electronic copy of the table will also be available from the USPTO upon request and payment of the fee set forth in 37 CFR 1.19(b)(3).

1. A conjugate comprising a nucleoside linked to one or more phosphonate groups; or a pharmaceutically acceptable salt or solvate thereof.

2. The conjugate of claim 1, or a pharmaceutically acceptable salt or solvate thereof, that is a compound of Formula 240: 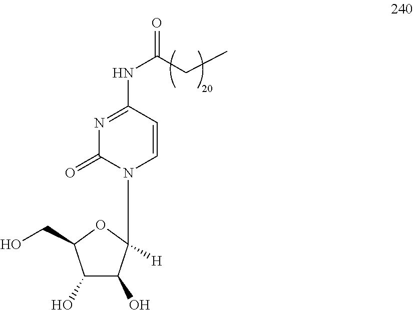
substituted with one or more groups A⁰, wherein:
A⁰ is A¹, A² or W³ with the proviso that the conjugate includes at least one A¹;
A¹ is: 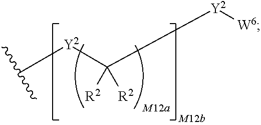
A² is: 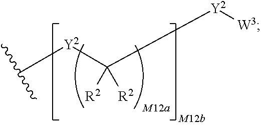
A³ is: 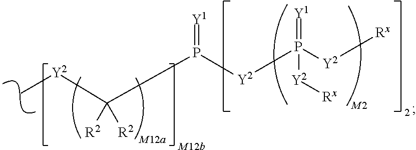
Y¹ is independently O, S, N(R^{x}), N(O)(R^{x}), N(OR^{x}), N(O)(OR^{x}), or N(N(R^{x})(R^{x}));
Y² is independently a bond, O, N(R^{x}), N(O)(R^{x}), N(OR^{x}), N(O)(OR^{x}), N(N(R^{x})(R^{x})), —S(O)_{M2}—, or —S(O)_{M2}—S(O)_{M2}—; and when Y² joins two phosphorous atoms Y² can also be C(R²)(R²);
R^{x} is independently H, R¹, R², W³, a protecting group, or the formula: 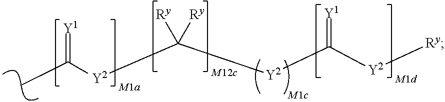
wherein:
R^{y} is independently H, W³, R² or a protecting group;
R¹ is independently H or alkyl of 1 to 18 carbon atoms;
R² is independently H, R¹, R³ or R⁴ wherein each R⁴ is independently substituted with 0 to 3 R³ groups or taken together at a carbon atom, two R² groups form a ring of 3 to 8 carbons and the ring may be substituted with 0 to 3 R³ groups;
R³ is R^{3a}, R^{3b}, R^{3c} or R^{3d}, provided that when R³ is bound to a heteroatom, then R³ is R^{3c} or R^{3d};
R^{3a} is F, Cl, Br, I, —CN, N₃ or —NO₂;
R^{3b} is Y¹;
R^{3c} is —R^{x}, —N(R^{x})(R^{x}), —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)(N(R^{x}(R^{x})), —SC(Y¹)R^{x}, —SC(Y¹)OR^{x}, —SC(Y¹)(N(R^{x})(R^{x})), —N(R^{x})C(Y¹)R^{x}, —N(R^{x})C(Y¹)OR^{x}, or —N(R^{x})C(Y¹)(N(R^{x})(R^{x}));
R^{3d} is —C(Y¹)R^{x}, —C(Y¹)OR^{x} or —C(Y¹)(N(R^{x})(R^{x}));
R⁴ is an alkyl of 1 to 18 carbon atoms, alkenyl of 2 to 18 carbon atoms, or alkynyl of 2 to 18 carbon atoms;
R⁵ is R⁴ wherein each R⁴ is substituted with 0 to 3 R³ groups;
W³ is W⁴ or W⁵;
W⁴ is R⁵, —C(Y¹)R⁵, —C(Y¹)W⁵, —SO_{M2}R⁵, or —SO_{M2}W⁵;
W⁵ is carbocycle or heterocycle wherein W⁵ is independently substituted with 0 to 3 R² groups;
W⁶ is W³ independently substituted with 1, 2, or 3 A³ groups;
M2 is 0, 1 or 2;
M12a is 1, 2, 3, 4, 5, 6, 7, 8, 9, 10, 11 or 12;
M12b is 0, 1, 2, 3, 4, 5, 6, 7, 8, 9, 10, 11 or 12;
M1a, M1c, and M1d are independently 0 or 1; and
M12c is 0, 1, 2, 3, 4, 5, 6, 7, 8, 9, 10, 11 or 12;

3. The conjugate of claim 2, or a pharmaceutically acceptable salt or solvate thereof, which has the formula:)
[DRUG]-(A⁰)ₙₙ
wherein: DRUG is a compound of formula 240; and nn is 1, 2, or 3.

4. The conjugate of claim 2 which has any one of formulae 18 and 19: 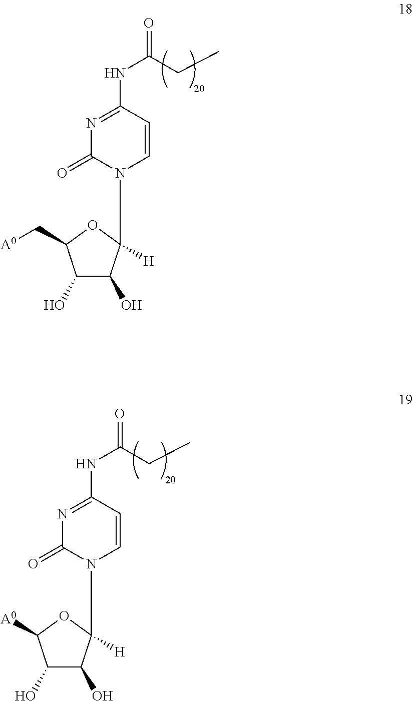
wherein:
A⁰ is A¹.

5-27. (canceled)

28. The conjugate of claim 2 wherein each A³ is of the formula:

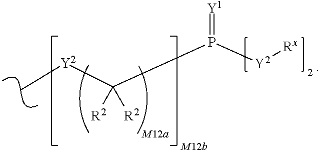

29. The conjugate of claim 2 wherein each A³ is of the formula:

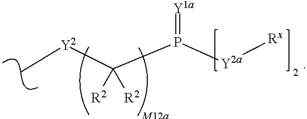

30. The conjugate of claim 2 wherein each A³ is of the formula: 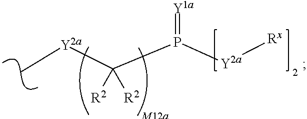
wherein:
Y^{1a} is O or S; and
Y^{2a} is O, N(R^{x}) or S.

31. The conjugate of claim 2 wherein each A³ is of the formula: 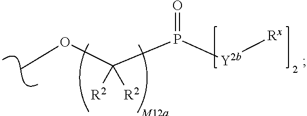
wherein Y^{2b} is O or N(R^{x}).

32. The conjugate of claim 2 wherein each A³ is of the formula: 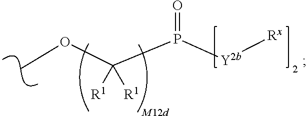
wherein:
Y^{2b} is O or N(R^{x}); and
M12d is 1, 2, 3, 4, 5, 6, 7 or 8.

33-56. (canceled)

57. The conjugate of claim 2 wherein each A³ is of the formula:

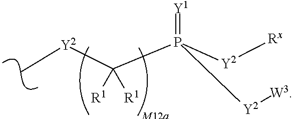

58. The conjugate of claim 2 wherein each A³ is of the formula: 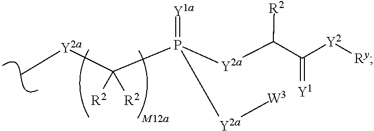
wherein:
Y^{1a} is O or S; and
Y^{2a} is O, N(R²) or S.

59. The conjugate of claim 2 wherein each A³ is of the formula: 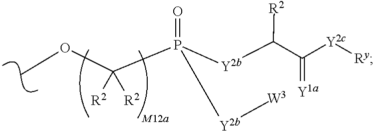
wherein:
Y^{1a} is O or S;
Y^{2b} is O, N(R²); and
Y^{2c} is O, N(R^{y}) or S.

60. The conjugate of claim 2 wherein each A³ is of the formula: 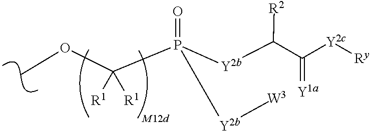
wherein:
Y^{1a} is O or S;
Y^{2b} is O or N(R²);
Y^{2d} is O or N(R^{y}); and
M12d is 1, 2, 3, 4, 5, 6, 7 or 8.

61. The conjugate of claim 2 wherein each A³ is of the formula: 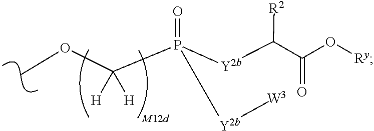
wherein:
Y^{2b} is O or N(R²); and
M12d is 1, 2, 3, 4, 5, 6, 7 or 8.

62. The conjugate of claim 2 wherein each A³ is of the formula: 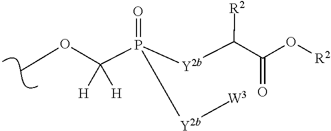
wherein Y^{2b} is O or N(R²).

63. The conjugate of claim 3 wherein A⁰ is of the formula: 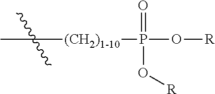
wherein each R is independently alkyl.

64-106. (canceled)

107. A pharmaceutical composition comprising a pharmaceutically acceptable excipient and a conjugate, or a pharmaceutically acceptable salt or solvate thereof, as described in claim 2.

108. A unit dosage form comprising a conjugate as described in claim 2, or a pharmaceutically acceptable salt or solvate thereof; and a pharmaceutically acceptable excipient.

109-114. (canceled)
